SEQUENCE LISTING (99 sequences)

SEQ ID NO: 1 - DNA, 3085 bp, Homo sapiens
ctcacgctctctttccctgccgcctgcctttcttttttcctttttttgtattggcgtcttggggctgttacacacacgcgcgctgtccattgcagcttacataaaggcgggcgcgattatgcaattatattgttagcgatatttcaagagcaatggctcgttttcttaggatttcaacacgaaggcatcatgcatttttgaaaaactagtattgagaataataccttgcaacgtaaagaatgttttttggtatttttacacaatctctactttgaccaaacgagtctggacagttttcttttaatggaaaataggagaaatggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcattttgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacagtgacagaccggaaagtgaagagtcagaactaactttggtctcagcgggagccagtggtgatgtaaaaatgcttccactgcaagtctgtggccccacaacgtgggcttaaagcagaagccccacagcctggcacgaaggccccgtctgggttaggtgactaaaagggcttcggccacaggcaggtcacagaaaggcaggtttcatttcttatcacataagagagatgagaaagcttttattcctttgaatattttttgaaggtttcagatgaggtcaacacaggtagcacagattttgaatctgtgtgcatatttgttactttacttttgctgtttatacttgagaccaacttttcaatgtgattcttctaaagcactggtttcaagaatatggaggctggaaggaaataaacattacggtacagacatggagatgtaaaatgagtttgtattattacaaatattgtcatctttttctagagttatcttctttattattcctagtctttccagtcaacatcgtggatgtagtgattaaatatatctagaactatcatttttacactattgtgaatatttggaattgaacgactgtatattgctaagagggcccaaagaattggaatcctccttaatttaattgctttgaagcatagctacaatttgtttttgcatttttgttttgaaagtttaacaaatgactgtatctaggcatttcattatgctttgaactttagtttgcctgcagtttcttgtgtagatttgaaaattgtataccaatgtgttttctgtagactctaagatacactgcactttgtttagaaaaaaaactgaagatgaaatatatattgtaaagaagggatattaagaatcttagataacttcttgaaaaagatggcttatgtcatcagtaaagtacctttatgttatgaggatataatgtgtgctttattgaattagaaaattagtgaccattattcacaggtggacaaatgttgtcctgttaatttataggagttttttggggatgtggaggtagttgggtagaaaaattattagaacattcacttttgttaacagtatttctcttttattctgttatatagtggatgatatacacagtggcaaaacaaaagtacattgcttaaaatatatagtgaaaaatgtcactatatcttcccatttaacattgtttttgtatattgggtgtagatttctgacatcaaaacttggacccttggaaaacaaaagttttaattaaaaaaaatccttgtgacttacaatttgcacaatatttcttttgttgtactttatatcttgtttacaataaagaattccctttggtaaaaaaaaaaaaaaa

SEQ ID NO: 2 - DNA, 649 bp, Homo sapiens
agaatacactcacaagccactccgctgctcgcctctccgccccgcgtccagctcgcccagctcgcccagcgtccgccgcgcctcgccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagtacacatgaagcagaagggaaagaagtaggcagaaatgagcagttcgctcctccctgataagagttgtccaaagggtcgcttaaggaatctgccccacagcttcccccatagaaggatttcatgagcagatcaggacacttagcaaatgtaaaaataaaatctaactctcatttgacaagcagagaaagaaaagttaaataccagataagcttttgatttttgtattgtttgcatccccttgccctcaataaataaagttcttttttagttcc

SEQ ID NO: 3 - DNA, 760 bp, Homo sapiens
gatggggcttttttccagatcttcgcgccgagagccggggacggccaacctgccggcatgcgataaaggccggggaatgtctagctggggccgcgctgagccgagtgcggacccgctttgtgtgcgcgtttgtttgtctccgctgcggtccgaaccgctgccgcgacccccgaggaggcggggcctccgcgcgcccggctccgcctggtgagggtttcacggcctctgctgccgccgccgccgccctcgtttgttccgttagatcgcgcagccccgaccgctgcacccggatcctagcaagccgggcgaggctgcccgggagccctcgatggccttcatttcacccaagcccgcttcttgctttccccggcgcttcccctcttttcctggttaacagcttatgggcggggagctcggcaaaactcagactaaaaacagaaaaagagaaaagaaaggacaaattcgatacacccgcgtcggtcctccagagtttgtgaaggggtgtaaacatgtcggagtctggggagatgagtgaatttggctacatcatggaattgatagctaaaggcaagatgccggattggagacgaggctaccgctgcagacagggctgtgggaagacgacggaacttgccacagcgacagacttctcccaaacaggaaataaaagtgggaagcattttaaaacctaaaaaaaaaaaaaaaaaaaaaaaaaaaaaaaaaaaaaaaaaaaaaaaaaaaaaaaaaaaaaaaaaaaaaa

SEQ ID NO: 4 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 5 - DNA, 14544 bp, Homo sapiens
cgtactggtccatagcctgttaggaaccaggctgcataacaggaggtgagtggcaggtgagtgaaatttcatctgtagttacagccactcctcatcactcgcattaccaccagagctccactccctgtcagatcagcggcggcattagattctcataggagctcgaaccctattctaaactgttcatgtgagggatctaggttgcaagctccctatgagaatctaatgcctgatgatctgtcacggtctcccatcacccctagatgggaccatctagttgcaggaaaacaagctcagggctcccactgattctacacgatggtgaattgtggaattatttcattatatatattacaatgtaataataatagaaataaagcacacaataaatgtaatgtgcttgaatcatcccgaaaccatcccaccctggtctgtgaaaaaattgtcttccatgaaaccagtccctggtgccaaaaacgttgaggaccactgctccacagaatctatcggtcactcttcctcccctcacccccttgccctaaaagcacaccctgcaaacctgccatgaattgacactctgtttctatcccttttccccttgtgtctgtgtctggaggaagaggataaaggacaagctgccccaagtcctagcgggcagctcgaggaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtgggggagtcagccgtgtatcatcggaggcggccgggcacatggcagggatgagggaaagaccaagagtcctctgttgggcccaagtcctagacagacaaaacctagacaatcacgtggctggctgcatgccctgtggctgttgggctgggcccaggaggagggaggggcgctctttcctggaggtggtccagagcaccgggtggacagccctgggggaaaacttccacgttttgatggaggttatctttgataactccacagtgacctggttcgccaaaggaaaagcaggcaacgtgagctgttttttttttctccaagctgaacactaggggtcctaggctttttgggtcacccggcatggcagacagtcaacctggcaggacatccgggagagacagacacaggcagagggcagaaaggtcaagggaggttctcaggccaaggctattggggtttgctcaattgttcctgaatgctcttacacacgtacacacacagagcagcacacacacacacacacacacatgcctcagcaagtcccagagagggaggtgtcgagggggacccgctggctgttcagacggactcccagagccagtgagtgggtggggctggaacatgagttcatctatttcctgcccacatctggtataaaaggaggcagtggcccacagaggagcacagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggtaggagaaaagcaaactccctccaacctcttacttcgggcttaaggcagagaactcgcctccccagaatctcctccctccatgatcccccgctattcctctattttcttttcctcggacctgcagccttgggtcgaccctgccctaggggtgactgcaggagagcagggaggatggtcaggcgtcaccaacaaccccatcacccagtaacaagaaccttgactctctcagtccctctgcatcaagacacttacccatttcccacctcatgcctgctaacttgaatgaaacaatcgctgggaaagcattaagagaattaaggctgggcactgtggctcatgcctgtaatcccagcactttgtgaggctgaggcaggcagataacttgagcccaggagtttgagaccagcctgggcaacatggcaaaaccctgctctcccaaaaaaatacaaaaattagctgggcgtgctggtgtgcctgtattcccagctacttgggaggctgaggtgggaggattgcttcagctggggaggcggaggctgcagggagccaagactgagccattgcacccagcctgggtgacagagcaagaccctgtctctaaaaatgaatgaaaggaaggaagaaagagagagaaagagagagaggaaagaaggaaggaagtaaagaagaaagaaagaaagaaagaggaaagaggaagaaagaaagaaaagaaagaaaagaaaagaaagcaaatttaaagcttatgcaaatcaaagatgttgtgataattgataattgagtctgggctaaattccccctgggctgcaaaggcagagagtggtaatgacttctcacctgcttttcttctaaggcttttttacgggacacagagggaagggagatggactggattccaagattcccacagggcaagatgggcgaagactccctgccactgcccggggataagtcagtctgagtgagacggagtgggatgggcttagaacctgaacatgtcatggtctcttcctgcaccttgccctagtgttcacttaccacctgcttgcaggaaacaagaagagcagggcccacagctggccagctcccctcccctcccgcctgtcttccagaacgattccttcaccagccctctttccattgctctaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatggtgagccacgggacaccaggggaggtgggtggcatgcagaacagacctaccagaagccaaggaaaggctggctctggcttagccgagccaagccccatacagctgtgctgcaggggccaccccatcttcttcccactacactccaagtcactggacccttgaatctccaagggtgtctgaccagtagatttaccgcttattcaccaccgtgtgatcttaacctcgttaagtttgcccatctacaaaatgaggattatttgctgtcctaaagaattcatgagccgggcgcggtggctcaaacgcctgtaatcccagcactttgggaggccaaggcgggcggatcatgaggtcaggagatcaagaccatcctggctaacacagtgaaactccatctctactaaaaatacaaaaaaaattagccaggcgtggtggcaggcgcctgtagtcccagctactcgggaggctgaggcaggagaatggcatgaacccaggaggcagagcttgcagtgagctgagatcgtgccactgcactccagcctgggcgacagagagagactccgtctcaaaaaaaaaaattaaaagaattcatggaattacacttgtgaaatacttagcatagccatcactataggaaaaaaatctaaggccaggcacagtgcctcatgcctgtaatctcagcactttcggagtttgaggcaggaggatcacccaaggctaggagttcaaggccagcctgggcaatacggtgaaaccccgtctctaataaaaatataaaaattagtctgatgaggtggtgcacctgtaatcccagctactcaggaagctgagacacaagaatcactttaacccgggaggtggaggtggcagtgagctgagatcacaccattgcactccagcctgggtgacagagtgagacctgtcaaaaaaaagaaaagaaagagagagagagagagaagagagagaaagaaagaagaagaaagaaagaaagagagagagagaaagaaagaaagaaacaaagaaacaaagaaagaaaggaaaagaaaaaaaaaactaaggccaggcaaggtggcttatgactgtaatttcagcactttggaagattgaggcaggaggatcacttgaggccagaagttcgagacaagactgagcaacagggagacccctgcctctacaaaaaaatttacaaattagccagatgtggtgacacatacctgtagtcccaactactcaggaggctgaggtgggaggatggcttgagcccaggagctggaggctgcagtgagctatgattgtaccactgcacttcagcctgggcaacaaagggaagccctgtctgaaaaaaaaaaaaaaagaaaaagaagaagaaagaaaatatttagggttcatccaggaggcagaggttgcagtaagctgacatcgcgccattgcactccagcctgggagacaagagcaaaactccaactcaaaaaaaaaaaaaaaaaaaaaacaggaagaaaatatttagggttcataacttaagaacagagaaaaatattctagcccaaagaaagggttgggatctgagacttttgaagaaaggaaggagatacagaaaagagatttcatcctggaatgaaatctccctccagagagccctgggaaagcacggtagcccccatccatcagagtggagccccttgtgggggaagtgggctcggctgggaaccctcaattcagcataagcctcacatgtcctctcctctctgtcccggtgcagacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtgagcaggcagggaaaggaaacccatttcctgggcctcaagagaaagggaatttggaaataaatccacatatcccagttgggtgcagtagttcacacctgtaatcccagcccaacactttgggaggtctaggcgagaggaaggcttgaggcctggagtttgagaccagcctggccaacataacaagacctcatctcttcaaaaaatttaaaaaccagccgggcatggtggtgcacacctgtagtcccagctacttgggaggctgaggtgggaggatcacttgagtccagcagttcaaggctgcagtgagctatgtttgcaccaccacactccagcctgagtcacagaacaagacctcatctctaaaaaacaaacaaaaaccaaatccacatatcctaaaaaatgctccttttcagcattctcttctctatggacaaagggctggatgctttaagaaccaaatcttaggctgggcacggtggctcacgcctctaatcctagcactttgagaggccaaggcgggcagattgcctgagcacaggagttcgagaccagcctggccaacatggtgaaaccctgtctctgtcaaaaatacaaaaaattagccaggtgtgttggcgcgtgcctataatcccagctgctcgggaggatgaggttcaaagaatcacttgaacccgggaggcagaggctgcagtgagctgagatcatgccactgcactccagcctgggtgacagagcaagactttgtctccaaaaaaaggaactagacgggttcatttaaacccctgactgcagccctttgacatacatccaattgaggactggggactccgggaaacatctaaaaggcttaaaaactttgtctaacttcagccgggcatggtggctcacacctgtaatcccagcactttgggaggctgaggcaggtggatcacaaggtcaggagtttgagacgagcctgaccaacatggtgaaaccccgtctctactaaaaatacaaaaattagccaggcatggtggcaggcgcctgtaatcccagctattcgggaggctgaggcaggagaattgcttgaaccccggagacagaggttgcagcgagccgagatcgcgccactgcactccagcctggcaatagagtgagactccatctcaaaacaacaacaacaacaacaacaacaacaaaatcgtctaacttcctgatcttcctgatcattgattttcccataggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatagtaagtccaagagccccttccccacagcccacagcaactgcatctcattcctggggtctcccaaggaatacccaaaatgtcaccctctgagggaggaagaccacagggaatgctcccctttaagggaggagagaccctagaatatactccagctttgacaaagatttcccaagcaggagacatcaggataatgggaacagaagacaggaggtttatcccatgaaggatgaagaagctgaaatccagagattccctcagggccacatttgtccacctgactccagggtctcatcttcgtgtgttgctagtgtgattacctggggatgagaaatcctgctgggggagttgaggttaagaggatgaggactccaggtgctgtggctcacgcctgtaatcccagcactttgggaggccaaggcaggtggatcaggagtttgaggtcaggagtttgagaccagcctggccaacatggtgaaaccctgtctctactaaaaatgcaaaaattagccaggtgtggtggcaggcgcctgtaatcccagctactcgggaggctgaggcaggagaatcacttgagcccgggaggtggaggttgcagtgagccgaacgaaattgagccacttcaccccagcctgggcaaaagagtgaaattccattcaaaaaaaaaaaaaaaaaaaaaaggatgaggactgggatgaactggtggctgggtgtggggaaaatggaagtgaaggaaggccaaaagagacagagaaggcctggcgcggcgactcacgcctataatcccagcactttgggaggctgagaagggggattgcttgaggccagaagttgaataccagtctgggcagcatagcaagaccctgcctctacaaaaaaaaaattttttttaattagccaggcttggtgacatgcatctgtagtctactcaagaagctgaggtgaggccaggcacggtggctcacgcctgtattcccagcactttgggaggtcaaggcgggtggatgacctgaggtcaggagttcaagaccagcctggccaacatggtgaaaccccatctgtataaaaatacaaaaattagctgggcatgatagcaggtgcctgtaattccagctactcaggaggctgaggtgggagaatctattgaacccgggagggggaggttgcagtgagccgagatcatgccattgcactccagcctgggcgacagagtgagactccttctcaaaacaaacaaacaaacaaacaaacaaaatacagaagctgaggcgggaggaacatttgaaccggattcggaggctgcagtgagctatgattgcaccactgcgctccagtctgtgtgacagtgagaccctgtctcttacacacacacacacacacacacacacacatgcacacacacagagagagagaaattagaagatactgaattggcagaagagaagggaaatagaaattaaaatactgaataggggagcagtgaacaggggatacccaaaagccaagagcgagagagagcctggcttccagaaatagtggagaagccaggagaactaggtgaaaacccagtgctgggttgccatcagcgagagctggagccatttccaacgaaccatcttgtcgtcttcacagctgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagtaagccaccccgctatctccccgacctaccaacccctctctcctggctccctaaagtcaccgcccccaggttgaatttcccagatctgtgatgcttgcaggacatgcatgtgtgggaggctgatgggaaactgtggcctgggtttgattatgagtcttgcaatcatccctccccctgtttctgctggagggcaggggacagctcttcctgaccacacccccacattgactatccccagaatacccagcaaaagcccccaaaaggagagtcagagaaatgagggaggtgggggcccaatcagtccacatctacttagggtcgccccatcagcacttccatccccaaccctttcaagtcaacatccaaacaaaagaaatcacttccaaggacggagcagctcaaagcgcagcttctagctggggttccaagaaagcagatttttcgaaatccttctgcagaaggaagcaaagagattttttgaaatctttctgcagaaggagaaggctggagctggggaactccagaattatagggaagcctcccaccacgctcatcccaaatttccggatgctataatgccaggcttggggaaagaggagaatttagttggttagctggtgcgtgctctcacttgcatcctctctcttcctcttttttttttttctcctctctctctggctcataaaaatggaggtaattagttgtgccctggtgagaagcagagagtgcacaaaggccccctgcttgagtcctcttcagggttagctctcagaaacacaatctgcagaacagatttttgttccaacatccttgcaggagaatttgcccttagcttcccccaccccagccaggctgaataaaattatgctgaaactactgtcttatttgaggaaagtaattagtcataggtgggagggggtggggagattgcagaagaatgttcatgaatattaggattttcagctctaaggggggactttgtaaacagctttagaagaagaaccaggccggctgggtgtggtggctcatgcctgtaatctcagcatttggggaggccaaggcgggcggatcacttgaggtcaggagtttgagaccagcctggccaacatggtgaaaccctgtctctattaaaaatacaaaaattagccagccgtggtagcgagcgcctatgatcccagctactccggaggctgaggccagagaatcacatgaacctgggaggtggaggctgcagtgagccgagatcacgccactgcactccagcctgggggacagagcaagaatctgtttcaaaaaaaaaaaaagaaaaataggaaggaaggaaggaaaggaaaggaaagaagagagagagaaagaaagagagagagagagagagaaagaaagaaagaaagaaagaaagaaagaaagaaagaaagaaagaaagaaagaaaaagaaaggaaagaaagaacgaacgaaccaggcctccctctccaaccttcacctccgtccctattctggccacttgattcgggggacacctggtaggggatggggaaaggtgggagctgccagccagaggggaccccggcttgagcagcctcttgctgctatctgcaggttctccctggagactgaagtcgacctcaggaagcccctagagaacctgggaatgaccgacatgttcagacagtttcaggctgacttcacgagtctttcaggtaagaagactttcctttgcattttctcaccccagtggactgcgggggcccctaagaggaaaaaggaacctctccttgagagcggcagctgatctaatcctgtatccacatctgtttcagaccaagagcctctccacgtcgcgcaggcgctgcagaaagtgaagatcgaggtgaacgagagtggcacggtggcctcctcatccacaggtgagtctggctcaggtgaggctccacgggtgtcgcctccatcgcccttcaggataactggtccccagacccggaaaggaccccgcagccctctcggcacagagcagctctgtctgtgctcagccatcacccactccccacctgtttctcagcctggaaaacgggcttgggaccatggaaccctgtttcctcgcctgatggctcctaagttccctgactgtgaaaaggcctcctaaagaaaaacccaagttgttcccacagtgggaagtaaacttaagaaacatgcttatcaggctgggcatggtggctcccacctgtaatcccagcgctttgggggaccaaggcaggtggatcacttgaggtcaggaattcgagaccagcctgggcaacatggcaaaaccctatctctactaaaaatacaaaaattaggcaggcgtggtggcatgtgcctgtagtcccagctacttgggaggctgaggcaggagaatcacttgaatccaggaggcagaggttgcagtgagccgagatcacgctgctgcactccagcctgggcaatagagcatgactctgaagaaaagaaagaaagaaagagagagagagagaaaagaaagaaagaaagaaagaaagaaagaaagaaagaaagaaagaaagaaagaaagaaagagaaagaaagagaagaaaagaaaagaaagagcttatcaataagcccttaaaggatttagataaatgtgtgtaagggaagagctgatccattgctaccaagctcctggaggaaaccaggtctcagaggatgtccctaaacttttaaggttcatattcaggaaaacaaacaacttccagctgggcttagtggctcacacctgtaatcccagcactttgggaggccgaggcaggaggatcgcttgagcccaggaatttgagaccagcctgggcaatataatgagactgtgctctacaaaaattagaaaaaaattagccaggcatggtggcatgcacctgtagccccagttacttgggagactgaggtgggaggatcacttgagcccatgagttcaaggctgcagtgagccatgaaggtgccactgcactcccgcctgggcgacagagggagaccctgtctctaagaaaaacggcgggggtgggggtggtgccagtgccagcatccctctgttctaagacattgtcccttctcttgcagctgtcatagtctcagcccgcatggcccccgaggagatcatcatggacagacccttcctctttgtggtccggcacaaccccacaggtgagcctggaacccatcacgttccacatcctcccacccattctttctctcaggaactagtcccgacagatgcagacatccctctatccctgagagggctctgggcagggaaccataaccctaccctgcttcctgtcccaagaggaggctaccttctatcacccacagacagtgccgggtccccgctctgtgactcaggcagctgcgactccagacagctcactcatctgcctagatctcagtccttccacccacatccagcctgatgagctgtcccactccttctgcttctcaacccccatggttcttccaccctcaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgagggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaatacgaagaatatgtcaggacagtcactgccttcaccttctccatttcacaccggtggtacaagaaatcagaagcctaggccaggtgtggtggttcatgcctgtaatcccagcactttgggaagccgaggtgggtggatcacctaaggtcaggagtttgagaccagcctggacaacatggtgaaaccccgtctctactaaaaatacaaaaattagccgggcgtggtggctggcgcctgtaatcccagctactcgggaggctgaggcaggagaatcacttgaagccaggaggcagaggttgcagtgagctgagattgcaccactgaactccaggctgggtggcagagcgagactccctctcaaaaaacaacaactacaaagacaacaacaaacccagaatcaaaatcctgttggtccatagacctcatgggtggaagagaccttcctacatccaggttggcccaacatgggggagtccatgaaatggtcacctcagctctgccacaagccccaaggataagttgattctgcccctgggaatcatcctcaaaaaggaaaaaaatgttcccctgccataaactttccacttatgcagatgggcctgctcgtaagtcactgtcactgtgggttcccaactctgttcatgacacttccttccagcaccaaatgcttcccacccctctactcccactccccattcttcaaacccagctcaagttccagttcctccacctaggacttcccatggatccagccaatatcactctcaggtccggcgcagtggcccacgcctgtaatctcagcactttgggaggccggggcaggaagattgcttgaggccaggagtttcagaccagcctggacaacatagtgagactc

SEQ ID NO: 6 - DNA, 7041 bp, Homo sapiens
ggtggtttgcagatcactgaggctggacaacgttcatggctctcgggtagaacctagtgaaacggccagaatgaattctatggacaggcacatccagcagaccaatgaccgactgcagtgcatcaagcagcacttacagaatcctgccaacttccacaatgccgccacggagctgctggactggtgcggagacccacgggccttccagcggcccttcgagcagagcctgatgggctgtttgacggtggtcagtcgggtggcagcccagcaaggctttgacctggacctcggctacagactgctggctgtgtgtgctgcaaaccgagacaagttcaccccgaagtctgccgccttgttgtcctcctggtgcgaagagctcggccgcctgctgctgctccgacatcagaagagccgccagagcgatccccctgggaaactccccatgcagccccctctcagctccatgagctccatgaaacccactctgtcgcacagtgatgggtcgttcccctatgactctgtcccttggcagcagaacaccaaccagcctcccggctccctttccgtggtcaccacggtttggggagtaaccaacacatcccagagccaggtccttgggaaccctatggccaatgccaacaaccccatgaatccaggcggcaaccccatggcgtcgggcatgaccaccagcaacccaggcctcaactccccacagtttgcggggcagcagcagcagttctcagccaaggctggccccgctcagccctacatccagcagagcatgtatggccggcccaactaccccggcagcgggggctttggggccagttaccctgggggtcctaacgcccccgcaggcatgggcatccctccgcacaccaggccgcctgctgacttcactcagcccgcggcagccgctgcagcagcggcagtggcagcagcagcagccacagctacagccacagccacggccactgtggcagccctgcaggagacacagaacaaggatataaaccagtatggaccgatgggtcccacccaggcgtataacagccaattcatgaaccagcccgggccgcgggggcctgcctccatggggggcagcatgaaccccgcgagcatggcggctggcatgacgccctcggggatgagcggccctcccatgggcatgaaccagccccggccgcccggcatcagcccctttggcacacacgggcagcggatgccccagcagacctacccgggcccccggccccagtcccttcctattcagaacataaagaggccataccctggagagcccaactatggaaaccagcaatatggaccaaacagccagttccccacccagccaggccagtacccagcccccaaccccccgaggccactcacctcccccaactacccaggacagaggatgcccagccagccgagctccgggcagtacccgccccccacggtcaacatggggcagtattacaagccagaacagtttaatggacaaaataacacgttctcgggaagcagctacagtaactacagccaagggaatgtcaacaggcctcccaggccggttcctgtggcaaattacccccactcacctgttccagggaaccccacaccccccatgacccctgggagcagcatccctccatacctgtcccccagccaagacgtcaaaccacccttcccgcctgacatcaagccaaatatgagcgctctgccaccacccccagccaaccacaatgacgagctgcggctcacattccctgtgcgggatggcgtggtgctggagcccttccgcctggagcacaacctggcggtcagcaaccatgtgttccacctgcggcccacggtccaccagacgctgatgtggaggtctgacctggagctgcagttcaagtgctaccaccacgaggaccggcagatgaacaccaactggcccgcctcggtgcaggtcagcgtgaacgccacgcccctcaccattgagcgcggcgacaacaagacctcccacaagcccctgcacctgaagcacgtgtgccagccgggccgcaacaccatccagatcaccgtcacggcctgctgctgctcccacctcttcgtgctgcagctggtacaccggccctccgtccgctctgtgctgcaaggactcctcaagaagcgcctcctgcccgcagagcactgtatcacgaaaatcaagcggaatttcagcagcgtggctgcctcctcgggcaacacgaccctcaacggggaggatggggtggagcagacggccatcaaggtgtctctgaagtgccccatcacattccggcgcatccagctgcctgctcgaggacacgattgcaagcatgtgcagtgctttgatctggagtcatacctgcagctgaattgcgagagagggacctggaggtgtcctgtgtgcaataaaaccgctctgctggagggcctggaggtggatcagtacatgtggggaatcctgaatgccatccaacactccgagtttgaagaggtcaccatcgatcccacgtgcagctggcggccggtgcccatcaagtcggacttacacatcaaggacgaccctgatggcatcccctccaagcggttcaagaccatgagtcccagccagatgatcatgcccaatgtcatggagatgatcgcagccctgggccccggcccgtccccctatcccctcccgcctcccccagggggcaccaactccaacgactacagcagccaaggcaacaactaccaaggccatggcaactttgacttcccccacgggaaccctggagggacatccatgaatgacttcatgcacgggcccccccagctctcccaccccccggacatgcccaacaacatggccgccctcgagaaacccctcagccaccccatgcaggaaactatgccacacgctggcagctctgaccagccccacccctccatacaacaaggtttgcacgtaccacaccccagcagccagtcagggcctccattacatcacagtggggctcctcctcctcctccttcccagcctccccggcagccgccacaggccgctcccagcagccatccacacagcgacctgacctttaacccctcctcagccttagagggtcaggccggagcgcagggagcgtccgacatgccggagccttcgctggatctccttcccgaactcacaaatcctgacgagctcctgtcttatctggacccccccgacctgccgagcaatagtaacgatgacctcctgtctctatttgagaacaactgagggccacccggtcggggccatccctccacactctgcatcctaccccacctacccaacacacttttccacctgggagcctgtgccctcagaccgccccgcaccagagccacgggctgtggggcggggagccctcccccgctgcagccctctcagaacagaggggtagggagggtgcaccagtgcaccaggaaggctgtgtgggtctggagcccacgtcccacctccacacccttggcttgggcccatgcccagcgcaggcctgaagaccaccctcccgagaggaaccagcccggtaagagggcacacgctgatgcggcttcccggtccctccgcgtgtgccgattccagatgaccttccagtgtccccaaggttcttccatcttctagactgtaaccctgcctccctgcttcctggtccagagcctccctccagtgactgtggagcctgagaaggcccccgggccccagcatgggccccgagccttggaggagcactggcagttggtggcagtgagaccagcccacccaccaccacccaccacagaaaagcacaaacctctgggaaagacaacgtctctcgggggccaggggtcatcggtttgacccctgacctataagccaagataccccataaacacactcagaaagcagagaaaaaggacaagagtctgtgtttgagagggggtctgccattcctgcttggggactggtggggaagagggccaggacatcttctgagccaggacgtccctgaggctccacctccaagctcagacagggcccaggcttggggaacagagagagcaggtgtacacccaaccaaagtgattgtgcccttggttggggggcgcgggcatataacctgtcagaagcaaacaggagcggcaacttctaactttgctccaagccactctctttttaaacagcaacaatttaaagctatgaagtcacctggagaaaaggaacgttgctcttggacagcaagcaaaccatttctctccgtctgttctgtttttctcctagtccctctcctgccacctctccaagacttccgtgggacacccacttccctctgtcctagttctctttgtccaatcagatggcaagggcagtgcgtggaaaggccggggaggtgcagaaaccagagcccagggcaatggtgtctgtccagcccctccctctgtccctgtgctccaagctgcccccggctgcagcccaggccatggacatgtgcaccagtatgtacctgcaggcatgggggggaggggggcgtgtttctgggcctgccccagacactgcccttggctgccagcctaccctgcctgcactcctccaccatcacaatctcacccaaactcctgctcactcaagcaaaagcagcctctggccttccctccaccgctttgctccatctggcttaccactctccagggcctcctggggagcctgtcctgtgttcactttgtttcaggctggtctgtgccccgtgagccacatggcctagggtgatgccaggttgtcccgtcactggggtcccatctgtaaattctttgcgcccttcccggctgctgcctggggccctttcctgctctcccgtccgctgtgggtggtccccagctctcctctgtgggttttaccggaaaggtggccccagctgttgacttccagtcactgtcccagacggcacaaggttttctgtaggaaagctgccattgccccggccccttttcttcctttgtcccgttgtcgaggttttttcaaatagcgtgttgttcagtatgcaaatcaattattttaagaatcgcttttgtaaatatctttgtgaatattttagtatcgtctttgataatattcaacattttcatgacctggttatagcctttgctggtgtttttaaaatacctggactcaatgacaaagaccgagtcttctttttttttaaacaaaaacaaaaaaagcaaccagggctatttgtacagttgaaggggtgaacagaatgggcggctgtgctgggagttggaagaccgggcagcccgctatttagagccatccctcagtcagctggcagggacaagccaacgccaggtagcatgtggccacccttgcccagtgtctgtggcctggcaagtggccacgccctgtgtcagaccatctgggaattaagctccagacagacttacagatgccttccttaggagttcttgcttcttgcgttgatactttgccccagaaaggcctgggattcattctggttcttatcagggtgtgtccacactctgctcacaggtggatccacggctttccagtgcggagagtcgagatgctccctgcagcccaggccccgggcacctcctgcaaccatctctgggctcagcacctgaggcgggtttcctgggtcccctctccagcaagcctccaccagcaagctcggcccagagcttcccttccggctggctctgaaccgtgcgtggtgcctacagcctgcagtctggagacaagctcttccggagtgctctgggagccaggccagggtgtgagggaggtgcagaggcatccggggcgggagcaagccccaggttgtgacaggtgcaggtagacaacgcccataaacagagatggtcctgaactctggagagatccttccctgatcctttcggacgactacttggagccataagtaacctcagcaaaaacgaggcctctgcaagccacttttccatgccaagcatccacccggcccacaggcatgtttctgccgccactccgcaagatggacagggagccagcaggcaggcgggaagggccaagtacaggcaatcacccccatcttcttggtttgaagctttatccatgtatcatgttccgtgtagccattttattttttaagaaactgctaatactttctccctaatggaagccctgatcccccagagagctacaggtctgctcccgacgggcctcgggcctgacccgtccacacagggccgtgtcaacagcagcgactcaagggacgtgtgtacatatgtaaatgagaaatagagacgtgtcaacagatgcattcatttctcttggaatgtgtattgtttttattttgcgaaacaaaacaaaacaaaaaaaaaagcttggaactccatcacgtggaaaaactagatcctgttggttatagcatttgtgagttctccacgtctgtctctctcgctcatgtaatatactctgaccctgagtggaaaggggtttttgttctgtttttattttacctacatgtactatttagcttcagtgtactagtcctgccacctgtgtatttttagggtgctatggaaataatgaaaagaaacggggatttcagaagaaaattgtaaccaaattcatactttgtataatttttgatatcatgatcacaggtgattcacacgtacacacataaacacacccaccagtgcagcctgaagtaactcccacagaaaccatcatcgtctttgtacatcgtatgtacaatgcaatcatttcatactttaaactggtcaaaaaactaattgtgatttctagtcttgcaaagctgtatgtagttagatgatgtgacaacctctaatatttatctaataaatatgtattcagatgaaacctgtatattaggtgttcatgtggttattttgtatttaaagatcaaattatttgactattgctagacatttctatactctgttgtaacactgaggtatctcatttgcccatgttaatttttttctaaataaattgacaaaaac

SEQ ID NO: 7 - DNA, 2015 bp, Homo sapiens
tcgctgcgaaggacatttgggctgtgtgtgcgacgcgggtcggaggggcagtcgggggaaccgcgaagaagccgaggagcccggagccccgcgtgacgctcctctctcagtccaaaagcggcttttggttcggcgcagagagacccgggggtctagcttttcctcgaaaagcgccgccctgcccttggccccgagaacagacaaagagcaccgcagggccgatcacgctgggggcgctgaggccggccatggtcatggaagtgggcaccctggacgctggaggcctgcgggcgctgctgggggagcgagcggcgcaatgcctgctgctggactgccgctccttcttcgctttcaacgccggccacatcgccggctctgtcaacgtgcgcttcagcaccatcgtgcggcgccgggccaagggcgccatgggcctggagcacatcgtgcccaacgccgagctccgcggccgcctgctggccggcgcctaccacgccgtggtgttgctggacgagcgcagcgccgccctggacggcgccaagcgcgacggcaccctggccctggcggccggcgcgctctgccgcgaggcgcgcgccgcgcaagtcttcttcctcaaaggaggatacgaagcgttttcggcttcctgcccggagctgtgcagcaaacagtcgacccccatggggctcagccttcccctgagtactagcgtccctgacagcgcggaatctgggtgcagttcctgcagtaccccactctacgatcagggtggcccggtggaaatcctgccctttctgtacctgggcagtgcgtatcacgcttcccgcaaggacatgctggatgccttgggcataactgccttgatcaacgtctcagccaattgtcccaaccattttgagggtcactaccagtacaagagcatccctgtggaggacaaccacaaggcagacatcagctcctggttcaacgaggccattgacttcatagactccatcaagaatgctggaggaagggtgtttgtccactgccaggcaggcatttcccggtcagccaccatctgccttgcttaccttatgaggactaatcgagtcaagctggacgaggcctttgagtttgtgaagcagaggcgaagcatcatctctcccaacttcagcttcatgggccagctgctgcagtttgagtcccaggtgctggctccgcactgttcggcagaggctgggagccccgccatggctgtgctcgaccgaggcacctccaccaccaccgtgttcaacttccccgtctccatccctgtccactccacgaacagtgcgctgagctaccttcagagccccattacgacctctcccagctgctgaaaggccacgggaggtgaggctcttcacatcccattgggactccatgctccttgagaggagaaatgcaataactctgggaggggctcgagagggctggtccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggtttttttttagttgccaacagttgtatgtttgctgattatttatgacctgaaataatatatttcttcttctaagaagacattttgttacataaggatgacttttttatacaatggaataaattatggcatttctattg

SEQ ID NO: 8 - DNA, 4086 bp, Homo sapiens
aatggagccgctgtcagcagaaccttctgccgccgccgccgccgccgccgtccctcctcttttttttcccggcagatctttgttgtgtgggagggcagcagggatggacttgagcttgcggatcccctgctagagcagccgcgctcggagaaggcgccgcagccgcgaggaggagccgccgccgccgcgcccgaggccccgccgcccgcggcctctgtcggcccgcgccccgctcgccccgtcgccccgtcgcccctcgcctccccgcagagtcccctcgcggcagcagatgtgtgtggggtcagcccacggcggggactatggtgaaattcccggcgctcacgcactactggcccctgatccggttcttggtgcccctgggcatcaccaacatagccatcgacttcggggagcaggccttgaaccggggcattgctgctgtcaaggaggatgcagtcgagatgctggccagctacgggctggcgtactccctcatgaagttcttcacgggtcccatgagtgacttcaaaaatgtgggcctggtgtttgtgaacagcaagagagacaggaccaaagccgtcctgtgtatggtggtggcaggggccatcgctgccgtctttcacacactgatagcttatagtgatttaggatactacattatcaataaactgcaccatgtggacgagtcggtggggagcaagacgagaagggccttcctgtacctcgccgcctttcctttcatggacgcaatggcatggacccatgctggcattctcttaaaacacaaatacagtttcctggtgggatgtgcctcaatctcagatgtcatagctcaggttgtttttgtagccattttgcttcacagtcacctggaatgccgggagcccctgctcatcccgatcctctccttgtacatgggcgcacttgtgcgctgcaccaccctgtgcctgggctactacaagaacattcacgacatcatccctgacagaagtggcccggagctggggggagatgcaacaataagaaagatgctgagcttctggtggcctttggctctaattctggccacacagagaatcagtcggcctattgtcaacctctttgtttcccgggaccttggtggcagttctgcagccacagaggcagtggcgattttgacagccacataccctgtgggtcacatgccatacggctggttgacggaaatccgtgctgtgtatcctgctttcgacaagaataaccccagcaacaaactggtgagcacgagcaacacagtcacggcagcccacatcaagaagttcaccttcgtctgcatggctctgtcactcacgctctgtttcgtgatgttttggacacccaacgtgtctgagaaaatcttgatagacatcatcggagtggactttgcctttgcagaactctgtgttgttcctttgcggatcttctccttcttcccagttccagtcacagtgagggcgcatctcaccgggtggctgatgacactgaagaaaaccttcgtccttgcccccagctctgtgctgcggatcatcgtcctcatcgccagcctcgtggtcctaccctacctgggggtgcacggtgcgaccctgggcgtgggctccctcctggcgggctttgtgggagaatccaccatggtcgccatcgctgcgtgctatgtctaccggaagcagaaaaagaagatggagaatgagtcggccacggagggggaagactctgccatgacagacatgcctccgacagaggaggtgacagacatcgtggaaatgagagaggagaatgaataaggcacgggacgccatgggcactgcagggacagtcagtcaggatgacacttcggcatcatctcttccctctcccatcgtattttgttcccttttttttgttttgttttggtaatgaaagaggccttgatttaaaggtttcgtgtcaattctctagcatactgggtatgctcacactgacggggggacctagtgaatggtctttactgttgctatgtaaaaacaaacgaaacaactgacttcatacccctgcctcacgaaaacccaaaagacacagctgcctcacggttgacgttgtgtcctcctcccctggacaatctcctcttggaaccaaaggactgcagctgtgccatcgcgcctcggtcaccctgcacagcaggccacagactctcctgtcccccttcatcgctcttaagaatcaacaggttaaaactcggcttcctttgatttgcttcccagtcacatggccgtacaaagagatggagccccggtggcctcttaaatttcccttccgccacggagttcgaaaccatctactccacacatgcaggaggcgggtggcacgctgcagcccggagtccccgttcacactgaggaacggagacctgtgaccacagcaggctgacagatggacagaatctcccgtagaaaggtttggtttgaaatgccccgggggcagcaaactgacatggttgaatgatagcatttcactctgcgttctcctagatctgagcaagctgtcagttctcacccccaccgtgtatatacatgagctaacttttttaaattgtcacaaaagcgcatctccagattccagaccctgccgcatgacttttcctgaaggcttgcttttccctcgcctttcctgaaggtcgcattagagcgagtcacatggagcatcctaactttgcattttagtttttacagtgaactgaagctttaagtctcatccagcattctaatgccaggttgctgtagggtaacttttgaagtagatatattacctggttctgctatccttagtcataactctgcggtacaggtaattgagaatgtactacggtacttccctcccacaccatacgataaagcaagacattttataacgataccagagtcactatgtggtcctccctgaaataacgcattcgaaatccatgcagtgcagtatatttttctaagttttggaaagcaggttttttcctttaaaaaaattatagacacggttcactaaattgatttagtcagaattcctagactgaaagaacctaaacaaaaaaatattttaaagatataaatatatgctgtatatgttatgtaatttattttaggctataatacatttcctattttcgcattttcaataaaatgtctctaatacaatacggtgattgcttgtgtgctcaacatacctgcagttgaaacgtattgtatcaatgaacattgtaccttattggcagcagttttataaagtccgtcatttgcatttgaatgtaaggctcagtaaatgacagaactatttttcattatgggtaactggggaataaatgggtcactggagtaggaatagaagtgcaagctggaaaggcaaaaatgagaaagaaaaaggcaggccctttgtgtctaccgttttcagtgctgtgtgatcatattgttcctcacagcaaaaaagaatgcaagggcataatgttagctgtgaacatgccagggttgcattcacattcctgggtacccagtgctgatggggtgtgcccacgtggggacatgtccttggcgtgcttcctcagagtggcttttcctccattaatacatatatgagtactgaaaaattaagttgcatagctgctttgcagtggtttcagaggcagatctgagaagattaaaaaaaaatctcaatgtatcagctttttttaaaggacattactagaaaattaaacagtattttttaacatgtgtgactttcatgcttctggggttggagcttaaagatccaaactgagaaagcaggccgggcatggtggctcatgcctgtaatcccaacactttgggaggccaaggagggtggatcacttaaggtcaggagtttgagaccagcctggccaacatggcaaaaccctgtctctactaaaaacataaaaattagctgggggtggtagcacatacctgtaatcccagctactcaggaggctgaggcaggagaatttgcttgatcctgggaggcagaggttgtagtgagccgagatcgcgccatcgcactccagcctgggtgacaagagcaaaactccatctc

SEQ ID NO: 9 - DNA, 1796 bp, Homo sapiens
ggtcagctgagttcgccggcccagggcaggcggggcccgagcctagcggtaacccccgggcagggcggggccgctcgcagactccatatgagattcacctcgcaggtggttccctcattcgagtgctccggcgcacagacccgcgccccgccgtctgcgagcctcccgagagccgtcccttcgtccggccctggagcattgcgtttgtcgccggtgtcgcagtgcgaggatggcgccgcgggtgtagcggctctctgcgcaggccgagtgggcccagagaagcgaggaactccgcagctcgtcgacacgtctcgtctcctgtcccaattcagggcttggtgaggtgactcgcggtcgcgggtgactcgccggcaggacactgcctggaacgcctggagcgcctcccactgcagacgtctgtccgcctccagccgctctcctctgacgggtcctgcctcagttggcggaatggcggccacgggagccaatgcagagaaagctgaaagtcacaatgattgccccgtcagacttttaaatccaaacatagcaaaaatgaaagaagatattctctatcatttcaatctcaccactagcagacacaatttcccagccttgtttggagatgtgaagtttgtgtgtgttggtggaagcccctcccggatgaaagccttcatcaggtgcgttggtgcagagctgggccttgactgcccaggtagagactatcccaacatctgtgcgggaactgaccgctatgccatgtataaagtaggaccggtgctgtctgtcagtcatggtatgggcattccttctatctcaatcatgttgcatgagctcataaagctgctgtactatgcccggtgctccaacgtcactatcatccgcattggcacttctggtgggataggtctggagcccggcactgtggtcataacagagcaggcagtggatacctgcttcaaggcagagtttgagcagattgtcctggggaagcgggtcatccggaaaacggaccttaacaagaagctggtgcaggagctgttgctgtgttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgcaatgtgctcagcgagtaccagcagaggccgcagcggctggtgagctacttcatcaagaagaaactgagcaaggcctgagcgctgccctgcacctccgcagacctgctgtgatgacttgccattaaaagcattgtccaaaatcccctgttgtgtggactttgagcacactttacacaagaatctagaaaatcagatcgcgattaagagacagagaatcttggattaaccgcatgggagatgttcttccttttgaagtttcattggagcattttcaatgatgttagcctgatttggggtttcttcaagaacattctaccaaatttttgtactatttctagggaaatttttcagactttaaaattctaatggtagtcagatttcatgtcactaaacaagaaatctgacaatagtgccaggaaactaatttcctgatacattaaaaaaattccatgcaaaaaaaaaaaaaaaaaaaa

SEQ ID NO: 10 - DNA, 3665 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 11 - DNA, 1912 bp, Homo sapiens
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 12 - DNA, 1588 bp, Homo sapiens
gaattcgcccttcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgag

SEQ ID NO: 13 - DNA, 1122 bp, Homo sapiens
gctcctcgccccgcgcctgcccccaggatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaaactctctataagtgttcagtgttcacatagcctttgtgcaaaaaaaaaaaaaaaaaaaaa

SEQ ID NO: 14 - DNA, 1869 bp, Homo sapiens
gtcacccccagcgggcgcgggccggagcacgggcacccagcatgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtagcctcgagagcaccccttgccggtgaaggatgcggcaggtgctctgtggatgagaggaaccatcgcaggatgacagctcccgggtccccaaacctgttcccctctgctactagccactgagaagtgcactttaagaggtgggagctgggcagacccctctacctcctccaggctgggagacagagtcaggctgttgcgctcccacctcagccccaagttccccaggcccagtggggtggccgggcgggccacgcgggaccgactttccattgattcaggggtctgatgacacaggctgactcatggccgggctgactgcccccctgccttgctccccgaggcctgccggtccttccctctcatgacttgcagggccgttgcccccagacttcctcctttccgtgtttctgaaggggaggtcacagcctgagctggcctcctatgcctcatcatgtcccaaaccagacacctggatgtctgggtgacctcactttaggcagctgtaacagcggcagggtgtcccaggagccctgatccgggggtccagggaatggagctcaggtcccaggccagccccgaagtcgccacgtggcctggggcaggtcactttacctctgtggacctgttttctctttgtgaagctagggagttagaggctgtacaaggcccccactgcctgtcggttgcttggattccctgacgtaaggtggatattaaaaatctgtaaatcaggacaggtggtgcaaatggcgctgggaggtgtacacggaggtctctgtaaaagcagacccacctcccagcgccgggaagcccgtcttgggtcctcgctgctggctgctccccctggtggtggatcctggaattttctcacgcaggagccattgctctcctagagggggtctcagaaactgcgaggccagttccttggagggacatgactaatttatcgatttttatcaatttttatcagttttatatttataagccttatttatgatgtatatttaatgttaatattgtgcaaacttatatttaaaacttgcctggtttctaaaaaaaaaaaaaaaaaa

SEQ ID NO: 15 - DNA, 1456 bp, Homo sapiens
caggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcatgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgcccgccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttacgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaaaaaaaaaaaaaaaaaaaaaaaaaa

SEQ ID NO: 16 - DNA, 3870 bp, Homo sapiens
gagcgccgggaccgcggcggcggggccgcggcgcgcattgcggagggcgcggagcgcaggagctgccgcctgccgggcagatccaagggggcagcacgcttcccgggagcgcccccgcctcctctccggggccgccgcaggctcggtgagcggttttatccctccggccggcaggctgggcgcgcaggggcgcgagcccccgcccggcgcgcagcagcaccatgggcacggtgctgtccctgtctcccagctaccggaaggccacgctgtttgaggatggcgcggccaccgtgggccactatacggccgtacagaacagcaagaacgccaaggacaagaacctgaagcgccactccatcatctccgtgctgccttggaagagaatcgtggccgtgtcggccaagaagaagaactccaagaaggtgcagcccaacagcagctaccagaacaacatcacgcacctcaacaatgagaacctgaagaagtcgctgtcgtgcgccaacctgtccacattcgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtgagcactgtagcctgcgtcatggctcaaggattcaatgcatttttaagaatttattattaaatcagttttgtgtacagtatgtgtctagcaaagccaccaagggcctcacctttcccacagtctctccctggggtttttttcatccctgccaagaactctgggcacttttgaactcacgagccttgcgcaaaacccagaagatgtattcagagccacccaggccactgacctcccactttggggaactcaaaggactgacctgcccctgccgcctgtgcccttgctgggtccagggtaggcaaggctgccggctgcaccctgtatggagctagagaggggcctctggctgcctggcccagggaggaattggggtttctggttggaggcccttttctggctcctgtgtggagttattcactctcccagaggctcctggagccagccaccctaactgagctgccagtggggtcgtgaggcaagatccccgccacccggggacatcttcaatctaggcgaggcgaagctgagcgggtctagtggaaagattgtgtctggtcgtttgaccacacaccgccctgatttgctgttttctttttttagggagaagggcttttctttagtggagaaatggaactcgcccccctacccccttgtctgctgctcccagccacgttggtggtattggccgatgagctggtttgactcattaatttctctcaatttgggtcccagctaaagaggtggggtgaagctggggacagctttcctgggtgagtttttcttttgaataatgtagtgcagtcaccctgtggcaaatgccaggacagctgcaggtgcccattagcaacgcctgccttctgggcaaggtgagggatgccataccttgagaccaagccggtgcccacctaagggctaaggcctcttgctggtgcacatgacatttgtcctgcagagctggggggaaggcgatgtggtgaccccacccccacccgttaatttaaagctgtttctaaacagttgagtttcttctaaagaggaagccttgcccagcaaggaccagtgagacagccggatcttggagacaattacaagacggggagtgagactcctgattgctctggaagcctgctgatttctacaactgatcatttgcagctgctggttttggtttccaccttaccctactggctgtaaaaacacaaatgtgtactttattgattttctttctaattctcccgcattggtggcttgggacttgggagagggagcaagccttcctccatggcccatcactcggctgtggagaacaaagaccaatgtgaagacactacagaggattctgtcttccaggcccagtccactggggagtgctggaatggggacctggggtggggaggcagagggtcactttacataggattaagttcgaggtggctaccgatttcagcacatgcactactgaaatttacacaaaaagaaagctgtgaaattgaagtcccaatttaagagtcctgaggcagaacctggtggctggaggcattcccagaggtggggaagagagcctgcccggccggagaacatctgccttgctgcacctgaggcccagcagagccgttcctgggactgtcagataatcggtgcagcggtggaaggagcctgcggctgctggcacagacttcacacagcacctcctctctgctgggtttccacacagcctgtcttcagatcctgctgccgcgtgcgaccagaggtgggaggcccctggtggcatggaagagggagggtcagtgccaagtctcaggaggagggcgcatgtgtgtatcaccctcagctggcggaacctggctgcgaactgtgcagttacgttgcatccacaggattccagttgcgtgtctgtttccttctctttctccgtatttatttttttattcttcggaggaggtggacatttcggaagtggtggggactaagggaagaactctctagttccctcagtgtgaagcctgtcgtgttctctccccttgcactggtcatcagtattgtgtaaaggaacaactgatatacttgagtgtgcaagcaaagaacccatttgccatgctgctatgaagactacttttagatcaacaataaaaaaaaacctacaaaaaaacctttattctttaattgttgcttttacggtgatattgtgcatgcaaaccaggagcattttgtgtcttaagaaaaataatcttagaacagatggctgtgaaaattacacccatgcacagaacaagccacaggaataatagttcaggatttggtttttctctttttcttgtaaacctggagggttgatatattctttccatgcagttattagaacttagttttgttccaacagttaaacttgcaatgaaaagaaaatgtgccatttttttcactcagaattattcatagctgtatatttgaaactgctaattacacacgtgtgatgtatgttggttttttagtgcaatttcttctgtagctattctttgaccaaactgtgggtattgttaatattaatttatatttgtctcattttgtatgtatgtgtagtgtgtttgtgagtatgtgtggtttataatctgacaaagtcatgaagctcagtttggctgtaatttaattccccttcccttatttttatttatttttgtactgtgctgattcaataaaatgcactgaccatccaaaaaaaaaaaaaaaaa

SEQ ID NO: 17 - DNA, 2406 bp, Homo sapiens
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 1977 bp, Homo sapiens
cactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagaacctcatctcaagccaggcctcgagctacttcattgacgctgccacagtcgacgacagtggagagtacaggtgccagacaaacctctccaccctcagtgacccggtgcagctagaagtccatgtcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagtaaaacaacaatacaggctagggatggtaatcctttaaacatacaaaaattgctcgtattataaattacccagtttagaggggaaaaaagaaaataattattcctaaacaaatggataagtagaattaatgattgaggcaggaccctacagagtgtgggaactgctggggatctagagaattcagtgggaccaatgaaagcatggctgagaaatagcagggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcagtcagttccaatgaggtggggatggagaagacaattgttgcttatgaaaaaaagctttagctgtctcctgttttgtaagctttcagtgcaacatttcttggttccaataaagcattttac

SEQ ID NO: 19 - DNA, 813 bp, Homo sapiens
acccgcgcctgaccgcggcagctcccaccatggcggagaccaagctccagctgtttgtcaaggcgagtgaggacggggagagcgtgggtcactgcccctcctgccagcggctcttcatggtcctgctcctcaagggcgtacctttcaccctcaccacggtggacacgcgcaggtccccggacgtgctgaaggacttcgcccccggctcgcagctgcccatcctgctctatgacagcgacgccaagacagacacgctgcagatcgaggactttctggaggagacgctggggccgcccgacttccccagcctggcgcctcgttacagggagtccaacaccgccggcaacgacgttttccacaagttctccgcgttcatcaagaacccggtgcccgcgcaggacgaagccctgtaccagcagctgctgcgcgccctcgccaggctggacagctacctgcgcgcgcccctggagcacgagctggcgggggagccgcagctgcgcgagtcccgccgccgcttcctggacggcgacaggctcacgctggccgactgcagcctcctgcccaagctgcacatcgtcgacacggtgtgcgcgcacttccgccaggcgcccatccccgcggagctgcgcggcgtacgccgctacctggacagcgcgatgcaggagaaagagttcaaatacacgtgtccgcacagcgccgagatcctggcggcctaccggcccgccgtgcacccccgctagcgccccaccccgcgtctgtcgcccaataaaggcatctttgtcgggagtgagggtgtcctgacatctgaagggc

SEQ ID NO: 20 - DNA, 3103 bp, Homo sapiens
cggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccatcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgttttttcctatgggtgttatcacctagctgaatgtttttctaaaggagtttatgttccattaaacgatttttaaaatgtacacttgaaaaaaaaaaaaaaaaa

SEQ ID NO: 21 - DNA, 4543 bp, Homo sapiens
cggcgggcggcgcgcacactgctcgctgggccgcggctcccgggtgtcccaggcccggccggtgcgcagagcatggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatagccaggaggccgagtggctgccggaggagcatgcgcagaggccagttaaagtagatcacctcctcgaacccactccggttccccgcaacccacagctcagctgcccatcccagtccctcgccgtccctcccaggtcgggcagtggaggctgctgtgaaaactctggtacgaatctcatccctcaactgagggccagggacccaggtgtgcctgtgctcctgcccaggagcagcttttggtctccttgggccctttttcccttccctcctttgtttacttatatatatattttttttaaacttaaattttgggtcaacttgacaccactaagatgattttttaaggagctgggggaaggcaggagccttcctttctcctgccccaagggcccagaccctgggcaaacagagctactgagacttggaacctcattgctaccacagacttgcactgaagccggacagctgcccagacacatgggcttgtgacattcgtgaaaaccaaccctgtgggcttatgtctctgccttagggtttgcagagtggaaactcagccgtagggtggcactgggagggggtgggggatctgggcaaggtgggtgattcctcccaggaggtgcttgaggccccgatggactcctgaccataatcctagccccgagacaccatcctgagccagggaacagccccagggttggggggtgccggcatctcccctagctcaccaggcctggcctctgggcagtgtggcctcttggctatttctgtgtccagttttggaggctgagttctggttcatgcagacaaagccctgtccttcagtcttctagaaacagagacaagaaaggcagacacaccgcggccaggcacccatgtgggcgcccaccctgggctccacacagcagtgtcccctgccccagaggtcgcagctaccctcagcctccaatgcattggcctctgtaccgcccggcagccccttctggccggtgctgggttcccactcccggcctaggcacctccccgctctccctgtcacgctcatgtcctgtcctggtcctgatgcccgttgtctaggagacagagccaagcactgctcacgtctctgccgcctgcgtttggaggcccctgggctctcacccagtccccacccgcctgcagagagggaactagggcaccccttgtttctgttgttcccgtgaatttttttcgctatgggaggcagccgaggcctggccaatgcggcccactttcctgagctgtcgctgcctccatggcagcagccagggacccccagaacaagaagaccccgcaggatccctcctgagctcggggggctctgccttctcaggccccgggcttcccttctccccagccagaggtggagccaagtggtccagcgtcactccagtgctcagctgtggctggaggagctggcctgtggcacagccctgagtgtcccaagccgggagccaacgaagccggacacggcttcactgaccagcggctgctcaagccgcaagctctcagcaagtgcccagtggagcctgccgcccccgcctgggcaccgggaccccctcaccatccagtgggcccggagaaacctgatgaacagtttggggactcaggaccagatgtccgtctctcttgcttgaggaatgaagacctttattcacccctgccccgttgcttcccgctgcacatggacagacttcacagcgtctgctcataggacctgcatccttcctggggacgaattccactcgtccaagggacagcccacggtctggaggccgaggaccaccagcaggcaggtggactgactgtgttgggcaagacctcttccctctgggcctgttctcttggctgcaaataaggacagcagctggtgccccacctgcctggtgcattgctgtgtgaatccaggaggcagtggacatcgtaggcagccacggccccgggtccaggagaagtgctccctggaggcacgcaccactgcttcccactggggccggcggggcccacgcacgacgtcagcctcttaccttcccgcctcggctaggggtcctcgggatgccgttctgttccaacctcctgctctgggacgtggacatgcctcaaggatacagggagccggcggcctctcgacggcacgcacttgcctgttggctgctgcggctgtgggcgagcatgggggctgccagcgtctgttgtggaaagtagctgctagtgaaatggctggggccgctggggtccgtcttcacactgcgcaggtctcttctgggcgtctgagctggggtgggagctcctccgcagaaggttggtggggggtccagtctgtgatccttggtgctgtgtgccccactccagcctggggaccccacttcagaaggtaggggccgtgtcccgcggtgctgactgaggcctgcttccccctccccctcctgctgtgctggaattccacagggaccagggccaccgcaggggactgtctcagaagacttgatttttccgtccctttttctccacactccactgacaaacgtccccagcggtttccacttgtgggcttcaggtgttttcaagcacaacccaccacaacaagcaagtgcattttcagtcgttgtgcttttttgttttgtgctaacgtcttactaatttaaagatgctgtcggcaccatgtttatttatttccagtggtcatgctcagccttgctgctctgcgtggcgcaggtgccatgcctgctccctgtctgtgtcccagccacgcagggccatccactgtgacgtcggccgaccaggctggacaccctctgccgagtaatgacgtgtgtggctgggaccttctttattctgtgttaatggctaacctgttacactgggctgggttgggtagggtgttctggcttttttgtggggtttttatttttaaagaaacactcaatcatccta

SEQ ID NO: 22 - DNA, 7037 bp, Homo sapiens
gctgcgtggagcggcggagccggagggaagcaaaggaccgtctgcgctgctgtccccgccccgcgcgctctgcgcccctcgtccctggcggtcgctccgaagctcagccctcttgcctgccccggagctgtcccgggctagccgagaagagagcggccggcaagtttgggcgcgcgcaggcggcgggccgcgggcactgggcgcctcgctggggcggggggaggtggctaccgctcccggcttggcgtcccgcgcgcacttcggcgatggcttttccgccgcggcgacggctgcgcctcggtccccgcggcctcccgcttcttctctcgggactcctgctacctctgtgccgcgccttcaacctagacgtggacagtcctgccgagtactctggccccgagggaagttacttcggcttcgccgtggatttcttcgtgcccagcgcgtcttcccggatgtttcttctcgtgggagctcccaaagcaaacaccacccagcctgggattgtggaaggagggcaggtcctcaaatgtgactggtcttctacccgccggtgccagccaattgaatttgatgcaacaggcaatagagattatgccaaggatgatccattggaatttaagtcccatcagtggtttggagcatctgtgaggtcgaaacaggataaaattttggcctgtgccccattgtaccattggagaactgagatgaaacaggagcgagagcctgttggaacatgctttcttcaagatggaacaaagactgttgagtatgctccatgtagatcacaagatattgatgctgatggacagggattttgtcaaggaggattcagcattgattttactaaagctgacagagtacttcttggtggtcctggtagcttttattggcaaggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatgatattgcaattgctgctccatatgggggtgaagataaaaaaggaattgtttatatcttcaatggaagatcaacaggcttgaacgcagtcccatctcaaatccttgaagggcagtgggctgctcgaagcatgccaccaagctttggctattcaatgaaaggagccacagatatagacaaaaatggatatccagacttaattgtaggagcttttggtgtagatcgagctatcttatacagggccagaccagttatcactgtaaatgctggtcttgaagtgtaccctagcattttaaatcaagacaataaaacctgctcactgcctggaacagctctcaaagtttcctgttttaatgttaggttctgcttaaaggcagatggcaaaggagtacttcccaggaaacttaatttccaggtggaacttcttttggataaactcaagcaaaagggagcaattcgacgagcactgtttctctacagcaggtccccaagtcactccaagaacatgactatttcaagggggggactgatgcagtgtgaggaattgatagcgtatctgcgggatgaatctgaatttagagacaaactcactccaattactatttttatggaatatcggttggattatagaacagctgctgatacaacaggcttgcaacccattcttaaccagttcacgcctgctaacattagtcgacaggctcacattctacttgactgtggtgaagacaatgtctgtaaacccaagctggaagtttctgtagatagtgatcaaaagaagatctatattggggatgacaaccctctgacattgattgttaaggctcagaatcaaggagaaggtgcctacgaagctgagctcatcgtttccattccactgcaggctgatttcatcggggttgtccgaaacaatgaagccttagcaagactttcctgtgcatttaagacagaaaaccaaactcgccaggtggtatgtgaccttggaaacccaatgaaggctggaactcaactcttagctggtcttcgtttcagtgtgcaccagcagtcagagatggatacttctgtgaaatttgacttacaaatccaaagctcaaatctatttgacaaagtaagcccagttgtatctcacaaagttgatcttgctgttttagctgcagttgagataagaggagtctcgagtcctgatcatatctttcttccgattccaaactgggagcacaaggagaaccctgagactgaagaagatgttgggccagttgttcagcacatctatgagctgagaaacaatggtccaagttcattcagcaaggcaatgctccatcttcagtggccttacaaatataataataacactctgttgtatatccttcattatgatattgatggaccaatgaactgcacttcagatatggagatcaaccctttgagaattaagatctcatctttgcaaacaactgaaaagaatgacacggttgccgggcaaggtgagcgggaccatctcatcactaagcgggatcttgccctcagtgaaggagatattcacactttgggttgtggagttgctcagtgcttgaagattgtctgccaagttgggagattagacagaggaaagagtgcaatcttgtacgtaaagtcattactgtggactgagacttttatgaataaagaaaatcagaatcattcctattctctgaagtcgtctgcttcatttaatgtcatagagtttccttataagaatcttccaattgaggatatcaccaactccacattggttaccactaatgtcacctggggcattcagccagcgcccatgcctgtgcctgtgtgggtgatcattttagcagttctagcaggattgttgctactggctgttttggtatttgtaatgtacaggatgggcttttttaaacgggtccggccacctcaagaagaacaagaaagggagcagcttcaacctcatgaaaatggtgaaggaaactcagaaacttaactgcagtttttaagttatgctacatcttgacccactagaattagcaactttattatagatttaaactttcttcatgaggagtaaaaatccaaggctttactgctgatagtgctaattggcattaaccacaaaatgagaattatatttgtcaaccttctccttataaataagttcagacatacatttaataacatagggtgacttgtgtttttaggtatttaaataataaaatttcaagggatagtttttattcaatgtatataagacaggtagtgcctgatttactactttatataaaatagtacctccttcagttactgtttctgatttaatgtacggaactttatttgttgttgttgttgttgttgttgttgttgttttaaagcagtccaaatttggaccttagcaatcatgtcttttgtataggtacttaatgttaatacatattacactacagtttacttttcagaatactaaagactttataactgcatgaacttggatttttttaatcactcatatggtagaattttataaacacatacatgataccatccaaattcttgcttttaataacaaaggtacaatattttgttttagtatgaaaatctggtagatcctattacacttctgtttatattaaatccacaatattttattacatttttaacttgtataaattttaggtcaaatccttcaagccaacctatactaaaaattagttccataatcacaaatggctcttttgtgtaattgtttaatttcacctgaatatcataatgcttaaagccatatggagttggaaattatttccaaagcatatttattccattgttttagtctggctatttacagtataaaaaaagcatttttattaaaatactgtgtagttctttgagatagttgcttatgcatatagtaagtattacattcttagagtagagcagagtttttagttagtattaatttattttcctccattcatgtacttttccttatatttccaaaactgttactgagaatgggtcaagatcagtgagaaatctttacagttgacaggaacctggaccccttaccccaactttatgagtaatgcttggaataaaaactcttaaggcaactcactgatttacttctagcaatagcatgatgttacaggaatattacctctgtttaagcaaggtaatgtgtaaaatcagtctcggctgtcagaataacttctaaaaggtatttttataagcagttcaagttactgaaaaccttttaaacctttctgaagttcgttagtataaattacttttctaggattattaataaaagccacataggtggcaagttgtagttttatatggctctgtagagtggtgaaccttctagaggaatatatgatttattcacagttcctcaaggcctggggatgatgatcagttatacctatttttgtgcaattacatcatgttgtacattagaaatggagagtttaatagctctttaactgctgtcctcattaggtaatgataaatatttcccttaaataattgactattttgctgtgttttaaaaatgattgaaatttatcttgccatatctcataatttcatgcacaagttgactgagctaatcttgagaatatattcgtaaaataggagcacatttagttgaggtatacaaggtaggactctagacaaaaccttctattttagctttagtgaatttcaaaagtaatgggtcttggagtatagatttttattagtagcttgaaagagcttaatcatatgcagtaagtatttttattaccaataaatttaaaattttttaagaaaaatatttttatcctagggccaagtgttgcctgccaccaatcagtaagttagtctataacaaattttaccctaacagttttaccacctagtaacagtcatttctgaaaatatgttggatagaaagtcactctttggcaaaagtgttagaatttgcttttgtgccatctattccttttatggcatctatcttgaaagtaatcttgtattggagattgaaagatgctgtaatttagaaattaacatgatatcttaaattacctttatgaaatatagttttgtataatagcatagattttccttcaaaaaatgaacatttatatatctacaaaaatatggagaagagtaatttgaaagcctactttctgaagaaaatggtgggatttttttttatcatgattaaatatcaaaaaattgccctatgaaaactttaaatctctaaaacatttgaaatactaccatatttgtgatttattgagaataaaaatccattttgaaatgtaaaatttttatgatctgattcagttttaagaaaacatgaatgaactagaagatattaaaaacatttgacattggtaagaaatattgatactgatattgatttttatataggtatttatttcagaattgatattttgagaaaaatacatgtgagtcattttttctgtttctcttttctcttaacgattatcactgtaattctgaatctgaaaggtaaaacaattagtcaaaatattattgccatcattctacctgtgttatgaaactacttattcatagttaattctcattaacacttacatttccataaagaaaactcaagtattaataaaagagactttactggcttaagagggctgtgaaagatttttgatagtgaatcatgaccctaagggagagatttgtgtgataaaagtattgtatataatagatcagcgatttttgtaaggcaaacagaatttgtaagttggcagatcttcctaagttgcaaaatgtaatgatgagcttggtggagaagaatgagtcgttcttggaatacctatgtgcagccactacccatctcaatgtcaccttgtttgcattcttggatagcttgtatatgtagtagtttgatgaataatttaaagaaaaacacctaaaatttgaaaaatgattgtaggatcaaaaaaggcagatgaaattacttaatactcagtgttttggagagtattccttttagtttgttggttggctggtttgaacgatagaaatatgcagcatgcaatatatgcttatatttcattttaatttctgatatataatgaacttcttgggagaggtactgaatctttgatgttttttgtcattgttctcaagtgcaatataacaatgtaaccaaatctagataatttcaaagttgtcattaatttagtaagcctaatataaacaaatatttgtattatttttgttagcaggaaagagtgattaagtgaggttatttacccctaaatggtccattctgcattgtatttcaggctggaaatgaattattctttaccagttttgaaacactttgaaatatcctaaggtaacttggaagctgtgtagtatatcaaattaatttgctacctaataacatagaaagtaaatatctttgtggtcacccacattgggtgagacagaaaatgaatctgttctaaaatttgtaatttgctaacttgatttgagttagtgaaaactggtacagtgttctgcttgatttacaacatgtaacttgtgactgtacaataaacataagcatatggtaccaaaaaaaaaaaaaaaa

SEQ ID NO: 23 - DNA, 2360 bp, Homo sapiens
gctacgcgggccacgctgctggctggcctgacctaggcgcgcggggtcgggcggccgcgcgggcgggctgagtgagcaagacaagacactcaagaagagcgagctgcgcctgggtcccggccaggcttgcacgcagaggcgggcggcagacggtgcccggcggaatctcctgagctccgccgcccagctctggtgccagcgcccagtggccgccgcttcgaaagtgactggtgcctcgccgcctcctctcggtgcgggaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccactgagagagacttgtgctcaaggaatcggctggggactgctacctctgagaagacacaaggtgatttcagactgcagaggggaaagacttccatctagtcacaaagactccttcgtccccagttgccgtctaggattgggcctcccataattgctttgccaaaataccagagccttcaagtgccaaacagagtatgtccgatggtatctgggtaagaagaaagcaaaagcaagggaccttcatgcccttctgattcccctccaccaaaccccacttcccctcataagtttgtttaaacacttatcttctggattagaatgccggttaaattccatatgctccaggatctttgactgaaaaaaaaaaagaagaagaagaaggagagcaagaaggaaagatttgtgaactggaagaaagcaacaaagattgagaagccatgtactcaagtaccaccaagggatctgccattgggaccctccagtgctggatttgatgagttaactgtgaaataccacaagcctgagaactgaattttgggacttctacccagatggaaaaataacaactatttttgttgttgttgtttgtaaatgcctcttaaattatatatttattttattctatgtatgttaatttatttagtttttaacaatctaacaataatatttcaagtgcctagactgttactttggcaatttcctggccctccactcctcatccccacaatctggcttagtgccacccacctttgccacaaagctaggatggttctgtgacccatctgtagtaatttattgtctgtctacatttctgcagatcttccgtggtcagagtgccactgcgggagctctgtatggtcaggatgtaggggttaacttggtcagagccactctatgagttggacttcagtcttgcctaggcgattttgtctaccatttgtgttttgaaagcccaaggtgctgatgtcaaagtgtaacagatatcagtgtctccccgtgtcctctccctgccaagtctcagaagaggttgggcttccatgcctgtagctttcctggtccctcacccccatggccccaggccacagcgtgggaactcactttcccttgtgtcaagacatttctctaactcctgccattcttctggtgctactccatgcaggggtcagtgcagcagaggacagtctggagaaggtattagcaaagcaaaaggctgagaaggaacagggaacattggagctgactgttcttggtaactgattacctgccaattgctaccgagaaggttggaggtggggaaggctttgtataatcccacccacctcaccaaaacgatgaaggtatgctgtcatggtcctttctggaagtttctggtgccatttctgaactgttacaacttgtatttccaaacctggttcatatttatactttgcaatccaaataaagataacccttattccataaaaaaaaaaaaaaaaaa

SEQ ID NO: 24 - DNA, 1546 bp, Homo sapiens
taactgtccaccagaaaggactgctctttgggtgagttgaacttcttccattatagaaagaattgaaggctgagaaactcagcctctatcatgtggaacagctctgacgccaacttctcctgctaccatgagtctgtgctgggctatcgttatgttgcagttagctggggggtggtggtggctgtgacaggcaccgtgggcaatgtgctcaccctactggccttggccatccagcccaagctccgtacccgattcaacctgctcatagccaacctcacactggctgatctcctctactgcacgctccttcagcccttctctgtggacacctacctccacctgcactggcgcaccggtgccaccttctgcagggtatttgggctcctcctttttgcctccaattctgtctccatcctgaccctctgcctcatcgcactgggacgctacctcctcattgcccaccctaagctttttccccaagttttcagtgccaaggggatagtgctggcactggtgagcacctgggttgtgggcgtggccagctttgctcccctctggcctatttatatcctggtacctgtagtctgcacctgcagctttgaccgcatccgaggccggccttacaccaccatcctcatgggcatctactttgtgcttgggctcagcagtgttggcatcttctattgcctcatccaccgccaggtcaaacgagcagcacaggcactggaccaatacaagttgcgacaggcaagcatccactccaaccatgtggccaggactgatgaggccatgcctggtcgtttccaggagctggacagcaggttagcatcaggaggacccagtgaggggatttcatctgagccagtcagtgctgccaccacccagaccctggaaggggactcatcagaagtgggagaccagatcaacagcaagagagctaagcagatggcagagaaaagccctccagaagcatctgccaaagcccagccaattaaaggagccagaagagctccggattcttcatcggaatttgggaaggtgactcgaatgtgttttgctgtgttcctctgctttgccctgagctacatccccttcttgctgctcaacattctggatgccagagtccaggctccccgggtggtccacatgcttgctgccaacctcacctggctcaatggttgcatcaaccctgtgctctatgcagccatgaaccgccaattccgccaagcatatggctccattttaaaaagagggccccggagtttccataggctccattagaactgtgaccctagtcaccagaattcaggactgtctcctccaggaccaaagtggccaggtaataggagaataggtgaaataacacatgtgggcattttcacaacaatctctccccagcctcccaaatcaagtctctccatcacttgatcaatgtttcagccctagactgcccaaggagtattattaattattaataaatgaattctgtgcttttaaaaaaaaaaaaataaaaaaagaaaaaaaaaaaaaaaaaaaaaaaaaaaaa

SEQ ID NO: 25 - DNA, 4131 bp, Homo sapiens
atcccgcccgcatacagcccgcatcccgccggggaagcgagcccagtccagcgctgcccgtccagtcctcgcccaagatttaaagcccgcaagttttgttcttgagaccagcgactttagctccgatgcgggaaggaaagccgacctccgatttggacatttaaagagctgggcttgaacttcgtgagtttcgctctaaactgcccttgaaatgaagctggacttggaggtggcatggaatattcacatgggagagccgcatgaggccgcccaccacgcttcctgaaggatgcccgtgtggaagaattttgacgtgccagtgtcctcgttctacagggtgttccattcttccgcaatctcagaaaaatgggactaaaagaaactattttgtaaaataagaagacttccatttttaatgaccaacatgtattaagatggacacctactctacgaaacacgaagttctatggtctcgaagaagcccgtgcctgtttaaaactgatcctaactaaaaacagacttgagtggatatgagaatgttggttagtggcagaagagtcaaaaaatggcagttaattattcagttatttgctacttgttttttagcgagcctcatgtttttttgggaaccaatcgataatcacattgtgagccatatgaagtcatattcttacagatacctcataaatagctatgactttgtgaatgataccctgtctcttaagcacacctcagcggggcctcgctaccaatacttgattaaccacaaggaaaagtgtcaagctcaagacgtcctccttttactgtttgtaaaaactgctcctgaaaactatgatcgacgttccggaattagaaggacgtggggcaatgaaaattatgttcggtctcagctgaatgccaacatcaaaactctgtttgccttaggaactcctaatccactggagggagaagaactacaaagaaaactggcttgggaagatcaaaggtacaatgatataattcagcaagactttgttgattctttctacaatcttactctgaaattacttatgcagttcagttgggcaaatacctattgtccacatgccaaatttcttatgactgctgatgatgacatatttattcacatgccaaatctgattgagtaccttcaaagtttagaacaaattggtgttcaagacttttggattggtcgtgttcatcgtggtgcccctcccattagagataaaagcagcaaatactacgtgtcctatgaaatgtaccagtggccagcttaccctgactacacagccggagctgcctatgtaatctccggtgatgtagctgccaaagtctatgaggcatcacagacactaaattcaagtctttacatagacgatgtgttcatgggcctctgtgccaataaaatagggatagtaccgcaggaccatgtgtttttttctggagagggtaaaactccttatcatccctgcatctatgaaaaaatgatgacatctcatggacacttagaagatctccaggacctttggaagaatgctacagatcctaaagtaaaaaccatttccaaaggtttttttggtcaaatatactgcagattaatgaagataattctcctttgtaaaattagctatgtggacacatacccttgtagggctgcgtttatctaatagtacttgaatgttgtatgttttcactgtcactgagtcaaacctggatgaaaaaaacctttaaatgttcgtctataccctaagtaaaatgaggacgaaagacaaatattttgaaagcctagtccatcagaatgtttctttgattctagaagctgtttaatatcacttatctacttcattgcctaagttcatttcaaagaatttgtatttagaaaaggtttatattattagtgaaaacaaaactaaagggaagttcaagttctcatgtaatgccacatatatacttgaggtgtagagatgttattaagaagttttgatgttagaataattgcttttggaaaataccaaatgaacgtacagtacaacatttcaaggaaatgaatatattgttagaccaggtaagcaagtttatttttgttaaagagcacttggtggaggtagtaggggcagggaaaggtcagcataggagagaaagttcatgaatctggtaaaacagtctcttgttcttaagaggagatgtagaaaaatgtgtacaatgttattataaacagacaaatcacgtcttaccacatccatgtagctactggtgttagagtcattaaaatacctttttttgcatcttttttcaaagtttaatgtgaacttttagaaaagtgattaatgttgccctaatactttatatgtttttaatggatttttttttaagtattagaaaatgacacataacacgggcagctggttgctcatagggtccttctctagggagaaaccattgttaattcaaataagctgattttaatgacgttttcaactggtttttaaatattcaatattggtctgtgtttaagtttgttatttgaatgtaatttacatagaggaatataataatggagagacttcaaatggaaagacagaacattacaagcctaatgtctccataattttataaaatgaaatcttagtgtctaaatccttgtactgattactaaaattaacccactcctccccaacaaggtcttataaaccacagcactttgttccaagttcagagttttaaattgagagcattaaacatcaaagttataatatctaaaacaatttatttttcatcaataactgtcagaggtgatctttattttctaaatatttcaaacttgaaaacagagtaaaaaagtgatagaaaagttgccagtttggggttaaagcatttttaaagctgcatgttccttgtaatcaaagagatgtgtctgagatctaatagagtaagttacatttattttacaaagcaggataaaaatgtggctataatacacactacctcccttcactacagaaagaactaggtggtgtctactgctagggagattatatgaaggccaaaataatgacttcagcaagagtgactgaactcactctaaggcctttgactgcagaggcacctgttagggaaaatcagatgtctcatataataaggtgatgtcggaaacacgcaaaacaaaacgaaaaaagatttctcagtatacacaactgaatgatgatacttacaatttttagcaggtagctttttaatgtttacagaaattttaatttttttctattttgaaatttgaggcttgtttacattgcttagataatttagaatttttaactaatgtcaaaactacagtgtcaaacattctaggttgtagttactttcagagtagatacagggttttagatcattacagtttaagttttctgaccaattaaaaaaacatagagaacaaaagcatatttgaccaagcaacaagcttataattaatttttattagttgattgattaatgatgtattgccttttgcccatatataccctgtgtatctatacttggaagtgtttaaggttgccattggttgaaaacataagtgtctctggccatcaaagtgatcttgtttacagcagtgcttttgtgaaacaattatttatttgctgaaagagctcttctgaactgtgtccttttaatttttgcttagaatagaatggaacaagtttaaatttcaaggaaatatgaaggcacttcctttttttctaagaaggaagttgctagatgattccttcatcacacttacttaaagtactgagaagagtatctgtaaataaaagggttccaaccttttaaaaaagaaggaaaaaactttttggtgctccagtgtagggctatctttttaaaaaatgtcaacaaagggaaaataaactatcagcttggatggtcacttgaatagaagatggttatacacagtgttattgttaaaatttttttaccttttggttggtttgcatcttttttccatattgttaattttataccaaaatgttaaatatttgtattacttgaattttgctcttgtatggcaaaataattagtgagtttaaaaaaaatctatagtttccaataaacaactgaaaaattatcatgaaaaaaaaa

SEQ ID NO: 26 - DNA, 4930 bp, Homo sapiens
aaacccgatctccttggacttgaatgaggaggaggaggcggcggcggcggcggcggcggaggcgctcggctggggaaagctagcggcagaggctcagccccggcggcagcgcgcgccccgctgccagcccattttccggacgccacccgcgggcactgccgacgcccccggggctgccgaggggaggccgggggggcgcagcggagcgcggtcccgcgcactgagccccgcggcgccccgggaacttggcggcgacccgagcccggcgagccggggcgcgcctcccccgccgcgcgcctcctgcatgcggggccccagctccgggcgccggccggagccccccccggccgcccccgagccccccgcgccccgcgccgcgccgccgcgccgtccatgcaccgcttgatgggggtcaacagcaccgccgccgccgccgccgggcagcccaatgtctcctgcacgtgcaactgcaaacgctctttgttccagagcatggagatcacggagctggagtttgttcagatcatcatcatcgtggtggtgatgatggtgatggtggtggtgatcacgtgcctgctgagccactacaagctgtctgcacggtccttcatcagccggcacagccaggggcggaggagagaagatgccctgtcctcagaaggatgcctgtggccctcggagagcacagtgtcaggcaacggaatcccagagccgcaggtctacgccccgcctcggcccaccgaccgcctggccgtgccgcccttcgcccagcgggagcgcttccaccgcttccagcccacctatccgtacctgcagcacgagatcgacctgccacccaccatctcgctgtcagacggggaggagcccccaccctaccagggcccctgcaccctccagcttcgggaccccgagcagcagctggaactgaaccgggagtcggtgcgcgcacccccaaacagaaccatcttcgacagtgacctgatggatagtgccaggctgggcggcccctgcccccccagcagtaactcgggcatcagcgccacgtgctacggcagcggcgggcgcatggaggggccgccgcccacctacagcgaggtcatcggccactacccggggtcctccttccagcaccagcagagcagtgggccgccctccttgctggaggggacccggctccaccacacacacatcgcgcccctagagagcgcagccatctggagcaaagagaaggataaacagaaaggacaccctctctagggtccccaggggggccgggctggggctgcgtaggtgaaaaggcagaacactccgcgcttcttagaagaggagtgagaggaaggcggggggcgcagcaacgcatcgtgtggccctcccctcccacctccctgtgtataaatatttacatgtgatgtctggtctgaatgcacaagctaagagagcttgcaaaaaaaaaaagaaaaaagaaaaaaaaaaaccacgtttctttgttgagctgtgtcttgaaggcaaaagaaaaaaaatttctacagtagtctttcttgtttctagttgagctgcgtgcgtgaatgcttattttcttttgtttatgataatttcacttaactttaaagacatatttgcacaaaacctttgtttaaagatctgcaatattatatatataaatatatataagataagagaaactgtatgtgcgagggcaggagtatttttgtattagaagaggcctattaaaaaaaaaagttgttttctgaactagaagaggaaaaaaatggcaatttttgagtgccaagtcagaaagtgtgtattaccttgtaaagaaaaaaattacaaagcaggggtttagagttatttatataaatgttgagattttgcactattttttaatataaatatgtcagtgcttgcttgatggaaacttctcttgtgtctgttgagactttaagggagaaatgtcggaatttcagagtcgcctgacggcagagggtgagcccccgtggagtctgcagagaggccttggccaggagcggcgggctttcccgaggggccactgtccctgcagagtggatgcttctgcctagtgacaggttatcaccacgttatatattccctaccgaaggagacaccttttcccccctgacccagaacagcctttaaatcacaagcaaaataggaaagttaaccacggaggcaccgagttccaggtagtggttttgcctttcccaaaaatgaaaataaactgttaccgaaggaattagtttttcctcttcttttttccaactgtgaaggtccccgtggggtggagcatggtgcccctcacaagccgcagcggctggtgcccgggctaccagggacatgccagagggctcgatgacttgtctctgcagggcgctttggtggttgttcagctggctaaaggttcaccggtgaaggcaggtgcggtaactgccgcactggaccctaggaagccccaggtattcgcaatctgacctcctcctgtctgtttcccttcacggatcaattctcacttaagaggccaataaacaacccaacatgaaaaggtgacaagcctgggtttctcccaggataggtgaaagggttaaaatgagtaaagcagttgagcaaacaccaacccgagcttcgggcgcagaattcttcaccttctcttcccctttccatctcctttccccgcggaaacaacgcttcccttctggtgtgtctgttgatctgtgttttcatttacatctctcttagactccgctcttgttctccaggttttcaccagatagatttggggttggcgggacctgctggtgacgtgcaggtgaaggacaggaaggggcatgtgagcgtaaatagaggtgaccagaggagagcatgaggggtggggctttgggacccaccggggccagtggctggagcttgacgtctttcctccccatgggggtgggagggcccccagctggaagagcagactcccagctgctaccccctcccttcccatgggagtggctttccattttgggcagaatgctgactagtagactaacataaaagatataaaaggcaataactattgtttgtgagcaacttttttataacttccaaaacaaaaacctgagcacagttttgaagttctagccactcgagctcatgcatgtgaaacgtgtgctttacgaaggtggcagctgacagacgtgggctctgcatgccgccagcctagtagaaagttctcgttcattggcaacagcagaacctgcctctccgtgaagtcgtcagcctaaaatttgtttctctcttgaagaggattctttgaaaaggtcctgcagagaaatcagtacaggttatcccgaaaggtacaaggacgcacttgtaaagatgattaaaacgtatctttcctttatgtgacgcgtctctagtgccttactgaagaagcagtgacactcccgtcgctcggtgaggacgttcccggacagtgcctcactcacctgggactggtatcccctcccagggtccaccaagggctcctgcttttcagacaccccatcatcctcgcgcgtcctcaccctgtctctaccagggaggtgcctagcttggtgaggttactcctgctcctccaacctttttttgccaaggtttgtacacgactcccatctaggctgaaaacctagaagtggaccttgtgtgtgtgcatggtgtcagcccaaagccaggctgagacagtcctcatatcctcttgagccaaactgtttgggtctcgttgcttcatggtatggtctggatttgtgggaatggctttgcgtgagaaaggggaggagagtggttgctgccctcagccggcttgaggacagagcctgtccctctcatgacaactcagtgttgaagcccagtgtcctcagcttcatgtccagtggatggcagaagttcatggggtagtggcctctcaaaggctgggcgcatcccaagacagccagcaggttgtctctggaaacgaccagagttaagctctcggcttctctgctgagggtgcaccctttcctctagatggtagttgtcacgttatctttgaaaactcttggactgctcctgaggaggccctcttttccagtaggaagttagatgggggttctcagaagtggctgattggaaggggacaagcttcgtttcaggggtctgccgttccatcctggttcagagaaggccgagcgtggctttctctagccttgtcactgtctccctgcctgtcaatcaccacctttcctccagaggaggaaaattatctcccctgcaaagcccggttctacacagatttcacaaattgtgctaagaaccgtccgtgttctcagaaagcccagtgtttttgcaaagaatgaaaagggaccccatatgtagcaaaaatcagggctgggggagagccgggttcattccctgtcctcattggtcgtccctatgaattgtacgtttcagagaaattttttttcctatgtgcaacacgaagcttccagaaccataaaatatcccgtcgataaggaaagaaaatgtcgttgttgttgtttttctggaaactgcttgaaatcttgctgtactatagagctcagaaggacacagcccgtcctcccctgcctgcctgattccatggctgttgtgctgattccaatgctttcacgttggttcctggcgtgggaactgctctcctttgcagccccatttcccaagctctgttcaagttaaacttatgtaagctttccgtggcatgcggggcgcgcacccacgtccccgctgcgtaagactctgtatttggatgccaatccacaggcctgaagaaactgcttgttgtgtatcagtaatcattagtggcaatgatgacattctgaaaagctgcaatacttatacaataaattttacaattctttggaaaaaaaaaaaaaaaaaa

SEQ ID NO: 27 - DNA, 2476 bp, Homo sapiens
atttttagtttgttgaagttcgtgactgcttcactctctcattcttagcttgaatttggaaatgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaagagccaagagaagtgcttgtctttggatgccaagttgctgaaaattaatagcacagctgatctggacttcatccagcaagcaatttcctattccagttttccattctggatggggctgtctcggaggaaccccagctacccatggctctgggaggacggttctcctttgatgccccacttatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtgaatttgaaggctctggaagaaaagaaaaaagtctttgagttttattctggaatttaagctattctttgtcacttgggtgccaaacatgagagcccagaaaactgtcatttagctggctgcagaactcctttgcagaaactggggttccaggtgcctggcacctttatgtcaacatttttgattctagctacctgtattatttcacctagcttgtcccaagcttccctgccagcctgaagtccattttcccctttttattttaaaatttgactcctcttcaagcttgaaaaccctctgaactcagtcttctttacctcattatcaccttcccctcacactcctaaaattgcatgaaagacagaacatggagaacttgctcaagtgcaggcagagagcaaaaaggggaaatatgtctgggaaaaagtgcacgtgaagaaacaaagaaggacagaggccattccgaaatcaagaaactcatgttcttaactttaaaaaaggtatcaatccttggtttttaaactgtggtccatctccagactctaccacttacggacagacagacagacagacacacacacacacacacacacacattttgggacaagtggggagcccaagaaagtaattagtaagtgagtggtcttttctgtaagctaatccacaacctgttaccacttcctgaatcagttattatttcttcatttttttttctaccagaggacagattaatagatttaacccttcacaacagttcttgttagaatcatgggatgtgtggcccagaggtaagaatagaatttctttccctaaagaacataccttttgtagatgaactcttctcaactctgttttgctatgctataattccgaaacatacaagacaaaaaaaatgaagacactcaatctagaacaaactaagccaggtatgcaaatatcgctgaatagaaacagatggaattagaaatatatcttctatttttaggcttctatttcctttccacccactcttcacaggctattctactttaaaggaagcctttttattttgctgcacacaatctagcaggaatctttttttttttttaagagctgtgtcatccttatgtaggcaagagatgtttgcttttgttaaaagctttattgagatataattaacataaaataaactgaacatatttaaagtgtactatttgataagttttcacaccttgtggagaacatgcatactacaattaagagagtgaacatatccatcatccctcaaagtgtcacaatgctcctcctgatgactcctccccagaaaaccaccaatcggctttcattttgcattttgtagttttatgtgaatggaatcatatagtatgtcttttttttttgtctggcttctttcactttgcataattattttgagattcatatgtctccatcttgatgctcgtatgaattcattcttttaaatgttgaatattcccttgtatggatataccacaattcatttacccatttacttgttgatgacatttgggttgttttagttttgggatattacaaataaagctgctgtgaacatttgtgtacaaaaaaaaaaaaaaaa

SEQ ID NO: 28 - DNA, 695 bp, Homo sapiens
acacaagaagggtaggagagaaaagccatggccgacaaggtcctgaaggagaagagaaagctgtttatccattccatgggtgaaggtacaataaatggcttactggatgaattattacagacaagggtgctgaaccaggaagagatggagaaagtaaaacgtgaaaatgctacagttatggataagacccgagctttgattgactccgttattccgaaaggggcacaggcatgccaaatttgcatcacatacatttgtgaagaagacagttacctggcagagacgctgggactctcagcagctcttcaggcagtgcaggacaacccagctatgcccacatgctcaagcccagaaggcagaatcaagctttgctttctagaagacgctcaaaggatatggaaacaaaagttgcagaggtgccatgttcagaatacaataataaagtggagtgaaagatatacttcaggcagttttgaaatgcagtggctttttcttagaaccaattttattgagcggttctggaggaacatacttctgctccccttgcataaaggatctctatatcctagaatcccaggtttaggtaaagagttgcaaactggaactcataaattatcatagtcttcacccagtctgtctcttagaatttatttcccactgacacaaacaagccattttgtctgcttgtataaccc

SEQ ID NO: 29 - DNA, 3988 bp, Homo sapiens
tgcaactccgagcgtgagtccaggctaaggggacgccggccggggaagaggcgcggcgggagaagcgaccgcagcgggagcccggccaccgagggctgccggcactaggcgcagagccgccagcgctcggaagcccgcggggtgcgggagcgggaacagacttcttttgtagaattatcctatggaatgatattttcataatgagtcaacaaggttacgtggctacacctccgtattctcagcctcagcctggaataggcctttctccacctcattatgggcactatggggatccgtcgcacacagcatctccaacaggtatgatgaagccagcagggcctttgggggccaccgccactaggggaatgttgcctccgggtcccccacctcctggaccccatcagtttggtcagaatggagctcatgccactggtcaccctccccaaagatttccaggccctccacctgtcaacaatgtggcatcctcacatgcaccataccaaccctctgcacaatcttcttatccaggtcctatatccacttcatctgtcacccagctgggcagccagctcagtgctatgcaaatcaacagctatggttcaggcatggctcctccaagccagggaccccctggccctctgtcagccacatcattgcagactcctccacgacctccacagccttccattttgcagcctggatctcaagttcttccaccaccacccaccacactcaatggtcctggtgcctcacctttgcctctaccaatgtacagaccagatgggctctctgggcctcctcctccaaatgcccagtaccagcccccacctcttccaggccagaccttgggtgctggatatcctccgcagcaggccaactctggtccccagatggcaggcgcacaactgtcttacccaggaggcttccctggaggtcctgcacagatggctggtccgccacagccccagaagaagctggatcctgactctatccctagcccaatccaggtgattgagaatgatagagccagcagaggaggacaagtttatgccaccaacaccagaggccagatccctcccctggtcactacagattgcatgatacaagaccaaggaaatgccagtcctcgattcatccgttgtacaacatactgttttccatgcacgtcagatatggctaagcaagctcagattccattagctgctgtcatcaagccctttgccaccattccttcaaatgagagtcccctttacttggtaaatcacggcgagagtggaccagtcagatgcaacaggtgcaaggcctacatgtgcccatttatgcagttcatcgaaggaggaaggagatatcagtgtggattttgcaactgtgtgaatgatgttccaccattctatttccaacatctggaccacattggaagaagactggaccactatgagaaaccagagttatctctaggatcttatgaatatgttgccactttggattattgcagaaagagtaagcctcccaacccaccagcctttatcttcatgattgatgtttcatatagtaacataaagaatggacttgtcaagctcatatgtgaagaactgaagaccatgctggaaaaaattccaaaggaagagcaagaagagacgtctgcaattcgagtgggttttatcacatataacaaagttctccatttctttaatgtgaagagtaatctggcccagcctcagatgatggtggtgactgatgttggagaagtctttgttcctttgttggatggtttccttgtcaactatcaagaatcccaatctgtgattcataatttgttggaccagattccagacatgtttgcagactctaatgaaaatgagactgtctttgcttctgtcatccaggctggcatggaagcactaaaggcagcagactgtcctgggaagctgttcatcttccattcttccttgccaactgctgaagcaccagggaagctcaaaaacagagatgacaaaaaactggttaatacagacaaagagaagatacttttccagccccaaacaaatgtctatgactcattggccaaggactgcgtggctcacggctgctctgtgacactcttcctctttcctagtcagtatgtggacgtggcctcgctggggctggttcctcagctcactggaggaaccctttacaaatacaacaatttccagatgcacttggatagacaacaatttttgaacgacctcagaaatgatattgaaaagaaaataggctttgatgctattatgagggttcgtaccagcacaggtttcagagccactgatttctttggtggaatcttgatgaacaacaccaccgatgtagaaatggctgccatcgattgtgacaaggcagtgaccgtggagttcaagcacgatgacaaactcagtgaagacagtggagccttaatccagtgtgctgtgctttacacgacaatcagtggtcaaagaagacttcggattcacaatcttggcttaaactgcagctctcagctagctgatctttataagagctgtgagacagatgctcttatcaacttctttgccaagtcagcttttaaagcagttctccaccagcctttgaaggtcatccgggaaattctagttaatcagactgcccatatgttggcatgttaccggaagaattgtgcaagtccttctgcagcaagccagcttattctaccagattccatgaaagtattgccagtgtacatgaattgcttgttgaaaaactgtgtactactcagcagaccagagatctcaactgatgaacgagcataccagagacagctggtcatgaccatgggtgtggctgactctcagcttttcttctacccacaacttctgcccatacacacgttagatgtcaagagtacaatgttacctgctgccgttcgttgctctgagtcccgtctttcagaagaaggaatattcttactggctaatggtctacacatgttcctgtggttgggagtaagcagcccaccagaactgatccaaggaatatttaatgtgccatcttttgcacatatcaacacagatatgacattgctgcctgaagtgggaaacccatactctcaacaactcagaatgataatgggtattatccaacaaaagaggccatattcaatgaagctcacaattgtaaagcagcgagaacaaccagaaatggttttccgacagttcctggtagaagacaaaggactttacggaggctcttcttatgtggatttcctttgttgtgttcacaaggagatctgtcagctgcttaattaattgaaacttctctgtcattgatgttgcatttccaaggagataatctccttcttggtgcctaattttctagatgataataggctagttttgatttcttgctcattttcagaataactttccaggaagagatggcatttagaacttcagctttggtgctcaggtataaagccaattaaggtacaattgtaccataaagggaacaatctgtttctgattgcacagtttctaatttttaaaactgatgtggtttgcatttcataaaaggcaaagtttacagaaccataaacattctcaattttctttatgctagacatataaattatttttcaaactgtatagatttggggtaaaaagttgtctcagttcctctcccaattgcaatgagaaaaaaaagcttaatttttacattatacttaattttctaaaaccatgtaactccattgaacatttttcaacttaaggtctgcatagcagacttttaataaccttgggatttatctggtagaacaatatgtgttctacatttttttcataattatatattgtgtatgttaaaactattttccagttgttttgtctgtaaaactgtctttatcaatatgcttaatggttctttgtacaattttgaaagtttctacctgtatataatggatgttaaccagtatcaataaatcacttcgtataatctt

SEQ ID NO: 30 - DNA, 5583 bp, Homo sapiens
ataatctatcccagatcctttcccagagagaaacttggcgatcacgttttcacatgatgctcacgctcagggcgcttcaattatccctccccacaaagataggtggcgcgtgtttcagggtctctcgtctctctcctacagaaaagaaaaagaaaaaaatgtcattagaagaggcgtaacacgtcagtccgtccccaggtttgtgtttcctggagtggccgaaagagatcagttctaacctgctctgcaggaataacggtcctgcctcccgacactcttggcgaggtttttgtacagtttgctccgggagctgtttcttcgcttccacctttttctcccccacacttcgcggcttcttcatgctttttcttctcaccatttctggccaaaactacaaacaagacttcgcagatcgagcctgcgtgctgccgaagcagggcgccgagtccatgcgaactgccatctgatccgctcttatcaatgaagcagccgatcatggcggatggcccccggtgcaagaggcgcaaacaagccaatcccaggaggaaaaacgtggtgaactatgacaatgtagtggacacaggttctgaaacagatgaggaagacaagcttcatattgctgaggatgacggtattgccaaccctctggaccaggagacgagtccagctagtgtgcccaaccatgagtcctccccacacgtgagccaagctctgttgccaagagaggaagaggaagatgaaataagggagggtggagtggaacacccctggcacaacaacgagattctacaagcctctgtagatggtccagaagaaatgaaggaagactatgacactatggggccagaagccacgatccagaccgcaattaacaatggtacagtgaagaatgcaaattgcacatcagattttgaggaatactttgccaaaagaaaactggaggaacgcgatggtcatgcagtcagcatcgaggagtaccttcagcgcagtgacacagccattatttacccagaagcccctgaggagctgtctcgccttggcacgccagaggccaatgggcaagaagaaaatgacctgccacctggaactccagatgcttttgcccaactgctgacctgcccctactgcgaccggggctacaagcgcttgacatcactgaaggagcacatcaagtaccgccacgagaagaatgaagagaacttttcctgccctctctgtagctacacgtttgcctaccgcacccagctcgagcggcatatggtgacacacaagccagggacagatcagcaccaaatgctaacccaaggagcaggtaatcgcaagttcaaatgcacagagtgtggcaaggccttcaaatataaacaccatctgaaagaacacctgcgaattcacagtggtgaaaaaccttacgagtgcccaaactgcaagaaacgtttctcccattctggttcctacagttcgcacatcagcagcaagaaatgtattggtttaatctctgtaaatggccgaatgagaaacaatatcaagacgggttcttcccctaattctgtttcttcttctcctactaattcagccattacccagttaagaaacaagttggagaatggaaaaccacttagtatgtctgaacagacaggcttacttaaaattaaaacagaaccactagacttcaatgactataaagttcttatggctacacacgggtttagtggcactagtccctttatgaatggtgggcttggagccaccagccctttaggagttcatccatctgctcagagtccaatgcagcacttaggtgtagggatggaagcccctttacttgggtttcccaccatgaatagtaatttaagtgaggtacaaaaggttctacagattgtggacaatactgtttccaggcaaaaaatggactgcaaggctgaagaaatttcaaagttgaaaggttatcacatgaaggatccatgctctcaacctgaggaacaaggagttacttctcctaatattccgcctgtcggtcttccggtagtgagtcataatggtgccactaaaagtattattgactatacgttggaaaaagtcaatgaagccaaagcttgcctccagagcttgactactgactcaaggagacagatcagtaatataaagaaagagaagctacgtactttaatagatttggtcactgatgacaaaatgattgagaaccacaacatatccactccattttcatgccagttctgtaaagaaagttttcctggccccatccctttgcatcagcatgaacgttacctttgtaagatgaatgaagagatcaaggcggtcctgcagcctcatgaaaacatagtccccaacaaagccggagtttttgttgataataaagccctcctcttgtcatctgtactttctgagaaaggaatgacaagccccatcaacccatacaaggaccacatgtctgtactcaaagcatactatgctatgaacatggagcccaactccgatgaactgctgaaaatttccattgctgtgggccttcctcaggaatttgtgaaggaatggtttgaacaacgaaaagtctaccagtactcaaattccaggtccccatccctggaaagaagctccaagccgttagctcccaacagtaaccctcccacaaaagactctttattacccaggtctcctgtaaaacctatggactccataacatcaccatctatagcagaactccacaacagtgttacgaattgtgatcctcctctcaggctaacaaaaccttcccattttaccaatattaaaccagttgaaaaattggaccactccaggagtaatactccttctcccttaaatctttcctccacatcttctaaaaactcccacagtagttcatacactccaaacagcttctcttctgaggagctccaggctgagcctttagacttgtcattaccaaaacaaatgaaagaacccaaaagtattatagccacaaagaacaaaacaaaagctagtagcatcagtttagatcataacagtgtttcttcctcatctgaaaactcagatgagcctctgaacttgacttttatcaagaaggaattttcaaattcaaataatctggacaacaaaagcactaacccagtgttcagcatgaacccatttagtgccaaacctttatacacagctcttccacctcaaagcgcatttccccctgctactttcatgccaccagtccagaccagtattcctgggctacgaccatacccaggactggatcagatgagcttcctaccacatatggcctacacctacccaactggagcagctacttttgctgatatgcagcaaaggagaaagtaccagcggaaacaaggatttcagggagaattgcttgatggagcacaagactacatgtcaggcctagatgatatgacagactccgactcctgtctgtctcgcaaaaagatcaagaagacagagagtggcatgtatgcatgtgacttatgtgacaagacattccagaaaagcagttcccttctgcgacataaatacgaacacacaggaaaaagaccacatcagtgtcagatttgtaagaaagcgtttaaacacaagcaccaccttatcgagcactcaaggcttcactcgggcgagaagccctatcagtgtgataaatgtggcaagcgcttctcacactcgggctcgtactcgcagcacatgaatcacaggtattcctactgcaagcgggaggcggaggagcgggaagcggcggagcgcgaggcgcgcgagaaagggcacttggaacccaccgagctgctgatgaaccgggcttacttgcagagcattacccctcaggggtactctgactcggaggagagggagagtatgccgagggatggcgagagcgagaaggagcacgagaaagaaggcgaggatggctacgggaagctgggcagacaggatggcgacgaggagttcgaggaggaagaggaagaaagtgaaaataaaagtatggatacggatcccgaaacgatacgagatgaagaagagactggagatcactccatggacgatagttcggaggatgggaaaatggaaaccaaatcagaccacgaggaagacaatatggaagatggcatgtaataaactactgcattttaagcttcctatttttttttccagtagtattgttacctgcttgaaaacactgctgtgttaagctgttcatgcacgtgcctgacgcttccaggaagctgtagagagggacagaaggggcggttcagccaagacagatgtagacggagttggagctgggtattgttaaaaactgcattatgcaaaaattttgtacagtgttaaggcctaaaaactgtgtggttcagagactaattcctgtgtttaatagcatttatactttaagcacaactagaaaattgtaagaattgcactctacttatgtatcactacaaactttaaaaaactatgtctaatttatattaatacattttaaaaaggtgcccgcactaccatacatcagtatttttattattattattgttattcctttttaatttaatgtgctcgcactacaatgcatcagtattatgattcctctgtactttcctttcgctattcatcaatttcccattttttttttcagcttaagtaaccacacaattttaggcctcaatttttttttttttctgtgaaggaacttgaagtgatgcatgtgtgaatttaagataccgaagtcttaaagtgacctggacgtgaaggaaaaagtaagatgagaaataaagaaagcctttgtaaggtggttttaaaagccttatatgcaaaccttttaatctgtgtttctgcaagtgccatccttgtacagtgttaagagggtaacatgggttacctttgcaccagcttcagtgttaagctcaccctgttctttgaagcacccatgtcagtattagaagaataggcagcagttccttagtttacatatgtttgtgcaattattttctgtacttttttgttcattaattttgtcagtattacaccaaactgtttttgcaacaaaaaaattttttttgcattcatttaattttaggtcaaataacattttatttatgtggctcattttatatttcctaattttatttatttcatactgtagtgtacagtattatagttcttcaatatatagatatattttagtaaaaaaggaacatgacgttgatcatttgggcaaattttacgtaaagagaagagcatttattgtgttttggaacattaattgtgagatgggatttttcaattttattattttatttttgtttttttccaattactggaaattccaaatttgggaacttttgatacgatcttgtgaaaacactgtattttcgactgaaaattccactttcttcatcttgttttttagctaaaaagagggactgttaaatacaatgtatgataccatgacaaaaatctttcctgaattgtctttgtaaaagtattattgaattttcaatttgtaatttcttttgaaaatgaccatgctcgaataaaaatgtagccaaactaaaaaaaaaaaaaaaaaaaaaaaaa

SEQ ID NO: 31 - DNA, 2957 bp, Homo sapiens
gaattcccaaacgtgcacaggggagtgagggcagggcgctcgcagggggcacgcagggagggcccagggcgccagggaggccgcgccgggctaatccgaaggggctgcgaggtcaggctgtaaccgggtcaatgtgtggaatattggggggctcggctgcagacttggccaaatggacgggactattaaggaggctctgtcggtggtgagcgacgaccagtccctctttgactcagcgtacggagcggcagcccatctccccaaggccgacatgactgcctcggggagtcctgactacgggcagccccacaagatcaaccccctcccaccacagcaggagtggatcaatcagccagtgagggtcaacgtcaagcgggagtatgaccacatgaatggatccagggagtctccggtggactgcagcgttagcaaatgcagcaagctggtgggcggaggcgagtccaaccccatgaactacaacagctatatggacgagaagaatggcccccctcctcccaacatgaccaccaacgagaggagagtcatcgtccccgcagaccccacactgtggacacaggagcatgtgaggcaatggctggagtgggccataaaggagtacagcttgatggagatcgacacatcctttttccagaacatggatggcaaggaactgtgtaaaatgaacaaggaggacttcctccgcgccaccaccctctacaacacggaagtgctgttgtcacacctcagttacctcagggaaagttcactgctggcctataatacaacctcccacaccgaccaatcctcacgattgagtgtcaaagaagacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactagaagcttcttctagctgaagcccatcctgcacacttactggatgctttggactcaacaggacatatgtggccttgaagggaagacaaaactggatgttctttcttgttggatagaacctttgtatttgttctttaaaaacattttttttaatgttggtaacttttgcttcctctacctgaacaaagagatgaataattccatgggccagtatgccagtttgaattctcagtctcctagcatcttgtgagttgcatattaagattactggaatggttaagtcatggttctgagaaagaagctgtacgttttctttatgtttttatgaccaaagcagtttcttgtcaatacacggggttcagtatgacacagaatcatggacttaacccgtcatgttctggtttgagatttagtgacaaatagaggtgggaagcttataatctaattttaggaggaccaaattcagcggatggcaactggaacattgattgtaaggccagtgaagttttcacccaactggaatttgatggaaagaaggtttgtgtgtttaagacgccaagggcattgcagaatccctctcagtggacagtatgcactcagctgaccactctctctagaaatagtcaagatatgaactaagaaattttaatgcaaatacatacattcctgaaagacggggaattaaattactaattttttttttttaaatgatgacagtggtcccagaacttggaaaagttgtagggatttctaaactcaagcagattcgcaagtgctgtgcgcttgtcagaccatcagaccagggccaaccaatcagaaggcaacttactgtataaattatgcagagttattttcctatatctcacagtattaaaaaataaataattaaaaattaagaataaataaacgagttgacctcggtcacaaaagcagttttactatcgaatcaatcgctgttatttttttttaatgtaatttgtacatcttttttcaatctgtacatttgggctgtcttgtatgtttttatgctcctttttaaaaagcataatatgcctatagctgaaaaggaaacagggctgtttaagtcactgacttatgagaaagcaaagcactggtacagttatttaacaggcatacacaagcagggaaaagataatccatttagatctttaatgctttggaaatgcgtgtaacagtactgcaataatcacagctctgggaaaaacaacgaaactttcccttgtggagaggagggattttcctgctctatataagcaacatatttttagacattaaaatatatataattttgcaggtaattgttgacttttttaactatattaagtgttaagctgacaactgtcaaagaagaccatgttgtaaaataatttgactaaataaatggttccttctctcaaaaaaaaaaaaa

SEQ ID NO: 32 - DNA, 1561 bp, Homo sapiens
cgcacgccacccgcccgccgcctgccagagctgctcggcccgcagccagggggacagcggctggtcggaggctcgcagtgctgtcggcgagaagcagtcgggtttggagcgcttgggtcgcgttggtgcgcggtggacacgagggaccccagttcccgcgagcagctccgcgccggccctgagagactaagctgaaactgctgctcagctcccaagatggtgccacccaaattgcatgtgcttttctgcctctgcggctgcctggctgtggtttatccttttgactggcaatacataaatcctgttgcccatatgaaatcatcagcatgggtcaacaaaatacaagtactgatggctgctgcaagctttggccaaactaaaatcccccggggaaatgggccttattccgttggttgtacagacttaatgtttgatcacactaataagggcaccttcttgcgtttatattatccatcccaagataatgatcgccttgacaccctttggatcccaaataaagaatatttttggggtcttagcaaatttcttggaacacactggcttatgggcaacattttgaggttactctttggttcaatgacaactcctgcaaactggaattcccctctgaggcctggtgaaaaatatccacttgttgttttttctcatggtcttggggcattcaggacactttattctgctattggcattgacctggcatctcatgggtttatagttgctgctgtagaacacagagatagatctgcatctgcaacttactatttcaaggaccaatctgctgcagaaataggggacaagtcttggctctaccttagaaccctgaaacaagaggaggagacacatatacgaaatgagcaggtacggcaaagagcaaaagaatgttcccaagctctcagtctgattcttgacattgatcatggaaagccagtgaagaatgcattagatttaaagtttgatatggaacaactgaaggactctattgatagggaaaaaatagcagtaattggacattcttttggtggagcaacggttattcagactcttagtgaagatcagagattcagatgtggtattgccctggatgcatggatgtttccactgggtgatgaagtatattccagaattcctcagcccctcttttttatcaactctgaatatttccaatatcctgctaatatcataaaaatgaaaaaatgctactcacctgataaagaaagaaagatgattacaatcaggggttcagtccaccagaattttgctgacttcacttttgcaactggcaaaataattggacacatgctcaaattaaagggagacatagattcaaatgcagctattgatcttagcaacaaagcttcattagcattcttacaaaagcatttaggacttcataaagattttgatcagtgggactgcttgattgaaggagatgatgagaatcttattccagggaccaacattaacacaaccaatcaacacatcatgttacagaactcttcaggaatagagaaatacaattaggattaaaataggtttttta

SEQ ID NO: 33 - DNA, 1110 bp, Homo sapiens
gacagagcccgggccacggagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgccttacaggaacagaagaggaaagagagacacagctgcagaggccacctggcttgcgcctaatgtgtttgagcatacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggcattttatgtctttcttgtaagatactgccttgtttaatgttaattatgcagtgtttccctctgtgttagagcagagaggtttcgatatttattgatgttttcacaaagaacaggaaaataaaatatttaaaaatat

SEQ ID NO: 34 - DNA, 2690 bp, Homo sapiens
ataaaaacccagaaagccccagaaacaaagacttcacggacaaagtcccttggaaccagagagaagccgggatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttctgactcagaccataggaggccaacccaaaataagcaggcgtgacctgccaggcacactgagccagcagcctggctctcccagccaggttctgactcttggcacagcatggagtcacagccacttgggatagagagggaatgtaatggtggcctggggcttctgaggcttctggggcttcagtcttttccatgaacttctcccctggtagaaagaagatgaatgagcaaaaccaaatattccagagactgggactaagtgtaccagagaagggcttggactcaagcaagatttcagatttgtgaccattagcatttgtcaacaaagtcacccacttcccactattgcttgcacaaaccaattaaacccagtagtggtgactgtgggctccattcaaagtgagctcctaagccatgggagacactgatgtatgaggaatttctgttcttccatcacctccccccccccgccaccctcccactgccaaagaacttggaaatagtgatttccacagtgactccactctgagtcccagagccaatcagtagccagcatctgcctccccttcactcccaccgcaggatttgggctcttggaatcctggggaacatagaactcatgacggaagagttgagacctaacgagaaatagaaatggggaactactgctggcagtggaactaagaaagcccttaggaagaatttttatatccactaaaatcaaacaattcagggagtgggctaagcacgggccatatgaataacatggtgtgcttcttaaaatagccataaaggggagggactcatcatttccatttacccttcttttctgactatttttcagaatctctcttcttttcaagttgggtgatatgttggtagattctaatggctttattgcagcgattaataacaggcaaaaggaagcagggttggtttcccttctttttgttcttcatctaagccttctggttttatgggtcagagttccgactgccatcttggacttgtcagcaaaaaaaaaaaataataataataataaggcctgctgtgtaagctgacagtatttgtagctgatagggggttgggaggaaagtgtctactaggagggtggggtgagattctgtgttgatgtaggaggccgagaaggcccttaactcaaagtagcttatttatccaaaatgttctggatgcatcatctccaaccaaggaccccttatttatcatgcctttgttctcttttccctcagatgtatatttctttaaaaataattttcctaataacaaaacttatttctaaaacagcttaaaaattcaaagaaaaaccccaaacactgacattacctacacttccactacccaaagacaaaatgtgcccactgtgtgcttttgagtgtattttcttttagtttgttttttgttgggtgcatatttatgataataacaatgatggacttcaattgtactcactgttctattgttggttttaattagcagcaagttgtgatcactttcccaggtgaataaatcatttcaaagcattaaaaaaaaaaaaaa

SEQ ID NO: 35 - DNA, 2035 bp, Homo sapiens
gaattccgggctccggggatgaggtcgcggccggcgggtcccgcgctgttgctgctgctgctcttcctcggagcggccgagtcggtgcgtcgggcccagcctccgcgccgctacaccccagactggccgagcctggattctcggccgctgccggcctggttcgacgaagccaagttcggggtgttcatccactggggcgtgttctcggtgcccgcctggggcagcgagtggttctggtggcactggcagggcgaggggcggccgcagtaccagcgcttcatgcgcgacaactacccgcccggcttcagctacgccgacttcggaccgcagttcactgcgcgcttcttccacccggaggagtgggccgacctcttccaggccgcgggcgccaagtatgtagttttgacgacaaagcatcacgaaggcttcacaaactggccgagtcctgtgtcttggaactggaactccaaagacgtggggcctcatcgggatttggttggtgaattgggaacagctctccggaagaggaacatccgctatggactataccactcactcttagagtggttccatccactctatctacttgataagaaaaatggcttcaaaacacagcattttgtcagtgcaaaaacaatgccagagctgtacgaccttgttaacagctataaacctgatctgatctggtctgatggggagtgggaatgtcctgatacttactggaactccacaaattttctttcatggctctacaatgacagccctgtcaaggatgaggtggtagtaaatgaccgatggggtcagaactcttcctgtcaccatggaggatactataactgtgaagataaattcaagccacagagcttgccagatcacaagtgggagatgtgcaccagcattgacaagttttcctggggctatcgtcgtgacatggcattgtctgatgttacagaagaatctgaaatcatttcggaactggttcagacagtaagtttgggaggcaactatcttctgaacattggaccaactaaagatggactgattgttcccatcttccaagaaaggcttcttgctgttgggaaatggctgagcatcaatggggaggctatctatgcctccaaaccatggcgggtgcaatgggaaaagaacacaacatctgtatggtatacctcaaagggatcggctgtttatgccatttttctgcactggccagaaaatggagtcttaaaccttgaatcccccataactacctcaactacaaagataacaatgctgggaattcaaggagatctgaagtggtccacagatccagataaaggtctcttcatctctctaccccagttgccaccctctgctgtccccgcagagtttgcttggactataaagctgacaggagtgaagtaatcatttgagtgcaagaagaaagaggcgctgctcactgttttcctgcttcagtttttctcttatagtaccatcactataatcaacgaacttctcttctccacccagagatggcttttccaacacattttaattaaaggaactgagtacattaccctgatgtctaaatggaccaaagatctgagatccattgtgattatatctgtatcaggtcagcagaagaaggaactgagcagttgaactctgagttcatcaattctaatatttggaaattatctacaatggaatcttccctctgttctctgataacctacttgcttactcaatgcctttaagccaagtcaccctgttgcctatgggaggaggtggaaggatttggcaagctcaaccacatgctatttagttagcatcagttgtcaccaacagtctttctgcaaagggcaggagagctttgggggaaaggaaaaggcttaccaggctgctatggtcaactcttcagaaattttcagagcaatctaaaagcgccaaaattcgctatgtttacagtgatactattaagaaaatgaatgtgattctgctctgtctttttaagtatgatcaaataaaaaatttgtacatcacaatcatttctacc

SEQ ID NO: 36 - DNA, 11185 bp, Homo sapiens
gctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacctaaagaggctacaaccatcgatttgagtatcctcgcagaaactgcatcacccagtttatccaaagaaccacaaatggtttctgatagaactacaccaatcatccctttagttgatgaattacctgtcattccaacagagttccctcccgtgggaaatattgtcagttttgaacagaaagccacagtccaacctcaggctatcacagatagtttagccaccaaattacccacacctactggcagtaccaagaagccctgggatatggatgactactcaccttctgcttcaggacctcttggaaagctagacatatcagaaattaaggaagaagtgctccagagtacaactggcgtctctcattatgctacggattcatgggatggtgtcgtggaagataaacaaacacaagaatcggttacacagattgaacaaatagaagtgggtcctttggtaacatctatggaaatcttaaagcacattccttccaaggaattccctgtaactgaaacaccattggtaactgcaagaatgatcctggaatccaaaactgaaaagaaaatggtaagcactgtttctgaattggtaaccacaggtcactatggattcaccttgggagaagaggatgatgaagacagaacacttacagttggatctgatgagagcaccttgatctttgaccaaattcctgaagtcattacggtgtcaaagacttcagaagacaccatccacactcatttagaagacttggagtcagtctcagcatccacaactgtttcccctttaattatgcctgataataatggatcatccatggatgactgggaagagagacaaactagtggtaggataacggaagagtttcttggcaaatatctgtctactacaccttttccatcacagcatcgtacagaaatagaattgtttccttattctggtgataaaatattagtagagggaatttccacagttatttatccttctctacaaacagaaatgacacatagaagagaaagaacagaaacactaataccagagatgagaacagatacttatacagatgaaatacaagaagagatcactaaaagtccatttatgggaaaaacagaagaagaagtcttctctgggatgaaactctctacatctctctcagagccaattcatgttacagagtcttctgtggaaatgaccaagtcttttgatttcccaacattgataacaaagttaagtgcagagccaacagaagtaagagatatggaggaagactttacagcaactccaggtactacaaaatatgatgaaaatattacaacagtgcttttggcccatggtactttaagtgttgaagcagccactgtatcaaaatggtcatgggatgaagataatacaacatccaagcctttagagtctacagaaccttcagcctcttcaaaattgccccctgccttactcacaactgtggggatgaatggaaaggataaagacatcccaagtttcactgaagatggagcagatgaatttactcttattccagatagtactcaaaagcagttagaggaggttactgatgaagacatagcagcccatggaaaattcacaattagatttcagccaactacatcaactggtattgcagaaaagtcaactttgagagattctacaactgaagaaaaagttccacctatcacaagcactgaaggccaagtttatgcaaccatggaaggaagtgctttgggtgaagtagaagatgtggacctctctaagccagtatctactgttccccaatttgcacacacttcagaggtggaaggattagcatttgttagttatagtagcacccaagagcctactacttatgtagactcttcccataccattcctctttctgtaattcccaagacagactggggagtgttagtaccttctgttccatcagaagatgaagttctaggtgaaccctctcaagacatacttgtcattgatcagactcgccttgaagcgactatttctccagaaactatgagaacaacaaaaatcacagagggaacaactcaggaagaattcccttggaaagaacagactgcagagaaaccagttcctgctctcagttctacagcttggactcccaaggaggcagtaacaccactggatgaacaagagggcgatggatcagcatatacagtctctgaagatgaattgttgacaggttctgagagggtcccagttttagaaacaactccagttggaaaaattgatcacagtgtgtcttatccaccaggtgctgtaactgagcacaaagtgaaaacagatgaagtggtaacactaacaccacgcattgggccaaaagtatctttaagtccagggcctgaacaaaaatatgaaacagaaggtagtagtacaacaggatttacatcatctttgagtccttttagtacccacattacccagcttatggaagaaaccactactgagaaaacatccctagaggatattgatttaggctcaggattatttgaaaagcccaaagccacagaactcatagaattttcaacaatcaaagtcacagttccaagtgatattaccactgccttcagttcagtagacagacttcacacaacttcagcattcaagccatcttccgcgatcactaagaaaccacctctcatcgacagggaacctggtgaagaaacaaccagtgacatggtaatcattggagaatcaacatctcatgttcctcccactacccttgaagatattgtagccaaggaaacagaaaccgatattgatagagagtatttcacgacttcaagtcctcctgctacacagccaacaagaccacccactgtggaagacaaagaggcctttggacctcaggcgctttctacgccacagcccccagcaagcacaaaatttcaccctgacattaatgtttatattattgaggtcagagaaaataagacaggtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcaactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagttgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccagg

SEQ ID NO: 37 - DNA, 1670 bp, Homo sapiens
attgagagtggctctaacaagtgccatttttccttgttagctttcatttctcagccctttacaagattaaaatagtctgcagtttaatctctccaaagctttacggacagtgattctgtcctaaacaagacagtgactccaggatttctgaagactattgtggaagaagcatccattaaggccaagctataacatcagaaatggtgaatgaatacaagaaaattcttttgctgaaaggatttgagctcatggatgattatcattttacatcaattaagtccttactggcctatgatttaggactaactacaaaaatgcaagaggaatacaacagaattaagattacagatttgatggaaaaaaagttccaaggcgttgcctgtctagacaaactaatagaacttgccaaagatatgccatcacttaaaaaccttgttaacaatcttcgaaaagagaagtcaaaagttgctaagaaaattaaaacacaagaaaaagctccagtgaaaaaaataaaccaggaagaagtgggtcttgcggcacctgcacccaccgcaagaaacaaactgacatcggaagcaagagggaggattcctgtagctcagaaaagaaaaactccaaacaaagaaaagactgaagccaaaaggaataaggtgtcccaagagcagagtaagcccccaggtccctcaggagccagcacatctgcagctgtggatcatcccccactaccccagacctcatcatcaactccatccaacacttcgtttactccgaatcaggaaacccaggcccaacggcaggtggatgcaagaagaaatgttccccaaaacgacccagtgacagtggtggtactgaaagcaacagcgccatttaaatacgagtccccagaaaatgggaaaagcacaatgtttcatgctacagtggccagtaagactcaatatttccatgtgaaagtcttcgacatcaacttgaaagagaaatttgtaaggaagaaggtcattaccatatctgattactctgaatgtaaaggagtaatggaaataaaggaagcatcatctgtgtctgactttaatcaaaattttgaggtcccaaacagaattatcgaaatagcaaataaaactcccaagatcagtcaactttacaagcaagcatctggaacaatggtgtatgggttgtttatgttacaaaagaaaagcgtacacaagaagaacacaatttatgaaatacaggataatacaggatccatggatgtagtggggagtggaaaatggcacaatatcaagtgtgagaaaggagataaacttcgactcttctgccttcaactgagaacagttgaccgcaagctgaaactggtgtgtggaagtcacagcttcatcaaggtcatcaaggccaagaaaaacaaggaaggaccaatgaatgttaattgaaatatgaaagctgaaatgcaacaaacaacttccgcttaaaacaattaagttgttaataactgtgattttgtaaatttcagtaattcatttaaatgatgtttcagtagatatattctagcatattaagagcttttataactgagttatagattagtttgctttctggaataaaattttcttcttatactcttccttttttttagatattacattttgcttttatgacattcacgaggcaaaaaaccg

SEQ ID NO: 38 - DNA, 4065 bp, Homo sapiens
gggaacaaacttcagaaggaggagagacaccgggcccagggcaccctcgcgggcggacccaagcagtgagggcctgcagccggccggccagggcagcggcaggcgcggcccggacctacgggaggaagccccgagccctcggcgggctgcgagcgactccccggcgatgcctcacaactccatcagatctggccatggagggctgaaccagctgggaggggcctttgtgaatggcagacctctgccggaagtggtccgccagcgcatcgtagacctggcccaccagggtgtaaggccctgcgacatctctcgccagctccgcgtcagccatggctgcgtcagcaagatccttggcaggtactacgagactggcagcatccggcctggagtgatagggggctccaagcccaaggtggccacccccaaggtggtggagaagattggggactacaaacgccagaaccctaccatgtttgcctgggagatccgagaccggctcctggctgagggcgtctgtgacaatgacactgtgcccagtgtcagctccattaatagaatcatccggaccaaagtgcagcaaccattcaacctccctatggacagctgcgtggccaccaagtccctgagtcccggacacacgctgatccccagctcagctgtaactcccccggagtcaccccagtcggattccctgggctccacctactccatcaatgggctcctgggcatcgctcagcctggcagcgacaagaggaaaatggatgacagtgatcaggatagctgccgactaagcattgactcacagagcagcagcagcggaccccgaaagcaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactggggcgcaacctctcgactcaccagacctaccccgtggtggcagatcctcactcacccttcgccataaagcaggaaacccccgaggtgtccagttctagctccaccccttcctctttatctagctccgcctttttggatctgcagcaagtcggctccggggtcccgcccttcaatgcctttccccatgctgcctccgtgtacgggcagttcacgggccaggccctcctctcagggcgagagatggtggggcccacgctgcccggatacccaccccacatccccaccagcggacagggcagctatgcctcctctgccatcgcaggcatggtggcaggaagtgaatactctggcaatgcctatggccacaccccctactcctcctacagcgaggcctggcgcttccccaactccagcttgctgagttccccatattattacagttccacatcaaggccgagtgcaccgcccaccactgccacggcctttgaccatctgtagttgccatggggacagtgggagcgactgagcaacaggaggactcagcctgggacaggccccagagagtcacacaaaggaatctttatttattacatgaaaaataaccacaagtccagcattgcggcacactccctgtgtggttaatttaatgaaccatgaaagacaggatgaccttggacaaggccaaactgtcctccaagactccttaatgaggggcaggagtcccagggaaagagaaccatgccatgctgaaaaagacaaaattgaagaagaaatgtagcccccagccggtacccaccaaaggagagaagaagcaatagccgaggaacttggggggatggcgaatggttcctgcccgggcccaaggggtgcacagggcacctccatggctccattattaacacaactctagcaattatggaccataagcacttccctccagcccacaagtcacagcctggtgccgaggctctcctcaccagccacccagggagtcacctccctcagcctcccgcctgccccacacggaggctctggctgtcctctttctccactccatttgcttggctctttctacacctccctcttgggcatgggctgagggctggagcgagtccctcagaaattccaccaggctgtcagctgacctcttttgcctgctgctgtgaaggtatagcaccaccccaggtcctcctgcagtgcggcatccccttggcagctgccgtcagccaggccagccccagggagcttaaaacagacattccacagggcctgggcccctgggaggtgaggtgtggtgtgcggcttcacccagggcagaacaaggcagaatcgcaggaaacccgcttccccttcctgacagctcctgccaagccaaatgtgcttcctgcagctcacgcccaccagctactgaagggacccaaggcaccccctgaagccagcgatagagggtccctctctgctccccagcagctcctgcccccaaggcctgactgtatatactgtaaatgaaactttgtttgggtcaagcttccttctttctaacccccagactttggcctctgagtgaaatgtctctctttgccctgtggggcttctctccttgatgcttctttctttttttaaagacaacctgccattaccacatgactcaataaaccattgctcttcatctcaggctttggggttggctggggaaggaggcatcccggggctgggctttctcccaagaacatcagagctgagtagccgacaaactcactttggggccgtgggctggaagggaccatctgatgccccagagctctggcttggccttctccctctgcctttaattcacgttgaacgctgggtacctcactcatcccaagttcttcaacactgagcaaatgcaaggatagcacagtactgagccaaccatagactccccacaaggagttgctgttgttattaacaggaagccagagaatcagcagggtgggttagtgagggatccgggaatagctgtgactggagcctgcataaacagctctgaagggagagagaagactgggctctcttgtgtgccaggcacagtatggaaggcttcatataagttaagctgaaattagccctgttttacatacagcttcattttacatatgaggaaactgaggctttgaaaaaaatgagatgtcttgtccaagatgaaaagtagtagattcaaccaagtcctcttactctaagcccaacgcttttacccaaaaccccagagtcctcatcagggatgccaaatggttctagacccagtggaggttctggagctgccactggggatttaatttcttttgatttgctaaagatttgacctgactgaatggagaggtagagtgtagtgtggccaggacaaggtgagggaggctgtagagacttagcactttaggccaaccacctccaggaaatctgggaaatgcaatgtgacagctcgggctctgcactccagggggctgtctggtgtccacatggaccttctccatgtgggacacagctggaacaagggggcaggggcctgcagctgggatgcccaggtgaatatgggcagctggacaaacaacactgggattgagtcagatagaaggggcccaaggactccagggctgggaggacagaggctgggagagagggctcttacctccttaggcctcccaaagagcggttagggatgctgccatggatggcatggcagggggaaccctcctggaagaaaatccatctcttctgaagggatctgagatgcggctggtttttcaatggcagaacttccctctgcagcgcgactccgaatccatgacatctgagagtcttcctgaccacaaacctctgggatcccgagggctccctacccaagaatcactttgagcacagcatcccaaggagcccatagagcgatcccttgcattcacagccacagcccctctggggacactctgtacccccggcagaccctttccaactcacaaccaataaaggggcttgggctgtgctttgactaaggtgaaaaaaaaaaa

SEQ ID NO: 39 - DNA, 5820 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttataaaaaaaaaaaaaaaaaaaaaaaaaaaaaaa

SEQ ID NO: 40 - DNA, 3108 bp, Homo sapiens
gaaatactcgtctctggtaaagtctgagcaggacagggtggctgactggcagatccagaggttcccttggcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttcca

SEQ ID NO: 41 - DNA, 2939 bp, Homo sapiens
atctgaaaaattaataattccttaattatcaaatatccattatttaaatttataattgtgtcataaatattgtcataaatagatttgctgttttaaagcttgttccttcattttctctgttttgttttagataaacattgtcataaatagatttgttgttttaaagcttgttccttcattttctctgattgttttgttttagattcagaggttacttatgcttgtttgttacatggatgttacatgtgtaatgggggatattggacttctagtgtactcatcacccatatactgaacactgtactcaaaagggattgaaagaaactaggaaacttggcaggaagatcattcttaagccaggaaaaaaatttttaatgctcacatgtgaacatgtgatggtcataccagaaggagcacccacctccctccctctgtgacagacacattttcttagccttcacctttccttctttcaagttgctgaaaatccacagtgtttctgttcatttgttactttcattctcacctatcttctctcttgctccatctaccagaacaataattccccatataatacttctcacttcacttttcaacgcaggacctcttgttggtctgatctgtttgtctgtccgctttatcaatattatcagatgtaagtttacatgaatacacacacatattcactaaactgaggggaaaaaatgccttgtaggtcataaaaaagcagggaaattcccaacaattcatatttgatccctggatccaggggtggcagcaataagcctgctttagatatttactccccattttatgatccggtggtttggtttttcaaatgatgatatggctcctttcgcaatgacttgatgtttaggaggtgtgcttcaataaatacattttaaaatcaacaatcaagttagagttgtacaaatggctctgaaatgtcccactacactgttagaccaagggcacagattgtgcttctgtactatttatcctagtatccctcggcatatattaactgctctaaaaatctccttggctacacgctgcatcaaatcaaagttaaatgttataccacctttctattctatttttaatattcaaagagggtgctcagattttagaacaaatttcaatgtttaagtacacacaaaaaaatcattaactcatatatttcaagagtaggaaatgggaactggtgttaaaactcttataacaaatgtcactgtcttaagggacagtgtttaaaaacgcatacctggccgggcgcggtggctcatgcctgtaatcccagcactttgggaggccgaggccggcggatcacaaggaaaacaaactcaggaagaaaaaggaaagcagaagtgatcaaggagagcgctcgagttgcaatattttcctttggctgctgacaggcagttactataaagcattgtgcatggacaccatcttcttgtattatacaagaaaggagtgtacctatcacacacagggggaaaaatgctcttttgggtgctaggcctcctaatcctctgtggttttctgtggactcgtaaaggaaaactaaagattgaagacatcactgataagtacatttttatcactggatgtgactcgggctttggaaacttggcagccagaacttttgataaaaagggatttcatgtaatcgctgcctgtctgactgaatcaggatcaacagctttaaaggcagaaacctcagagagacttcgtactgtgcttctggatgtgaccgacccagagaatgtcaagaggactgcccagtgggtgaagaaccaagttggggagaaaggtctctggggtctgatcaataatgctggtgttcccggcgtgctggctcccactgactggctgacactagaggactacagagaacctattgaagtgaacctgtttggactcatcagtgtgacactaaatatgcttcctttggtcaagaaagctcaagggagagttattaatgtctccagtgttggaggtcgccttgcaatcgttggagggggctatactccatccaaatatgcagtggaaggtttcaatgacagcttaagacgggacatgaaagcttttggtgtgcacgtctcatgcattgaaccaggattgttcaaaacaaacttggcagatccagtaaaggtaattgaaaaaaaactcgccatttgggagcagctgtctccagacatcaaacaacaatatggagaaggttacattgaaaaaagtctagacaaactgaaaggcaataaatcctatgtgaacatggacctctctccggtggtagagtgcatggaccacgctctaacaagtctcttccctaagactcattatgccgctggaaaagatgccaaaattttctggatacctctgtctcacatgccagcagctttgcaagactttttattgttgaaacagaaagcagagctggctaatcccaaggcagtgtgactcagctaaccacaaatgtctcctccaggctatgaaattggccgatttcaagaacacatctccttttcaaccccattccttatctgctccaacctggactcatttagatcgtgcttatttggattgcaaaagggagtcccaccatcgctggtggtatcccagggtccctgctcaagttttctttgaaaaggagggctggaatggtacatcacataggcaagtcctgccctgtatttaggctttgcctgcttggtgtgatgtaagggaaattgaaagacttgcccattcaaaatgatctttaccgtggcctgccccatgcttatggtccccagcatttacagtaacttgtgaatgttaagtatcatctcttatctaaatattaaaagataagtcaaacattaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - DNA, 1900 bp, Homo sapiens
acaactctcagaggagcattgcccgtcagacagcaactcagagaataaccagagaacaaccagattgaaacaatggaggatctttgtgtggcaaacacactctttgccctcaatttattcaagcatctggcaaaagcaagccccacccagaacctcttcctctccccatggagcatctcgtccaccatggccatggtctacatgggctccaggggcagcaccgaagaccagatggccaaggtgcttcagtttaatgaagtgggagccaatgcagttacccccatgactccagagaactttaccagctgtgggttcatgcagcagatccagaagggtagttatcctgatgcgattttgcaggcacaagctgcagataaaatccattcatccttccgctctctcagctctgcaatcaatgcatccacagggaattatttactggaaagtgtcaataagctgtttggtgagaagtctgcgagcttccgggaagaatatattcgactctgtcagaaatattactcctcagaaccccaggcagtagacttcctagaatgtgcagaagaagctagaaaaaagattaattcctgggtcaagactcaaaccaaaggcaaaatcccaaacttgttacctgaaggttctgtagatggggataccaggatggtcctggtgaatgctgtctacttcaaaggaaagtggaaaactccatttgagaagaaactaaatgggctttatcctttccgtgtaaactcggctcagcgcacacctgtacagatgatgtacttgcgtgaaaagctaaacattggatacatagaagacctaaaggctcagattctagaactcccatatgctggagatgttagcatgttcttgttgcttccagatgaaattgccgatgtgtccactggcttggagctgctggaaagtgaaataacctatgacaaactcaacaagtggaccagcaaagacaaaatggctgaagatgaagttgaggtatacataccccagttcaaattagaagagcattatgaactcagatccattctgagaagcatgggcatggaggacgccttcaacaagggacgggccaatttctcagggatgtcggagaggaatgacctgtttctttctgaagtgttccaccaagccatggtggatgtgaatgaggagggcactgaagcagccgctggcacaggaggtgttatgacagggagaactggacatggaggcccacagtttgtggcagatcatccttttctttttcttattatgcataagataaccaactgcattttatttttcggcagattttcctcaccctaaaactaagcgtgctgcttctgcaaaagatttttgtagatgagctgtgtgcctcagaattgctatttcaaattgccaaaaatttagagatgttttctacatatttctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtaccctccatgcccttctgtaataaatatctggaaaaaacattaaacaataggcaaatatatgttatgtgcatttctagaaatacataacacatatatatgtctgtatcttatattcaattgcaagtatataataaataaacctgcttccaaacaac

SEQ ID NO: 43 - protein, 469 aa, Homo sapiens
MEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 44 - protein, 97 aa, Homo sapiens
MPSQMEHAMETMMFTFHKFAGDKGYLTKEDLRVLMEKEFPGFLENQKDPLAVDKIMKDLDQCRDGKVGFQSFFSLIAGLTIACNDYFVVHMKQKGKK

SEQ ID NO: 45 - protein, 60 aa, Homo sapiens
MSESGEMSEFGYIMELIAKGKMPDWRRGYRCRQGCGKTTELATATDFSQTGNKSGKHFKT

SEQ ID NO: 46 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 47 - protein, 402 aa, Homo sapiens [sequence omitted]

SEQ ID NO: 48 - protein, 1067 aa, Homo sapiens
MNSMDRHIQQTNDRLQCIKQHLQNPANFHNAATELLDWCGDPRAFQRPFEQSLMGCLTVVSRVAAQQGFDLDLGYRLLAVCAANRDKFTPKSAALLSSWCEELGRLLLLRHQKSRQSDPPGKLPMQPPLSSMSSMKPTLSHSDGSFPYDSVPWQQNTNQPPGSLSVVTTVWGVTNTSQSQVLGNPMANANNPMNPGGNPMASGMTTSNPGLNSPQFAGQQQQFSAKAGPAQPYIQQSMYGRPNYPGSGGFGASYPGGPNAPAGMGIPPHTRPPADFTQPAAAAAAAAVAAAAATATATATATVAALQETQNKDINQYGPMGPTQAYNSQFMNQPGPRGPASMGGSMNPASMAAGMTPSGMSGPPMGMNQPRPPGISPFGTHGQRMPQQTYPGPRPQSLPIQNIKRPYPGEPNYGNQQYGPNSQFPTQPGQYPAPNPPRPLTSPNYPGQRMPSQPSSGQYPPPTVNMGQYYKPEQFNGQNNTFSGSSYSNYSQGNVNRPPRPVPVANYPHSPVPGNPTPPMTPGSSIPPYLSPSQDVKPPFPPDIKPNMSALPPPPANHNDELRLTFPVRDGVVLEPFRLEHNLAVSNHVFHLRPTVHQTLMWRSDLELQFKCYHHEDRQMNTNWPASVQVSVNATPLTIERGDNKTSHKPLHLKHVCQPGRNTIQITVTACCCSHLFVLQLVHRPSVRSVLQGLLKKRLLPAEHCITKIKRNFSSVAASSGNTTLNGEDGVEQTAIKVSLKCPITFRRIQLPARGHDCKHVQCFDLESYLQLNCERGTWRCPVCNKTALLEGLEVDQYMWGILNAIQHSEFEEVTIDPTCSWRPVPIKSDLHIKDDPDGIPSKRFKTMSPSQMIMPNVMEMIAALGPGPSPYPLPPPPGGTNSNDYSSQGNNYQGHGNFDFPHGNPGGTSMNDFMHGPPQLSHPPDMPNNMAALEKPLSHPMQETMPHAGSSDQPHPSIQQGLHVPHPSSQSGPPLHHSGAPPPPPSQPPRQPPQAAPSSHPHSDLTFNPSSALEGQAGAQGASDMPEPSLDLLPELTNPDELLSYLDPPDLPSNSNDDLLSLFENN

SEQ ID NO: 49 - protein, 367 aa, Homo sapiens
MVMEVGTLDAGGLRALLGERAAQCLLLDCRSFFAFNAGHIAGSVNVRFSTIVRRRAKGAMGLEHIVPNAELRGRLLAGAYHAVVLLDERSAALDGAKRDGTLALAAGALCREARAAQVFFLKGGYEAFSASCPELCSKQSTPMGLSLPLSTSVPDSAESGCSSCSTPLYDQGGPVEILPFLYLGSAYHASRKDMLDALGITALINVSANCPNHFEGHYQYKSIPVEDNHKADISSWFNEAIDFIDSIKNAGGRVFVHCQAGISRSATICLAYLMRTNRVKLDEAFEFVKQRRSIISPNFSFMGQLLQFESQVLAPHCSAEAGSPAMAVLDRGTSTTTVFNFPVSIPVHSTNSALSYLQSPITTSPSC

SEQ ID NO: 50 - protein, 492 aa, Homo sapiens
MVKFPALTHYWPLIRFLVPLGITNIAIDFGEQALNRGIAAVKEDAVEMLASYGLAYSLMKFFTGPMSDFKNVGLVFVNSKRDRTKAVLCMVVAGAIAAVFHTLIAYSDLGYYIINKLHHVDESVGSKTRRAFLYLAAFPFMDAMAWTHAGILLKHKYSFLVGCASISDVIAQVVFVAILLHSHLECREPLLIPILSLYMGALVRCTTLCLGYYKNIHDIIPDRSGPELGGDATIRKMLSFWWPLALILATQRISRPIVNLFVSRDLGGSSAATEAVAILTATYPVGHMPYGWLTEIRAVYPAFDKNNPSNKLVSTSNTVTAAHIKKFTFVCMALSLTLCFVMFWTPNVSEKILIDIIGVDFAFAELCVVPLRIFSFFPVPVTVRAHLTGWLMTLKKTFVLAPSSVLRIIVLIASLVVLPYLGVHGATLGVGSLLAGFVGESTMVAIAACYVYRKQKKKMENESATEGEDSAMTDMPPTEEVTDIVEMREENE

SEQ ID NO: 51 - protein, 310 aa, Homo sapiens
MAATGANAEKAESHNDCPVRLLNPNIAKMKEDILYHFNLTTSRHNFPALFGDVKFVCVGGSPSRMKAFIRCVGAELGLDCPGRDYPNICAGTDRYAMYKVGPVLSVSHGMGIPSISIMLHELIKLLYYARCSNVTIIRIGTSGGIGLEPGTVVITEQAVDTCFKAEFEQIVLGKRVIRKTDLNKKLVQELLLCSAELSEFTTVVGNTMCTLDFYEGQGRLDGALCSYTEKDKQAYLEAAYAAGVRNIEMESSVFAAMCSACGLQAAVVCVTLLNRLEGDQISSPRNVLSEYQQRPQRLVSYFIKKKLSKA

SEQ ID NO: 52 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 53 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 54 - protein, 465 aa, Homo sapiens
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 55 - protein, 317 aa, Homo sapiens [sequence omitted]

SEQ ID NO: 56 - protein, 240 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRK

SEQ ID NO: 57 - protein, 418 aa, Homo sapiens
MQALVLLLCIGALLGHSSCQNPASPPEEGSPDPDSTGALVEEEDPFFKVPVNKLAAAVSNFGYDLYRVRSSMSPTTNVLLSPLSVATALSALSLGAEQRTESIIHRALYYDLISSPDIHGTYKELLDTVTARQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 58 - protein, 307 aa, Homo sapiens
MGTVLSLSPSYRKATLFEDGAATVGHYTAVQNSKNAKDKNLKRHSIISVLPWKRIVAVSAKKKNSKKVQPNSSYQNNITHLNNENLKKSLSCANLSTFAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 59 - protein, 290 aa, Homo sapiens
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 60 - protein, 233 aa, Homo sapiens
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 61 - protein, 236 aa, Homo sapiens
MAETKLQLFVKASEDGESVGHCPSCQRLFMVLLLKGVPFTLTTVDTRRSPDVLKDFAPGSQLPILLYDSDAKTDTLQIEDFLEETLGPPDFPSLAPRYRESNTAGNDVFHKFSAFIKNPVPAQDEALYQQLLRALARLDSYLRAPLEHELAGEPQLRESRRRFLDGDRLTLADCSLLPKLHIVDTVCAHFRQAPIPAELRGVRRYLDSAMQEKEFKYTCPHSAEILAAYRPAVHPR

SEQ ID NO: 62 - protein, 426 aa, Homo sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 63 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 64 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHIFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 65 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 66 - protein, 396 aa, Homo sapiens
MWNSSDANFSCYHESVLGYRYVAVSWGVVVAVTGTVGNVLTLLALAIQPKLRTRFNLLIANLTLADLLYCTLLQPFSVDTYLHLHWRTGATFCRVFGLLLFASNSVSILTLCLIALGRYLLIAHPKLFPQVFSAKGIVLALVSTWVVGVASFAPLWPIYILVPVVCTCSFDRIRGRPYTTILMGIYFVLGLSSVGIFYCLIHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMCFAVFLCFALSYIPFLLLNILDARVQAPRVVHMLAANLTWLNGCINPVLYAAMNRQFRQAYGSILKRGPRSFHRLH

SEQ ID NO: 67 - protein, 378 aa, Homo sapiens
MRMLVSGRRVKKWQLIIQLFATCFLASLMFFWEPIDNHIVSHMKSYSYRYLINSYDFVNDTLSLKHTSAGPRYQYLINHKEKCQAQDVLLLLFVKTAPENYDRRSGIRRTWGNENYVRSQLNANIKTLFALGTPNPLEGEELQRKLAWEDQRYNDIIQQDFVDSFYNLTLKLLMQFSWANTYCPHAKFLMTADDDIFIHMPNLIEYLQSLEQIGVQDFWIGRVHRGAPPIRDKSSKYYVSYEMYQWPAYPDYTAGAAYVISGDVAAKVYEASQTLNSSLYIDDVFMGLCANKIGIVPQDHVFFSGEGKTPYHPCIYEKMMTSHGHLEDLQDLWKNATDPKVKTISKGFFGQIYCRLMKIILLCKISYVDTYPCRAAFI

SEQ ID NO: 68 - protein, 287 aa, Homo sapiens
MHRLMGVNSTAAAAAGQPNVSCTCNCKRSLFQSMEITELEFVQIIIIVVVMMVMVVVITCLLSHYKLSARSFISRHSQGRRREDALSSEGCLWPSESTVSGNGIPEPQVYAPPRPTDRLAVPPFAQRERFHRFQPTYPYLQHEIDLPPTISLSDGEEPPPYQGPCTLQLRDPEQQLELNRESVRAPPNRTIFDSDLMDSARLGGPCPPSSNSGISATCYGSGGRMEGPPPTYSEVIGHYPGSSFQHQQSSGPPSLLEGTRLHHTHIAPLESAAIWSKEKDKQKGHPL

SEQ ID NO: 69 - protein, 273 aa, Homo sapiens
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 70 - protein, 97 aa, Homo sapiens
MADKVLKEKRKLFIHSMGEGTINGLLDELLQTRVLNQEEMEKVKRENATVMDKTRALIDSVIPKGAQACQICITYICEEDSYLAETLGLSAGPIPGN

SEQ ID NO: 71 - protein, 1032 aa, Homo sapiens
MSQQGYVATPPYSQPQPGIGLSPPHYGHYGDPSHTASPTGMMKPAGPLGATATRGMLPPGPPPPGPHQFGQNGAHATGHPPQRFPGPPPVNNVASSHAPYQPSAQSSYPGPISTSSVTQLGSQLSAMQINSYGSGMAPPSQGPPGPLSATSLQTPPRPPQPSILQPGSQVLPPPPTTLNGPGASPLPLPMYRPDGLSGPPPPNAQYQPPPLPGQTLGAGYPPQQANSGPQMAGAQLSYPGGFPGGPAQMAGPPQPQKKLDPDSIPSPIQVIENDRASRGGQVYATNTRGQIPPLVTTDCMIQDQGNASPRFIRCTTYCFPCTSDMAKQAQIPLAAVIKPFATIPSNESPLYLVNHGESGPVRCNRCKAYMCPFMQFIEGGRRYQCGFCNCVNDVPPFYFQHLDHIGRRLDHYEKPELSLGSYEYVATLDYCRKSKPPNPPAFIFMIDVSYSNIKNGLVKLICEELKTMLEKIPKEEQEETSAIRVGFITYNKVLHFFNVKSNLAQPQMMVVTDVGEVFVPLLDGFLVNYQESQSVIHNLLDQIPDMFADSNENETVFASVIQAGMEALKAADCPGKLFIFHSSLPTAEAPGKLKNRDDKKLVNTDKEKILFQPQTNVYDSLAKDCVAHGCSVTLFLFPSQYVDVASLGLVPQLTGGTLYKYNNFQMHLDRQQFLNDLRNDIEKKIGFDAIMRVRTSTGFRATDFFGGILMNNTTDVEMAAIDCDKAVTVEFKHDDKLSEDSGALIQCAVLYTTISGQRRLRIHNLGLNCSSQLADLYKSCETDALINFFAKSAFKAVLHQPLKVIREILVNQTAHMLACYRKNCASPSAASQLILPDSMKVLPVYMNCLLKNCVLLSRPEISTDERAYQRQLVMTMGVADSQLFFYPQLLPIHTLDVKSTMLPAAVRCSESRLSEEGIFLLANGLHMFLWLGVSSPPELIQGIFNVPSFAHINTDMTLLPEVGNPYSQQLRMIMGIIQQKRPYSMKLTIVKQREQPEMVFRQFLVEDKGLYGGSSYVDFLCCVHKEICQLLN

SEQ ID NO: 72 - protein, 1214 aa, Homo sapiens
MKQPIMADGPRCKRRKQANPRRKNVVNYDNVVDTGSETDEEDKLHIAEDDGIANPLDQETSPASVPNHESSPHVSQALLPREEEEDEIREGGVEHPWHNNEILQASVDGPEEMKEDYDTMGPEATIQTAINNGTVKNANCTSDFEEYFAKRKLEERDGHAVSIEEYLQRSDTAIIYPEAPEELSRLGTPEANGQEENDLPPGTPDAFAQLLTCPYCDRGYKRLTSLKEHIKYRHEKNEENFSCPLCSYTFAYRTQLERHMVTHKPGTDQHQMLTQGAGNRKFKCTECGKAFKYKHHLKEHLRIHSGEKPYECPNCKKRFSHSGSYSSHISSKKCIGLISVNGRMRNNIKTGSSPNSVSSSPTNSAITQLRNKLENGKPLSMSEQTGLLKIKTEPLDFNDYKVLMATHGFSGTSPFMNGGLGATSPLGVHPSAQSPMQHLGVGMEAPLLGFPTMNSNLSEVQKVLQIVDNTVSRQKMDCKAEEISKLKGYHMKDPCSQPEEQGVTSPNIPPVGLPVVSHNGATKSIIDYTLEKVNEAKACLQSLTTDSRRQISNIKKEKLRTLIDLVTDDKMIENHNISTPFSCQFCKESFPGPIPLHQHERYLCKMNEEIKAVLQPHENIVPNKAGVFVDNKALLLSSVLSEKGMTSPINPYKDHMSVLKAYYAMNMEPNSDELLKISIAVGLPQEFVKEWFEQRKVYQYSNSRSPSLERSSKPLAPNSNPPTKDSLLPRSPVKPMDSITSPSIAELHNSVTNCDPPLRLTKPSHFTNIKPVEKLDHSRSNTPSPLNLSSTSSKNSHSSSYTPNSFSSEELQAEPLDLSLPKQMKEPKSIIATKNKTKASSISLDHNSVSSSSENSDEPLNLTFIKKEFSNSNNLDNKSTNPVFSMNPFSAKPLYTALPPQSAFPPATFMPPVQTSIPGLRPYPGLDQMSFLPHMAYTYPTGAATFADMQQRRKYQRKQGFQGELLDGAQDYMSGLDDMTDSDSCLSRKKIKKTESGMYACDLCDKTFQKSSSLLRHKYEHTGKRPHQCQICKKAFKHKHHLIEHSRLHSGEKPYQCDKCGKRFSHSGSYSQHMNHRYSYCKREAEEREAAEREAREKGHLEPTELLMNRAYLQSITPQGYSDSEERESMPRDGESEKEHEKEGEDGYGKLGRQDGDEEFEEEEEESENKSMDTDPETIRDEEETGDHSMDDSSEDGKMETKSDHEEDNMEDGM

SEQ ID NO: 73 - protein, 452 aa, Homo sapiens
MDGTIKEALSVVSDDQSLFDSAYGAAAHLPKADMTASGSPDYGQPHKINPLPPQQEWINQPVRVNVKREYDHMNGSRESPVDCSVSKCSKLVGGGESNPMNYNSYMDEKNGPPPPNMTTNERRVIVPADPTLWTQEHVRQWLEWAIKEYSLMEIDTSFFQNMDGKELCKMNKEDFLRATTLYNTEVLLSHLSYLRESSLLAYNTTSHTDQSSRLSVKEDPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 74 - protein, 441 aa, Homo sapiens
MVPPKLHVLFCLCGCLAVVYPFDWQYINPVAHMKSSAWVNKIQVLMAAASFGQTKIPRGNGPYSVGCTDLMFDHTNKGTFLRLYYPSQDNDRLDTLWIPNKEYFWGLSKFLGTHWLMGNILRLLFGSMTTPANWNSPLRPGEKYPLVVFSHGLGAFRTLYSAIGIDLASHGFIVAAVEHRDRSASATYYFKDQSAAEIGDKSWLYLRTLKQEEETHIRNEQVRQRAKECSQALSLILDIDHGKPVKNALDLKFDMEQLKDSIDREKIAVIGHSFGGATVIQTLSEDQRFRCGIALDAWMFPLGDEVYSRIPQPLFFINSEYFQYPANIIKMKKCYSPDKERKMITIRGSVHQNFADFTFATGKIIGHMLKLKGDIDSNAAIDLSNKASLAFLQKHLGLHKDFDQWDCLIEGDDENLIPGTNINTTNQHIMLQNSSGIEKYN

SEQ ID NO: 75 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 76 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 77 - protein, 461 aa, Homo sapiens
MRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNSSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 78 - protein, 3396 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 79 - protein, 407 aa, Homo sapiens
MVNEYKKILLLKGFELMDDYHFTSIKSLLAYDLGLTTKMQEEYNRIKITDLMEKKFQGVACLDKLIELAKDMPSLKNLVNNLRKEKSKVAKKIKTQEKAPVKKINQEEVGLAAPAPTARNKLTSEARGRIPVAQKRKTPNKEKTEAKRNKVSQEQSKPPGPSGASTSAAVDHPPLPQTSSSTPSNTSFTPNQETQAQRQVDARRNVPQNDPVTVVVLKATAPFKYESPENGKSTMFHATVASKTQYFHVKVFDINLKEKFVRKKVITISDYSECKGVMEIKEASSVSDFNQNFEVPNRIIEIANKTPKISQLYKQASGTMVYGLFMLQKKSVHKKNTIYEIQDNTGSMDVVGSGKWHNIKCEKGDKLRLFCLQLRTVDRKLKLVCGSHSFIKVIKAKKNKEGPMNVN

SEQ ID NO: 80 - protein, 450 aa, Homo sapiens
MPHNSIRSGHGGLNQLGGAFVNGRPLPEVVRQRIVDLAHQGVRPCDISRQLRVSHGCVSKILGRYYETGSIRPGVIGGSKPKVATPKVVEKIGDYKRQNPTMFAWEIRDRLLAEGVCDNDTVPSVSSINRIIRTKVQQPFNLPMDSCVATKSLSPGHTLIPSSAVTPPESPQSDSLGSTYSINGLLGIAQPGSDKRKMDDSDQDSCRLSIDSQSSSSGPRKHLRTDAFSQHHLEPLECPFERQHYPEAYASPSHTKGEQGLYPLPLLNSTLDDGKATLTPSNTPLGRNLSTHQTYPVVADPHSPFAIKQETPEVSSSSSTPSSLSSSAFLDLQQVGSGVPPFNAFPHAASVYGQFTGQALLSGREMVGPTLPGYPPHIPTSGQGSYASSAIAGMVAGSEYSGNAYGHTPYSSYSEAWRFPNSSLLSSPYYYSSTSRPSAPPTTATAFDHL

SEQ ID NO: 81 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 82 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 83 - protein, 319 aa, Homo sapiens
MLFWVLGLLILCGFLWTRKGKLKIEDITDKYIFITGCDSGFGNLAARTFDKKGFHVIAACLTESGSTALKAETSERLRTVLLDVTDPENVKRTAQWVKNQVGEKGLWGLINNAGVPGVLAPTDWLTLEDYREPIEVNLFGLISVTLNMLPLVKKAQGRVINVSSVGGRLAIVGGGYTPSKYAVEGFNDSLRRDMKAFGVHVSCIEPGLFKTNLADPVKVIEKKLAIWEQLSPDIKQQYGEGYIEKSLDKLKGNKSYVNMDLSPVVECMDHALTSLFPKTHYAAGKDAKIFWIPLSHMPAALQDFLLLKQKAELANPKAV

SEQ ID NO: 84 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 85 - DNA, 24 bp, Artificial Sequence
  Primer
tttctgctgtcttgggtgcattgg

SEQ ID NO: 86 - DNA, 24 bp, Artificial Sequence
  Primer
accacttcgtgatgattctgccct

SEQ ID NO: 87 - DNA, 26 bp, Artificial Sequence
  Synthetic oligonucleotide
ntgctgctctacctccaccatgccan

SEQ ID NO: 88 - DNA, 24 bp, Artificial Sequence
  Primer
agtctggtccttgcactcctgttt

SEQ ID NO: 89 - DNA, 24 bp, Artificial Sequence
  Primer
tgtcctgcatgagatctgtctgct

SEQ ID NO: 90 - DNA, 25 bp, Artificial Sequence
  Synthetic oligonucleotide
nagcatggcgagcatggcggctatn

SEQ ID NO: 91 - DNA, 24 bp, Artificial Sequence
  Primer
gaaggttgtgaaatcaagcaggcg

SEQ ID NO: 92 - DNA, 24 bp, Artificial Sequence
  Primer
aagtgcccttgacttagtggtggt

SEQ ID NO: 93 - DNA, 26 bp, Artificial Sequence
  Synthetic oligonucleotide
nccgcttggtttgaaggcagctttgn

SEQ ID NO: 94 - DNA, 24 bp, Artificial Sequence
  Primer
tgctggtgaatgccctctacttca

SEQ ID NO: 95 - DNA, 24 bp, Artificial Sequence
  Primer
agagacagtgctgccgtctgattt

SEQ ID NO: 96 - DNA, 22 bp, Artificial Sequence
  Synthetic oligonucleotide
acggccagtggaagactccctn

SEQ ID NO: 97 - DNA, 18 bp, Artificial Sequence
  Primer
ccacccatggcaaattcc

SEQ ID NO: 98 - DNA, 19 bp, Artificial Sequence
  Primer
tcgctcctggaagatggtg

SEQ ID NO: 99 - DNA, 24 bp, Artificial Sequence
  Synthetic oligonucleotide
nggcaccgtcaaggctgagaacgn

(19) United States
(12) Patent Application Publication
(10) Pub. No.: US 2009/0074790 A1
(43) Pub. Date: Mar. 19, 2009

(54) Methods for measuring transforming growth factor beta (TGF-beta) receptor signaling activity and uses thereof

(76) Inventors: Michael Reiss, Princeton, NJ (US); Judy Kleinstein, Woodbridge, CT (US)

Correspondence Address: KLAUBER & JACKSON, 411 HACKENSACK AVENUE, HACKENSACK, NJ, 07601, US

(21) Appl. No.: 11/489,287

(22) Filed: Jul. 19, 2006

Related U.S. Application Data
(63) Continuation-in-part of application No. PCT/US05/01703, filed on Jan. 20, 2005.
(60) Provisional application No. 60/537,719, filed on Jan. 20, 2004.

(51) Int. Cl. A61K 39/395; G01N 33/53; C12Q 1/68; C40B 30/00; C40B 40/06

(52) U.S. Cl. 424/158.1; 435/7.72; 435/6; 506/7; 506/16

(57) ABSTRACT

Methods are disclosed for determining the optimal biologic dose of a TGFβ receptor kinase inhibitor for administration to patients in need of such therapy and for monitoring the effectiveness of therapy with a TGFβ receptor kinase inhibitor in patients receiving such therapy. Kits comprising antibodies and reagents useful in such methods are also disclosed.

### CROSS-REFERENCE TO RELATED APPLICATIONS

This application is a Continuation-in-Part of International Application PCT/US2005/001703, filed Jan. 20, 2005, which in turn, claims benefit of priority under 35 U.S.C. 119(e) to Provisional Application Ser. No. 60/537,719, filed Jan. 20, 2004. Applicants claim the benefit of 35 U.S.C. 120 as to said International Application, and all of said applications are incorporated by reference herein in their entireties.

### GOVERNMENT RIGHTS CLAUSE

The research leading to the present invention was supported by Grant No. NIH 1U01CA94431. Accordingly, the Government has certain rights in the invention.

### FIELD OF THE INVENTION

The invention relates generally to the field of diagnostics, and more particularly to methods for determining the optimal biologic dose of a Transforming Growth Factor-beta (TGFβ) receptor (TβR) kinase inhibitor (TβKI) for administration to patients in need of such therapy and for monitoring the effectiveness of therapy with a TβR receptor kinase inhibitor in patients receiving such therapy. Kits comprising antibodies and reagents useful in such methods are also disclosed.

### BACKGROUND OF THE INVENTION

TGF-β is a highly conserved multifunctional cytokine that regulates a multitude of cellular functions during development as well as in adult organisms (Massague, J. et al.(2000), Genes & Development, Vol. 14:627-644). There are three mammalian isoforms of TGF-β (TGF-β 1-3), which are structurally nearly identical. TGF-β family members are secreted as latent precursor molecules (LTGF-β) requiring activation to form the mature form for receptor binding and subsequent activation of signal transduction pathways.

Activation is a very complex process and involves conformational changes of LTGF-β induced either by cleavage of the precursor by various proteases or by physical interaction of the precursor with other proteins, such as thrombospondin-1, leading to the active mature form (Roberts, A. B. et al (1998), Miner Electrolyte Metab 24: 111-119). Upon activation, TGF-β family members initiate their cellular action by binding to serine/threonine kinase receptors.

The TGF-β receptor family consists of two structurally similar subfamilies, type I and type II receptors. Type I receptors have a region rich in glycine and serine residues (GS domain) that precedes the receptor kinase domain (Huse M. et al (1999), Cell 96: 425-436). Type I and type II receptors act in sequence. Following its extracellular activation, TGF-β binds primarily to the type II receptor (TβR-II), followed by the recruitment of the type I receptor (TβR-I) into a configuration with two TβR-II molecules and a single TGF-β dimer. Once this ternary complex forms, the TβR-II kinase phosphorylates specific serine residues located at the juxtamembrane GS domain of TβR-I, which, in turn, activates the TβR-I serine-threonine kinase. This is the key step in transducing all of TGFβ signals, thus positioning TβR-I as the gatekeeper of the TGFβ signaling pathway (Massague, J. et al., (2000), Genes & Development, Vol. 14:627-644).

Following ligand activation, signaling from TβRI to the nucleus occurs predominantly by phosphorylation of cytoplasmic mediators belonging to the Smad family (Massague J. (2000), Genes Dev 14:627-644). In particular, two of these proteins, Smad2 and Smad3, become transiently associated with and phosphorylated by the activated TβR-I receptor kinase at the last two Ser residues of the C-terminal SSXS motif in the Mad-homology-2 (MH2) domain. (Abdollah, S. et al. (1997), J Biol Chem, 272:27678-27685). Phosphorylated receptor-associated R-Smads form heteromeric complexes with the common mediator Smad, Smad4, which are then translocated to the nucleus, (Pierreux, C. E. et al., (2000), Mol Cell Biol, 20:9041-9054) where they interact with DNA and other components of the transcriptional machinery to regulate the expression of TGFβ target genes (Massague, J. et al. (2000), EMBO J., 19:1745-1754)

In self-renewing epithelia TGFβ appears to fulfill two major functions. TGFβ plays a key role in maintaining the balance between cell renewal and cell differentiation and loss (Massague, J. et al. (2000), Genes & Development, 14:627-644). For example, in transgenic mouse models, constitutive expression of TGFβ1 or Smad2 in keratinocytes results in disordered epidermal proliferation and differentiation (Cui, W. et al. (1995), Genes Dev, 9:945-955; Ito, Y. et al. (2001), Dev Biol, 236:181-194). TGFβ also mediates the response to tissue injury. Injury results in a rapid locally increased activation of TGFβ, which induces epithelial cells to assume a fibroblastoid and dispersed phenotype (epithelial-to-mesenchymal transdifferentiation, EMT) and to produce ECM components of what later becomes a scar (Roberts, A. B. et al. (2001), Chest, 120:43S47S). Normally, this process is self-limited in space and time, allowing epithelial cells to revert back to their cohesive epitheloid phenotype (Barcellos-Hoff, M. H. (1998), Radiat Res, 150:S109-120, 1998). However, in chronic inflammatory conditions, loss of epithelial structures and the associated fibrosis have been attributed to persistent activation of TGFβ (Border, W. A. et al. (1994), N. Engl. J. Med., 331:1286-1292). Moreover, not only do many cancers retain the ability to engage this TGFβ-mediated repair function, but in some it becomes constitutively activated (Piek, E. et al. (2001), Adv Cancer Res, 83:1-54). Thus, in this case, TGFβ acquires the properties of an oncogene.

TGFβ demonstrates both autocrine and paracrine tumor-promoting effects, the latter including stimulation of tumor angiogenesis and inhibition of anti-tumor immunity. While TGFβ1 clearly plays a major role in vasculogenesis during embryonic development (Pepper, M. S. (1997), Cytokine Growth Factor Rev, 8:21-43) and for the establishment and maintenance of blood vessel wall integrity (Kulkarni, A. B. et al. (1993), Proc Natl Acad Sci USA, 90:770-774, the role of TGFβs in the process of tumor angiogenesis is less clear (Pepper, M. S. (1997), Cytokine Growth Factor Rev, 8:21-43). One possible explanation for these discrepant observations is that, in most of the settings in which TGFβ appeared to induce angiogenesis, this was preceded by the induction of an inflammatory reaction (Pepper, M. S. (1997), Cytokine Growth Factor Rev, 8:21-43). Thus, TGFβ-associated angiogenesis in vivo may, in fact, be context-dependent.

Several lines of experimental evidence support the notion that tumor-associated TGFβ allows tumor cells to escape from immune surveillance (reviewed in (Letterio, J. J. et al. (1997), Clin Immunol Immunopathol, 84:244-250): First, bioactive TGFβ has been shown to block the clonal expansion of activated lymphocytes (Gorelik, L. et al. (2000), J Immunol, 165:4773-4777). Conversely, TGFβ1-null mice develop a multi-system autoimmune disorder characterized by a deficiency of epidermal dendritic (Langerhans) cells and hyperactivation of most immune cell populations (Kulkarni, A. B. et al. (1993), Proc Natl Acad Sci USA, 90:770-774). These studies suggest that blocking TGFβ-mediated immune suppression is a potentially useful strategy to enhance antitumor immune responses (Park, J. A. et al. (1997), Cancer Gene Therapy, 4:42-50, 1997).

Accordingly, the testing of TGFβ receptor kinase inhibitors for inhibition of tumor cell growth and progression is an active area for research. However, there are no known means to determine the optimal dose levels of such inhibitors that are necessary for achieving the desired effect, nor is there a means for monitoring the effectiveness of therapy with such inhibitors in patients receiving such therapy. Accordingly, there is a need for development of sensitive and reliable assays in patients under consideration for such therapy. The present application addresses these needs.

### SUMMARY OF THE INVENTION

In its broadest aspect, the present invention relates to diagnostic approaches to be utilized in patients who are candidates for therapy with Transforming Growth Factor-beta (TGFβ) receptor kinase inhibitors. In particular, it is an object of the present invention to provide for methods for determining a biologically effective dose of a Transforming Growth Factor-beta (TGFβ) receptor kinase inhibitor (TβKI) or for determining the optimal biologic dose of a Transforming Growth Factor-beta (TGFβ) receptor kinase inhibitor (TβKI) for administration to a patient in need of such therapy, or for monitoring the effectiveness of therapy with a TGFβ receptor kinase inhibitor in patients receiving such therapy, or for determining whether a patient would be responsive to such therapy. It is also an object of the present invention to determine the effect of TGFβ receptor kinase inhibitors on TGFβ signaling and to screen for novel drug candidates that modulate TGFβ signaling. Diagnostic kits and kits for screening for potential drug candidates are also disclosed.

Accordingly, a first aspect of the invention provides for a method for determining the optimal biologic dose of a Transforming Growth Factor-beta (TGFβ) receptor kinase inhibitor for administration to a patient in need of such therapy, comprising the steps of:
a) obtaining a tissue or cell sample from said patient prior to initiation of therapy to establish baseline levels of TGFβ receptor kinase activity;
b) processing said sample to enable release of phosphorylated Smad2 and -3 (pSmad2/3) from the cells within the sample;
c) contacting said processed sample with a solid substrate to allow binding of the released pSmad2/3 to said substrate;
d) measuring the amount of pSmad2/3 in said sample by detecting said pSmad2/3 with an antibody specific for pSmad2/3;
e) obtaining a tissue sample from the patient after treatment with a TGFβ receptor kinase inhibitor given at various doses; and repeating steps b) through d);
f) comparing the levels of pSmad2/3 in the tissue sample obtained in step e) to the level of pSmad2/3 in the sample obtained in step a);
   wherein a decrease in the levels of pSmad2/3 compared to baseline levels is indicative of achieving the optimal dose of the TGFβ receptor kinase inhibitor.

In a particular embodiment, the tissue or cell sample is selected from the group consisting of tumor tissue, skin, whole blood, peripheral blood mononuclear cells (PBMC), gingiva, colon, endometrium and any other accessible tissue or cell of the human body. In another particular embodiment, the method for measuring the amount of pSmad2/3 in said sample is by detecting said pSmad2/3 with an antibody specific for pSmad2/3. The method of detecting may be accomplished by use of an immunoassay. In a further particular embodiment, the immunoassay is an enzyme linked immunoassay, a radioimmunoassay, or a Western blot assay. In yet another particular embodiment, the antibody specific for pSmad2/3 is selected from the group consisting of a polyclonal antibody, a monoclonal antibody, a single chain antibody, a human or humanized antibody, and Fab fragments thereof. The antibody may be a chimeric antibody. The antibody may be produced in animals, including but not limited to horses, goats, sheep, mice, rats, rabbits and guinea pigs. In another particular embodiment, the patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

A second aspect of the invention provides for a method for monitoring the effectiveness of therapy with a Transforming Growth Factor-beta (TGFβ) receptor kinase inhibitor in patients receiving such therapy, comprising the steps of:
a) obtaining a tissue or cell sample from said patient prior to initiation of therapy to establish baseline levels of TGFβ receptor kinase activity;
b) processing said sample to enable release of pSmad2/3 from the cells within the sample;
c) contacting said processed sample with a solid substrate to allow binding of the released pSmad2/3 to said substrate;
d) measuring the amount of pSmad2/3 in said sample by detecting said pSmad2/3 with an antibody specific for pSmad2/3;
e) obtaining a tissue sample from the patient after treatment with a TGFβ receptor kinase inhibitor given at various doses; and repeating steps b) through d);
f) comparing the levels of pSmad2/3 in the tissue sample obtained in step e) to the level of pSmad2/3 in the sample obtained in step a);
   wherein a decrease in the levels of pSmad2/3 to compared baseline levels is reflective of the effectiveness of therapy with a TGFβ receptor kinase inhibitor.

In a particular embodiment, the tissue or cell sample is selected from the group consisting of tumor tissue, skin, whole blood, peripheral blood mononuclear cells (PBMC), gingiva, colon, endometrium and any other accessible tissue or cell of the human body. In another particular embodiment, the method for measuring the amount of pSmad2/3 in said sample is by detecting said pSmad2/3 with an antibody specific for pSmad2/3. The method of detecting may be accomplished by use of an immunoassay. In another particular embodiment, the immunoassay is an enzyme linked immunoassay, a radioimmunoassay, or a Western blot assay. In yet another particular embodiment, the antibody specific for pSmad2/3 is selected from the group consisting of a polyclonal antibody, a monoclonal antibody, a single chain antibody, a human or humanized antibody, and Fab fragments thereof. They may be chimeric antibodies. They may be produced in animals, including but not limited to horses, goats, sheep, mice, rats, rabbits and guinea pigs. In another particular embodiment, the patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

A third aspect of the invention provides for a method for determining the optimal biologic dose of a TGFβ receptor kinase inhibitor for administration to a patient in need of such therapy, comprising the steps of:
a) obtaining a plasma sample from said patient prior to initiation of therapy to establish baseline levels of TGFβ receptor kinase activity;
b) contacting said sample with TGFβ-responsive test cells in vitro; wherein said cells are pretreated with TGFβ at a dose sufficient to activate TGFβ receptor kinase activity;
c) processing said cells to enable release of pSmad2/3 from the cells;
d) contacting the extract from said processed cells with a solid substrate to allow binding of the released pSmad2/3 to said substrate;
e) measuring the amount of pSmad2/3 in said extract using an antibody specific for pSmad2/3;
f) obtaining a plasma sample from the patient after treatment with a TGFβ receptor kinase inhibitor given at various doses; and repeating steps b) through e);
g) comparing the levels of pSmad2/3 from test cells incubated with plasma samples from step f) to the level of pSmad2/3 from test cells incubated with plasma samples from step a);
   wherein a decrease in the levels of pSmad2/3 compared to baseline levels is indicative of achieving the optimal biologic dose of the TGFβ receptor kinase inhibitor.

In a particular embodiment, the TGFβ responsive test cells are selected from the group consisting of Sweig cells, BALB/MK cells, HKc/HPV16 cells, Mink lung cells, HaCaT cells, MDA-MB-231 cells, and MDA-MB435 cells and any other human or rodent, epithelial or lymphoid cell line in which TGFβ reproducibly induces phosphorylation of Smad2/3 in a dose-dependent manner. In a particular embodiment, the method for measuring the amount of pSmad 2/3 in the extract is through use of an antibody specific for pSmad2/3. In another particular embodiment, the antibody may be used in an immunoassay. The immunoassay is an enzyme linked immunoassay, a radioimmunoassay, or a Western blot assay. In yet another particular embodiment, the antibody specific for pSmad2/3 is selected from the group consisting of a polyclonal antibody, a monoclonal antibody, a single chain antibody, a human or humanized antibody, and Fab fragments thereof. In yet another particular embodiment, the patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

A fourth aspect of the invention provides for a method for monitoring the effectiveness of therapy with a TGFβ receptor kinase inhibitor in patients receiving such therapy, comprising the steps of:
a) obtaining a plasma sample from said patient prior to initiation of therapy to establish baseline levels of TGFβ receptor kinase activity;
b) contacting said sample with TGFβ responsive test cells in vitro; wherein said cells are pretreated with TGFβ at a dose sufficient to activate TGFβ receptor kinase activity;
c) processing said cells to enable release of pSmad2/3 from the cells;
d) contacting the extract from said processed cells with a solid substrate to allow binding of the released pSmad2/3 to said substrate;
e) measuring the amount of pSmad2/3 in said extract using an antibody specific for pSmad2/3;
f) obtaining a plasma sample from the patient after treatment with a TβR-1 receptor kinase inhibitor given at various doses; and repeating steps b) through e);
g) comparing the levels of pSmad2/3 from test cells incubated with plasma samples from step f) to the level of pSmad2/3 from test cells incubated with plasma samples from step a);
   wherein a decrease in the levels of pSmad2/3 compared to baseline levels is reflective of the effectiveness of therapy with a TGFβ receptor kinase inhibitor.

In a particular embodiment, the TGFβ responsive test cells are selected from the group consisting of Sweig cells, BALB/MK cells, HKc/HPV16 cells, Mink lung cells, HaCaT cells, MDA-MB-231 cells, and MDA-MB-435 cells and any other human or rodent, epithelial or lymphoid cell line in which TGβ reproducibly induces phosphorylation of Smad2/3 in a dose-dependent manner. In a particular embodiment, the method for measuring pSmad2/3 is through use of an antibody specific for pSmad2/3. In another particular embodiment, the levels of pSmad2/3 are measured through use of an immunoassay. In another particular embodiment, the immunoassay is an enzyme linked immunoassay, a radioimmunoassay, or a Western blot assay. In yet another particular embodiment, the antibody specific for pSmad2/3 is selected from the group consisting of a polyclonal antibody, a monoclonal antibody, a single chain antibody, a human or humanized antibody, and Fab fragments thereof. In yet another particular embodiment, the patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

In another particular embodiment, the TGFβ responsive cells may be maintained in suspension and the antibody to pSmad2/3 associated with the cells may be detected using a flow cytometer or a fluorescence activated cell sorter. In yet another particular embodiment, the method of detection of the antibody may be accomplished through use of various detection methods, including, but not limited to use of radiolabels, enzymes, and other chromophores or fluorescent reagents that allow for detection using microscopic techniques or through use of flow cytometric techniques known to those skilled in the art.

A fifth aspect of the invention provides for a method of identifying by high throughput screening a therapeutic agent that inhibits TGFβ receptor kinase activity, comprising contacting TGFβ responsive cells with said agent, and detecting the binding of an antibody specific for pSmad2/3 as described herein, or a derivative of fragment thereof, wherein the inability to detect binding of the antibody to pSmad2/3 is indicative of an active TGFβ receptor kinase inhibitory agent. In a particular embodiment, the antibody specifically binds to phosphorylated Smad2/3, and the binding occurs only if the agent in question does not inhibit the TGFβ receptor kinase activity. The method comprises contacting said TGFβ responsive cells with said agent and determining whether said agent prevents the phosphorylation of Smad2/3, as measured by the detection (or lack thereof) of bound anti-pSmad2/3 antibody. In one embodiment, the anti-pSmad2/3 antibody may be detected by a second antibody conjugated to an enzyme, a radioisotope or any other molecule that may be detected by fluorescence or the like. In another embodiment, the method comprises the steps of:
a) incubating a culture of TGFβ responsive cells with increasing concentrations of a test agent, or with control culture medium, for a time sufficient to allow binding of TGFβ to its receptors and to activate the receptor kinases;
b) fixing and permeabilizing the cells in order to allow for antibody binding to the phosphorylated Smad2/3 molecules;
c) incubating the cells with an antibody specific for phosphorylated Smad2/3 (pSmad2/3) for a time sufficient to allow binding of the antibody to pSmad2/3;
d) detecting and quantitating the amount of pSmad2/3 antibody bound by incubating with a labeled second antibody having specificity for the pSmad2/3 antibody;
e) comparing the amount of labeled second antibody bound to TGFβ responsive cells without test compound to the amount of labeled second antibody bound to TGFβ responsive cells with test compound; and
   wherein the amount of labeled antibody bound correlates inversely with the potential of the test compound for inhibiting TGFB receptor kinase activity.

In a particular embodiment, the TGFβ responsive test cells are selected from the group consisting of Sweig cells, BALB/MK cells, HKc/HPV16 cells, Mink lung cells, HaCaT cells, MDA-MB-231 cells, and MDA-MB-435 cells and any other human or rodent, epithelial or lymphoid cell line in which TGFβ reproducibly induces phosphorylation of Smad2/3 in a dose-dependent manner. In yet another particular embodiment, the antibody specific for pSmad2/3 is selected from the group consisting of a polyclonal antibody, a monoclonal antibody, a single chain antibody, a human or humanized antibody, and Fab fragments thereof.

A sixth aspect of the invention provides for methods of using such agents identified by the methods described herein to treat a subject suffering from a TGFβ-dependent disease or condition and accordingly is a candidate for therapy with a TGFβ receptor kinase inhibitor. In addition, the methods described herein would aid in predicting those patients who would be most responsive to the therapies described as TβR kinase inhibitors. These diseases or conditions refer to pathologic conditions that depend on the activity of one or both TGFβ receptor kinases. TGFβ receptor kinases either directly or indirectly participate in the signal transduction pathways of a variety of cellular activities including proliferation, adhesion and migration, and differentiation. Diseases associated with TGFβ receptor kinase activities include cancer (eg. the proliferation of tumor cells and the pathologic neovascularization that supports solid tumor growth), ocular neovascularization (diabetic retinopathy, age-related macular degeneration, and the like), inflammation (psoriasis, rheumatoid arthritis, and the like), pulmonary fibrosis, liver cirrhosis, chronic glomerulonephritis, and keloids. Furthermore, patients receiving radiation therapy, patients having arterial restenosis, or patients having atherosclerosis may also benefit from such therapy. In a particular embodiment, such agents are provided in the form of a pharmaceutical composition with a pharmaceutically acceptable carrier for treatment of subjects in need of such therapy. In another particular embodiment the subject to be treated is a mammal, preferably a human, although use of the agents for treatment of such conditions in other mammals is also conceived.

A seventh aspect of the invention provides for a diagnostic test kit for determining the optimal biologic dose of a TGFβ receptor kinase inhibitor to be administered to a patient in need of such therapy, or for monitoring the effectiveness of therapy with a TGFβ receptor kinase inhibitor in patients receiving such therapy, or for predicting whether a subject is a candidate for therapy with a TGFβ receptor kinase inhibitor comprising,
a) a predetermined amount of an antibody specific for pSmad2/3;
b) a predetermined amount of a specific binding partner of said antibody;
c) buffers and other reagents necessary for monitoring detection of antibody bound to pSmad2/3 in a bodily sample; and
d) directions for use of said kit;
   wherein either said antibody or said specific binding partner are detectably labeled.

An eighth aspect of the invention provides for methods of treatment of patients suffering from a TGFβ-dependent disease or condition by treatment of the patients with a pharmaceutical composition comprising the anti-pSmad2/3 antibodies described herein and a pharmaceutically acceptable carrier, or other antibodies, fragments, analogs or mimics thereof that affect downstream signaling events. In a particular embodiment, the antibody would be a polyclonal or monoclonal antibody. In another particular embodiment, the antibody would be a human or humanized antibody. In a further particular embodiment, the antibody would be delivered to cells having Fc receptors to allow for binding and internalization of the antibody. In yet another particular embodiment, the antibody would be an F(ab) or F(ab)₂ fragment or other antigen/epitope binding fragment thereof. In a further particular embodiment, the antibody would be delivered to cells by first permeabilizing the cells to allow entry of the antibody or fragment thereof.

Inhibitors of the TGFβ-β signaling pathway may prove useful for treatment of patients suffering from cancer or other proliferative disorders in which this pathway plays a crucial role. It is also important to note that there are differences between treating a patient with conventional cytotoxic therapies and the therapies that are currently under investigation which target this signaling pathway. For example, in the case of conventional non-targeted cytotoxic chemotherapeutic agents, the selection of dose has been usually based on the maximally tolerated dose. This same principle does not apply for targeted therapies, where an optimal biologic dose would be preferred. The definition of optimal dose may be established based on pharmacokinetic end points or, preferably, by demonstrating the desired effect on the target molecules in vivo, in the matter of the present invention, the Transforming Growth Factor-beta (TGFβ) receptor kinases. The assays provided here may be useful for determining the optimal biological dose or biologically effective dose by comparing the effects of a kinase inhibitor on, for example, the gene expression profile described herein or on the level of phosphorylated Smad 2 and 3.

A ninth aspect of the invention provides a method for determining the effect of a TGF-β receptor kinase inhibitor on modulation of TGF-β signaling. In a particular embodiment, the method comprises the steps of:
a) providing a cell that expresses one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1.
b) determining the baseline level of expression of one or more of the genes from step a) in the cell;
c) treating the cell with TGF-β alone or with TGF-β plus a TGF-β receptor kinase inhibitor;
d) isolating RNA from the cell of step c); and
e) analyzing the RNA from step d) to determine whether any one or more genes from step a) were up-regulated or down-regulated by treating the cell with TGF-β plus a TGF-β receptor kinase inhibitor, as compared to a cell treated with TGF-β alone;
   wherein a change in the level of expression of one or more of the genes from step a) in the TGF-β treated cell compared to the cell treated with TGF-β plus a receptor kinase inhibitor is indicative that the TGF-β receptor kinase inhibitor modulates TGF-β signaling.

In a particular embodiment, the cell is a tumor cell, a peripheral blood mononuclear cell (PBMC) a skin cell, a bone marrow cell, a cell obtained from a gingival biopsy, a cell obtained from the colon, a cell obtained from the endometrium and any other accessible tissue or cell of the human body. In another particular embodiment, the PBMC is a lymphocyte or a monocyte. In another particular embodiment, the lymphocyte is a T cell, or a B cell.

In another particular embodiment, the one or more genes that are down-regulated in the presence of a TGF-β receptor kinase inhibitor are selected from the group consisting of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1.

In another particular embodiment, the one or more genes that are up-regulated in the presence of a TGF-β receptor kinase inhibitor are selected from the group consisting of COP1, SEC24D, ZFHX1B, FLI1, PLA2G7, CXCL2, CCR1, FUCA1, CSPG2, MNDA, PAX8, THBS1, CX3CR1, DHRS9 and SERPINB2.

In another particular embodiment, the RNA is analyzed using a method selected from the group consisting of gene expression microarray analysis or by polymerase chain reaction (PCR.). In a more particular embodiment, the PCR is quantitative real-time PCR.

In another particular embodiment, the cell is treated with TGF-β alone or TGF-β plus a receptor kinase inhibitor for a time period ranging from about 0 to 24 hours. In another particular embodiment, the concentration of TGF-β ranges from about 0 pM to about 400 pM. In another particular embodiment, the inhibitor of TGF-β receptor kinase is added at a concentration ranging from about 0 uM to about 2 uM. In another particular embodiment, the effect of a TGF-β receptor kinase inhibitor on TGF-β signaling and/or changes in gene expression resulting from exposure of the cell to TGF-β is both time and dose dependent. In another particular embodiment, the changes in gene expression in the cell are dependent on the activity of the TGF-β type 1 receptor kinase.

A tenth aspect of the invention provides a method for determining a biologically effective dose of a TGF-β receptor kinase inhibitor, or for determining the effectiveness of therapy with a TGF-β receptor kinase inhibitor in patients receiving such therapy, or for determining whether a TGF-β receptor kinase inhibitor would be effective in treating a patient in need of such therapy. In one particular embodiment, the method comprises the steps of:
a) obtaining a tissue or cell sample from a patient prior to initiation of therapy with a TGF-β receptor kinase inhibitor to establish a baseline level of one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2, and SERPINE1;
b) obtaining a tissue or cell sample from a patient during the course of therapy with a TGF-β receptor kinase inhibitor and after therapy has ended to establish a change in the level of one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1;
c) treating the cell with TGF-β or with a vehicle control;
d) isolating RNA from the cell of step c); and
e) analyzing the RNA from step d) to determine whether any one or more genes from step a) were up-regulated or down-regulated following treatment with a TGF-β receptor kinase inhibitor;
   wherein a change in the level of expression of one or more of the genes from step a) in a cell in the presence of a TGF-β receptor kinase inhibitor indicates that the one or more genes may be used as a biomarker for determining the biologically effective dose of a TGF-β receptor kinase inhibitor, or for determining the effectiveness of therapy with a TGF-β receptor kinase inhibitor in patients receiving such therapy, or for determining whether a TGF-β receptor kinase inhibitor would be effective in treating a patient in need of such therapy.

In one particular embodiment, the cell is a tumor cell, a peripheral blood mononuclear cell (PBMC) a skin cell, a bone marrow cell, a cell obtained from a gingival biopsy, a cell obtained from the colon, a cell obtained from the endometrium and any other accessible tissue or cell of the human body. In another particular embodiment, the PBMC is a lymphocyte or a monocyte. In another particular embodiment, the lymphocyte is a T cell, or a B cell.

In another particular embodiment, the one or more genes that are down-regulated in the presence of a TGF-β receptor kinase inhibitor are selected from the group consisting of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1.

In another particular embodiment, the one or more genes that are up-regulated in the presence of a TGF-β receptor kinase inhibitor are selected from the group consisting of COP1, SEC24D, ZFHX1B, FLI1, PLA2G7, CXCL2, CCR1, FUCA1, CSPG2, MNDA, PAX8, THBS1, CX3CR1, DHRS9 and SERPINB2.

In another particular embodiment, the RNA is analyzed using a method selected from the group consisting of gene expression microarray analysis or by polymerase chain reaction (PCR.). In another particular embodiment, the PCR is quantitative real-time PCR. In another particular embodiment, the changes in gene expression in the cell are dependent on the activity of the TGF-β type 1 receptor kinase.

An eleventh aspect of the invention provides a method of determining the ability of a drug candidate to inhibit TGF-β signaling, the method comprising:
a) providing a cell that expresses one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1;
b) adding to the cell either TGF-β alone, or TGF-β plus a drug candidate;
c) processing the cell to release nucleic acid and cytoplasmic proteins from the cell;
d) determining the expression level of one or more of the genes from step a);
e) comparing the expression level of one or more of the genes in the cell treated with TGF-β alone with the expression level of one or more of the genes in a cell treated with TGF-β plus the drug candidate to determine:
   (i) whether expression of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1 is decreased in the cell treated with TGF-β plus a drug candidate relative to a cell not treated with the drug candidate, or
   (ii) whether expression of COP1, SEC24D, ZFHX1B, FLI1, PLA2G7, CXCL2, CCR1, FUCA1, CSPG2, MNDA, PAX8, THBS1, CX3CR1, DHRS9 and SERPINB2 is increased in the cell treated with TGF-β plus a drug candidate relative to a cell not treated with the drug candidate; and
wherein the drug candidate is identified as a potential inhibitor of TGF-β signaling if the expression level of a gene listed in (i) is decreased and/or the expression level of a gene listed in (ii) is increased.

In one embodiment, the method further comprises:
f) contacting the processed cell with a solid substrate to allow binding of released Smad2/3 to the substrate;
g) measuring the amount of pSmad2/3 in the cell sample by detecting the pSmad2/3 with an antibody specific for pSmad2/3;
h) comparing the levels of pSmad2/3 in the cell sample obtained from the cell treated with TGF-β alone or from the cell treated with TGF-β plus the drug candidate;
wherein a decrease in the level of pSmad2/3 in the presence of the drug candidate compared to the level of pSmad2/3 in the absence of the drug candidate is indicative that the drug candidate is an inhibitor of TGF-β signaling.

In another embodiment, the expression level of a plurality of genes is determined and compared. In yet another embodiment, the expression level of at least five genes is determined and compared. In another particular embodiment, the expression level is determined from the amount of transcript expressed by the one or more genes. In another particular embodiment, the expression level is determined from the amount of protein expressed by the one or more genes.

In yet another particular embodiment, the expression level is determined by gene expression microarray analysis, protein expression microarray analysis, polymerase chain reaction, quantitative polymerase chain reaction, or by enzyme-linked immunosorbent assay detection of a protein product of the one or more genes.

A twelfth aspect of the invention provides a diagnostic test kit for determining the effect of a TGF-β receptor kinase inhibitor on modulation of TGF-β signaling, or for determining a biologically effective dose of a TGF-β kinase inhibitor, or for determining the effectiveness of therapy with a TGF-β receptor kinase inhibitor in patients receiving such therapy, or for identifying a TGF-β receptor kinase inhibitor that would be effective in treating a patient in need of such therapy, comprising:

a) one or more nucleic acids encoding one or more of the proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2; and SERPINE1;

b) reagents useful for monitoring the expression level of the one or more nucleic acids or proteins encoded by the nucleic acids of step a);

c) instructions for use of the kit.

In one embodiment, the kit comprises at least five nucleic acids encoding at least five proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, and SERPINB2.

In another embodiment, the kit comprises at least ten nucleic acids encoding at least ten proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, and SERPINB2.

The kits may contain one or more components used in the methods of this invention, and may contain instructions for use. In addition to the specific components listed below, the kits may contain other components useful for performing the methods of the invention, such as RNA or DNA polymerase, buffers, reagents, and other components known to the art. Thus, the invention includes a kit for amplifying all or a portion of at least one target nucleic acid in a sample containing a plurality of nucleic acids, including either DNAs, or RNAs, each kit comprising one or more containers: (a) a primer for first-strand cDNA synthesis comprising a sequence which anneals to a selected nucleotide sequence of the target nucleic acid sequence (e.g., mRNA); (b) a primer for second-strand cDNA synthesis which produces a second-strand cDNA comprising either an RNA polymerase promoter at the 5′ end of its sense strand, or a PCR primer site at the 5′ end of its antisense strand, or both (for the same sense method) or a PCR primer site at the 5′ end of its sense strand, or an RNA polymerase promoter at the 5′ end of its antisense strand or both (for the antisense method); (c) a first PCR primer comprising an RNA polymerase promoter sequence; and (d) a second PCR primer comprising a PCR primer site sequence; (e) adenosine, cytosine, guanine, and thymine deoxyribonucleotide triphosphates; and (f) adenosine, cytosine, guanine, and uracil ribonucleotide triphosphates.

One particular embodiment of a kit of the invention may also include probes which are specific for the genes encoding one or more of the proteins of interest. Such probes may be labeled (e.g., fluorescently labeled) to facilitate their use in real time detection of amplicons produced during the course of PCR amplification.

A thirteenth aspect of the invention provides a kit for determining the effect of a TGF-β receptor kinase inhibitor on modulation of TGF-β signaling, or for determining a biologically effective dose of a TGF-β kinase inhibitor, or for determining the effectiveness of therapy with a TGF-β receptor kinase inhibitor in patients receiving such therapy, or for identifying a TGF-β receptor kinase inhibitor that would be effective in treating a patient in need of such therapy, comprising:

a first plurality of oligonucleotides, comprising the nucleic acid sequences of five or more SEQ ID NOs; 1-42, or the complements thereof, and a second plurality of oligonucleotides, comprising mismatch oligonucleotides corresponding to the first plurality of oligonucleotides, and wherein each oligonucleotide is attached to a solid support in a determinable location.

In one particular embodiment, the solid support is a plurality of beads. In another particular embodiment, the solid support is glass.

A fourteenth aspect of the invention provides an array of oligonucleotides comprising the nucleic acid sequences of SEQ ID NOs; 1 through 42 attached to a solid support in a determinable location of the array.

A fifteenth aspect of the invention provides a method for diagnosing a disease or condition associated with activated TGF-β signaling in a patient, or for determining whether a patient is prone to developing such disease or condition, comprising the steps of:

(a) obtaining a biological sample from the patient;

(b) releasing nucleic acids from said biological sample;

(c) performing PCR in the presence of a set of primers specific for any one of SEQ ID NOs: 1 through 42 and labeled probes that recognize and bind to any one of SEQ ID NOS: 1 through 42 under conditions wherein the presence or level of a nucleic acid sequence that is modulated as a result of TGF-β signaling results in an amplified and labeled PCR product; and

(d) detecting the presence of a labeled PCR product, wherein the presence of a labeled PCR product indicates the presence of a nucleic acid sequence associated with TGF-β signaling; and

wherein the presence of the labeled PCR product is indicative of the presence of a disease or condition associated with TGF-β signaling.

In one embodiment, the disease or condition is a neoplastic or hyperproliferative disease or a disease or condition characterized by an immunodeficiency, or a depressed immune response.

Thus, as noted above, methods are provided for determining the presence of one or more of the genes set forth in SEQ ID NOs: 1-42 in a biological sample. Utilizing such methods as described herein, one of skill in the art can generate accurate and rapid results, which can provide same day results from test samples. Such methods may be utilized to detect the presence of a desired target nucleic acid molecule within a biological sample. Representative examples of biological samples include cells or tissue including for example, a tumor cell, a peripheral blood mononuclear cell (PBMC) a skin cell, a bone marrow cell, a cell obtained from a gingival biopsy, a cell obtained from the colon, a cell obtained from the endometrium and any other accessible tissue or cell of the human body. In another particular embodiment, the PBMC is a lymphocyte or a monocyte. In another particular embodiment, the lymphocyte is a T cell, or a B cell.

Methods for generating target nucleic acid molecules may be readily accomplished by one of ordinary skill in the art given the disclosure provided herein and general knowledge of such procedures (see generally, Sambrook et al., Molecular Cloning: A Laboratory Manual (2d ed.), Cold Spring Harbor Laboratory Press, 1989). As noted above, within one aspect of the present invention the target nucleic acid molecule is reacted with a complementary single-stranded nucleic acid probe. Preferably, probes are designed which hybridize with one or more genes selected from the group consisting of SEQ ID NOs 1 through 42. Although within various embodiments of the invention a single-stranded probe is utilized to react or hybridize to a single-stranded target sequence, the above-described methods should not be limited to situations wherein complementary probe and target sequences pair to form a duplex.

Single stranded nucleic acid molecules may be synthesized or obtained and/or prepared directly from a target cell or organism utilizing standard techniques (see, e.g., Sambrook et al., “Molecular Cloning: A Laboratory Manual”, Cold Spring Harbor, 1989), or prepared utilizing any of a wide variety of a techniques, including for example, PCR, or reverse transcription of RNA.

Other objects and advantages will become apparent from a review of the ensuing detailed description and attendant claims taken in conjunction with the following illustrative drawings. All references cited in the present application are incorporated herein in their entirety.

### BRIEF DESCRIPTION OF THE DRAWINGS

FIG. 1: Outline of the TGFβ Cell Signaling Pathway TGFβ binds the type II receptor (TPR-II) and recruits the type I receptor, which is then phosphorylated by the TPR-II kinase. The active TPR-I then phosphorylates the R-Smads (Smad2 and Smad3), which form heteromeric complexes with the Co-Smad, Smad4. R-Smad-Smad4 heteromeric complexes translocate to the nucleus, where they interact with specific DNA sequences in conjunction with a variety of DNA binding proteins to regulate transcriptional responses of TGFβ target genes.

FIG. 2: Demonstrates that pSmad2 levels increase as a function of TβR-I kinase activity. Using purified recombinant constitutively active TβR-I kinase and recombinant GST-Smad2 fusion protein in in vitro kinase assays, the pSmad2 antibody detects a band of approximately 58 kDa, the density of which is proportional to the amount of active enzyme.

FIG. 3: Demonstrates that pSmad2 and -3 levels increase as a function of TGFβ concentration in whole cells. Both antibodies are able to detect pSmad2 and -3 in human keratinocytes treated with as little as 1.25 pM TGFβ, and the signal is proportional to the TGFβ concentration used.

FIG. 4: Demonstrates that pSmad levels increase as a function of time to TGFβ exposure. Increases in pR-Smad levels can be detected as early as 5 minutes following the addition of 100 pM TGFβ to the culture medium, and maximal levels are achieved at approximately 1 hour

FIG. 5: Effects of a quinazoline class TβR-I kinase inhibitor, TβKI, on growth of human keratinocytes. TGFβ potently inhibits growth of human keratinocytes in a dose-dependent manner, with an IC₅₀ of approximately 5 pM. Moreover, pre-incubation of the cells with the TβR-I kinase inhibitor, TβKI, completely blocks TGFβ-induced growth arrest, indicating that the response is mediated by TβR-I.

FIG. 6: Demonstrates that TGFβ-induced EMT of human keratinocytes is blocked by the TβKI. A. Morphological changes; B. Redistribution of F-actin and E-cadherin

FIG. 7: Demonstrates the ability of TGFβ to activate TGFβ receptors in Sweig human, Epstein-Barr virus immortalized, lymphoblastoid cells. Sweig human lymphoblastoid cells were treated with varying concentrations of TGFβ1 for 1 h. pSmad2 and Smad2 levels in cell lysates were determined by Western blot. TGFβ treatment induced Smad2 phosphorylation in a dose-dependent manner.

FIG. 8: Demonstrates that the TβR-I kinase inhibitor, TβKI, blocks TGFβ-induced Smad2 phosphorylation in Sweig cells. Sweig human lymphoblastoid cells were treated with TβKI (1 μM) or vehicle only (control) for 15 min, followed by the addition of TGFβ1 (100 pM) or vehicle only for 1 h. pSmad2 and Smad2 levels in cell lysates were determined by Western blot. Pre-treatment of cells with TβKI inhibited TGFβ-induced as well as basal levels of Smad2 phosphorylation.

FIG. 9: Demonstrates the sensitivity of TβR-I kinase activity in Sweig cells to the TβKI. Sweig human lymphoblastoid cells were treated with varying concentrations of TβKI or vehicle only (0) for 15 min, followed by the addition of TGFβ1 (100 pM) for 1 h. pSmad2 and Smad2 levels in cell lysates were determined by Western blot as described in “Materials & Methods”. Pre-treatment of cells with TβKI inhibited TGFβ-induced Smad2 phosphorylation in a dose-dependent manner, with an estimated IC₅₀ of 30 nM.

FIG. 10: Demonstrates that freshly isolated PBMC in short-term could be used to assess the activity of TβKIs in blood. Freshly isolated human PBMCs from healthy volunteers were treated with TβKI (1 μM) or vehicle only (control) for 15 min, followed by the addition of TGFβ1 (100 pM) or vehicle only for 1 h. pSmad2 and Smad2 levels in cell lysates were determined by Western blot. PBMCs expressed high levels of pSmad2, which was not increased further by the addition of exogenous TGFβ. Pre-treatment of cells with TβKI reduced pSmad2 levels.

FIG. 11: Demonstrates that pSmad2 levels were reduced by TβKI in a dose-dependent manner using freshly isolated human PBMCs from healthy volunteers which were treated with varying concentrations of TβKI or vehicle only (0) for 15 min, followed by the addition of TGFβ1 (100 pM) for 1 h. pSmad2 and Smad2 levels in cell lysates were determined by Western blot. Pre-treatment of cells with TβKI inhibited TGFβ-induced Smad2 phosphorylation in a dose-dependent manner, with an estimated IC₅₀ of 100 nM.

FIG. 12: Demonstrates the results of a series of mixing experiments to simulate the effects of TβKI in blood on circulating PBMCs. TβKI was dissolved in 150 mM NaCl, which was then mixed 1:1 with human plasma. Freshly isolated human PBMCs from healthy volunteers were then treated with varying concentrations of plasma TβKI or vehicle only (0) for 15 min, followed by the addition of TGFβ1 (100 pM) for 1 h. pSmad2 and Smad2 levels in cell lysates were determined by Western blot. Pre-treatment of cells with plasma TβKI inhibited TGFβ-induced Smad2 phosphorylation in a dose-dependent manner, with an estimated IC₅₀ of 40 μM.

FIG. 13: A. Confluent BxPC-3 pancreatic cancer cells were incubated overnight with a pan-specific anti-TGFβ neutralizing antibody at the indicated concentrations, and pSmad2 and Smad2 levels were assayed by western blot. B. pSmad2/Smad2 ratios were determined from integrated optical densities of bands on western blots. Treatment of cultures with pan-specific TGFβ neutralizing antibody resulted in a dose-dependent reduction in specific pSmad2 levels.

FIG. 14. Effects of exogenous TGFβ on Smad2 phosphorylation in human PBMCs: Isolated PBMCs were treated with TGFβ at a concentration of 100 pM for 2 hours. pSmad2 and total Smad2 levels were determined by Western blot analysis as described in “Materials and Methods”. PBMCs in serum-containing medium expressed detectable levels of pSmad2 even in the absence of TGFβ. Exogenous TGFβ caused a further increase in pSmad2 levels, indicating that mononuclear cells are responsive to TGFβ. The addition of 1 μM SD-093 and/or SD-208, 15 min prior to TGFβ treatment, was effective at reducing the pSmad2 signal. Total Smad2 levels, on the other hand, were unchanged among the different treatment conditions.

FIG. 15. Effects of SD-093 and SD-208 on pSmad2 levels in PBMCs: Human mononuclear cells, were plated in chambers of two 6-well culture dishes at 8×10⁴ cells per well, and exposed to SD-093 or SD-208 or vehicle alone at concentrations of 0 nM, 15 nM, 31 nM, 62.5 nM, 125 nM and 250 nM, for 2 hours at 37° C., 5% CO₂ atmosphere in the presence of serum-containing medium. pSmad2 levels in cell lysates were detected by Western blotting as described in “Materials and Methods”. A decrease in pSmad2 signal was seen with increasing dose of SD-093 or SD-208 with an estimated IC₅₀ of 60 nM and 70 nM respectively.

FIG. 16. Dephosphorylation of pSmad2 by SD-093 and SD-208 in PBMCs: PBMCs were treated with 1 μM SD-093 or SD-208 or vehicle alone (untreated) in the presence of serum-containing medium over for the times indicated. pSmad2 and total Smad2 levels in cell lysates were determined by Western blot analysis as described in “Materials and Methods”. Treatment of the cells with SD-093 or SD-208 caused a decrease in pSmad2 levels with increasing time, with a half life of 70 min, whereas total Smad 2 levels were unaffected.

FIG. 17. Effects of SD-093 on pSmad2 and pSmad3 levels in nuclear and cytoplasmic fractions of PBMCs: Nuclear and cytoplasmic fractions of PBMCs were isolated following treatment of the cells with 1 μM SD-093 for 2 hours in the presence of serum. Phospho-Smad2 and -Smad3 levels and total Smad2 and Smad3 levels were determined by Western blot analysis as described in “Materials and Methods”. Strong pSmad2 and pSmad3 levels were seen in the nucleus and this signal was reduced with increasing time of SD-093 treatment. Total Smad2 and Smad3 levels were seen mostly in the cytoplasm and this signal was not affected by SD-093.

FIG. 18. Summary of TGFβ-regulated genes in human PBMCs: Following microarray analysis, the number of genes showing significant expression changes was given for each time point. Sections in pink represent genes induced with TGFβ treatment (100 pM) and suppressed with SD-093. Green sections represent genes that were down-regulated with TGFβ and induced with SD-093 treatment. Genes in overlapping sections of each diagram were common between the different time points. A greater number of genes were TGFβ-regulated at 19.5 hours compared to the earlier time points.

FIG. 19. TGFβ-regulated gene expression in PBMCs as a function of time: Isolated PBMCs were treated with 100 pM TGFβ, 90 nM SD-093, both, or vehicle alone (control) for 0, 2 and 19.5 hours. RNA was isolated immediately after PBMC isolation (baseline control) and after 2 and 19.5 hours of treatment. Following microarray analysis, genes were selected that were common between the different time points, and the effect of TGFβ was examined in these genes. There was a greater response to TGFβ at the later time point (t=19.5 hours) compared to 2 hours.

FIG. 20. Effects of SD-093 on TGFβ-regulated gene expression in PBMCs: Isolated PBMCs were treated with 100 pM TGFβ, 90 nM SD-093, both, or vehicle alone (control) for 0, 2 and 19.5 hours. RNA was isolated immediately after PBMC isolation (baseline control) and after 2 and 19.5 hours of treatment. Following microarray analysis, genes were selected that were common between the different time points, and the effect of SD-093 was examined in these genes. TGFβ-induced gene expression was inhibited with SD-093 treatment 20A), and TGFβ-repressed genes had an induction in gene expression when treated with the inhibitor (20B). A greater number of genes were affected by SD-093 at 19.5 hours compared to the earlier time point.

FIG. 21. Quantitative real-time PCR validation of microarray data in PBMCs: PBMCs were treated with 100 pM TGFβ, 90 nM SD-093, 270 nM SD-093, or vehicle alone (control) for 0, 2 and 19.5 hours. Changes in gene expression were detected with real-time PCR as described in “Materials and Methods”. The figure shows similar trends of expression for four genes (OSM, PAI-1, VEGF and OLR-1) selected from the microarray data set, compared to the GAPDH signal, which served as a control. The ratios of each selected gene to GAPDH for t=0 is 1.0. For the 2-hour and 19.5-hour time points, each of these genes was induced with TGFβ treatment and suppressed with SD-093 in a dose-dependent manner.

FIG. 22. Effects of SD-093 on expression patterns of TGFβ-regulated genes in human PBMCs: Isolated PBMCs were treated with 100 pM TGFβ (0 nM SD-093), 90 nM SD-093, 270 nM SD-093, or vehicle only (control) for 0, 2 and 19.5 hours. Realtime PCR revealed that that ratios of the mRNA levels of each of the selected genes (OSM, PAI-1, VEGF and OLR-1) from the microarray data set to the GAPDH signal, which served as a control, was significantly induced with TGFβ treatment and inhibited by SD-093 dose-dependently. SD-093 (90 nM) caused an inhibition of the TGFβ-induced gene expression and 270 nM SD-093 suppressed gene expression to a greater degree, inhibiting some levels of basal signaling as well.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the methods and materials are now described. All publications mentioned herein are incorporated herein by reference in their entirety.

### DEFINITIONS

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

A “therapeutically effective amount” is an amount sufficient to decrease or prevent the symptoms associated with the disorders or other related conditions contemplated for therapy with the compositions of the present invention. For example, the disorders contemplated for treatment with the agents identified by the methods of the present invention include, but are not limited to TGFβ-dependent diseases or conditions, such as cancers, pulmonary fibrosis, liver cirrhosis, keloids, chronic glomerulonephritis, angiogenesis, patients receiving radiation therapy, patients having arterial restenosis, ocular neovascularization (diabetic retinopathy, age-related macular degeneration, and the like) and inflammation (psoriasis, rheumatoid arthritis, and the like).

A “biologically effective dose”, or “biologically effective amount” as used herein, refers to an amount of an agent sufficient to modulate signaling by Transforming Growth Factor-beta (TGF-β), as evidenced by the ability of such agent to either up-regulate or down-regulate particular genes as described in the present invention, or as related to an effect on pSmad2/3 levels, or to have an effect on one or more biological functions of TGF-β including but not limited to immune regulation, angiogenesis, tumor metastasis, wound repair, epithelial cell growth, or tumor cell growth. In the present application, for example, a “biologically effective amount” or a “biologically effective dose” of a TGF-β receptor kinase inhibitor may range from about 0.01 μM to about 2 μM.

“Treatment” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted, or to reduce the severity of one or more symptoms or sequelae associated with a disease or condition. Moreover, “treatment”, as used herein, covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; or (c) relieving the disease or condition, or ameliorating at least one symptom associated with the disease or condition.

The term “treating”, and the like are used herein to generally mean obtaining a desired pharmacologic and/or physiologic effect. When used in the context of in vitro methods, the term “treating” refers to exposing or contacting a cell or tissue with a particular agent.

A kinase is a protein that acts as an enzyme to transfer a phosphate group onto another protein. A “kinase inhibitor” blocks the action of such a protein. As used herein, the TGF-β kinase inhibitors may be referred to as either “kinase inhibitors”, TGF-β kinase inhibitors”, “receptor kinase inhibitors”, or “TβKI”, all of which are used interchangeably. Exemplary TGF-β kinase inhibitors, such as those identified in the present application as SD-093 or SD-208, are described in the following references: Kapoun A M, Gaspar N J, Wang Y, et al. TGF{beta} R1 kinase activity, but not p38 activation is required for TGF{beta}R1-induced myofibroblast differentiation and pro-fibrotic gene expression. Mol Pharmacol (2006); Denton C P, Lindahl G E, Khan K, et al. Activation of key profibrotic mechanisms in transgenic fibroblasts expressing kinase-deficient type II Transforming growth factor-{beta} receptor (T{beta}RII{delta}k). J Biol Chem (2005); 280(16):16053-65; Hayashi T, Hideshima T, Nguyen A N, et al. Transforming growth factor beta receptor I kinase inhibitor down-regulates cytokine secretion and multiple myeloma cell growth in the bone marrow microenvironment. Clin Cancer Res (2004); 10(22):7540-6; Bonniaud P, Margetts P J, Koib M, et al. Progressive transforming growth factor beta1-induced lung fibrosis is blocked by an orally active ALK5 kinase inhibitor. Am J Respir Crit Care Med (2005);171(8):889-98; Uhl M, Aulwurm S, Wischhusen J, et al. SD-208, a novel transforming growth factor beta receptor I kinase inhibitor, inhibits growth and invasiveness and enhances immunogenicity of murine and human glioma cells in vitro and in vivo. Cancer Res (2004);64(21):7954-61; Subramanian G, Schwarz R E, Higgins L, et al. Targeting endogenous transforming growth factor beta receptor signaling in SMAD4-deficient human pancreatic carcinoma cells inhibits their invasive phenotype1. Cancer Res (2004); 64(15):5200-11; Ge R, Rajeev V, Ray P, et al. Inhibition of growth and metastasis of mouse mammary carcinoma by selective inhibitor of Transforming Growth Factor-β type I receptor kinase in vivo. Clin Cancer Res (2006);In Press; Ge R, Rajeev V, Subramanian G, et al. Selective inhibitors of type I receptor kinase block cellular transforming growth factor-beta signaling, Biochem Pharmacol (2004);68(1):41-50. The effect of a kinase inhibitor as related to the present invention is determined by looking for a statistically significant difference between a cell or tissue that is untreated compared to one that is treated with a kinase inhibitor. In addition, in the case of in vivo analysis, the effect of a kinase inhibitor is measured by determining whether there is a significant difference between the assay measurement before, during and after treatment with the kinase inhibitor.

The “optimal biologic dose” as defined in the present application refers to a dose of a TGFβ receptor kinase inhibitor that may be established based on pharmacokinetic end points or, preferably, by demonstrating the desired effect on the target molecule in vivo, in the matter of the present invention, the TβR kinases. Such “optimal biologic dose” results in achieving the preferred endpoint for which the kinase inhibitors have been proposed for use, primarily as inhibitors of tumor cell proliferation, or for inhibition of other conditions in which the Transforming Growth Factor-beta signaling pathway plays a role, as described above.

A “subject who is a candidate for therapy with a TGFβ receptor kinase inhibitor” is one suffering from a TGFβ-dependent disease or condition. These diseases or conditions refer to pathologic conditions that depend on the activity of one or more TGFβ receptor kinases. TGFβ receptor kinases either directly or indirectly participate in the signal transduction pathways of a variety of cellular activities including proliferation, adhesion and migration, and differentiation. Diseases associated with TGFβ receptor kinase activities include the proliferation of tumor cells, the pathologic neovascularization that supports solid tumor growth, ocular neovascularization (diabetic retinopathy, age-related macular degeneration, and the like), pulmonary fibrosis, liver cirrhosis, chronic glomerulonephritis and inflammation (psoriasis, rheumatoid arthritis, and the like). Patients receiving radiation therapy, patients having arterial restenosis and patients having keloids are also candidates for therapy with a TGFβ receptor kinase inhibitor.

By “effectiveness of therapy” is meant that upon treating a patient with a TGFβ receptor kinase inhibitor, one can determine whether the treatment has resulted in the desired outcome. For example, in the case of treating a cancer patient with the inhibitor, one may observe a decrease in the tumor size or cellular proliferation or metastasis associated with tumor progression.

“Arterial restenosis” refers to a reoccurrence of a blockage in a blood vessel within six months at the same location where a previous intervention was performed. This reoccurrence is related to the treatment technique. If the reoccurrence happens later than six months, it is believed to be progression of the arteriosclerosis. Extensive keloids may become binding, limiting mobility. They may cause cosmetic changes and affect the appearance.

“Glomerulonephritis” refers to inflammation of the glomeruli in the kidney. Most often, it is caused by an auto-immune disease, but it can also result from infection. Symptoms include decreased urine production, swelling of the feet and excess protein in the urine or blood in the urine.

“Pulmonary fibrosis” is a chronic inflammation and progressive fibrosis of the pulmonary alveolar walls, with steadily progressive dyspnea, resulting finally in death from lack of oxygen or right heart failure. Familial pulmonary fibrosis (FPF) is a rare form of idiopathic pulmonary fibrosis (IPF) which is a type of interstitial lung disease. Interstitial lung diseases result from damage to the interstitium of the lung. The interstitium is the tissue wall between the air sacs, or alveoli, of the lung. Normally, this is a thin tissue layer with just a few cells in it, consisting of white blood cells and fibroblasts. For most causes of interstitial lung disease, something is believed to damage the lining of the alveoli, leading to inflammation in the interstitium. In addition, the fibroblasts in this layer then begin producing collagen, or scar tissue, in response to this damage. Some of the more common causes include connective tissue diseases including scleroderma, polymyositis-dermatomyositis, systemic lupus erythematosus, rheumatoid arthritis, ankylosing spondylitis. Alternatively, it may be treatment or drug-induced caused by any of the following: antibiotics ((furantoin, sulfasalazine); antiarrhythmics (amiodarone, tocainide, propranolol); anti-inflammatory agents (gold, penicillamine); anticonvulsants (phenyloin); chemotherapy agents (mitomycin C, bleomycin, busulfan, cyclophosphamide, Azathioprine, BCNU, methotrexate); therapeutic radiation; oxygen; cocaine. Primary diseases that may result in Pulmonary fibrosis include sarcoidosis; eosinophilic granuloma; amyloidosis; lymphangitic carcinoma; bronchoalveolar carcinoma; pulmonary lymphoma; adult respiratory distress syndrome; acquired immunodeficiency syndrome (AIDS); bone marrow transplantation; postinfectious; respiratory bronchiolitis; eosinophilic pneumonia; diffuse alveolar hemorrhage syndrome. Other occupational and environmental risk factors for pulmonary fibrosis include inorganic dusts; asbestosis; silicosis; coal worker's pneumoconiosis; talc pneumoconiosis.

“Liver cirrhosis” is the result of chronic liver disease that causes scarring of the liver (fibrosis—nodular regeneration) and liver dysfunction. This often has many complications, including accumulation of fluid in the abdomen ascites, bleeding disorders, coagulopathy, increased pressure in the blood vessels (portal hypertension), and confusion or a change in the level of consciousness hepatic encephalopathy.

“Keloids” are an overgrowth of scar tissue at the site of a skin injury. Keloids occur from such skin injuries as surgical incisions, traumatic wounds, vaccination sites, burns, chickenpox, acne, or even minor scratches.

The term “antibody” as used herein includes intact molecules as well as fragments thereof, such as Fab and F(ab′)₂, which are capable of binding the epitopic determinant. Antibodies that bind the proteins of the present invention can be prepared using intact polypeptides or fragments containing small peptides of interest as the immunizing antigen attached to a carrier molecule. Commonly used carriers that are chemically coupled to peptides include bovine or chicken serum albumin, thyroglobulin, and other carriers known to those skilled in the art. The coupled peptide is then used to immunize the animal (e.g, a mouse, rat or rabbit). The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be a single chain antibody. (See, e.g., Curiel et al., U.S. Pat. No. 5,910,486 and U.S. Pat. No. 6,028,059). The antibody may be prepared in, but not limited to, mice, rats, rabbits, goats, sheep, swine, dogs, cats, or horses.

Structurally, the simplest naturally occurring antibody (e.g., IgG) comprises four polypeptide chains, two heavy (H) chains and two light (L) chains inter-connected by disulfide bonds. It has been shown that the antigen-binding function of an antibody can be performed by fragments of a naturally-occurring antibody. Thus, these antigen-binding fragments are intended to be encompassed by the term “antibody homologue”. Examples of binding fragments include (i) a Fab fragment consisting of the VL, VH, CL and CH1 regions; (ii) a Fd fragment consisting of the VH and CH1 regions; (iii) a Fv fragment consisting of the VL and VH regions of a single arm of an antibody, (iv) a dAb fragment, which consists of a VH region; (v) an isolated complimentarity determining region (CDR); and (vi) a F(ab′)₂ fragment, a bivalent fragment comprising two Fab fragments linked by a disulfide bridge at the hinge region.

Furthermore, although the two regions of the Fv fragment are coded for by separate genes, a synthetic linker can be made that enables them to be made as a single chain protein (referred to herein as single chain antibody or a single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antibody homologue”. Other forms of recombinant antibodies, such as chimeric, humanized and bispecific antibodies are also within the scope of the invention.

The term “antibody combining site”, as used herein refers to that structural portion of an antibody molecule comprised of a heavy and light chain variable and hypervariable regions that specifically binds (immunoreacts with) antigen.

The terms “bind”, “immunoreact” or “reactive with” in its various forms is used herein to refer to an interaction between an antigenic determinant-containing molecule (i.e., antigen) and a molecule containing an antibody combining site, such as a whole antibody molecule or a portion thereof, or recombinant antibody molecule (i.e., antibody homologue).

The term “monoclonal antibody” or “monoclonal antibody composition”, as used herein, refers to a population of antibody molecules that contain only one species of an antigen binding site capable of immunoreacting with a particular epitope of an antigen. A monoclonal antibody composition thus typically displays a single binding affinity for a particular antigen with which it immunoreacts.

“Analog” or “mimic” as used herein, refers to a chemical compound, a nucleotide, a protein, or a polypeptide, or an antibody that possesses similar or identical activity or function(s) as the chemical compounds, nucleotides, proteins, polypeptides or antibodies having the desired activity and therapeutic effect of the present invention, but need not necessarily comprise a sequence that is similar or identical to the sequence of the preferred embodiment, or possess a structure that is similar or identical to the agents of the present invention. As used herein, a nucleic acid or nucleotide sequence, or an amino acid sequence of a protein or polypeptide is “similar” to that of a nucleic acid, nucleotide or protein or polypeptide having the desired activity if it satisfies at least one of the following criteria: (a) the nucleic acid, nucleotide, protein or polypeptide has a sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleic acid, nucleotide, protein or polypeptide sequences having the desired activity as described herein (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues); or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the polypeptides of the present invention having the desired therapeutic effect. As used herein, a polypeptide with “similar structure” to that of the preferred embodiments of the invention refers to a polypeptide that has a similar secondary, tertiary or quarternary structure as that of the preferred embodiment. The structure of a polypeptide can be determined by methods known to those skilled in the art, including but not limited to, X-ray crystallography, nuclear magnetic resonance, and crystallographic electron microscopy.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

As used herein “Arrays” or “Microarrays” or “gene chip assays” or “gene expression microarrays” refers to an array of distinct polynucleotides or oligonucleotides synthesized on a substrate, such as paper, nylon or other type of membrane, filter, chip, glass slide, or any other suitable solid support. In one embodiment, the microarray is prepared and used according to the methods described in U.S. Pat. No. 5,837,832, Chee et al., PCT application WO95/11995 (Chee et al.), US20060134665; US20060110752; US20060147966; US20060147963; US20060142951; Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522. Arrays or microarrays are commonly referred to as “DNA chips”. As used herein, arrays/microarrays may be interchangeably referred to as detection reagents or kits.

Microarrays are known in the art and consist of a surface to which probes that correspond in sequence to genes or gene products (e.g., cDNAs, mRNAs, cRNAs, polypeptides, and fragments thereof), can be specifically hybridized or bound at a known position. The microarray is an array (i.e., a matrix) in which each position represents a discrete binding site for a gene or gene product (e.g., a DNA or protein), and in which binding sites are present for most or almost all of the genes in the organism's genome.

As used herein, the terms “nucleic acid”, “polynucleotide” and “oligonucleotide” refer to primers, probes, and oligomer fragments used for detection, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

The term “primer” may refer to more than one primer and generally refers to an oligonucleotide, whether occurring naturally, as in a purified restriction digest, or produced synthetically, which is capable of acting as a point of initiation of DNA synthesis when annealed to a nucleic acid template and placed under conditions in which synthesis of a primer extension product which is complementary to the template is catalyzed. Such conditions include the presence of four different deoxyribonucleoside triphosphates and a polymerization-inducing agent such as a DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification.

As used herein, an “oligonucleotide primer” refers to a single stranded DNA or RNA molecule that is hybridizable (eg. capable of annealing) to a nucleic acid template and is capable of priming enzymatic synthesis of a second nucleic acid strand. Alternatively, or in addition, oligonucleotide primers, when labeled directly or indirectly (e.g., bound by a labeled secondary probe which is specific for the oligonucleotide primer) may be used effectively as probes to detect the presence of a specific nucleic acid in a sample. Oligonucleotide primers useful according to the invention are between about 10 to 100 nucleotides in length, preferably about 17-50 nucleotides in length and more preferably about 17-40 nucleotides in length and more preferably about 17-30 nucleotides in length. Oligonucleotide probes useful for the formation of a cleavage structure according to the invention are between about 17-40 nucleotides in length, preferably about 17-30 nucleotides in length and more preferably about 17-25 nucleotides in length.

“Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA.

The “polymerase chain reaction (PCR)” technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment (i.e, an amplicon) whose termini are defined by the 5′ ends of the primers. PCR is reported to be capable of producing a selective enrichment of a specific DNA sequence by a factor of 10⁹. The PCR method is also described in Saiki et al., 1985, Science, 230:1350.

As used herein, “probe” refers to a labeled oligonucleotide primer, which forms a duplex structure with a sequence in the target nucleic acid, due to complementarity of at least one sequence in the probe with a sequence in the target region. Such probes are useful for identification of a target nucleic acid sequence in a cell or tissue sample according to the invention, including one or more of the nucleic acid sequences as set forth in SEQ ID NOs: 1-42. Pairs of single-stranded DNA primers can be annealed to sequences within a target nucleic acid sequence or can be used to prime DNA synthesis of a target nucleic acid sequence.

“Label” or “labeled moiety capable of providing a signal” refers to any atom or molecule which can be used to provide a detectable (preferably quantifiable) signal, and which can be operatively linked to a nucleotide or nucleic acid. Labels may provide signals detectable by fluorescence, radioactivity, colorimetry, gravimetry, X-ray diffraction or absorption, magnetism, enzymatic activity, mass spectrometry, binding affinity, hybridization radiofrequency and the like.

As used herein, “sample” refers to any substance containing or presumed to contain a nucleic acid of interest (a target nucleic acid sequence such as the genes of the present invention) or which is itself a nucleic acid containing or presumed to contain a target nucleic acid sequence of interest. The term “sample” thus includes a sample of nucleic acid (genomic DNA, cDNA, RNA), cell, organism, tissue, fluid, or substance including but not limited to, for example, whole blood, plasma, serum, blood cells, such as peripheral blood mononuclear cells, lymphocytes, including T cells and B cells, or samples of in vitro cells or cell culture constituents.

As used herein, “target nucleic acid sequence” refers to a region of a nucleic acid that is to be either replicated, amplified, and/or detected. In one embodiment, the “target nucleic acid sequence” resides between two primer sequences used for amplification. In other cases the target may be a nucleic acid that is not amplified.

“Subject” or “patient” refers to a mammal, preferably a human, in need of treatment for a condition, disorder or disease.

Two DNA sequences are “substantially homologous” or “substantially similar” when at least about 50% (preferably at least about 75%, and most preferably at least about 90, or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al.; DNA Cloning, Vols. I & II; Nucleic Acid Hybridization.

Similarly, two amino acid sequences are “substantially homologous” or “substantially similar” when greater than 50% of the amino acids are identical, or functionally identical. Preferably, the similar or homologous sequences are identified by alignment using, for example, the GCG (Genetics Computer Group, Program Manual for the GCG Package, Version 7, Madison, Wis.) pileup program.

The terms “protein”, “polypeptide” and “peptide” are used interchangeably herein when referring to a gene product and indicates a molecular chain of amino acids linked through covalent and/or noncovalent bonds. The terms do not refer to a specific length of the product. Thus, peptides, oligopeptides and proteins are included within the definition of polypeptide. The terms include post-expression modifications of the polypeptide, for example, glycosylations, acetylations, phosphorylations and the like. In addition, protein fragments, analogs, mutated or variant proteins, fusion proteins and the like are included within the meaning of polypeptide.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Encoded by” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences which are immunologically identifiable with a polypeptide encoded by the sequence. Thus, an antigen “polypeptide,” “protein,” or “amino acid” sequence may have at least 60% similarity, preferably at least about 75% similarity, more preferably about 85% similarity, and most preferably about 95% similarity, to a polypeptide or amino acid sequence of an antigen.

As used herein, the term “modulate” or “modulates” refers to either an up-regulation or a down-regulation of the genes or gene products as described in the present invention.

Standard molecular biology techniques known in the art and not specifically described herein may be found in a variety of standard laboratory manuals including: Sambrook et al., Molecular Cloning: A Laboratory Manual, Cold Springs Harbor Laboratory; New York (1992).

### General Description of the Invention

Transforming Growth Factor-β (TGFβ) is a secreted extracellular protein that binds to and activates specific cell surface receptors. Receptor activation leads to transmission of the signal to the cell nucleus. In epithelial cells, TGFβ signaling induces two types of cellular response: First, it causes cell cycle arrest and/or apoptosis. Secondly, it orchestrates the cell's response to tissue injury. Specifically, TGFβ induces epithelial cells to assume a dispersed, fibroblastoid and motile phenotype (epithelial-to-mesenchymal transdifferentiation, EMT) and to produce extracellular matrix components of what later becomes the scar. Normally, this process is self-limited in space and time, allowing cells to revert back to their cohesive epithelioid phenotype. Escape from TGFβ's growth suppressive functions is an early event in the development of most cancers. However, cancer cells may retain the EMT response. Moreover, in late stage tumors TGFβ signaling acquires oncogenic properties by constitutively inducing EMT, which results in a highly invasive and metastatic spindle-cell tumor phenotype. Thus, TGFβ's homeostatic (tumor suppressive) and EMT functions often become uncoupled during malignant progression. Moreover, it is likely through this homeostatic function that TGFβ suppresses tumor development, and that its loss is an early event in epithelial carcinogenesis. For example, mice that are homozygous for a hypomorphic allele of the latent TGFβ binding protein, LTBP-4, fail to express pSmad2 in the colon and lung and are prone to developing colon cancer, supporting the idea of a tissue specific failure of TGFβ's homeostatic function (Sterner-Kock, A. et al. (2002), Genes Dev, 16:2264-2273). Finally, it has been recently been observed that most human breast, colon and HNSCC cancers continue to express pSmad2 (Xie, W. et al. (2002), Cancer Res. 62:497-505; Xie W. et al. (The Cancer J., 9:302-312, 2003). It is postulated herein that, as these tumors are actively growing, they must have escaped from TGFβ-mediated growth arrest.

In addition, TGFβ produced by tumors contributes to angiogenesis by acting on endothelial cells, and suppresses anti-tumor immunity by its actions on immune cells. Thus, under these conditions, blocking TGFβ action may represent a potent anti-cancer strategy.

### Therapeutic Uses for TβR Kinase Inhibitors

Binding of TGFβ to its type II receptor turns on a kinase enzyme located within the cytoplasmic tail of the TGFβ type II receptor (TβR-II). This kinase then phosphorylates the TGFβ type I receptor (TβR-I). This turns on a kinase enzyme located within the cytoplasmic tail of the TβR-I receptor. This kinase then phosphorylates cytoplasmic proteins called Smads (Smad2 and -3), which, in turn, transmit TGFβ's signals to the nucleus. Accordingly, therapeutic compounds could be developed to strongly and selectively block the activity of either of the two TβR receptor kinases. These compounds could potentially be used as anti-cancer drugs as well as to treat chronic inflammatory conditions associated with scarring. When these drugs will be ready to be tested in patients, a major challenge will be to develop a dosing schedule that will achieve effective inhibition of the TβR kinase enzymes in vivo, without causing significant side effects.

Rationale for Development of Targeted TβR Kinase Inhibitors

As summarized above, there is considerable experimental support for the idea that, in many cases, even though TGFβ no longer induces cell cycle arrest, cancer cells remain responsive to TGFβ's effects on cell-cell and cell-matrix interactions, and that these autocrine effects enhance the invasive and metastatic properties of the tumor cells. In addition, many cancers produce or induce bioactive TGFβs, which, in turn, supports the infrastructure of stromal cells and tumor microvasculature, and suppresses anti-tumor immune cell function. Each of these effects promotes cancer progression. Based on these observations, our central hypothesis is that blocking the effects of tumor-associated bioactive TGFβ on normal cells will inhibit cancer progression. Proof of concept has already been provided by studies showing that neutralizing antibodies to TGFβ, the TGFβ-binding proteoglycan, decorin, anti-sense TGFβ oligonucleotides, soluble TβR-II, or dominant-negative TGFβ receptor mutants are all capable of inducing tumor regression (Pepper, M. S. (1997), Cytokine Growth Factor Rev, 8:21-43; Ananth, S. et al. (1999), Cancer Res, 59:2210-2216; Fakhrai, H. et al (1996), Proc. Natl. Acad. Sci. USA, 93:2909-2914; Park, J. A. et al. (1997), Cancer Gene Therapy, 4:42-50; Tzai, T. S. et al. (1998), Anticancer Res, 18:1585-1589; Won, J. et al. (1999), Cancer Res, 59:1273-1277; Engel, S. et al. (1999), J Neuroimmunol, 99:13-18; Yang, Y. A. et al. (2002), J. Clin. Invest., 109:1607-1615). However, the lack of an adequate supply of neutralizing antibodies or of decorin, and technical difficulties associated with the use of peptides or oligonucleotides therapeutically in vivo have thwarted the further development of any of these modalities for clinical use.

Targeting the TGFβ Signaling Pathway Itself

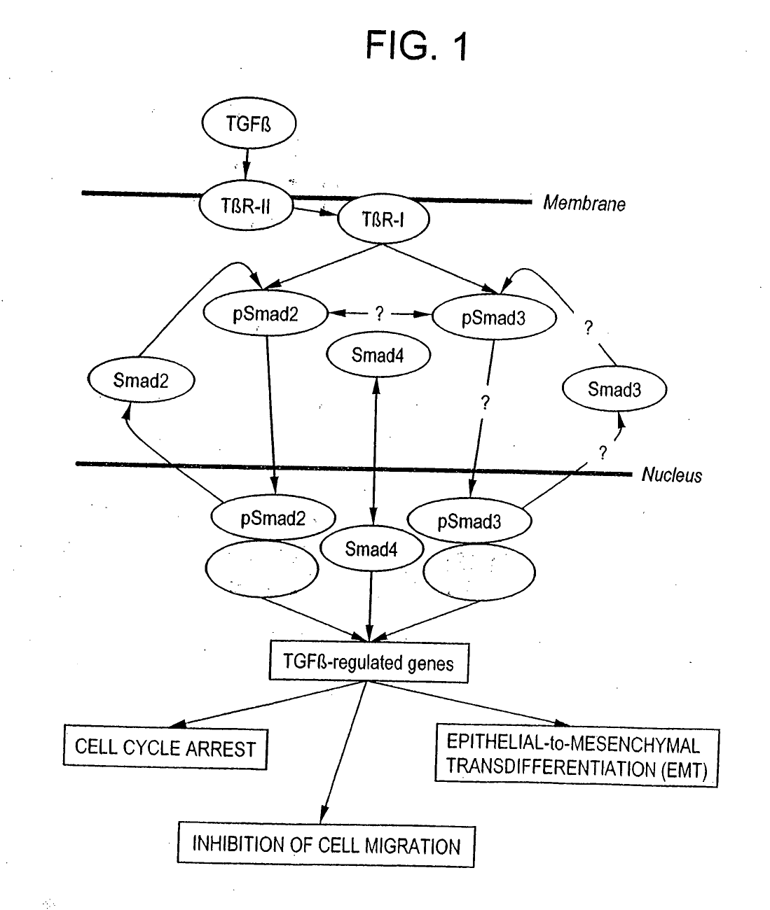

In all likelihood, the most effective and selective approach to blocking TGFβ signaling would be the development of small molecules that antagonize the TGFβ signaling pathway itself (Reiss, M. (1999), Microbes Infect. 1:1327-1347). TGFβ cell surface receptors represent both the point of greatest vulnerability in the TGFβ signaling pathway and the best target. Several different experimental studies have demonstrated convincingly that loss or mutational inactivation of either the TβR-I or -II receptors results in complete abrogation of all TGFβ-mediated responses in many different cell types (Massague, J. (1998), Annu. Rev. Biochem., 67:753-791). Secondly, the receptors are the only component of the signaling pathway that is truly specific for TGFβ (FIG. 1). Whether cells respond to TGFβs or to related members of the TGFs superfamily, such as activins or BMPs, is largely a function of the cell type-specific expression of TGFβ-, activin- or BMP receptors. However, all three groups of ligands depend on Smad4 for nuclear localization of the transcription complex and induction/repression of gene expression. Furthermore, both TGFβ and activin responses are mediated by Smad2 and -3. Therefore, inhibition of Smad4 will affect responsiveness to all 3 groups of ligands, inactivation of Smad2 or -3 will affect TGFβ- and activin- but not BMP-dependent responses, and only inhibition of the TβR receptors will selectively affect responses to TGFβs. Finally, inactivation of either Smad2, Smad3 of Smad4 individually does not always result in complete loss of TGFβ signaling, suggesting that alternate post-receptor pathways for transduction of some of TGFβ's signals may exist (Dai, J. L. et al. (1999), Mol Carcinog, 26:37-43; Ashcroft, G. S. et al. (1999), Nat Cell Biolog, 1:260-266). Work has been done to test a series of highly selective and potent TβR-I kinase inhibitors (TβKIs) that effectively block all TGFβ signaling in vitro, and to validate their use as anti-cancer agents. The use of small organic molecules, such as the quinazolines described in U.S. Pat. No. 6,476,031, incorporated herein by reference in its entirety, are contemplated for use in treatment of the conditions described herein. Alternative strategies for targeting TGFβ signaling include the use of antisense oligonucleotides, neutralizing antibodies, and soluble TβR-II exoreceptor molecules.

It is thus desirable to be able to measure TβR-I (and, indirectly, TβR-II)-kinase activity in cells by making use of antibodies that specifically detect the phosphorylated form of Smad2 (pSmad2) or Smad3 (pSmad3) induced by the TβR-I kinase. As demonstrated herein, the level of pSmad2 in cells accurately reflects the level of TβR-I kinase activity and, hence, the ability of the cells to respond to TGFβ. The assays disclosed in the present application can be applied to the clinical testing of this class of kinase inhibitors in two different ways:
1. Tissue (e.g. skin or gingival biopsies) or cells (e.g. peripheral blood mononuclear cells, PBMC) can be obtained from patients, exposed to TGFβ in vitro, and assayed for the level of pSmad2 by Western blot, slot- or dot blot. In individuals who have received effective doses of a TβR kinase inhibitor (TβKI), pSmad2 levels would be reduced
2. Plasma can be collected from patients treated with a TβKI, and incubated with TGF-β-responsive test cells in vitro in the presence or absence of TGFβ. In this case, the pSmad2 levels in the test cells should be reduced in proportion to the concentration of TβKI present in plasma or other body fluids.

These two types of assays can be utilized to optimize dosing schedules of TβKIs during the development of these agents for clinical use, as well as to monitor patients on treatment once the drugs have been approved for clinical use.

In addition, the present invention provides for a panel or a set of genes that are modulated in tissues or cells in the presence of TGF-β and demonstrates that the addition of particular receptor kinase inhibitors to these tissues or cells alters the expression of this panel of genes. More particularly, a set of genes that appears to be significantly induced by TGF-β is shown in Table 3 and a set of genes that appears to be significantly repressed by TGF-β are shown in Table 4. Furthermore, it appears that particular kinase inhibitors are effective at down-regulating the gene set/panel as shown in Table 3, and act to up-regulate the gene set/panel as shown in Table 4.

### Diagnostic Methods

Accordingly, it is an object of the present invention to provide methods for determining a biologically effective dose and for determining the optimal biologic dose of a Transforming Growth Factor-beta (TGFβ) receptor kinase inhibitor for administration to a patient in need of such therapy, or for monitoring the effectiveness of therapy with a TGFβ receptor kinase inhibitor in patients receiving such therapy, or for determining whether a kinase inhibitor would be effective in treating a patient in need of such therapy. The methods may take the form of a tissue or cell based assay or a cell free assay. The methods may also take the form of a gene expression microarray, a protein microarray or a PCR assay, in particular, a quantitative real time PCR assay.

In one particular embodiment, the steps involve the following:
a) obtaining a tissue or cell sample from said patient prior to initiation of therapy to establish baseline levels of TGFβ receptor kinase activity;
b) processing said sample to enable release of phosphorylated Smad2 and -3 (pSmad2/3) from the cells within the sample;
c) contacting said processed sample with a solid substrate to allow binding of the released pSmad2/3 to said substrate;
d) measuring the amount of pSmad2/3 in said sample by detecting said pSmad2/3 with an antibody specific for pSmad2/3;
e) obtaining a tissue or cell sample from the patient after treatment with a TGFβ receptor kinase inhibitor given at various doses; and repeating steps b) through d);
f) comparing the levels of pSmad2/3 in the tissue sample obtained in step e) to the level of pSmad2/3 in the sample obtained in step a);

wherein a decrease in the levels of pSmad2/3 compared to baseline levels is indicative of achieving the optimal dose of the TGFβ receptor kinase inhibitor.

In another particular embodiment, the steps involve the following:
a) obtaining a plasma sample from said patient prior to initiation of therapy to establish baseline levels of TGFβ receptor kinase activity;
b) contacting said sample with TGFβ-responsive test cells in vitro; wherein said cells are pretreated with TGFβ at a dose sufficient to activate TGFβ receptor kinase activity;
c) processing said cells to enable release of pSmad2/3 from the cells;
d) contacting the extract from said processed cells with a solid substrate to allow binding of the released pSmad2/3 to said substrate;
e) measuring the amount of pSmad2/3 in said extract by detecting said pSmad2/3 with an antibody specific for pSmad2/3;
f) obtaining a plasma sample from the patient after treatment with a TGFβ receptor kinase inhibitor given at various doses; and repeating steps b) through e);
g) comparing the levels of pSmad2/3 from test cells incubated with plasma samples from step f) to the level of pSmad2/3 from test cells incubated with plasma samples from step a);
   wherein a decrease in the levels of pSmad2/3 compared to baseline levels is indicative of achieving the optimal biologic dose of the TGFβ receptor kinase inhibitor.

In another particular embodiment, the steps involve the following:
a. providing a cell that expresses one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1.
b. determining the baseline level of expression of one or more of the genes from step a) in the cell;
c. treating the cell with TGF-β alone or with TGF-β plus a TGF-β receptor kinase inhibitor;
d. isolating RNA from the cell of step c); and
e. analyzing the RNA from step d) to determine whether any one or more genes from step a) were up-regulated or down-regulated by treating the cell with TGF-β plus a TGF-β receptor kinase inhibitor, as compared to a cell treated with TGF-β alone;
   wherein a change in the level of expression of one or more of the genes from step a) in the TGF-β treated cell compared to the cell treated with TGF-β plus a receptor kinase inhibitor is indicative that the TGF-β receptor kinase inhibitor modulates TGF-β signaling.

In another particular embodiment, the steps involve the following:
a) obtaining a tissue or cell sample from a patient prior to initiation of therapy with a TGF-β receptor kinase inhibitor to establish a baseline level of one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2, and SERPINE1;
b) obtaining a tissue or cell sample from a patient during the course of therapy with a TGF-β receptor kinase inhibitor and after therapy has ended to establish a change in the level of one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1;
c) treating the cell with TGF-β or with a vehicle control;
d) isolating RNA from the cell of step c); and
e) analyzing the RNA from step d) to determine whether any one or more genes from step a) were up-regulated or down-regulated following treatment with a TGF-β receptor kinase inhibitor;

wherein a change in the level of expression of one or more of the genes from step a) to step b) in a tissue or cell sample indicates that the one or more genes may be used as a biomarker for determining the biologically effective dose of a TGF-β receptor kinase inhibitor, or for determining the effectiveness of therapy with a TGF-β receptor kinase inhibitor in patients receiving such therapy, or for determining whether a TGF-β receptor kinase inhibitor would be effective in treating a patient in need of such therapy.

In a particular embodiment, the tissue or cell sample is selected from the group consisting of, but not limited to, tumor tissue, skin, bone marrow, whole blood, peripheral blood mononuclear cells (PBMC), gingiva, colon, endometrium and any other accessible tissue or cell of the human body. In another particular embodiment, the method for measuring the amount of pSmad2/3 in said sample is by detecting said pSmad2/3 with an antibody specific for pSmad2/3. The method of detecting may be accomplished by use of an immunoassay. In a further particular embodiment, the immunoassay is an enzyme linked immunoassay, a radioimmunoassay, or a Western blot assay. In yet another particular embodiment, the antibody specific for pSmad2/3 is selected from the group consisting of a polyclonal antibody, a monoclonal antibody, a single chain antibody, a human or humanized antibody, and Fab fragments thereof. They may be chimeric antibodies. They may be produced in animals, including but not limited to horses, goats, sheep, mice, rats, rabbits and guinea pigs. In another particular embodiment, the patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

In another particular embodiment, the assay method may be selected from a gene chip assay, such as a gene expression microarray, a protein microarray, or a PCR assay, more particularly a quantitative real time PCR assay.

Defining the optimal biologic dose of TβKIs: In the case of conventional non-targeted cytotoxic chemotherapeutic agents, the selection of dose has been usually based on the maximally tolerated dose. This same principle does not apply for targeted therapies, where an optimal biologic dose would be preferred instead. The definition of optimal dose may be established based on pharmacokinetic end points or, preferably, by demonstrating the desired effect on the target molecule in vivo, in the matter of the present invention, the TβR-I kinase.

For example, in clinical trials of tyrosine kinase inhibitors (TβKIs), such as the EGF-receptor antagonist, ZD1839, investigators have examined serial skin biopsies for evidence of target enzyme inhibition (Massague J. et al (2000), Genes & Development 14: 627-644; Yu L. et al. (2002), Embo J. 21: 3749-3759; Miyazawa K. et al. (2002), Genes Cells 7:1191-1204). In addition to its ease of access, skin was the tissue selected to perform pharmacodynamic studies because of the important role that EGFR plays in skin biology. Furthermore, support for a role of EGFR in skin biology was provided by the observation that patients treated with ZD1839 and other EGFR TK inhibitors or with blocking anti-EGFR monoclonal antibodies developed skin reactions, suggesting that EGFR inhibition results in alteration of normal skin homeostasis. In clinical dose-finding studies of ZD1839, the drug suppressed EGFR phosphorylation in all EGFR-expressing cells (Massague J. et al (2000), Genes & Development 14: 627-644). In addition, ZD1839 inhibited MAPK activation and reduced keratinocyte proliferation index. Concomitantly, ZD1839 increased the expression of p27(KIP1) and maturation markers, and increased apoptosis. Thus, ZD1839 inhibited EGFR activation and downstream receptor-dependent processes in vivo. Most importantly, these effects were profound at doses well below the one producing unacceptable toxicity, a finding that strongly supports pharmacodynamic assessments to select optimal doses instead of a maximum-tolerated dose for definitive efficacy and safety trials (Massague J. et al (2000), Genes & Development 14: 627-644).

Accordingly, the current application addresses the need for a rapid and more efficient means of assessing and monitoring the effects of TβR kinase inhibitors by measuring pSmad2 levels in serial tissue biopsies (e.g. skin, gingival, colon, endometrium), or blood cells, such as peripheral blood mononuclear cells, including lymphocytes or monocytes, or by assessing the level of expression of a particular set or panel of genes whose expression is modulated in tissues or cells after exposure to TGF-β. This set or panel of genes is shown in Tables 3 and 4. It is believed that this panel or set of genes may be useful as a biomarker for monitoring signaling by TGF-β and the inhibition of signaling through the use of specific TGF-β receptor kinase inhibitors.

Besides skin, blood represents the most easily accessible source of normal human nucleated cells. Moreover, blood can be obtained serially at a much greater frequency than skin biopsies. Furthermore, freshly isolated human peripheral blood mononuclear cells (PBMC) express high levels of pSmad2, and also demonstrate a change in gene expression profile upon exposure to TGF-β. In addition, exposure of PBMC to TβKI in vitro results in inhibition of TβR kinase activity and subsequent dephosphorylation of pSmad2. Furthermore, exposure of PBMC in vitro to TGF-β in addition to treatment with a particular kinase inhibitor also results in up or down-regulation of the gene set of Tables 3 and 4. Accordingly, the present application provides a rapid, efficient and accurate means to assess TβKI activity in vivo by measuring pSmad2 levels in PBMC as described herein, or by assessing the effect of a kinase inhibitor on up or down-regulation of the genes that are modulated by TGF-β.

Monitoring of patients on TβKI therapy: Besides helping define the optimal biologic dose range of TβKIs, the bioassays described in the present application are likely to be useful to monitor individual patients on TβKI therapy, in order to ensure that they are receiving the optimal dose of the drug. In this case, serial isolation of PBMCs or serial tissue biopsies can be used as described above. However, it may also be possible to substitute an assay in which plasma from patients is tested for TβR-I kinase inhibitory activity against test cells (such as cultured Sweig lymphocytes or other TGFβ-sensitive test cells (Xu, L. et al. (2002), Mol Cell 10:271-282) in vitro. The mixing studies described herein indicate that this will be feasible.

Potential clinical applications of TGFβ receptor kinase inhibitors: At this point, TGFβ receptor kinase inhibitors are being developed for a number of different clinical uses. Besides advanced cancer (Massague, J. et al. (2000), Genes & Development, 14:627-644), these include, treatment of chronic inflammatory conditions in order to prevent fibrosis (e.g. pulmonary fibrosis, liver cirrhosis, chronic glomerulonephritis, (Yu, L. (2002), Embo J, 21:3749-3759), prevention of radiation-induced fibrosis; (Miyazawa, K. et al. (2002), Genes Cells, 7:1191-1204), arterial restenosis; (Abdollah S. (1997), J Biol Chem, 272:27678-27685) and prevention of keloids. The methods and bioassays described herein will be able to provide a rapid, efficient and accurate means for defining optimal dosing schedules and to monitor patients on therapy in each of these disease categories.

Thus, the antibodies of the present invention, which specifically recognize and bind to phosphorylated Smads are capable of use in connection with various diagnostic techniques, including immunoassays, such as a radioimmunoassay, using for example, an antibody to the phosphorylated Smad that has been labeled by either radioactive addition, reduction with sodium borohydride, or radioiodination.

In an immunoassay, a control quantity of the antibodies, may be prepared and labeled with an enzyme, a specific binding partner and/or a radioactive element, and may then be introduced into a tissue or cell sample. After the labeled material or its binding partner(s) has had an opportunity to react with sites within the sample, the resulting mass may be examined by known techniques, which may vary with the nature of the label attached. For example, antibodies against specifically phosphorylated Smads may be selected and appropriately employed in the exemplary assay protocol, for the purpose of following phosphorylated protein as described above.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

In the instance of monitoring an effect of a kinase inhibitor on TGF-β signaling, one may utilize any method known to those skilled in the art to measure the effect of TGF-β on a gene profile and likewise look for the effect of a TGF-β kinase inhibitor on the level of expression of these genes. One may envision a kit prepared specifically for looking at the expression profile using one or more of the genes shown in Tables 3 or 4 and establish a specific gene chip assay for monitoring patients either undergoing treatment with a kinase inhibitor or patients who are candidates for treatment with such kinase inhibitors. The kit may take the form of those known to those skilled in the art, such as those developed by Affymetrix (www.affymetrix.com).

Alternatively, in one embodiment, the kit may comprise one or more nucleic acids encoding one or more of the proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2; and SERPINE1;

b) reagents useful for monitoring the expression level of the one or more nucleic acids or proteins encoded by the nucleic acids of step a);

c) instructions for use of the kit.

In a particular embodiment, the kit may contain five or more nucleic acids as selected from those shown above.

In another particular embodiment, the kit may contain ten or more nucleic acids as selected from those shown above.

In another particular embodiment, the kit may contain a first plurality of oligonucleotides, comprising the nucleic acid sequences of five or more SEQ ID NOs; 1-42, or the complements thereof, and a second plurality of oligonucleotides, comprising mismatch oligonucleotides corresponding to the first plurality of oligonucleotides, and wherein each oligonucleotide is attached to a solid support in a determinable location.

In another particular embodiment, the present invention provides for an array of oligonucleotides comprising the nucleic acid sequences of SEQ ID NOs; 1 through 42 attached to a solid support in a determinable location of the array. Such an array may be useful in a clinical setting where it is desirable to determine the effectiveness of therapy in a patient with a kinase inhibitor or who may be a candidate for treatment with such a kinase inhibitor.

Pharmacodynamic measures of TβR kinase activity and inhibition: For conventional non-targeted cytotoxic chemotherapeutic agents, the selection of dose has been usually based on the maximally tolerated dose. This same principle does not apply for targeted therapies, where an optimal biologic dose would be preferred instead. The definition of optimal dose may be established based on pharmacokinetic end points or, preferably, by demonstrating the desired effect on the target molecule, in our case, the TβR-I kinase.

For example, in clinical trials of tyrosine kinase inhibitors, such as the EGF-receptor antagonist, ZD1839, investigators have examined serial skin biopsies for evidence of target enzyme inhibition (Albanell, J. et al. (2002), J Clin Oncol, 20:110-124; Herbst, R. S. et al. (2002), Clin Oncol, 20:3815-3825; Baselga, J. et al J. (2002), J Clin Oncol, 20:4292-4302). In addition to its ease of access, skin was the tissue selected to perform pharmacodynamic studies because of the important role that EGFR plays in skin biology. Furthermore, support for a role of EGFR in skin biology was provided by the observation that patients treated with ZD1839 and other EGFR TK inhibitors or with blocking anti-EGFR MAbs developed skin reactions, suggesting that EGFR inhibition results in alteration of normal skin homeostasis. In clinical dose-finding studies of ZD1839, the drug suppressed EGFR phosphorylation in all EGFR-expressing cells. (Albanell, J. et al. (2002), J Clin Oncol, 20:110-124). In addition, ZD1839 inhibited MAPK activation and reduced keratinocyte proliferation index. Concomitantly, ZD1839 increased the expression of p27(KIP1) and maturation markers, and increased apoptosis. These effects were observed at all dose levels below those causing dose-limiting toxicities. Thus, ZD1839 inhibited EGFR activation and downstream receptor-dependent processes in vivo. These effects were profound at doses well below the one producing unacceptable toxicity, a finding that strongly supports pharmacodynamic assessments to select optimal doses instead of a maximum-tolerated dose for definitive efficacy and safety trials (Albanell, J. et al. (2002), J Clin Oncol, 20:110-124). Basal cells in normal human epidermis, oropharyngeal mucosa, colon epithelium and endometrium normally express pSmad2, as detected by immunohistochemistry (Xie, W. et al. (2002), Cancer Res., 62:497-505; Xie, W. et al. (2003), The Cancer J., 9:302-312); Parekh, T. V. et al. (2002), Cancer Res, 62:2778-2790; Wen Xie and Michael Reiss, unpublished observations). Based on these findings, it may be able to assess and monitor the effects of TβR-I kinase inhibitors by measuring pSmad2 levels (either by immunohistochemistry or western blotting) in serial tissue biopsies (e.g. skin, gingival, colon, endometrium).

Besides skin, blood represents the most easily accessible source of normal human nucleated cells. Moreover, blood can be obtained serially at a much greater frequency than skin biopsies. As shown herein, freshly isolated human peripheral blood mononuclear cells (PBMC) express high levels of pSmad2 In addition, exposure of PBMC to TβKI in vitro results in inhibition of TβR-I kinase activity and subsequent dephosphorylation of pSmad2. Therefore, it may be able to assess TβKI activity in vivo by measuring pSmad2 levels in PBMC as described herein. It has also been shown that the expression of certain genes are altered in PBMCs after exposure to TGF-β. Moreover, some of these same genes (Tables 3 and 4) are modulated in the presence of particular kinase inhibitors. Thus, this gene set may serve as biomarkers for monitoring therapy with a kinase inhibitor in vivo.

### Screening Methods

It is a further object of the invention to provide for a method of identifying, by high throughput screening, a therapeutic agent that inhibits TGFβ receptor kinase activity.

In one embodiment, the method comprises contacting TGFβ responsive cells with an agent (a candidate drug), and detecting the binding of an antibody specific for pSmad2/3 as described herein, or a derivative of fragment thereof, wherein the inability to detect binding of the antibody to pSmad2/3 is indicative of an active TGFβ receptor kinase inhibitory agent. In a particular embodiment, the antibody specifically binds to phosphorylated Smad2/3, and the binding occurs only if the agent in question does not inhibit the TGFβ receptor kinase activity. The method comprises contacting said TGFβ responsive cells with said agent and determining whether said agent prevents the phosphorylation of Smad2/3, as measured by the detection (or lack thereof) of bound anti-pSmad2/3 antibody. In one embodiment, the anti-pSmad2/3 antibody may be detected by a second antibody conjugated to an enzyme, a radioisotope or any other molecule that may be detected by fluorescence or the like. In another embodiment, the method comprises the steps of:
a) incubating a culture of TGFβ responsive cells with increasing concentrations of a test agent, or with control culture medium, for a time sufficient to allow binding of TGFβ to its receptors and to activate the receptor kinases;
b) fixing and permeabilizing the cells in order to allow for antibody binding to the phosphorylated Smad2/3 molecules;
c) incubating the cells with an antibody specific for phosphorylated Smad2/3 (pSmad2/3) for a time sufficient to allow binding of the antibody to pSmad2/3;
d) detecting and quantitating the amount of pSmad2/3 antibody bound by incubating with a labeled second antibody having specificity for the pSmad2/3 antibody;
e) comparing the amount of labeled second antibody bound to TGFβ responsive cells without test compound to the amount of labeled second antibody bound to TGFβ responsive cells with test compound; and
   wherein the amount of labeled antibody bound correlates inversely with the potential of the test compound for inhibiting TGFB receptor kinase activity.

In one particular embodiment, the TGFβ responsive cells may be selected from the group consisting of Sweig cells, BALB/MK cells, HKc/HPV16 cells, Mink lung cells, HaCaT cells, MDA-MB-231 cells, and MDA-MB-435 cells and any other human or rodent, epithelial or lymphoid cell line in which TGFβ reproducibly induces phosphorylation of Smad2/3 in a dose-dependent manner.

In another embodiment, the screening may be done using a gene expression microassay (gene chip assay) or a polymerase chain reaction assay, more particularly a quantitative real time PCR assay. The steps may involve:

a) providing a cell that expresses one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1;

b) adding to the cell either TGF-β alone, or TGF-β plus a drug candidate;

c) processing the cell to release nucleic acid and cytoplasmic proteins from the cell;

d) determining the expression level of one or more of the genes from step a);

e) comparing the expression level of one or more of the genes in the cell treated with TGF-β alone with the expression level of one or more of the genes in a cell treated with TGF-β plus the drug candidate to determine:
(i) whether expression of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1 is decreased in the cell treated with TGF-β plus a drug candidate relative to a cell not treated with the drug candidate, or
   (ii) whether expression of COP1, SEC24D, ZFHX1B, FLI1, PLA2G7, CXCL2, CCR1, FUCA1, CSPG2, MNDA, PAX8, THBS1, CX3CR1, DHRS9 and SERPINB2 is increased in the cell treated with TGF-β plus a drug candidate relative to a cell not treated with the drug candidate.

The drug candidate is identified as a potential inhibitor of TGF-β signaling if the expression level of a gene listed in (i) is decreased and/or the expression level of a gene listed in (ii) is increased in comparison to a cell not treated with a potential kinase inhibitor.

In one embodiment, the cell is a tumor cell, a peripheral blood mononuclear cell (PBMC) a skin cell, a bone marrow cell, a cell obtained from a gingival biopsy, a cell obtained from the colon, a cell obtained from the endometrium and any other accessible tissue or cell of the human body.

In another particular embodiment, an agent identified by the methods described herein as a TGF-β receptor kinase inhibitor would act to down-regulate one or more genes selected from the group consisting of KLF10, S100A10, TRIM36, JUN, RAI17, DUSP1, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1 in comparison to a cell not treated with a potential kinase inhibitor.

In another particular embodiment, an agent identified by the methods described herein as a TGF-β receptor kinase inhibitor would act to up-regulate one or more genes selected from the group consisting of COP1, SEC24D, ZFHX1B, FLI1, PLA2G7, CXCL2, CCR1, FUCA1, CSPG2, MNDA, PAX8, THBS1, CX3CR1, DHRS9 and SERPINB2 in comparison to a cell not treated with a potential kinase inhibitor.

Nucleic Acid Sequences Useful in the Invention

The invention provides for methods of detecting or measuring a panel or set of target nucleic acid sequences for determining the effect of a TGF-β receptor kinase inhibitor on modulation of TGF-β signaling, or for determining a biologically effective dose of a TGF-β receptor kinase inhibitor, or for determining the effectiveness of therapy with a TGF-β receptor kinase inhibitor in patients receiving such therapy, or for determining whether a TGF-β receptor kinase inhibitor would be effective in treating a patient in need of such therapy. The panel or set of target nucleic acid sequences are provided in SEQ ID NOs: 1-42. The invention may also utilize specific oligonucleotide primers for amplifying a particular template nucleic acid sequence and specific probes for identifying the panel or set of target sequences. The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength, the temperature, and incidence of mismatched base pairs.

Nucleic Acid Probes and Primers

Primers and Probes Useful for Practicing the Methods of the Invention

The invention provides specific oligonucleotide primers and probes useful for detecting or measuring a nucleic acid, and for amplifying a template nucleic acid sequence. Oligonucleotide primers useful according to the invention may be single-stranded DNA or RNA molecules that are hybridizable to a template nucleic acid sequence and prime enzymatic synthesis of a second nucleic acid strand. The primer is complementary to a portion of a target molecule present in a pool of nucleic acid molecules. It is contemplated that oligonucleotide primers according to the invention may be prepared by synthetic methods, either chemical or enzymatic. Alternatively, such a molecule or a fragment thereof may be naturally-occurring, and is isolated from its natural source or purchased from a commercial supplier. Oligonucleotide primers and probes are generally 5 to 100 nucleotides in length, ideally from 17 to 40 nucleotides, although primers and probes of different lengths may also be used . Primers for amplification are preferably about 17-25 nucleotides. Primers useful according to the invention are also designed to have a particular melting temperature (Tm) by the method of melting temperature estimation. Commercial programs, including Oligo™, Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65° C. and more preferably between about 50° and 60° C. Preferably, the Tm of a probe useful according to the invention is 7° C. higher than the Tm of the corresponding amplification primers.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of the primer to a second nucleic acid molecule. These factors, which include primer length, nucleotide sequence and/or composition, hybridization temperature, buffer composition and potential for steric hindrance in the region to which the primer is required to hybridize, will be considered when designing oligonucleotide primers according to the invention.

A positive correlation exists between primer length and both the efficiency and accuracy with which a primer will anneal to a target sequence. In particular, longer sequences have a higher melting temperature (T_{M}) than do shorter ones, and are less likely to be repeated within a given target sequence, thereby minimizing promiscuous hybridization. Primer sequences with a high G-C content or that comprising palindromic sequences tend to self-hybridize, as do their intended target sites, since unimolecular, rather than bimolecular, hybridization kinetics are generally favored in solution.

However, it is also important to design a primer that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with primer annealing efficiency, as does the concentration of organic solvents, e.g. formamide, that might be included in a priming reaction or hybridization mixture, while increases in salt concentration facilitate binding. Under stringent annealing conditions, longer hybridization probes, or synthesis primers, hybridize more efficiently than do shorter ones, which are sufficient under more permissive conditions. Stringent hybridization conditions typically include salt concentrations of less than about 1M, more usually less than about 500 mM and preferably less than about 200 mM. Hybridization temperatures range from as low as 0° C. to greater than 22° C., greater than about 30° C., and (most often) in excess of about 37° C. Longer fragments may require higher hybridization temperatures for specific hybridization. As several factors affect the stringency of hybridization, the combination of parameters is more important than the absolute measure of a single factor. Oligonucleotide primers can be designed with these considerations in mind and synthesized according to methods known to those skilled in the art.

Oligonucleotide Primer Design Strategy

The design of a particular oligonucleotide primer for the purpose of sequencing, PCR, or for use in identifying target nucleic acid molecules of GBS involves selecting a sequence that is capable of recognizing the target sequence, but has a minimal predicted secondary structure. The design of a primer is facilitated by the use of readily available computer programs, developed to assist in the evaluation of the several parameters described above and the optimization of primer sequences. Examples of such programs are “Primer Express” (Applied Biosystems), “PrimerSelect” of the DNAStar™. “PrimerSelect” of the DNAStar™ software package (DNAStar, Inc.; Madison, Wis.), OLIGO 4.0 (National Biosciences, Inc.), PRIMER, Oligonucleotide Selection Program, PGEN and Amplify (described in Ausubel et al., 1995, Short Protocols in Molecular Biology, 3rd Edition, John Wiley & Sons).

It is well known by those with skill in the art that oligonucleotides can be synthesized with certain chemical and/or capture moieties, such that they can be coupled to solid supports. Suitable capture moieties include, but are not limited to, biotin, a hapten, a protein, a nucleotide sequence, or a chemically reactive moiety. Such oligonucleotides may either be used first in solution, and then captured onto a solid support, or first attached to a solid support and then used in a detection reaction. An example of the latter would be to couple a downstream probe molecule to a solid support, such that the 5′ end of the downstream probe molecule comprised a fluorescent quencher. The target nucleic acid could hybridize with the solid-phase downstream probe oligonucleotide, and a liquid phase upstream primer could also hybridize with the target molecule. This would cause the solid support-bound fluorophore to be detectable. Different downstream probe molecules could be bound to different locations on an array. The location on the array would identify the probe molecule, and indicate the presence of the template to which the probe molecule can hybridize.

Synthesis

The primers themselves are synthesized using techniques that are also well known in the art. For example, oligonucleotides are prepared by a suitable chemical synthesis method, including, for example, the phosphotriester method described by Narang et al., 1979, Methods in Enzymology, 68:90, the phosphodiester method disclosed by Brown et al., 1979, Methods in Enzymology, 68:109, the diethylphosphoramidate method disclosed in Beaucage et al., 1981, Tetrahedron Letters, 22:1859, and the solid support method disclosed in U.S. Pat. No. 4,458,066, or by other chemical methods using either a commercial automated oligonucleotide synthesizer (which is commercially available) or VLSIPS™ technology.

Probes

As used herein, the term “probe” refers to a labeled oligonucleotide which forms a duplex structure with a sequence in the target nucleic acid, due to complementarity of at least one sequence in the probe with a sequence in the target region. The probe, preferably, does not contain a sequence complementary to sequence(s) used in the primer extension (s). Generally the 3′ terminus of the probe will be “blocked” to prohibit incorporation of the probe into a primer extension product. “Blocking” can be achieved by using non-complementary bases or by adding a chemical moiety such as biotin or a phosphate group to the 3′ hydroxyl of the last nucleotide, which may, depending upon the selected moiety, serve a dual purpose by also acting as a label for subsequent detection or capture of the nucleic acid attached to the label. Blocking can also be achieved by removing the 3′-OH or by using a nucleotide that lacks a 3′-OH such as dideoxynucleotide.

In certain embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will be of particular utility. Longer contiguous identical or complementary sequences, e.g., those of about 20, 30, 40, 50, 100, 200, 500, 1000 (including all intermediate lengths) and even up to full length sequences also be of use in certain embodiments.

The ability of such nucleic acid probes to specifically hybridize to a sequence of interest will enable them to be of use in detecting the presence of complementary sequences in a given sample.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in PCR assays. This would allow a gene product, or fragment thereof, to be analyzed, in various samples, including but not limited to biological samples. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequence set forth in SEQ ID NOs: 1 through 42 or to any continuous portion of the sequence, from about 15-25 nucleotides in length up to and including the full length sequence, that one wishes to utilize as a probe or primer.

Small polynucleotide segments or fragments may be readily prepared by, for example, directly synthesizing the fragment by chemical means, as is commonly practiced using an automated oligonucleotide synthesizer.

For hybridization techniques, a partial sequence may be labeled (e.g., by nick-translation or end-labeling with ³²P) using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in a known region of a gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Probes of the present invention may also have one or more detectable markers attached to one or both ends. The marker may be virtually any molecule or reagent which is capable of being detected, representative examples of which include radioisotopes or radiolabeled molecules, fluorescent molecules, fluorescent antibodies, enzymes, or chemiluminescent catalysts. Within certain embodiments of the invention, the probe may contain one or more labels such as a fluorescent or enzymatic label (e.g., quenched fluorescent pairs, or, a fluorescent label and an enzyme label), or a label and a binding molecule such as biotin (e.g., the probe, either in its cleaved or uncleaved state, may be covalently or non-covalently bound to both a label and a binding molecule (see also, e.g., U.S. Pat. No. 5,731,146).

As noted above, the probes of the present invention may also be linked to a solid support either directly, or through a chemical linker. Representative examples of solid supports include silicaceous, cellulosic, polymer-based, or plastic materials.

Methods for constructing such nucleic acid probes may be readily accomplished by one of ordinary skill in the art, given the disclosure provided herein. Particularly preferred methods are described for example by: Matteucci and Caruthers, J. Am. Chem. Soc. 103:3185, 1981; Beaucage and Caruthers, Tetrahedron Lett. 22:1859-1862, 1981; U.S. Pat. Nos. 4,876,187 and 5,011,769; Ogilvie et al., Proc. Natl. Acad. Sci. USA 85:8783-8798, 1987; Usman et al., J. Am. Chem. Soc. 109:7845-7854, 1987; Wu et al., Tetrahedron Lett. 29:4249-4252, 1988; Chaix et al., Nuc. Acids Res. 17:7381-7393, 1989; Wu et al., Nuc. Acids Res. 17:3501-3517, 1989; McBride and Caruthers, Tetrahedron Lett. 24:245-248, 1983; Sinha et al., Tetrahedron Lett. 24:5843-5846, 1983; Sinha et al., Nuc. Acids Res. 12:4539-4557, 1984; and Gasparutto et al., Nuc. Acids Res. 20:5159-5166, 1992.

Detection Reactions

A wide variety of cycling reactions for the detection of a desired target nucleic acid molecule may be readily performed according to the general steps set forth above (see also, U.S. Pat. Nos. 5,011,769 and 5,403,711).

In another embodiment, Cycle ProbeTechnology (CPT) can be used for detecting amplicons generated by any target amplification technology. For example CPT enzyme immunoassay (CPT-EIA) can be used for the detection of PCR amplicons. CPT allows rapid and accurate detection of PCR amplicons. CPT adds a second level of specificity which will prevent detection of non-specific amplicons and primer-dimers. The PCR-CPT method may also be used for mismatch gene detection. Other variations of this assay include ‘exponential’ cycling reactions such as described in U.S. Pat. No. 5,403,711 (see also U.S. Pat. No. 5,747,255).

A lateral flow device (strip or dipstick) as described in U.S. Pat. Nos. 4,855,240 and 4,703,017. Other suitable assay formats including any of the above assays which are carried out on solid supports such as dipsticks, magnetic beads, and the like (see generally U.S. Pat. Nos. 5,639,428; 5,635,362; 5,578,270; 5,547,861; 5,514,785; 5,457,027; 5,399,500; 5,369,036; 5,260,025; 5,208,143; 5,204,061; 5,188,937; 5,166,054; 5,139,934; 5,135,847; 5,093,231; 5,073,340; 4,962,024; 4,920,046; 4,904,583; 4,874,710; 4,865,997; 4,861,728; 4,855,240; 4,847,194 and 6,130,098).

Polynucleotide Amplification Techniques

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCRT, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™ bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Q beta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992, incorporated herein by reference in its entirety), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA.

PCT Intl. Pat. Appl. Publ. No. WO 89/06700, incorporated herein by reference in its entirety, discloses a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

The invention also provides a kit for generating a signal indicative of the presence of a target nucleic acid sequence in a sample, wherein the target nucleic acid sequences are selected from those identified in SEQ ID NOs: 1-42, comprising a nucleic acid polymerase, a primer, a probe and a suitable buffer. In a particular embodiment, the invention also provides a kit for generating a signal indicative of the presence of a target nucleic acid sequence selected from those identified in SEQ ID NOs: 1-42 in a sample comprising one or more nucleic acid polymerases, primers and probes and a suitable buffer.

In another particular embodiment the kit further comprises a labeled nucleic acid complementary to the target nucleic acid sequence.

Further features and advantages of the invention are as follows. The claimed invention provides a method of generating a signal to detect and/or measure a set or panel of genes in a sample wherein the generation of a signal is an indication of the presence of one or more of the nucleic acids of SEQ ID NOs: 1-42 in a sample. The claimed invention also provides a PCR based method and a gene expression microarray method for detecting and/or measuring these nucleic acids in a sample of tissue or cells comprising generating a signal as an indication of the presence of one or more of these nucleic acids. The claimed invention also allows for the detection of one or more of these gene sequences by quantitative real time-PCR.

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.).

Production of a Nucleic Acid

The invention provides for nucleic acids to be detected and or measured, and for amplification (DR. REISS, MUST THE NUCLEIC ACID SEQUENCES BE AMPLIFIED TO AID IN THEIR DETECTION?) of a target nucleic acid sequence for identification of genes found in tissue or cells obtained for measuring the effect of TGF-β kinase inhibitors on TGF-β signaling or for determining the effect of a kinase inhibitor in patients being treated with such kinase inhibitors or for determining whether a patient who is a candidate for such therapy would be responsive to such therapy. The methods also provide for screening for novel kinase inhibitors using the gene sequences that are modulated by TGF-β, such as those described in SEQ ID NOs: 1-42.

Polymerase Chain Reaction (PCR)

Nucleic acids of the invention may be amplified from genomic DNA or other natural sources by the polymerase chain reaction (PCR). PCR methods are well-known to those skilled in the art.

PCR provides a method for rapidly amplifying a particular DNA sequence by using multiple cycles of DNA replication catalyzed by a thermostable, DNA-dependent DNA polymerase to amplify the target sequence of interest. PCR requires the presence of a target nucleic acid sequence to be amplified, two single stranded oligonucleotide primers flanking the sequence to be amplified, a DNA polymerase, deoxyribonucleoside triphosphates, a buffer and salts.

PCR, is performed as described in Mullis and Faloona, 1987, Methods Enzymol., 155: 335, herein incorporated by reference.

The polymerase chain reaction (PCR) technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment whose termini are defined by the 5′ ends of the primers. PCR is reported to be capable of producing a selective enrichment of a specific DNA sequence by a factor of 10⁹. The PCR method is also described in Saiki et al., 1985, Science 230:1350.

PCR is performed using template DNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of a suitable buffer, 0.4 μl of 1.25 μM dNTP, 2.5 units of Taq DNA polymerase (Stratagene) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is generally used. Initial denaturation of the template molecules normally occurs at between 92° C. and 99° C. for 4 minutes, followed by 20-40 cycles consisting of denaturation (94°-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

In a particular embodiment of the present invention, the PCR procedure may be a real-time PCR procedure. Moreover, the PCR procedure employed may use the materials and methodology outlined in U.S. Pat. No. 6,130,098, incorporated herein by reference in its entirety.

Detection methods generally employed in standard PCR techniques use a labeled probe with the amplified DNA in a hybridization assay. Preferably, the probe is labeled, e.g., with ³²P, biotin, horseradish peroxidase (HRP), etc., to allow for detection of hybridization.

In a particular embodiment of the present invention, the probe utilized recognizes the sequence amplified between the primers, allowing real-time detection by using fluorescence measurements. A further embodiment of the present invention includes a pair of PCR amplification primers specific for a portion of one or more of the genes of SEQ ID NOs: 1-42.

Other means of detection include the use of fragment length polymorphism (PCR FLP), hybridization to allele-specific oligonucleotide (ASO) probes (Saiki et al., 1986, Nature 324:163), or direct sequencing via the dideoxy method (using amplified DNA rather than cloned DNA). The standard PCR technique operates (essentially) by replicating a DNA sequence positioned between two primers, providing as the major product of the reaction a DNA sequence of discrete length terminating with the primer at the 5′ end of each strand. Thus, insertions and deletions between the primers result in product sequences of different lengths, which can be detected by sizing the product in PCR-FLP. In an example of ASO hybridization, the amplified DNA is fixed to a nylon filter (by, for example, UV irradiation) in a series of “dot blots”, then allowed to hybridize with an oligonucleotide probe labeled with HRP under stringent conditions. After washing, terramethylbenzidine (TMB) and hydrogen peroxide are added: HRP oxidizes the hydrogen peroxide, which in turn oxidizes the TMB to a blue precipitate, indicating a hybridized probe.

Oligonucleotide Design for Real-Time PCR Assays

There are several different approaches to real-time PCR. SYBR green detection is utilized with real time PCR because multiple reactions can be set-up rapidly and inexpensively using standard oligonucleotides. Real-time PCR relies on the fluorescent quantification of PCR product during each cycle of amplification. Specific detection systems, such as molecular beacons and Taqman assays rely on the synthesis of a fluorescently labeled detection oligonucleotide. These specific assays have the advantage of specificity, but the disadvantage of added expense and a delay in obtaining the fluorescently labeled detection oligonucleotides. Assay of PCR product through the use of the fluorescent dye SYBR green allows the reaction to be based on standard oligonucleotides. Because SYBR green will detect any PCR product, including non-specific products and primer-dimers, careful oligonucleotide design for the reaction is required.

Primers should be designed, if possible, within 1 kb of the polyadenylation site. Amplicons of 100-200 bp are ideal for real time applications. It is advantageous to design the primers to have the same melting temperature so that PCR with different primer sets can be performed in the same run. Primers that are 20-mers with 55% GC content and a single 3′-G or C can be used. Candidate primers are tested for specificity by BLAST and for folding and self annealing using standard DNA analysis software. Primer pairs are first tested for specificity and absence of primer-dimer formation (low molecular weight products) by PCR followed by gel electrophoresis. Designing each primer pair takes about one hour.

Real Time PCR

Real-time PCR requires a specialized thermocycler with fluorescent detection. A variety of commercial instruments are available. The ABI Prism 7700 allows assays to be performed in 96 well plate format. Good PCR technique is required to avoid contamination of subsequent reactions. This includes isolating PCR products and plasmids from RNA preparation and reaction setup. A dedicated bench for RNA isolation and PCR reaction set-up and dedicated pipettors should be maintained. Aerosol resistant pipette tips are used.

Commercial kits for SYBR green based PCR reactions are available from Applied Biosystems and perform reliably (SYBR Green PCR Core Reagents, P/N 430-4886; SYBR Green PCR Master Mix, P/N 4309155).

“Hot start” taq polymerase may be used. Platinum Taq, (Life Technologies), and Amplitaq gold, (Applied Biosystems), both perform well. The 10×SYBR Green I may be prepared by diluting 10111 of the stock 10,000× concentrate (Cat# S-7563, Molecular Probes, Eugene, Oreg.) into 10 ml Tris-HCl, pH 8.0, and is stored in 0.5 ml aliquots at −20° C. 15 μl of the master mix are aliquoted into 0.2-mL MicroAmp optical tubes (P/N N801-0933, Applied Biosystems). Alternatively, a 96-well optical reaction plate (P/N 4306737, Applied Biosystems) can be used. Five μl of the first strand cDNA is then added to the tube and the solution is mixed by repeat pipetting. This achieves a final concentration reaction containing 20 mM Tris-, 50 mM KCl, 3 mM MgCl₂, 0.5×SYBR Green I, 200 μM dNTPs, 200 μM each of forward and reverse primers, approximately 500 pg first strand cDNA, and 0.5 units Taq polymerase.

The reaction tubes are covered with MicroAmp optical caps (P/N N801-0935, Applied Biosystems) using a cap installing tool (P/N N801-0438, Applied Biosystems). The contents are collected to the bottom of the tube by brief centrifugation in a Sorvall RT-6000B benchtop centrifuge fitted with a microplate carrier (PN 11093, Sorvall). The tubes are then placed in the ABI 7700 thermocycler and incubated at 95° C. for 2 minutes (10 minutes if using Amplitaq gold) to activate the enzyme and denature the DNA template. Forty cycles of PCR amplification are then performed as follows: Denature 95° C. for 15 seconds, Anneal 55° C. for 20 seconds, Extend 72° C. for 30 seconds.

This protocol works well for amplicons up to 500 base pairs. For longer amplicons, the extension step should be adjusted accordingly (approximately 1 minute per kb). Either the FAM or the SYBR channel can be used for fluorescence detection of SYBR Green I. Fluorescent emission values are collected every 7 seconds during the extension step. Data are analyzed using Sequence Detector version 1.7 software (Applied Biosystems). In order to obtain the threshold cycle (C_{T}) values, the threshold is set in the linear range of a semi-log amplification plot of ΔRn against cycle number. This ensures that the C_{T} is within the log phase of the amplification. Here the ΔRn is the fluorescence emission value minus baseline fluorescence value. When the PCR is at 100% efficiency, the C_{T} decreases by 1 cycle as the concentration of DNA template doubles.

In order to confirm that the correct amplicon is made, the amplified products are analyzed by agarose gel electrophoresis and visualized by ethidium bromide staining. A good reaction yields a single band of the expected size and has no smearing or primer-dimer formation.

To generate a standard curve for each primer pair, 10-fold serial dilutions are made from a plasmid with known number of copies of the gene. The C_{T} of each dilution is determined, and is plotted against the log value of the copy number. Amplification efficiency of each primer pair is obtained by the slope of regression. A 100% efficient PCR has a slope of −3.32. The number of copies in the samples is extrapolated by its C_{T} value using the respective standard curve.

In a more particular embodiment of the invention, RNA was isolated from PBMC, and was used in quantitative real-time PCR analysis using the QuantiTect Probe RT-PCR kit (QIAGEN Inc., Valencia, Calif.). For PCR, 50 μl reactions were set up with 85 ng of RNA, 0.4 μM primer, 0.2 μM dual labeled probe, 0.5 μl of QuantiTect Reverse Transcriptase Mix and QuantiTect Probe RT-PCR Master Mix. The real-time PCR was performed in a Mx4000 Multiplex Quantitative PCR System (stratagene) with each data point performed in triplicate. Five RNA targets were quantified, including SERPINE 1 (or PAI-I), OSM, VEGF, OLR-1 and the control gene glyceraldehyde-3-phosphate dehydrogenase (GAPDH). Standard curves for all five genes were generated using serial dilution of RNA isolated from baseline-control cells. The mRNA amounts for each gene in the individual RNA samples, was calculated from the standard curves. Appropriate gene-specific primers used for the analysis were designed using Integrated DNA Technologies PrimerQuest.

The following primers and probes were used:

VEGF:
Forward

(SEQ ID NO: 85)

5′-TTT CTG CTG TCT TGG GTG CAT TGG-3′,
Reverse

(SEQ ID NO: 86)

5′-ACC ACT TCG TGA TGA TTC TGC CCT-3′,
Probe

(SEQ ID NO: 87)

5′-FAM-TTG CTG CTC TAC CTC CAC CAT GCC AA-BHQ-3′;
OSM:
Forward

(SEQ ID NO: 88)

5′-AGT CTG GTC CTT GCA CTC CTG TTT-3′,
Reverse

(SEQ ID NO: 89)

5′-TGT CCT GCA TGA GAT CTG TCT GCT-3′,
Probe

(SEQ ID NO: 90)

5′-FAM-AAG CAT GGC GAG CAT GGC GGC TAT A-BHQ-3′;
OLR1:
Forward

(SEQ ID NO: 91)

5′-GAA GGT TGT GAA ATC AAG CAG GCG-3″
Reverse

(SEQ ID NO: 92)

5′-AAG TGC CCT TGA CTT AGT GGT GGT-3′,
Probe

(SEQ ID NO: 93)

5′-FAM-ACC GCT TGG TTT GAA GGC AGC TTT GA-BHQ-3′;
SERPINE1:
Forward

(SEQ ID NO: 94)

5′-TGC TGG TGA ATG CCC TCT ACT TCA-3′,
Reverse

(SEQ ID NO: 95)

5′-AGA GAC AGT GCT GCC GTC TGA TTT-3′,
Probe

(SEQ ID NO: 96)

5′-FAM-ACG GCC AGT GGA AGA CTC CCT T-BHQ-3′;
GAPDH:
Forward

(SEQ ID NO: 97)

5′-CCA CCC ATG GCA AAT TCC-3′,
Reverse

(SEQ ID NO: 98)

5′-TCG CTC CTG GAA GAT GGT G-3″
Probe

(SEQ ID NO: 99)

5′-FAM-TGG CAC CGT CAA GGC TGA GAA CGT-BHQ-3′.

### Kits

It is a further object of the invention to provide for a diagnostic test kit for determining a biologically effective dose of a TGFβ receptor kinase inhibitor or the optimal biologic dose of a TGFβ receptor kinase inhibitor to be administered to a patient in need of such therapy, or for monitoring the effectiveness of therapy with a TGFβ receptor kinase inhibitor in patients receiving such therapy, or for predicting whether a subject is a candidate for therapy with a TGFβ receptor kinase inhibitor.

In one embodiment, the method comprises the steps of:
a) providing a predetermined amount of an antibody specific for pSmad2/3;
b) providing a predetermined amount of a specific binding partner of said antibody;
c) providing buffers and other reagents necessary for monitoring detection of antibody bound to pSmad2/3 in a bodily sample; and
d) providing directions for use of said kit;
   wherein either said antibody or said specific binding partner are detectably labeled.

The present invention includes an assay system which may be prepared in the form of a test kit for the quantitative analysis of the extent of the presence of the phosphorylated forms of the TGFβ receptor kinases, or to identify drugs or other agents that may block their activity. The system or test kit may comprise a labeled component prepared by one of the radioactive and/or enzymatic techniques discussed herein, coupling a label to the antibodies, and one or more additional immunochemical reagents, at least one of which is a free or immobilized ligand, capable either of binding with the labeled component, its binding partner, one of the components to be determined or their binding partner(s).

In another embodiment, the invention provides a diagnostic test kit for determining the effect of a TGF-β receptor kinase inhibitor on modulation of TGF-β signaling, or for determining a biologically effective dose of a TGF-β kinase inhibitor, or for determining the effectiveness of therapy with a TGF-β receptor kinase inhibitor in patients receiving such therapy, or for identifying a TGF-β receptor kinase inhibitor that would be effective in treating a patient in need of such therapy, comprising:

a) one or more nucleic acids encoding one or more of the proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2; and SERPINE1;

b) reagents useful for monitoring the expression level of the one or more nucleic acids or proteins encoded by the nucleic acids of step a);

c) instructions for use of the kit.

In another embodiment, the kit comprises at least five nucleic acids encoding at least five proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, and SERPINB2.

In another embodiment, the kit comprises at least ten nucleic acids encoding at least ten proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, and SERPINB2.

In yet another embodiment, the kit for determining the effect of a TGF-β receptor kinase inhibitor on modulation of TGF-β signaling, or for determining a biologically effective dose of a TGF-β kinase inhibitor, or for determining the effectiveness of therapy with a TGF-β receptor kinase inhibitor in patients receiving such therapy, or for identifying a TGF-β receptor kinase inhibitor that would be effective in treating a patient in need of such therapy, comprises: a first plurality of oligonucleotides, comprising the nucleic acid sequences of five or more SEQ ID NOs; 1-42, or the complements thereof, and a second plurality of oligonucleotides, comprising mismatch oligonucleotides corresponding to the first plurality of oligonucleotides. Each oligonucleotide is attached to a solid support in a determinable location. In another embodiment, the solid support is a plurality of beads. In yet another embodiment, the solid support is glass.

### Use of Microarrays for Determining Gene Expression Levels

The invention also provides for an array of oligonucleotides comprising the nucleic acid sequences of one or more of SEQ ID NOs; 1 through 42 attached to a solid support in a determinable location of the array. Microarrays may be used for determining gene expression levels and may be prepared by methods known in the art, or they may be custom made by companies, e.g., Affymetrix (Santa Clara, Calif.) (see www.affymetrix.com). Numerous articles describe the different microarray technologies, (e.g., Shena, et al., Tibtech, (1998), 16: 301; Duggan, et al., Nat. Genet., (1999), 21:10; Bowtell, et al., Nat. Genet., (1999), 21:25; Hughes, et al., Nat. Biotechn., (2001), 19:342). While many of the microarrays utilize nucleic acids and relevant probes for the analysis of gene expression profiles, protein arrays, in particular, antibody arrays or glycosylation arrays also hold promise for studies related to protein or glycoprotein expression from biological samples (see for example, RayBiotech, Inc. at www.raybiotech.com/product.htm, Panomics at www.panomics.com, Clontech Laboratories, inc. at www.clontech.com, Procognia in Maidenhead, UK and Qiagen at www.qiagen.com.

### Other Methods for Determining Gene Expression Levels

In certain embodiments, it is sufficient to determine the expression of one or only a few genes, as opposed to hundreds or thousands of genes. Although microarrays may be used in these embodiments, various other methods of detection of gene expression are available.

For example, as noted above, the modulation of gene expression can be performed using a RT-PCR or Real Time-PCR assay. Total RNA is extracted using procedures known to those skilled in the art and subjected to reverse transcription using an RNA-directed DNA polymerase, such as reverse transcriptase isolated from AMV, MoMuLV or recombinantly produced. The cDNAs produced can be amplified in the presence of Taq polymerase and the amplification monitored in an appropriate apparatus in real time as a function of PCR cycle number under the appropriate conditions that yield measurable signals, for example, in the presence of dyes that yield a particular absorbance reading when bound to duplex DNA. The relative concentrations of the mRNAs corresponding to chosen genes can be calculated from the cycle midpoints of their respective Real Time-PCR amplification curves and compared between cells exposed to a candidate therapeutic relative to a control cell in order to determine the increase or decrease in mRNA levels in a quantitative fashion.

In other methods, the level of expression of a gene is detected by measuring the level of protein encoded by the gene. In the case of polypeptides which are secreted from cells, the level of expression of these polypeptides may be measured in biological fluids. While methods such as immunoprecipitation, ELISA, Western blot analysis, or immunohistochemistry using an agent, e.g., an antibody, that specifically detects the protein encoded by the gene may be contemplated, other more sensitive and quantitative methods are preferred, as described below.

The invention is not limited to a particular assay procedure, and therefore is intended to include both homogeneous and heterogeneous procedures. General techniques to be used in performing the various immunoassays noted above are known to those of ordinary skill in the art.

### EXAMPLES

The following examples are put forth so as to provide those of ordinary skill in the art with a complete disclosure and description of how to make and use the antibodies and diagnostic procedures described herein, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### pSmad2 Expression as a Measure of TβR-I Kinase Activity-Cell Lines, Tissues

Preparation of Activation State-Specific Anti-Smad Antibodies: in Order to be Able to assess the state of TGFβ receptor signaling in cells and tissues, polyclonal rabbit antibodies specifically directed against the phosphorylated (activated) forms of the R-Smads, Smad2 and -3 were generated. Synthetic peptides comprising the C-terminal 13 amino acids of the R-Smads, in which two phosphoserine residues were incorporated at the extreme C-terminus (Smad2: KMGSPSVRCSS^{p}MS^{p} (SEQ ID NO: 1); Smad3:KMGSPSIRCSS^{p}VS^{p} (SEQ ID NO: 2)), coupled to keyhole limpet hemocyanin (KLH) as carrier protein were used as immunogen. The antiserum was affinity-purified by negative selection using a KLH-agarose column, followed by chromatography using Affigel-10 (BioRad) matrix-coupled unphosphorylated Smad2 and Smad3 peptides. The final purification step consisted of a positive selection using Affigel-10-coupled pSmad2 or pSmad3 phosphopeptides. The high specificity and sensitivity of the anti-pSmad antibodies were confirmed by ELISA (Eickelberg, O. et al. (2002), J Biol Chem, 277:823-829). As demonstrated herein, these pSmad antibodies are uniquely suited for Western blotting, immunoprecipitation, as well as immunohistochemistry.

### Results

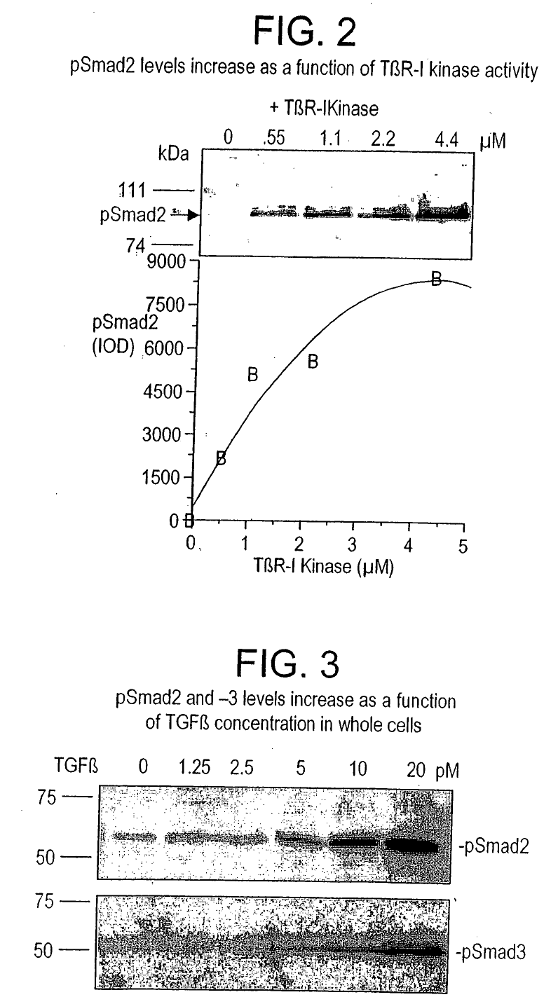

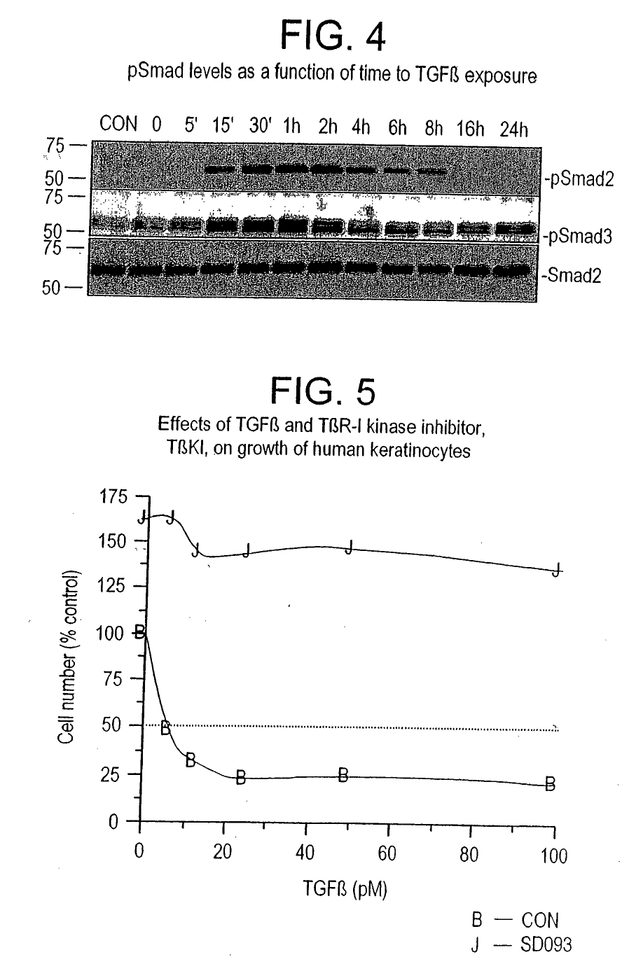

Using purified recombinant constitutively active TβR-I kinase and recombinant GST-Smad2 fusion protein in in vitro kinase assays, the pSmad2 antibody detects a band of approximately 58 kDa, the density of which is proportional to the amount of active enzyme (FIG. 2). As shown in FIG. 3, both antibodies are able to detect pSmad2 and -3 in keratinocytes treated with as little as 1.25 pM TGFβ, and the signal is proportional to the TGFβ concentration used. Moreover, increases in pR-Smad levels can be detected as early as 5 minutes following the addition of 100 pM TGFβ to the culture medium, and maximal levels are achieved at approximately 1 hour (FIG. 4). Thus, in cells with fully functional TGFβ receptors, the intracellular levels of pR-Smads appear to vary as a function of the time and concentration of TGFβ exposure. Testing the prediction that loss of receptor function would abrogate this response was then carried out. Among 7 SCC cell lines and 10 breast cancer cell lines, treatment with TGFβ induced phosphorylation of Smad2 with the exception of two SCC cell lines that carry inactivating mutations of the TβR-II receptor, and a breast cancer cell line with transcriptional inactivation of TβR-II (Xie, W. et al. (2002), Cancer Res., 62:497-505; Yan, W. et al. (2000), Oncol Res, 12:157-167, 2000). These findings indicated that it may be possible to utilize pSmad2 immunostaining to identify tumor specimens that have lost receptor function. Proof of concept was provided in a study of normal endometrium and endometrial carcinoma specimens (Parekh, T. V. et al. (2002), Cancer Res, 62:2778-2790). While immunostainable pSmad2 was easily detectable in the epithelial cells of normal endometrium, pSmad2 staining was weak or undetectable in all endometrial carcinomas (n=22), with intermediate levels of staining found in atypical glandular hyperplasias (Parekh, T. V. et al. (2002), Cancer Res, 62:2778-2790). Moreover, loss of pSmad2 expression correlated with loss of TβR-II expression and TGFβ resistance of primary endometrial carcinoma cells in vitro

### Example 2

### Effect of the TβKI on Inhibition of TGFβ-Induced Smad2 Phosphorylation, -Cellular Responses and -Regulated Gene Expression

As shown in FIG. 5, TGFβ potently inhibits growth of human keratinocytes in a dose-dependent manner, with an IC₅₀ of approximately 5 pM. Moreover, pre-incubation of the cells with the TβR-I kinase inhibitor, TβKI, completely blocks TGFβ-induced growth arrest, indicating that the response is mediated by TβR-I. Surprisingly, TβKI treatment by itself is sufficient to increase the growth of HKc/HPV16 cells by >50%. Similarly, we consistently observe a low level of pSmad2 in untreated cells, which is dramatically increased by the addition of exogenous TGFβ, and is largely eliminated by pretreatment of the cells with TβKI (FIG. 5).

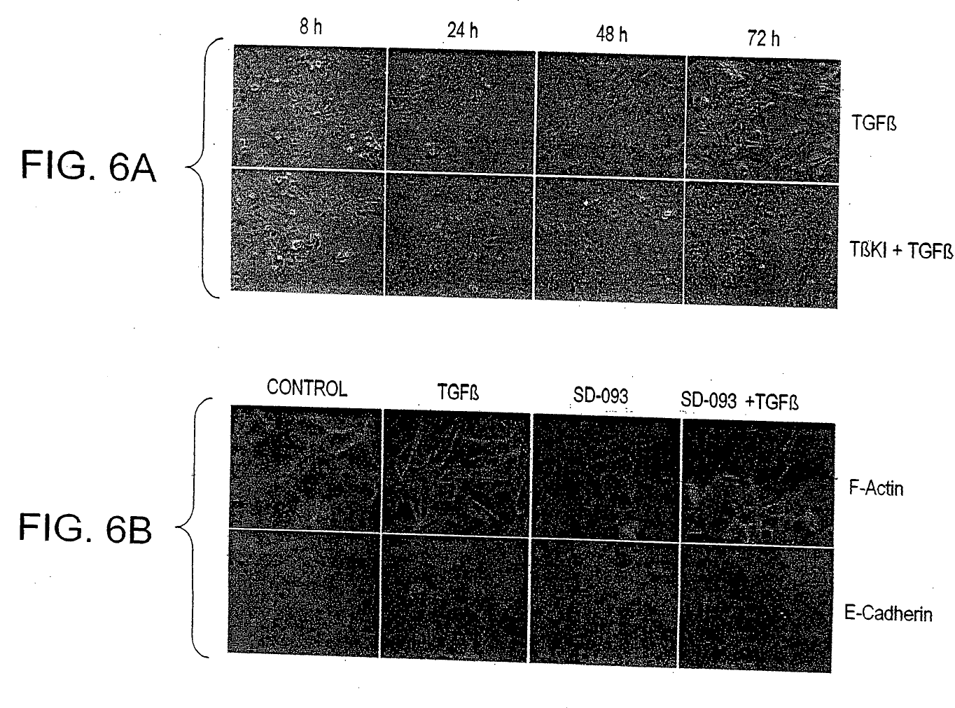

These findings indicate that, even in the absence of exogenous TGFβ, a basal level of active TGFβ signaling is going on in cultured keratinocytes, which controls cell growth. This is consistent with the presence of pSmad2 in normal self-renewing lining and glandular epithelial tissues (Xie, W. et al. (2002), Cancer Res., 62:497-505; Xie, W. et al. (2002), Cancer Res., 62:497-505; Xie, W. et al. (2003), The Cancer J., 9:302-312), and the loss of pSmad2 and epithelial hyperproliferation seen in animals with TGFβ activation defects (Sterner-Kock, A. et al. (2002), Genes & Development, 16:2264-2273). Besides growth arrest, treatment of human keratinocytes with TGFβ induces EMT (FIG. 6). During this process, cells detach from each other and acquire a spindly, fibroblastoid morphology (FIG. 6), while the tight junction protein, E-cadherin, is redistributed from the cell membranes to the cytoplasm, and F-actin is rearranged from predominantly cortical bundles to form stress fibers across the cytoplasm (FIG. 6). This process is detectable approximately 24 h following the addition of TGFβ, and peaks at 72 h. Moreover, is also completely blocked by pre-treating the cells with TβKI, indicating that it is dependent on TβR-I kinase activity.

### Example 3

### Effects of TβKIs on TGFβ-Regulated Genes

TGFβ regulates a broad range of target genes. In order to determine whether blocking TβR-I kinase activity by TβKIs inhibited TGFβ-regulated gene expression, we carried out transient transfection assays using a number of different reporter gene assays in Mv1Lu mink lung epithelial cells, which are exquisitely sensitive to TGFβ. Three different firefly luciferase reporter gene constructs were used: pSBE4 in which 4 tandem repeats of a Smad4-specific DNA binding element (SBE) drive luciferase; p3TP-Lux, which contains TGFβ-response elements from the collagenase and PAI-1 gene promoters as well as 3 tetradecanoyl phorbol acetate-response elements; and p15P751-luc (Dr. X. F. Wang, Duke University), which contains the INK4B gene. TβKIs inhibited TGFβ-induced activation of p3TP-Lux in a dose-dependent manner (not shown). The difference in activity between the compounds paralleled the difference in potency as TβR-I kinase inhibitors. Moreover, inhibition of TβR-I signaling with TβKI blocked the activation of all three reporter gene constructs by TGFβ in a dose-dependent manner. Thus, TβKIs broadly block the effects of TGFβ on target genes, and this activity is proportional to their potency as kinase inhibitors.

### Results

The TβKIs of the present invention may be quinazoline compounds such as those set forth in U.S. Pat. No. 6,476,031, incorporated by reference herein in its entirety.

Quinazoline-derived serine-threonine kinase inhibitors were tested against purified TβR-I kinase in in vitro kinase assays, as well as for their ability to inhibit TGFβ-induced phosphorylation of Smad2 in cultured cells. The relative and absolute potencies of the different compounds as determined in the in vitro kinase assay was highly predictive of their efficacy in cultured cells, indicating the excellent intracellular uptake and activity of this class of compounds.

TβKIs inhibit TGFβ-induced Smad2 phosphorylation in whole cells: Pre-treatment of MDA-MB-435 breast cancer cells with TβKIs inhibited TGFβ-induced Smad2 phosphorylation in a dose-dependent manner, with IC₅₀ values as low as 20-40 nM. Similar results were obtained using two other breast cancer cell lines, MDA-MB-231 and ZR-75-1 (data not shown). Thus, TRKIs effectively enter into cells and are capable of inhibiting the target enzyme in vivo.

### Example 4

### Effects of TGFβ and TβR-I Kinase Inhibitor on Smad2 Phosphorylation in Sweig Lymphoblastoid Cells

### Reagents

Recombinant human TGFβ1 was purchased from Austral Biologicals (San Ramon, Calif.). TGFβ1 stock solution (1 μg/μl in 4 mM HCl, 1 mg/ml BSA) was stored at −70° C. TβR-I kinase inhibitors were obtained from Scios, Inc. Purified phospho-Smad antibodies were produced as previously described (Yan, W., Vellucci, V. F., and Reiss, M. (2000), Oncol Res, 12: 157-167; Eickelberg, O., Centrella, M., Reiss, M., Kashgarian, M., and Wells, R. G. (2002), J Biol Chem, 277: 823-829; Liu, C., Gaca, M. D., Swenson, E. S., Vellucci, V. F., Reiss, M., and Wells, R. G. (2003), J Biol Chem, 278: 11721-11728.

### Cell Culture

Sweig Epstein-Barr virus-immortalized lymphoblastoid cells were maintained at 37° C. in medium composed of RPMI (GIBCO-BRL) supplemented with 10% (v/v) FBS and 2.5 mM Glutamax (GIBCO-BRL). Freshly isolated PBMC were maintained in short-term culture in medium composed of RPMI (GIBCO-BRL) supplemented with 5% (v/v) FBS.

### Isolation of Peripheral Blood Mononuclear Cells:

Peripheral blood mononuclear cells (PBMC) were isolated from whole blood. Blood (7-10 ml) was drawn from healthy volunteers using a butterfly needle and syringe, and immediately transferred to a sterile glass tube containing 0.117 ml of 15% (w/v) K₃EDTA. The blood was then mixed with an equal volume of 150 mM NaCl. Diluted blood was then layered over twice the volume of Nycoprep (Density 1.077 g/ml, Axis-Shield) and subjected to centrifugation in a swinging bucket rotor at 800 g for 30 minutes at 20° C., Following centrifugation, the mononuclear cell fraction was carefully aspirated from the plasma/Nycoprep interface using a Pasteur pipette. The PBMC were washed twice using Hank's Buffered Salt solution (Gibco BRL), resuspended in RPMI (Gibco BRL) supplemented with 5% (v/v) FBS (Sigma), transferred to 6-well tissue culture cluster dishes, and incubated at 37° C. Cell numbers were determined using a Coulter model Z2 particle counter (BD Systems), and their morphology checked by Giemsa staining.

### Smad/pSmad Detection by Western Blot

Cultured cells were collected by centrifugation and resuspended in lysis buffer composed of 150 mM NaCl, 10 mM Tris-HCl (pH 8.0), 1 mM EGTA, 1% (v/v) Triton-X-100, supplemented with 1 tablet of Complete Mini (Roche Diagnostics) for every 10 ml of buffer. Cells were lysed by subjecting them to three cycles of freezing and thawing using a dry ice-ethanol mixture, followed by incubation on ice for 45 minutes. Protein mixtures were resolved by SDS-polyacrylamide gel (12%) electrophoresis and transferred to nitrocellulose membranes. Membranes were pre-incubated with 5% (w/v) Carnation milk powder in TBS-T buffer (pH 7.6), and then incubated with either 1 μg/ml anti-pSmad2 polyclonal rabbit antibody, or 0.625 μg/ml rabbit anti-Smad2 antibody (Zymed, San Francisco, Calif.) overnight at 4° C. Blots were then washed with TBS-T and milk and TBS-T alone ×2, and treated with peroxidase-conjugated goat anti-rabbit antibody (Calbiochem) at a 1:2000 dilution in blocking solution for one hour at 20° C. The membranes were then washed with TBS-T and milk and then TBS-T alone ×3 and were covered with ECL Western blotting detection reagent (Amersham Biosciences, kkkkk, UK) at a 1:1 ratio, exposed to X-ray film (Kodak X-omat) and developed. Films were digitized using an Epson 2400 flatbed scanner, and subjected to densitometry using ImageJ software (Version 1.27, NIH).

### Results

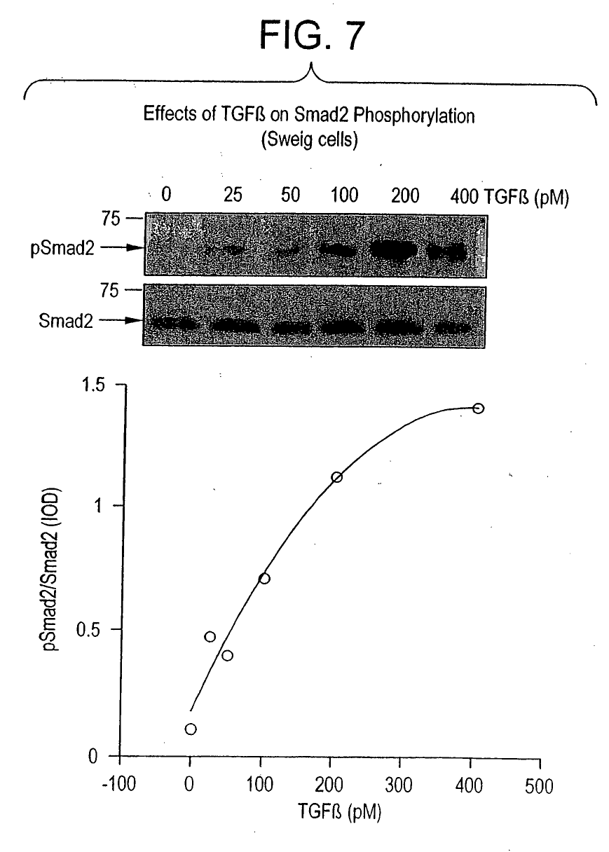

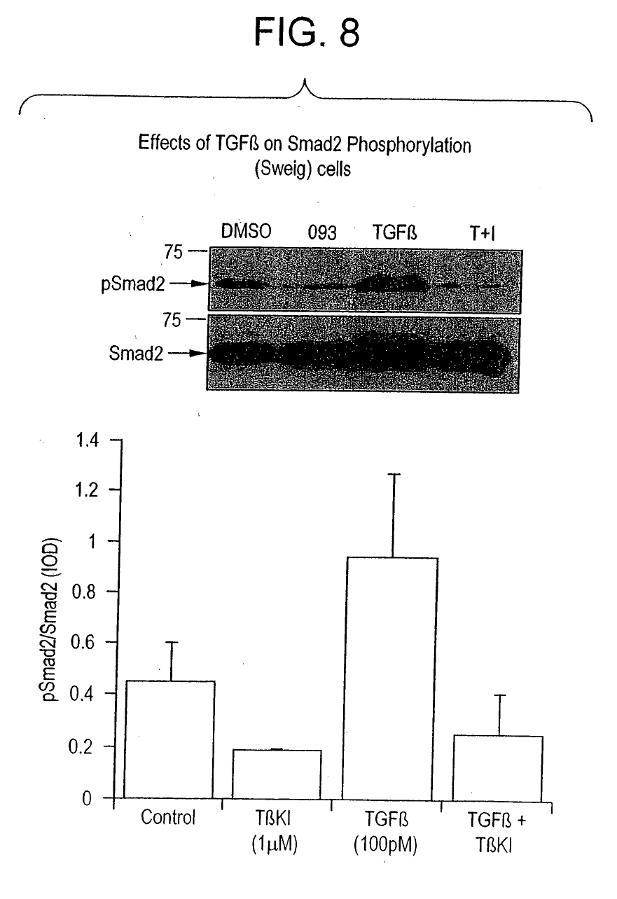

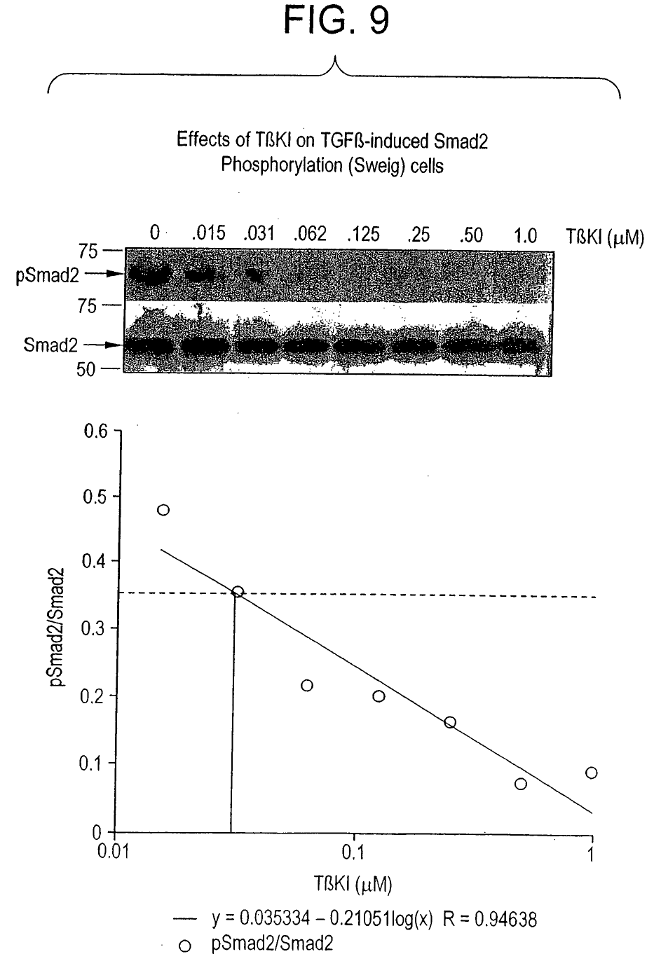

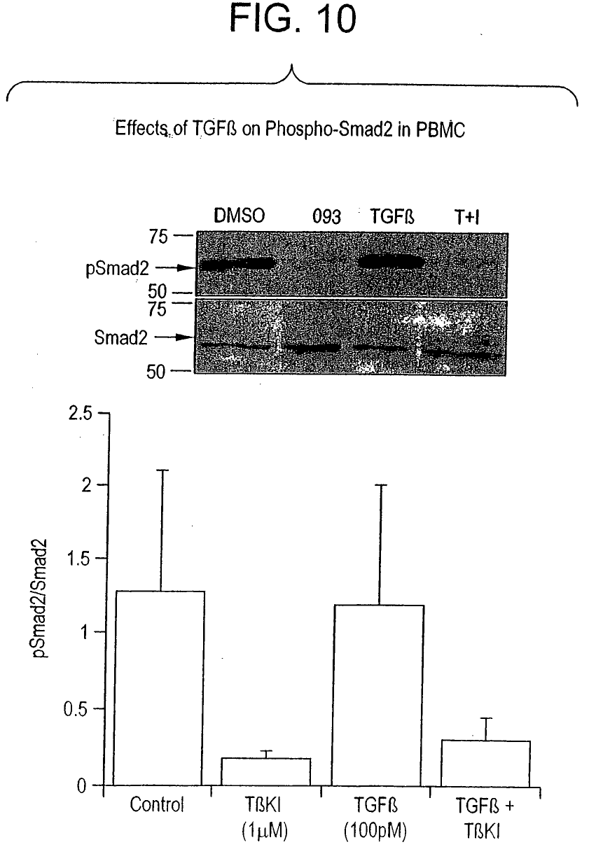

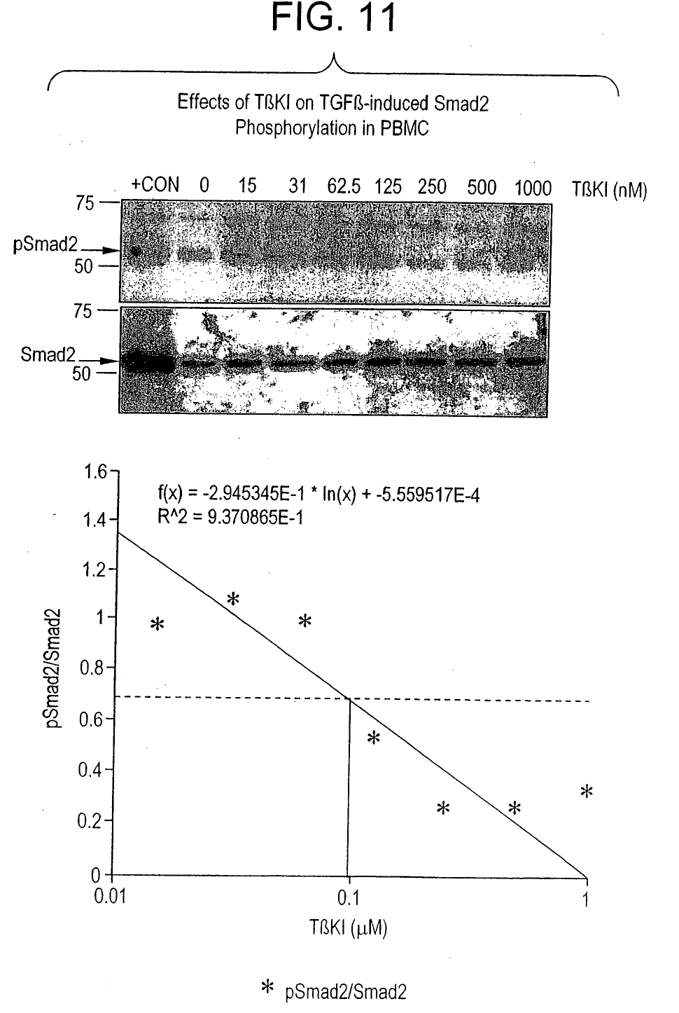

In order to determine whether immortalized lymphocytes might be utilized to assay the activity of TβKIs in plasma, the ability of TGFβ to activate TGFβ receptors in Sweig human, Epstein-Barr virus immortalized, lymphoblastoid cells was examined (FIG. 7). Cells were exposed to different concentrations of TGFβ for 2 hours. The levels of phosphorylated Smad2 (pSmad2) were then determined in cell extracts by immunoblotting using the specific anti-pSmad2 rabbit antibody. As shown in FIG. 7, TGFβ induces phosphorylation of Smad2 in a dose-dependent manner, even at the lowest concentration tested (25 pM). This finding indicates that Sweig cells express a functionally intact TβR system, as well as the TβR-I kinase substrate, Smad2. It was then determined whether the TβR-I kinase inhibitor, TβKI, could block TGFβ-induced Smad2 phosphorylation in Sweig cells. As shown in FIG. 8, exponentially growing Sweig cells express a low but detectable level of pSmad2, which was decreased by treating cells with TβKI, presumably because blocking TβR-I kinase activity allows pre-existing pSmad2 to be dephosphorylated. This finding indicates that a low level of TβR signaling is ongoing in Sweig cells in culture, even in the absence of exogenous TGFβ. As expected, pre-treatment of cells with the TβKI, completely blocked TGFβ-induced Smad2 phosphorylation (FIG. 8). In order to determine the sensitivity of TβR-I kinase activity in Sweig cells to the TβKI, TGFβ-treated Sweig cells were pre-incubated with varying doses of TβKI and pSmad2 levels assayed by immunoblotting (FIG. 9). As shown in FIG. 9, TβKI was able to inhibit TβR-I kinase activity in a dose-dependent manner with an estimated IC₅₀ of 30 nM. These findings are entirely consistent with our previous studies of the effects of TβKI on cellular responses, Smad2 phosphorylation and TGFβ-mediated transcriptional regulation in other cell lines.

### Example 5

### PBMC-Smad2 Expression and Activation-Effects of TGFβ and TβKI

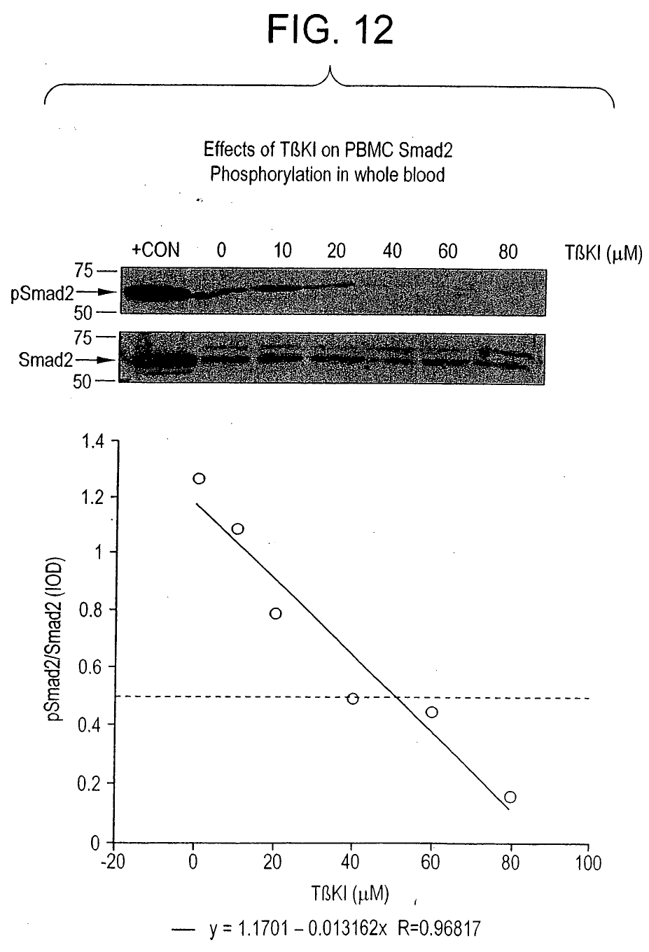

In order to determine whether freshly isolated PBMC in short-term could be used to assess the activity of TβKIs in blood (and could, therefore, be used as surrogate marker cells for tissue exposure to TβKIs), PBMC from Ca-EDTA-anticoagulated blood obtained from healthy volunteers were isolated using Nycoprep® density gradient centrifugation. Following treatment of freshly isolated PBMC with TβKI, TGFβ, TβKI followed by TGFβ or vehicle only, pSmad2 and Smad levels were determined in cell lysates by Western blotting as described above (FIG. 10). As shown in FIG. 10, freshly isolated PBMC expressed easily detectable levels of pSmad2, indicating that the TβR system was activated in these cells. To determine whether TβRs are activated in circulating PBMC or that activation takes place during the isolation procedure, a number of different blood drawing techniques were compared. Although attempts at minimizing the trauma associated with phlebotomy (and the associated platelet degranulation with release of TGFβ), did not alter the basal level of pSmad2 expression in PBMC (data not shown), the possibility cannot be excluded that the observed Smad2 phosphorylation is the result of release and activation of TGFβ as a result of the phlebotomy. Interestingly, the addition of exogenous TGFβ to PBMC in culture failed to increase pSmad2 levels any further (FIG. 10), indicating that TβRs were already maximally activated in control cells. Furthermore, as expected, inhibition of the TβR-I kinase by treatment with TβKI for 135 min resulted in an approximately 80% reduction on pSmad2 levels (FIG. 10), presumably as a result of dephosphorylation by a nuclear phosphatase (t_{1/2}˜90 min). As shown in FIG. 11, pSmad2 levels were reduced by TβKI in a dose-dependent manner, with an estimated IC₅₀ of 100 nM. Finally, a series of mixing experiments were performed to simulate the effects of TβKI in blood on circulating PBMCs (FIG. 12). In these experiments, TβKI was pre-dissolved in 150 mM NaCl, which was then used to dilute freshly drawn peripheral blood. PBMC were then isolated as described above, and pSmad2 levels determined. As shown in FIG. 12, TβKI reduced pSmad2 levels in a dose-dependent manner. However, approximately 400-fold higher concentrations of TβKI added to plasma were required to achieve the same degree of inhibition as seen when the drug was added directly to PBMCs in culture (FIG. 11). Thus, a significant proportion of TβKI is inactivated by binding to plasma proteins.

### Example 6

### Effect of Anti-TGFβ Antibody on Levels of pSmad2 in BxPC-3 Pancreatic Cancer Cells

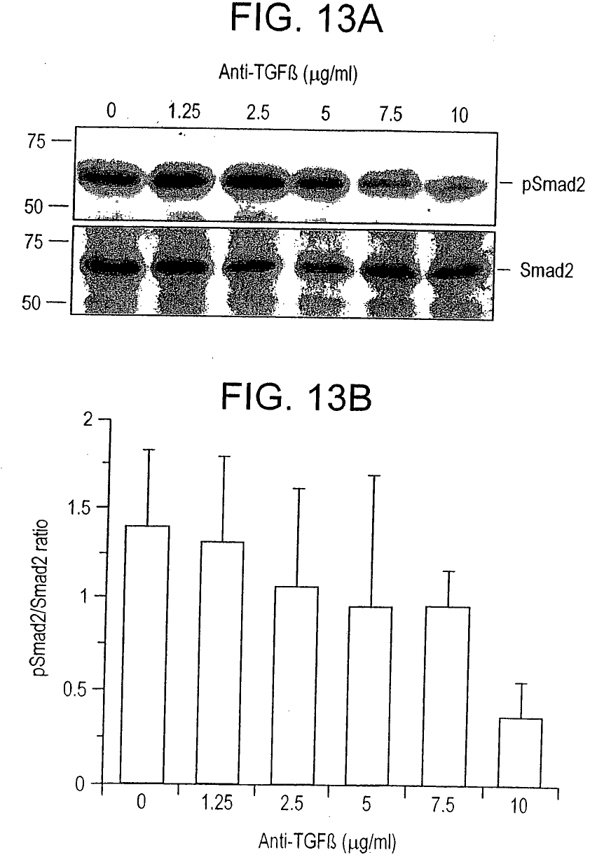

BxPC-3 pancreatic cancer cells express constitutively elevated levels of pSmad2 as a result of excessive production and secretion of active TGFβ. To demonstrate that excessive production and/or extracellular activation of TGFβ was responsible for activation of the TβR receptors in an autocrine manner, the cell cultures were treated with TGFβ neutralizing antibody and the effect on pSmad2 levels was measured. As shown in FIG. 13, overnight incubation of cells with a pan-specific anti-TGFβ neutralizing antibody resulted in a dose-dependent reduction in specific pSmad2 levels up to a maximum of 80%. Thus, extracellular biologically active TGFβ appeared to be in large part responsible for activating the TβR system and raising pSmad2 levels in BxPC3 cells. Conversely, this experiment demonstrates that treatment of cells with anti-TGFβ antibody is capable of turning off receptor kinase activity, resulting in lowering intracellular pSmad2 levels.

### Example 7

### Effect of TGF Beta Antagonists on Signaling in Peripheral Blood Mononuclear Cells-Studies on Gene Expression Profiling

### Materials and Methods

### Antibodies and Reagents

Rabbit polyclonal anti-Smad2 (1:500) and anti-Smad3 (1:500) antibodies were obtained from Zymed Laboratories (South San Francisco, Calif.). The activated (phosphorylated) forms of Smads 2 and 3 were detected using the anti-phospho-Smad2 (pSmad2, 1:1000) or anti-phospho-Smad3 (pSmad3, 1:1000) antibodies respectively, both of which were produced in our laboratory (Yan, W. et al., Oncol. Res. (2000), 12(3):157-167). The secondary antibody employed, anti-rabbit IgG H+L chain specific (goat) peroxidase conjugate (1:2000) was obtained from Calbiochem (San Diego, Calif.). The CD3 Hu, CD19 Hu, and CD14 Hu mouse IgG antibodies conjugated with FITC fluorescent dyes, which were used during flow cytometric analysis, were obtained from BD Biosciences-Pharmingen (Transduction Laboratories, San Diego, Calif.). Human recombinant TGFβ1 (1 ng/μl) (Austral Biologicals, San Ramon, Calif.) was dissolved in 4 mM HCl, 1 mg/ml bovine serum albumin (BSA, Sigma, St. Louis, Mo.) and stored at −70° C. SD-093 and SD-208 (Scios, Inc., Sunnyvale, Calif.) inhibitors of TGFβ type I receptor kinase, were dissolved in DMSO at 10 mM stock solution and were stored at −70° C. as well. For experiments, the inhibitor stock solutions were diluted with DMSO and serum-free medium (RPMI 1640 with 25 mM HEPES buffer with L-glutamine) making sure that the final concentration of DMSO did not exceed 1% (v/v).

### Sample Collection and Processing

Peripheral blood mononuclear cells (PBMC) were isolated from peripheral blood obtained from healthy volunteers following written informed consent according to a protocol approved by the UMDNJ Institutional Review Board. Blood samples were collected from each subject by venipuncture using a 23-gauge butterfly needle (Becton-Dickinson, Franklin Lakes, N.J.) into a 10 ml BD Vacutainer® green-top blood collection tube containing sodium heparin anticoagulant. Venipuncture was performed with the volunteer's arm in a downward position to reduce risk of any anticoagulant backflow into the donor's circulation. After each successive blood-draw, the collection tube was inverted multiple times to mix the blood with the chemical additive. Whole blood was then diluted 1:1 with 0.9% Sodium Chloride Irrigation USP (NaCl, B. Braun Medical Inc., Irvine, Calif.), layered onto a density gradient medium of Nycoprep 1.077 (Nycomed Pharma AS Diagnostics, Oslo, Norway) and centrifuged for 60 min at 800 rpm at 20° C. in a horizontal rotor (swing-out head) to separate out the mononuclear cell fraction from the red blood cells (RBC), platelets, and granulocytes according to the manufacturer's protocol.

The relatively low density of mononuclear cells makes it possible to isolate them from whole blood. During centrifugation, the PBMC form a distinct white layer at the top of the Nycoprep, whereas the RBC and denser blood components are collected at the bottom of the tube. The mononuclear cell fraction was collected by pipetting out the cell layer using a 9 inch glass Pasteur pipette and transferred to a conical centrifuge tube containing 30 ml of Hanks Balanced Salt Solution (HBSS) (Gibco, Grand Island, N.Y.) in which the cells were washed twice with HBSS by centrifuging for 45 min followed by 15 min at 1000 rpm and at 20° C., to isolate the cell pellet.

### Cell Counting and Cell Viability

The isolated PBMC were resuspended in RPMI 1640 medium containing 25 mM HEPES buffer with L-glutamine (Gibco, Grand Island, N.Y.) and supplemented with 5% (v/v) fetal bovine serum (FBS, Sigma, St. Louis, Mo.). Cell numbers were determined using a model Z2 Coulter particle counter and size analyzer (Beckman Coulter Inc., Miami, Fla.), and histograms of cell numbers by cell size distribution were plotted with the Z2 AccuComp software. The cells were tested for viability using the Vi-CELL 1 .00 cell viability analyzer (Beckman Coulter Inc., Miami, Fla.), which was also used to confirm the cell counts determined by the Coulter counter.

### Cell Culture and Drug Exposure

Following confirmation of cell counts and viability, 2 ml of the PBMC suspension, containing between 2-5×10⁶ cells, were plated into the chambers of 6-well culture dishes and incubated for 60 min at 37° C., 5% (v/v) CO2 atmosphere to allow the cells to adjust to the environment (temperature and medium RPMI 1640 supplemented with 5% FBS). Treatment was initiated, using DMSO alone or SD-093 or SD-208 (at various doses or over different periods of time depending on the specific experiment), incubated for 15 min at 37° C., 5% CO₂ and cells were then treated with 100 pM TGFβ or vehicle only for 2 or 19.5 hours. Following treatment, the cells were collected by centrifugation, washed with ice-cold PBS and lysed to isolate protein or RNA, depending on the experiment at hand.

### Protein Extraction and Western Blotting

PBMC cultures in 6-well dishes (Corning Inc., Corning, N.Y.) were collected, washed twice with ice-cold phosphate buffered solution (PBS, pH 7.4, Gibco), transferred to 15 ml conical centrifuge tubes and centrifuged at 1000 rpm for 10 min at 4° C. The cells were lysed in 200 μl lysis buffer [containing 10 mM Tris-HCl (pH 8.0), 150 mM NaCl, 1 mM EGTA, 1% Triton X-100 and Complete Mini Protease Inhibitor Cocktail (Roche Diagnostics Corporation, Indianapolis, Ind.)], for 40 min on ice. The cell lysates were then vortexed vigorously and centrifuged at 10,000 rpm for 10 min at 4° C. Concentrations of the total protein in the lysates were determined using a standard Bradford assay (Bio-Rad Laboratories, Hercules, Calif.). Protein lysates were resolved by western blot analysis using 12% (w/v) SDS polyacrylamide gels (Bio-Rad, Hercules, Calif.). Equal amounts of protein were separated by SDS-PAGE and were transferred to a nitrocellulose membrane (Bio-Rad Laboratories) using a semi-dry transfer blotter (Owl Separation Systems, Portsmouth, N.H.). Following transfer, the membranes were treated with blocking buffer consisting of TBS, 5% (w/v) Carnation nonfat dry milk and 0.1% (v/v) Tween 20, for 30 min at room temperature. The filters were then incubated with the primary antibody overnight at 4° C., and the following day were incubated for 1 hour at room temperature with 1:2000 dilutions of the secondary antibody (horseradish peroxidase-linked goat anti-rabbit IgG antibody). Bands were detected by using the ECL system (Amersham Biosciences, England) and visualized by exposing the membrane to high performance chemiluminescence film (Amersham Biosciences, England). Blots were scanned using an Epson Perfection 2400 photo scanner, and Image J v. 1.29 software (NIH, USA) was used to determine the optical density of each band derived from the scanned images.

### Separation of PBMC Nuclear and Cytoplasmic Fractions

The buffers needed for nuclear and cytoplasmic extract (PBS/Phosphatase Inhibitors, 1× hypotonic buffer, and complete lysis buffer), were prepared using the Nuclear Extract Kit (Activ Motif, catalog #40010). Following treatment, the cells' nuclear and cytoplasmic fractions were collected as per the manufacturer's protocol and stored at −70° C.

### Giemsa Staining of PBMC

Human PBMC isolated from whole blood were stained with Wright-Giemsa Stain 0.4% (w/v) buffered at pH 6.8 in methanol (Sigma, St. Louis, Mo.) to microscopically distinguish their individual cell types. Wright-Giemsa stain, containing a combination of acidic and basic dyes, differentially stains the granules, cytoplasm, and nuclei of various different blood cell types. A drop of mononuclear cells was, thus, placed on Fisherbrand superfrost microscope slides (Fisher Scientific, Pittsburgh, Pa.), a smear was done and allowed to air-dry. The smear was then horizontally stained by dripping the Wright-Giemsa stain over the slide with a plastic Pasteur pipette for 5 min at room temperature. Afterwards, the slide was immersed in tap water for 10 min, rinsed off (again with tap water) and air-dried. Once dry, the glass slides were viewed using a Nikon microscope (Micron-Optics, division of Sylvax Scientific Inc., Cedar Knolls, N.J.) equipped with a model DKC 5000 Sony digital camera that was used to capture the images.

### Immunofluorescence Staining of PBMC for Flow Cytometric Analysis

A fluorescence-activated cell sorter (FACS) was used to distinguish different cell populations after the cells had been labeled with fluorescently tagged antibodies directed against specific cell surface molecules referred to as CD antigens. In this study, we used CD3 Hu, mouse IgG antibody associated with FITC fluorescent dye, which binds to the CD3 antigen on the surface of human T cells. Similarly, CD 19 Hu fluorescent antibody binds to the CD19 antigen on the surface of human B cells, and CD14 Hu fluorescent antibody binds the CD14 antigen on surface of human monocytes. PBMC were isolated from whole blood, washed twice with Hanks solution and the cell pellet was resuspended with medium RPMI 1640-5% FBS. Following incubation of the plated cells at 37° C., 5% CO₂ atmosphere for 2 hours and for 19.5 hours, the cells were stained by incubation with 20 μl of each fluorescent antibody for 30 min at 4° C. and in the dark. Each treatment condition was divided into four equal cell suspensions in Eppendorf tubes, at a concentration of 2.5×10⁵ cells per tube. For each condition, one tube was unstained for use as the control, and cells in each of the other three tubes were stained with the CD3, CD19 and CD14 Hu antibodies respectively. Once all samples were stained, the cells were fixed with 4% Paraformaldehyde (containing PBS) and stored at 4° C. The different blood cell populations of each condition were then analyzed using a model FC500 flow cytometer (Beckman Coulter Inc.) using CXP software which gave graphical presentations of the size and fluorescence of each cell type, along with the percentage of each cell type present.

### RNA Isolation

Following treatment of PBMC for 0, 2, or 19.5 hours, RNA samples were obtained from about 3-5×10⁶ cells in each of the treatment conditions, resulting in an average RNA yield of 5-7 μg for each sample. Total RNA was extracted from mononuclear blood cells using the Quiagen Rneasy Mini Kit (Quiagen Inc., Valencia, Calif.) according to the manufacturer's protocol. Any possible genomic DNA contamination in the samples was removed using DNase I from the RNase-free DNase Set (Quiagen, catalog #79254). RNA was eluted into RNase-free water and quantified using a model DU640 spectrophotometer (Beckman Coulter Inc.). The isolated RNA was frozen at −70° C. to be used for later analysis.

### Microarray Analysis of Gene Expression in PBMC

Isolated human mononuclear cells were treated with TGFβ1 (100 pM), SD-093 (90 nM), both, or vehicle alone, for 2 hours or for 19.5 hours at 37° C., 5% CO₂ atmosphere. RNA was isolated immediately after PBMC isolation (baseline control) and following each time point (2-hour and 19.5-hour) from every treatment condition using the RNeasy mini kit and the on-column DNase I digestion option. The extracted RNA was given to the CINJ Microarray Core Facility and the RNA quality was accessed on an RNA chip using Agilent Bioanalyzer (Agilent Technologies). Isolated total RNA was processed as recommended by Affymetrix, Inc. In brief, cDNA was synthesized from the total RNA using the Superscript double stranded cDNA synthesis kit (Invitrogen Corp., Carlsbad, Calif.) and poly (T)-nucleotide primers. Using the ds cDNA as template biotin labeled cRNA was generated from an in vitro transcription reaction using the BioArray High-Yield RNA Transcript Labeling kit (T7) (Enzo Diagnostics). The cRNA was fractionated to 35-200 bases length using Affymetrix protocols and hybridized to the HG-U133 Plus 2.0 Gene Chip®, which allowed the interrogation of about 55,000 transcripts simultaneously, at 45° C. for 16 hours in an Affymetrix GeneChip Hybridization Oven 320. Each Gene Chip® was then washed and stained with streptavidin-phycoerythrin (SAPE) using Affymetrix Fluidics Station 400 and scanned on a Hewlett-Packard Gene Array scanner. Scanned image profiles were analyzed using Microarray Suite 5.0 software (MAS 5.0, Affymetrix Inc.). Scaling and normalization were carried out using the 100 Normalization Control probe set included in the HG-U133 plus 2.0 chip set. The Wilcoxon's Signed Rank test was used for pairwise comparisons of expression signals between chips, using a p-value of 0.0025 as cutoff for significant change. Comparison analysis was used to compare and detect changes in the gene expression profiles of any two Gene Chip® arrays; a baseline and an experimental file (which was compared to the baseline), and the array files were exported to Microsoft Excel. We were interested in investigating expression profiles of genes regulated by TGFβ and affected by the SD-093 inhibitor. To identify genes regulated by TGFβ, we used the control expression profiles as the baseline when comparing to it the TGFβ-treated cells (experimental file). On the other hand, to access the effect of SD-093 on these TGFβ regulated genes, expression profiles of TGFβ-treated cells were used as the baseline and profiles from cells treated with the inhibitor were taken as the experimental file. To highlight the distinct expression profiles in the chosen comparisons, we selected those genes that were significantly (p 0.0025) induced (or repressed) in one comparison, and which had the opposite effect in the other comparison. Furthermore, by comparing the signal log ratio of genes at 2 hours and at 19.5 hours, we were able to determine the degree and direction of change of the expression profiles at the two time points. (A signal log ratio of 1.0 indicated a 2-fold increase whereas −1.0 indicated a decrease by 2-fold, and zero meant no change). Once the gene lists were derived, the gene probe IDs were examined on Netaffyx Analysis Center, an online resource that allowed us to access biological annotations from the public domain of Affymetrix. The information obtained from Netaffyx helped us determine which genes are currently known and what their molecular function is and which biological process they are assigned to, based on the Gene Ontology (GO) database. With the help of this provided information, the numbers of genes expressed at 0, 2, and 19.5 hours were compared to each other to examine similarities in gene expression of both the early and late time points. Finally, lists of the gene expression changes were created for each time point and compared using Venn diagrams created using Canvas v. 8.0 (Deneba Systems, Inc.) as well as bar graphs created by Deltagraph v. 5.0 (SPSS Inc. and Red Rock Software, Inc.).

### Quantitative Real-Time PCR

RNA was isolated from PBMC that had undergone treatment conditions identical to those in the microarray experiment, and was used in quantitative real-time PCR analysis using the QuantiTect Probe RT-PCR kit (QIAGEN Inc., Valencia, Calif.). For PCR, 50 μl reactions were set up with 85 ng of RNA, 0.4 μM primer, 0.2 μM dual labeled probe, 0.5 μl of QuantiTect Reverse Transcriptase Mix and QuantiTect Probe RT-PCR Master Mix. The real-time PCR was performed in a Mx4000 Multiplex Quantitative PCR System (stratagene) with each data point performed in triplicate. Five RNA targets were quantified, including SERPINE 1 (or PAI-I), OSM, VEGF, OLR-1 and the control gene glyceraldehyde-3-phosphate dehydrogenase (GAPDH). Standard curves for all five genes were generated using serial dilution of RNA isolated from baseline-control cells. The mRNA amounts for each gene in the individual RNA samples, was calculated from the standard curves. Appropriate gene-specific primers used for the analysis were designed using Integrated DNA Technologies PrimerQuest. The following primers and probes were used:

VEGF:
Forward

(SEQ ID NO: 85)

5′-TTT CTG CTG TCT TGG GTG CAT TGG-3′,
Reverse

(SEQ ID NO: 86)

5′-ACC ACT TCG TGA TGA TTC TGC CCT-3′,
Probe

(SEQ ID NO: 87)

5′-FAM-TTG CTG CTC TAC CTC CAC CAT GCC AA-BHQ-3′;
OSM:
Forward

(SEQ ID NO: 88)

5′-AGT CTG GTC CTT GCA CTC CTG TTT-3′,
Reverse

(SEQ ID NO: 89)

5′-TGT CCT GCA TGA GAT CTG TCT GCT-3′,
Probe

(SEQ ID NO: 90)

5′-FAM-AAG CAT GGC GAG CAT GGC GGC TAT A-BHQ-3′;
OLR1:
Forward

(SEQ ID NO: 91)

5′-GAA GGT TGT GAA ATC AAG CAG GCG-3″
Reverse

(SEQ ID NO: 92)

5′-AAG TGC CCT TGA CTT AGT GGT GGT-3′,
Probe

(SEQ ID NO: 93)

5′-FAM-ACC GCT TGG TTT GAA GGC AGC TTT GA-BHQ-3′;
SERPINE1:
Forward

(SEQ ID NO: 94)

5′-TGC TGG TGA ATG CCC TCT ACT TCA-3′,
Reverse

(SEQ ID NO: 95)

5′-AGA GAC AGT GCT GCC GTC TGA TTT-3′,
Probe

(SEQ ID NO: 96)

5′-FAM-ACG GCC AGT GGA AGA CTC CCT T-BHQ-3′;
GAPDH:

(SEQ ID NO: 97)

Forward 5′-CCA CCC ATG GCA AAT TCC-3′,
Reverse

(SEQ ID NO: 98)

5′-TCG CTC CTG GAA GAT GGT G-3″
Probe

(SEQ ID NO: 99)

5′-FAM-TGG CAC CGT CAA GGC TGA GAA CGT-BHQ-3′.

### Results

### A. PBMC Isolation and Characterization

In order to determine which specific cell populations were present in isolated PBMCs, Giemsa-stained smears were examined. The basic cell types that comprise the mononuclear cells were identified visually by their differential staining morphology. Monocytes had a kidney-shaped nucleus, which was stained a blue-purple color, whereas lymphocytes (classified as small and large) had rounded nuclei and often little cytoplasm. This made it possible to view the stained blood smears and count the lymphocytes and monocytes on each slide. Cells were counted from six different areas of four slides (500 cells were counted each time). Density gradient centrifugation eliminated almost all granulocytes, and yielded a preparation of >95% lymphocytes (Table 1). Following 2-hour incubation of the isolated PBMCs at 37° C. in 5% CO₂ atmosphere, the preparation contained an average of 97.35% lymphocytes, 1.55% monocytes, and 1.1% granulocytes (Table 1). Following overnight incubation the population contained, on average, 99.5% lymphocytes, 0.3% monocytes and 0.2% granulocytes (Table 1). In summary, lymphocytes represented the predominant cell population present in isolated PBMCs, and this was not affected by short-term culture in vitro.

In order to determine the subtypes present in isolated mononuclear cell preparations, the PBMCs were stained with fluorescent antibodies (CD3, CD 19 and CD 14) and analyzed by FACS (Table 2). Consistent with the results from the blood smear counts, lymphocytes were found to represent the main cell population in PBMCs. Over 65% of these were T cells, 10-15% B-cells, whereas the fraction of monocytes was found to be very low (less than 2%) (Table 2). Even though the proportion of T-cells seemed to increase somewhat in short-term culture, the difference was not significant. Overnight studies, in which PBMC were plated on a 10 cm plastic dish and incubated overnight at 37° C., confirmed that the composition of cells did not change significantly. The percentage of T cells was found to be approximately 73.88% and that of B cells about 11.15%, with monocytes remaining below 2% (1.67%) (Table 2). The remaining cells present, which did not express CD3, CD 19, or CD 14, may represent natural killer cells (also lymphocytes, that are similar to cytotoxic T cells), which have been found in a similar study to represent approximately 10% of PBMCs (McLaren, P. J. et al, Clin. Diagn. Lab. Immunol. (2004), 11(5):977-982). Collectively, the results from the PBMC smears, as well as those obtained from flow cytometric analysis, indicate that lymphocytes, and specifically T cells, were the main constituents of our isolated human mononuclear cell preparations.

### B. Smad Signaling in Human PBMC

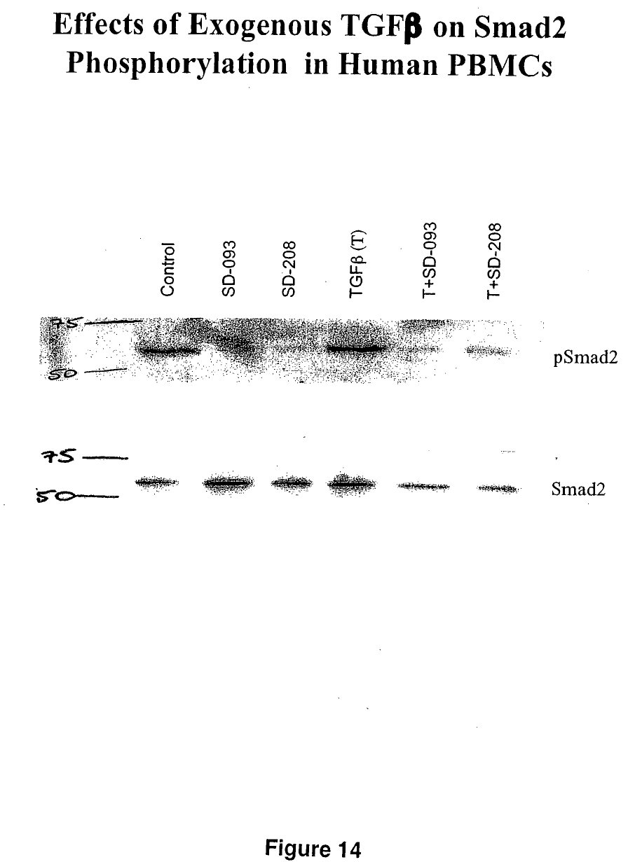

Isolated mononuclear cells expressed detectable levels of pSmad2 even in the absence of exogenous TGFβ (FIG. 14). However, pSmad2 was not detectable if cells were incubated in the absence of FBS (data not shown). Thus, the “basal” activation of Smad2 was induced by active TGFβ present in FBS. This conclusion was further supported by the fact that incubation of cells in the presence of SD-093 for 135 min resulted in loss of pSmad2, presumably because it was dephosphorylated once TβR-I kinase activity was shut off (Pierreux, C. E. et al. Mol. Cell. Biol. (2000), 2(8):9041-9054).

### C. Effects of TGFβ on Human PBMC

### Phosphorylation of R-Smads:

Treatment of cultured human mononuclear cells with exogenous TGFβ (100 pM) caused a further increase in the levels of pSmad2 (FIG. 14). Moreover, pSmad2 levels were also detected in the presence of FBS; therefore, only serum was used to activate pSmads in subsequent experiments.

### TGFβ Receptor Signaling; Effects of TβR-I Kinase Inhibitors on R-Smad Phosphorylation:

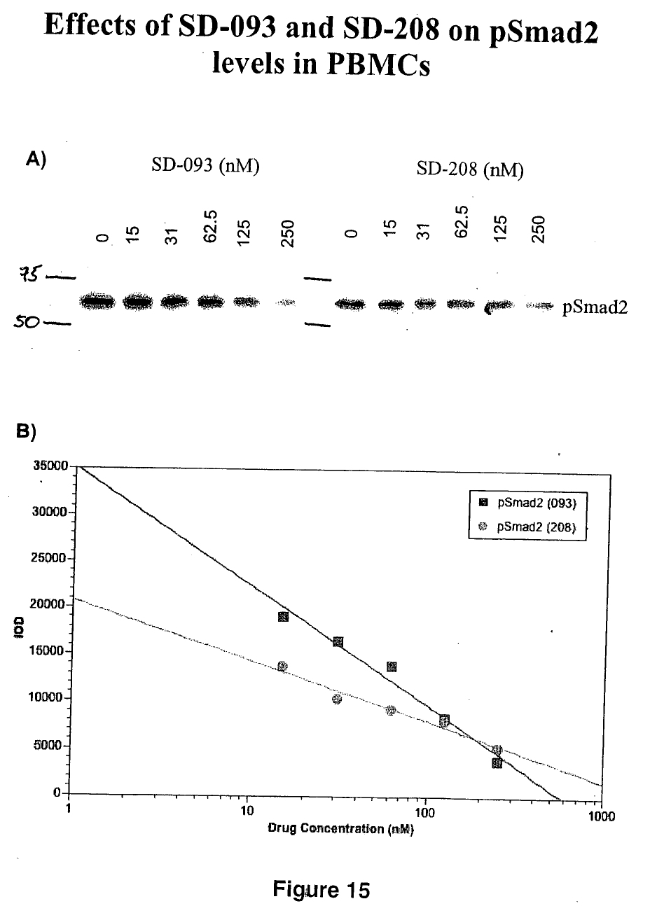

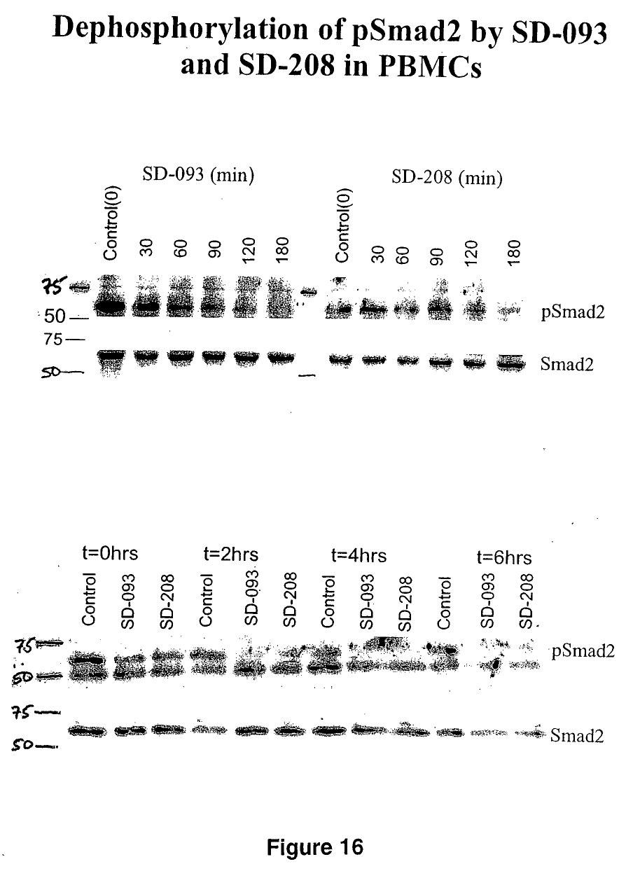

Upon serum treatment, human PBMC expressed phosphorylated Smad2. This pSmad2 signal was reduced after just 2 hours of treatment with the inhibitors, SD-093 and SD-208 in a dose-dependent manner (FIG. 15A). The estimated IC₅₀ of SD-093 and SD-208 were 60 nM and 70 nM respectively (FIG. 15B). These agents are therefore potent inhibitors of Smad phosphorylation. Shutting of TβR-I kinase activity results in dephosphorylation of pSmad-2 with a t_{1/2} of 70 minutes (FIG. 16).

Nuclear Localization of pSmad2 and pSmad3:

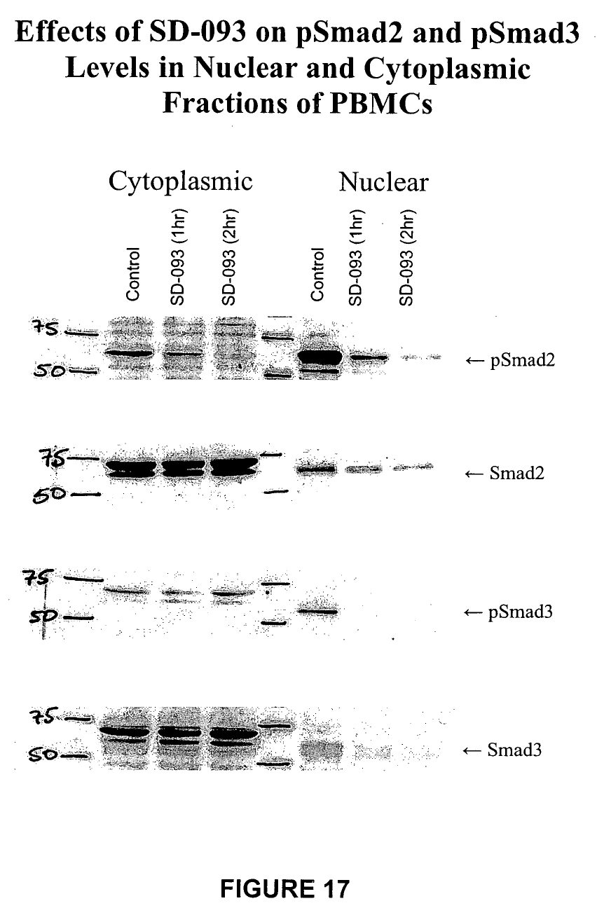

Nuclear and cytoplasmic fractions separated from PBMC, which were cultured in serum containing medium for 3 hours, revealed that almost all pSmad2 signal was present in the nucleus. The level of nuclear pSmad2 decreased with increasing time of SD-093 treatment and most of Smad2 shuttled back to the cytoplasm (FIG. 17). Similarly, almost all of pSmad3 was found in the nucleus, and treatment with SD-093 resulted in complete loss of the pSmad3 signal and disappearance of Smad3 from the nuclear fraction (FIG. 17). In order to standardize conditions and ensure that we would always maximally activate TβR signaling, TGFβ was added to serum in the subsequent experiments.

### Gene Expression in PBMC

The effects of partial inhibition of TβR-I kinase activity on TGFβ-regulated gene expression profiles were determined using Affymetrix Gene Chips®. PBMC were treated with 100 pM TGFβ (to ensure maximal TGFβ signaling) with or without 90 nM SD-093 for 0, 2 and 19.5 hours. RNA was isolated and subjected to genechip analysis; data were analyzed using Microarray Suite (5.0). In order to determine which genes were regulated by TβR-I, we identified those genes that were significantly up- or down-regulated by TGFβ and which displayed significant changes in the opposite direction when treated with TGFβ+SD-093.

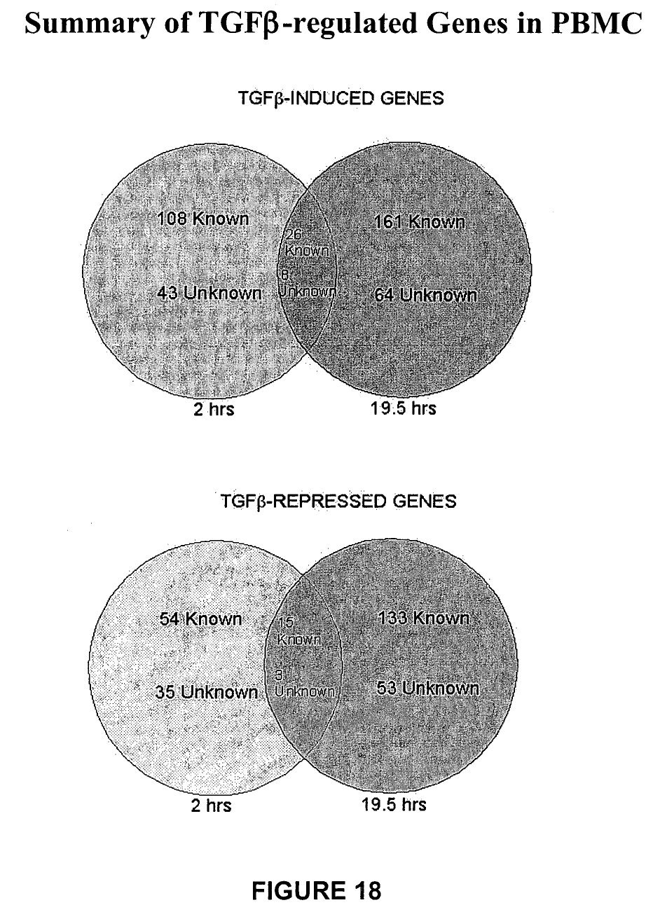

Lists of changes in gene expression (both up- and down-regulated) when treating with TGFβ versus when treating with TGFβ+SD-093 were generated and compared by Venn diagrams (FIG. 18). The Venn diagrams show that a larger number of genes are responsive to TGFβ treatment and inhibited by SD-093 at the later time point. Specifically, 108 known genes were induced and 54 genes were down-regulated by TGFβ at 2 hours whereas, 161 genes were up-regulated and 133 genes were downregulated by TGFβ at 19.5 hours. These genes were assigned to Biological processes using the NetAffx Microarray Data Mining Tool (www.affymetrix.com). As shown in (Tables 3, 4), TGFβ-regulated genes were primarily involved in cell proliferation, development, apoptosis, transcription and immune response.

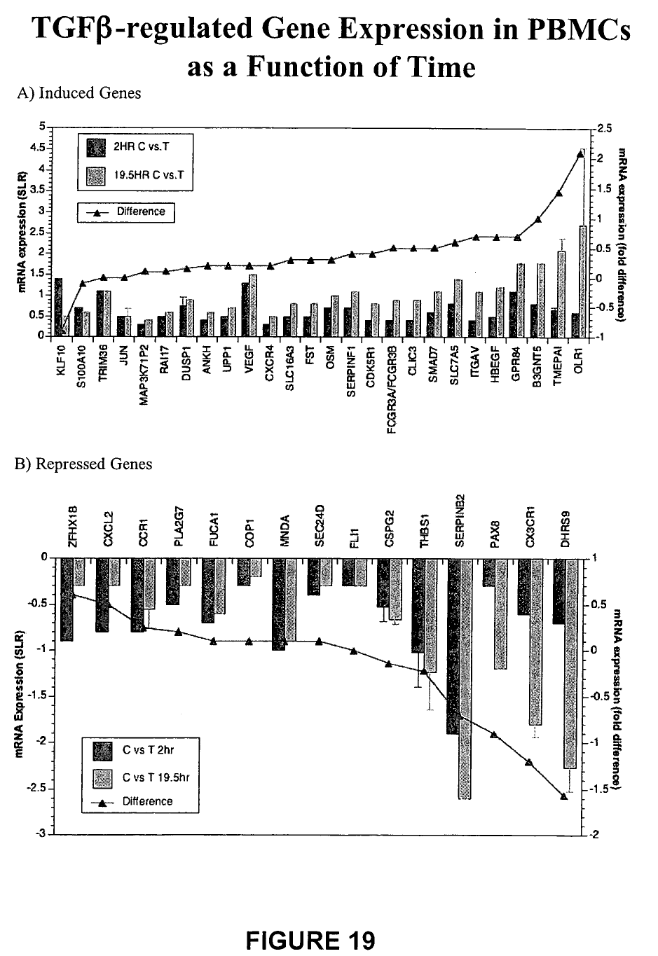

Moreover, we examined the genes that were common between the different time points and compared the degree of inhibition by SD-093 in each case, to determine which genes were affected by the inhibitor to a greater extent. We found that 26 of the known induced genes (Table 3) and 15 of the known repressed genes (Table 4) were commonly represented at both the 2-hour and 19.5-hour time points. Among these TGFβ-regulated genes were genes involved in development, TGFβ receptor signaling, regulation of transcription, apoptosis, immune response, and regulation of cell proliferation (Table 3, 4). For the vast majority of these genes, the magnitude of TGFβ-induced change was greater at 19.5 hours than at 2 hours (FIG. 19).

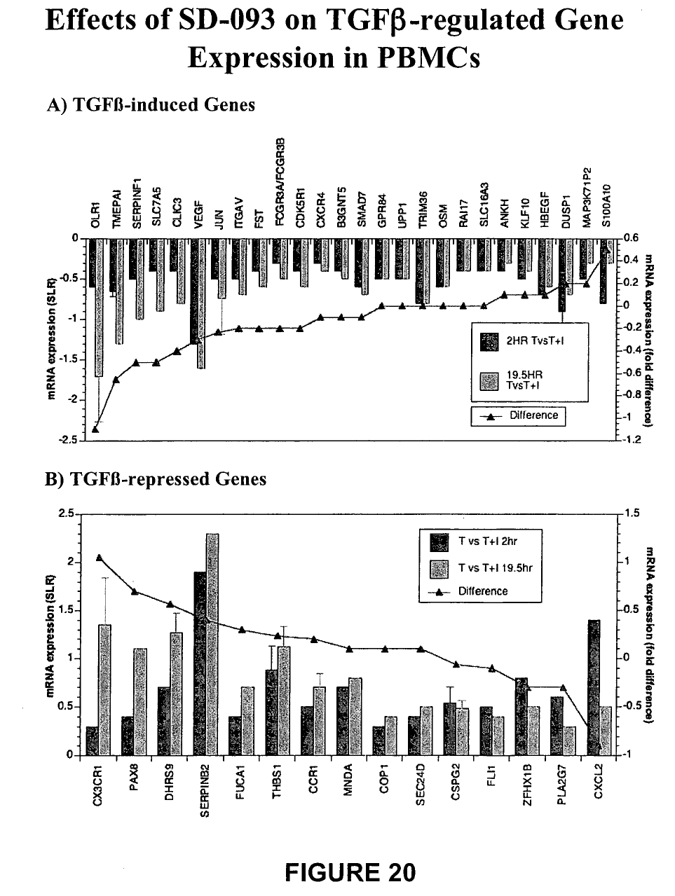

Treatment with SD-093 was effective at reversing the TGFβ-regulated gene expression (FIG. 20). Moreover, the genes that were most strongly regulated by TGFβ appeared to also be most sensitive to inhibition by SD-093, and vice versa (FIG. 20).

### E. Validation of Microarray Expression Profiling

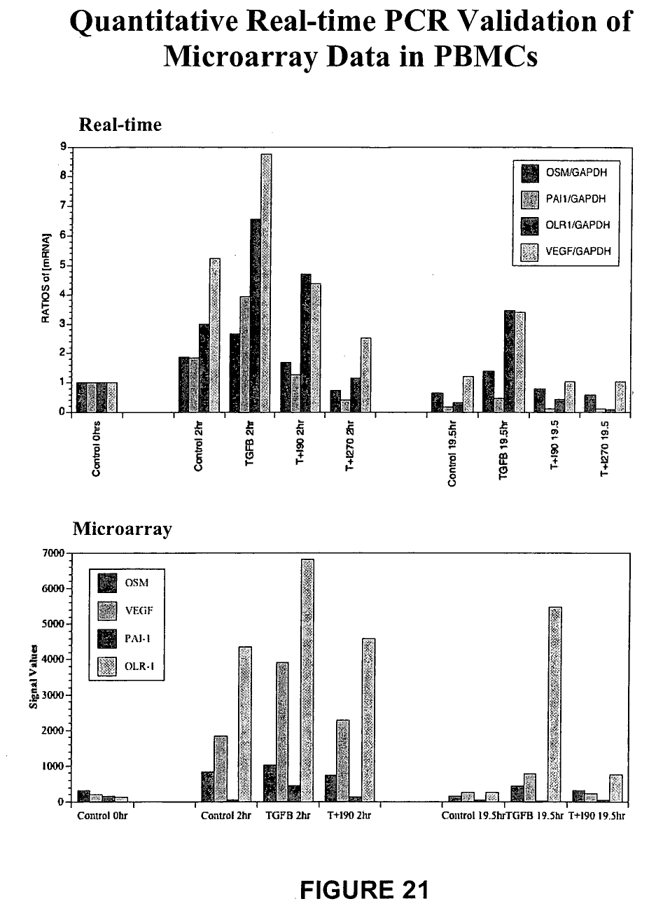

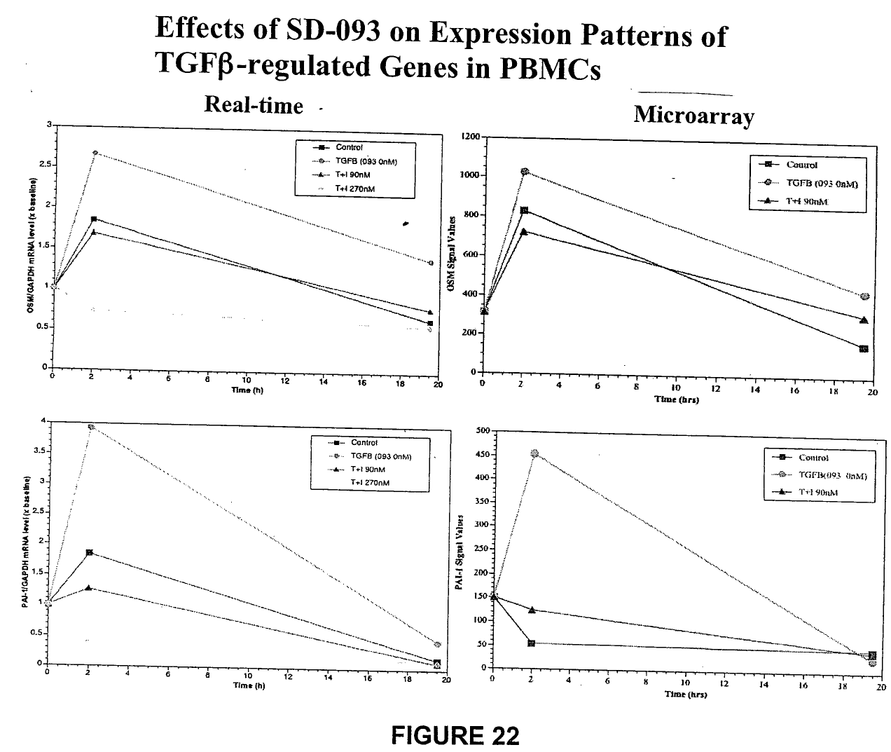

The results of PBMC gene expression obtained from the array were validated using the quantitative real-time PCR technique. Quantitative real-time PCR was performed for four of the 26 genes identified in our microarray study as being up-regulated at both 2 hours and 19.5 hours (VEGF, OSM, OLR-1, and PAI-1) and compared to the control gene GAPDH. The results obtained from real-time PCR were in excellent agreement with the expression profiles derived from the microarray analysis (FIGS. 21, 22). TGFβ treatment (100 pM) caused an induction in the expression of these genes. To examine the degree of up-regulation by TGFβ, the ratios of mRNA levels for the TGFβ conditions were compared to their levels at time 0 hours. This shows that there was a greater TGFβ induced up-regulation in gene expression at 2 hours compared to the 19.5 hour time point (FIG. 21). However, when we compared the level of induction by TGFβ to the level induced by serum-containing medium alone (as was done for the genechip experiments), the magnitude of the effect of TGFβ was greater at 19.5 hours than at 2 hours. As can be seen in FIG. 22, the effect of serum was essentially eliminated after overnight incubation, while the effect of exogenous TGFβ remains detectable, at least for three of the four genes.

Moreover, changes in gene expression patterns were associated with SD-093 exposure, in a time- and dose-dependent manner. As can be seen in FIGS. 21 and 22, treatment with 270 nM SD-093 completely blocked up-regulation of all four genes by serum or TGFβ at 2 hours, and significantly reduced mRNA levels to below baseline after 19.5 hours.

These results support that freshly isolated human mononuclear cells have intact TGFβ signaling that is readily activated by TGFβ; this is reflected both in the phosphorylation of R-Smads and the regulation of mRNA expression in a large number of genes. More importantly, we saw that TGFβ signaling, both at the level of Smad activation and gene expression, is effectively shut off by chemical inhibitors of TβR-I kinase in a dose- and time-dependent manner. Dephosphorylation was essentially complete by 2 hours; any additional up- or down-regulation of genes induced by serum or exogenous TGFβ was completely eliminated at 19.5 hours.

In summary, our results indicate that TGFβ induces a wide range of gene expression changes in human peripheral blood lymphocytes, that are likely important for their immune function. Moreover, TβR-I kinase inhibitor effectively counteracts TGFβ effects. Finally, our results suggest that freshly isolated human PBMC can be used to monitor effects of TGFβ antagonists in patients.

### Discussion

TGFβ is a potent cytokine that has influence upon immunosuppressive as well as proinflammatory processes (Jachimczak, P. et al. Cell. Immunol. (1995), 165(1):125-133), as it has inhibitory effects on the proliferation and differentiation of immune cells (Ronger-Savle, S. et al. J. Invest. Dermatol. (2005), 124(1):116-118). This is important since the immune system is able to recognize antigens expressed on human tumors and to mount a protective immune response (Monti, P. et al. J. Immunol. (2004), 172(12):7341-7349). In the case of malignancy, however, tumors are tolerated, progress, and ultimately kill their host.

Cancer impacts the immune response of a host in a number of ways. Dysfunction of tumor-infiltrating lymphocytes and circulating tumor-specific T cells may be early events in tumor progression. Defects include alterations in T cell receptor signaling events, reduced proliferation and increased apoptosis (Xu, T. et al. Cancer Res.(2004), 64(10):3661-3667).

Due to the dual functions of TGFβ, both in tumor suppression and promotion, and because the major components of mononuclear cells are lymphocytes, which play an essential role in the immune response and also secrete TGFβ, we decided to investigate the role of TGFβ in human peripheral blood mononuclear cells.

In order to determine the composition of PBMCs, and to make sure that we were mainly studying lymphocytes, we wanted to determine the fraction of monocytes present in the PBMC samples. Giemsa staining revealed that lymphocytes were the most abundant cell type present (ranging from 97-100%) whereas monocytes represented only 0-1.9% of the given cells (Table 1). In order to confirm the results obtained from the PBMC smears, the mononuclear cells were stained with fluorescent antibodies (CD3, CD 19, and CD 14) and analyzed via flow cytometry (FCM) at three different time points to ensure that the composition of the different cell populations did not change over time. PBMC cultures were composed predominantly of T lymphocytes (73.88%) and B lymphocytes represented 11.15% of the existing populations (Table 2), which is in agreement with previous findings done on PBMCs (McLaren, P. J. et al., Clin. Diagn. Lab. Immunol. (2004), 11(5):977-982). Monocyte fractions, on the other hand, were found to be very low (less than 2%). Combined, these results verified that in all the studies which followed, we were investigating the gene expression in mainly lymphocytes (and specifically T cells), and that the overall composition of the present cell populations remained constant. This was a crucial finding, since microarrays are sensitive to the representation of different cell types in a sample (Baechler, E. C., Genes Immunol. (2004), 5(5):347-353).

In this study, in order to investigate TGFβ signaling in PBMCs, we initially examined Smad expression in these cells. Mononuclear cells were treated with (or without) TGFβ and TβR-I kinase inhibitor SD-093 and/or SD-208 at various doses and over different time periods. The changes in Smad expression were confirmed by Western Blot analysis. In our experiments, short-term culture in serum-containing medium induced pSmad2 in PBMCs, which decreased with increasing time (and/or dose) of the inhibitor (FIG. 3). It must be noted, however, that in the absence of exogenous TGFβ and serum, no pSmad2 was detectable. Moreover, we found that exogenous TGFβ treatment (100 pM) further increased the phosphorylation of Smad2 (FIG. 2). These results are consistent with the recent findings described by Sebestyen et al. (Sebestyen, A. et al. Cytokine, (2005), 30(5):228-235) who also studied the expression and activity of TGFβ1 signaling components in isolated human PBMCs from blood of healthy individuals and B-cell lymphoma patients. These investigators showed that TGFβ-treated lymphoma cells expressed phosphorylated Smad2 and 3, nuclear translocation of the R-Smads, and increased expression of the Smad-dependent TGFβ induced gene, TIEG, proving that Smad signals were effectively transmitted. In our study, strong signals for pSmad2 and pSmad3 were seen in the nucleus of normal PBMCs cultured in serum, and these signals decreased with increasing time of SD-093 treatment (FIG. 5). In the case of pSmad3, expression was completely abolished after just 1 hour of treatment with the inhibitor. Total Smad213 levels, on the other hand, were observed mostly in the cytoplasm, as also demonstrated by Li et al. (Li, X. et al, World J. Gastroenterol. (2005), 11(1):61-68). Addition of TβR-I kinase inhibitor in our experiments with exogenous TGFβ treatment was again capable of reversing the effect of TGFβ. The phosphorylated R-Smad expression was eliminated after 2 hours of inhibitor treatment, whereas total Smad levels remained unaffected, proving that SD-093 and SD-208 are potent inhibitors of Smad phosphorylation. As shown by Inman et al. (Inman, G. J., et al. Mol. Cell. (2002), 10(2):283-294) and Pierreux et al. (Pierreux, C E, et al. Mol. Cell. Biol. (2000), 20(23):9041-9054), inhibition of TβR-I kinase results in rapid dephosphorylation of pSmads. Isolated human mononuclear cells, therefore, have an intact TGFβ signaling system, which is shut off by chemical inhibitors of the type I receptor kinase in a dose- and time-dependent manner. Specifically, we showed that TGFβ activates the type I receptor kinase, and that the inhibitor was able to effectively block this activation.

For gene expression analysis, PBMC samples were incubated for 0, 2, and 19.5 hours with 100 pM TGFβ and 90 nM SD-093. This allowed us to observe gene expression profiles in the mononuclear cells over time, determine which genes are responsive to TGFβ, and how they are affected by short- and long-term exposure to inhibitor treatment. Among the genes significantly up-regulated with TGFβ treatment, were vascular endothelial growth factor (VEGF), oncostatin M (OSM), oxidized low density lipoprotein (lectin-like) receptor-1 (OLRI), and plasminogen activator inhibitor-1 (PAI1), which we used (along with the control gene GAPDH) to do quantitative real-time PCR for validation of the microarray results. The induction of these genes are consistent with several other studies that report the significance of these genes in TGFP signaling and pathological conditions, including cancer progression (Teraoka, H. et al. Br. J. Cancer, (2001), 85(4):612-617; Ikeda, Y. et al, Hyperten. Res. (2004), 27(2):119-128; Aldridge, S. E. et al, Br. J. Cancer (2005), 92(8):1531-1537; Nightingale, J. et al. J. Am. Soc. Nephrol. (2004), 15(1):21-32; Draude, G. et al. Am. J. Physiol. Heart Cire Physiol. (2000), 278(4):H1042-1048); Kanasaki, K. et al. J. Am. Soc. Nephrol. (2003), 14(4): 863-872; Dong, C. et al. J. Heart Lung Transplant, (2002), 21(9):999-1008; Wakahara, K. et al. J. Cell Biochem. (2004), 93(3):437-453).

TGFβ has been shown to decrease peripheral blood mononuclear lymphocyte attachment to cancer cells, thus inducing immunosuppression by the escape of cancer cells from immunosurveillance, and also increased VEGF production, which is known to promote angiogenesis (Teraoka, H. et al. Br. J. Cancer (2001), 85(4): 612-617). Angiogenesis is an important function both in health, as it restores blood flow to tissues after injury, and in disease, in which blood vessels grow excessively or insufficiently. Angiogenesis is mediated by specific growth factors that stimulate the migration and proliferation of vascular cells and fibroblasts (Blotnick, S. et al. Proc. Natl. Acad. Sci. USA, (1994), 91(8):2890-2894) as, for example, VEGF and TGFβ. In the case of cancer, angiogenesis allows tumor cells to infiltrate into the blood vessels, adhere to endothelial cells, escape into the circulation, and metastasize to distant sites (Teraoka, H. et al. Br. J. Cancer (2001), 85(4): 612-617).

One of the earliest steps in the development of another pathological condition, atherosclerosis, is lipid accumulation in the vessel wall, which depends on the uptake of low density lipoprotein (LDL) by macrophages and their transformation into foam cells (Draude, G. et al. Am. J. Physiol. Heart Cire. Physiol. (2000), 278(4):H1042-1048; Salomonsson, L. et al. Eur. J. Clin. Invest. (2002), 32(10): 767-774). In agreement with our study, TGFβ was shown to up-regulate the lectin-like oxidized low density lipoprotein receptor-1 (Draude, G. et al. Am. J. Physiol. Heart Cire. Physiol. (2000), 278(4):H1042-1048) and another study revealed that oxidized LDL increases VEGF expression in macrophages, which in turn induces angiogenesis (Salomonsson, L. et al. Eur. J. Clin. Invest. (2002), 32(10): 767-774). In this case, VEGF may, by promoting vascular permeability, enhance the progression of atherosclerotic plaques.

Another recent report showed that activated PBMCs release a cytokine, Oncostatin M (OSM), which induces epithelial to myofibroblast transdifferentiation (Nightingale, J. et al. J. AM. Soc. Nephrol. (2004) 15(1): 21-32). In this study, OSM induced loss of E-cadherin expression, the cells lost their epithelial morphologic features, and displayed elongated, spindle-shaped morphology. Interestingly, OSM was also significantly up-regulated in our microarray experiment when PBMCs were treated with TGFβ, which is also known to induce EMT. This data, in combination with the previously described studies, may propose a possible mechanism of metastasis during cancer progression. As previously described, cancer cells detach from the primary tumor, infiltrate into the blood vessels and adhere to endothelial cells. TGFβ is secreted locally by the cancer cells and cells within the tumor microenvironment, inducing VEGF production and thus promoting angiogenesis, and the activated PBMCs release OSM, which induces EMT and may help cancer cells invade surrounding tissue.

Also consistent with our findings, TGFβ-induced PAI-1 (also known as SERPINE1) up-regulation has also been documented in numerous other studies (Kanasaki, K. et al. J. Am. Soc. Nephrol. (2003), 14(4): 863-872; Dong, C. et al. J. Heart Lung Transplant, (2002), 21(9):999-1008; Wakahara, K. et al. J. Cell Biochem. (2004), 93(3):437-4531). For instance, TGFβ is one of the key cytokines in the progression of renal disease, as it plays a major role in matrix protein accumulation and collagen deposition (Border, W. A. Curr. Opin. Nephrol. Hyperten. (1994), 3(1):54-58) TGFβ stimulates the expression of ECM proteins such as collagens, laminin, and fibronectin, while it suppresses the expression of ECM-degrading proteases, and increases the synthesis of ECM protease inhibitors, including PAI-1 (Kanasaki, K. et al. J. Am. Soc. Nephrol. (2003), 14(4)). Therefore, TGFβ signaling can provide a therapeutic target for the inhibition of progressive renal disease. TGFβ-induced PAI-1 expression is also associated with arteriosclerosis, as PAI-1 inhibits proteolysis by preventing the conversion of plasminogen to plasmin, and thus, inhibits ECM degradation contributing to ECM accumulation associated with arteriosclerosis (Dong, C. et al. J. Heart Lung Transplant, (2002), 21(9):999-1008). PAI-1 induction has also been reported in cancer. For instance, TGFβ induced a fivefold up-regulation of PAI-1 expression in human ovarian cancer cells via the Smad signaling pathway, as described by Wakahara et al. (Wakahara, K. et al. J. Cell Biochem. (2004), 93(3):437-4531) In contrast, however, to the negative effects associated with PAI-1 induction, positive attributes have also been observed in pathological conditions. Oncostatin M, as well as TGFβ, both have been seen to regulate the expression of PAI-1 in human cardiac myocytes, which is important for cardiac repair after myocardial infarction (Macfelda, K. et al. J. Mol. Cell. Cardiol. (2002), 34(12):1681-1691). In particular, Macfelda et al. provide evidence that human adult cardiac myocytes express PAI-1 in vitro, and this expression is significantly up-regulated (up to fivefold) by the inflammatory mediators TGFβ and OSM in a dose-dependent way. This induction was also confirmed on the level of PAI-1 mRNA expression.

To confirm the validity of changes in the gene expression patterns from our microarray study, the genes mentioned above (VEGF, OSM, PA11 and OLR1) were selected, and were subjected to further analysis. Quantitative real-time PCR was carried out for these genes, and the GAPDH signal served as a control. The real-time PCR results were in accordance with the expression profiles obtained from the microarray analysis (FIG. 9, 10). We found that the TGFβ-regulated gene expression changes were associated with SD-093 exposure, in a time- and dose-dependent manner. The addition of exogenous TGF, (100 pM) caused an induction in gene expression. SD-093 was effective at suppressing the expression of these genes. Specifically, a greater change in gene expression was observed with increasing time and/or dose of the inhibitor. Not surprising, at the 2 hour time point we saw some partial inhibition at submaximal concentration of SD-093, although inhibition was complete at 270 nM. This proved that the gene expression changes were dependent on the activity of TGFβ type I receptor kinase. Consistent with our results of Smad phosphorylation observed from Western Blot analysis, the SD-093 inhibitor was able to effectively block the activation of the TβR-I kinase, which is induced by TGFβ, time- and dose-dependently. These observations have implications for the development of clinical assays for inhibitor action in vivo. Specifically, time of collection after the last dose needs to be standardized if one wants to measure dose-dependent effects.

The ability to profile gene expression is a very efficient approach to screen genes expressed in particular cells, in this case lymphocytes. Microarrays are a very powerful tool, as gene expression profiles have facilitated rapid progression in identifying molecular pathways associated with human malignancies and other disease (Baechler, E. C. Genes Immunol. (2004), 5(5):347-353). Also, targeted destructions or introductions of genes have been a successful strategy for assessing the role of cytokines, cytokine receptors, signaling molecules and transcription factors in lymphocyte development and function (Goh, S. H. et al. Genomics, (2000), 70(1):1-18). Therefore, monitoring of gene and protein expression in PBMCs has significant potential for monitoring the pharmacodynamic properties and mechanisms of action of TGFβ antagonists in (human) patients.

TABLE 1
Composition of PBMC by Giemsa Staining

| | | Average | | |
|---|---|---|---|---|
| | Average % | % PBMC post | Average | Average |
| | WBC whole blood | Granulocyte | % PBMC | % PBMC |
| Cell Type | t = 0 | removal t = O | t = 2 hr | t = 19.5 hr |
| Lymphocytes | 29 | 95.33 | 97.35 | 99.5 |
| | St. Dev. 11.314 | St. Dev. 2.212 | St. Dev. 0.885 | St. Dev. 0.707 |
| Monocytes | 6 | 3.86 | 1.55 | 0.3 |
| | St. Dev. 3.536 | St. Dev. 2.082 | St. Dev. 0.443 | St. Dev. 0.424 |
| Granulocytes | 61 | 0.8 | 1.1 | 0.2 |
| | St. Dev. 14.849 | St. Dev. 0.400 | St. Dev. 0.577 | St. Dev. 0.283 |

Smears of PBMCs isolated from whole blood were stained horizontally with Giemsa as described in “Materials and Methods”. The percentages of specific cell populations present in the smears were identified visually at 0, 2, and 19.5 hours by their differential staining morphology and compared to the Robert Wood Johnson laboratory list of reference ranges for leukocytes in a complete blood count. Lymphocytes represented the predominant cell population in the isolated mononuclear cells.

TABLE 2
Composition of PBMC by FACS

| | Average % PBMC | Average % PMBC | Average % PMBC |
|---|---|---|---|
| Antigen | t = 0 hr | t = 2 hr | t = 19.5 hr |
| CD3 | 68.55 | 76.28 | 76.83 |
| | St. Dev. 12.233 | St. Dev. 10.21 8 | St. Dev. 10.288 |
| CD19 | 12.85 | 10.28 | 10.31 |
| | St. Dev. 6.859 | St. Dev. 6.053 | St. Dev. 5.392 |
| CD14 | 1.85 | 1.41 | 1.76 |
| | St. Dev. 0.071 | St. Dev. 0.301 | St. Dev. 0.088 |

Freshly isolated PBMCs were stained with the fluorescent antibodies CD3, CD 19 and CD14, and analyzed via flow cytometry as described in “Materials and Methods”. The cells were analyzed immediately following PBMC isolation (t=O), and following 2 hour and 19.5 hour incubation in two separate experiments. This gave us six values for each of the given cell types: T lymphocytes (CD3), B lymphocytes (CD19) and monocytes (CD14). Lymphocytes, and in specific Tcells, represented the most abundant cell population in the isolated PBMCs.

TABLE 3
Genes Significantly Induced by TGFβ

| Gene Symbol/ | | | | SEQ ID | |
|---|---|---|---|---|---|
| GenBank | | | | NO | SEQ ID |
| accession | Biological | Molecular | Name of | Nucleic | NO |
| numbers | Process | Function | Gene | acid | Protein |
| KLF 10/ | Negative | Transcription | Kruppel-like | 1 | 43 |
| NM_001032282 | regulation of | factor activity | factor | | |
| NP_001027453 | transcription, | Zinc ion | | | |
| | negative | binding | | | |
| | regulation of cell | | | | |
| | proliferation, | | | | |
| | TGFb receptor | | | | |
| | signaling | | | | |
| | pathway, cell-cell | | | | |
| | signaling, DNA- | | | | |
| | dependent, | | | | |
| | skeletal | | | | |
| | development | | | | |
| S100A10/ | | Calcium ion | S100 calcium | 2 | 44 |
| NM_002966 | | binding | binding | | |
| NP_002957 | | | protein A10 | | |
| TRIM36 | Protein | Ubiquitin- | Tripartite | 3 | 45 |
| NM_001017397 | ubiquitination | protein ligase | motif- | | |
| NP_001017397 | | activity | containing 36 | | |
| | | Zinc ion | | | |
| | | binding | | | |
| JUN | Regulation of | Transcription | v-jun | 4 | 46 |
| NM_002228 | transcription | factor activity | sarcoma | | |
| NP_002219 | DNA-dependent | RNA | virus 17 | | |
| | | polymerase II | oncogene | | |
| | | transcription | homolog | | |
| | | factor activity | | | |
| SERPINE1 | Anti-apoptosis | Serine-type | Serine- | 5 | 47 |
| AAH10860 | | endopeptidase- | cysteine | | |
| AF386492 | | inhibitor | proteinase | | |
| | | activity | inhibitor | | |
| | | Plasminogen | clade E, | | |
| | | activator | member 1 | | |
| | | activity | | | |
| RAI17 | Regulation of | Zinc ion | Retinoic | 6 | 48 |
| NM_020338 | transcription | binding | acid-induced | | |
| NP_065071 | DNA-dependent | | | | |
| DUSP1 | Protein amino | Non-membrane | Dual | 7 | 49 |
| NM_004417 | acid | spanning | specificity | | |
| NP_004408 | dephosphorylation | protein tyrosine | phosphatase | | |
| | Response to | phosphatase | | | |
| | oxidative stress, | activity | | | |
| | cell cycle | Hydrolase | | | |
| | | activity, MAP | | | |
| | | kinase | | | |
| | | phosphatase | | | |
| | | activity | | | |
| ANKH | Skeletal | Inorganic | Ankylosis, | 8 | 50 |
| NM_054027 | development, | phosphate | progressive | | |
| NP_473368 | transport, | transporter | homolog | | |
| | phosphate | activity, | | | |
| | transport, | phosphate and | | | |
| | perception of | inorganic | | | |
| | sound, | diphosphate | | | |
| | locomotory | transporter | | | |
| | behavior, | activity | | | |
| | regulation of | | | | |
| | bone | | | | |
| | mineralization | | | | |
| UPP1 | Nucleoside | Uridine | Uridine | 9 | 51 |
| NM_003364 | metabolism | phosphorylase | phosphorylase 1 | | |
| NP_003355 | Nucleotide | activity, | | | |
| | metabolism | Transferase | | | |
| | | activity, | | | |
| | | transferring | | | |
| | | glycosyl groups | | | |
| VEGF | Regulation of cell | VEGF receptor | Vascular | 10 | 52 |
| NM_001025366 | cycle, | binding | endothelial | | |
| NP_001020537 | angiogenesis, | Growth factor | growth factor | | |
| | vasculogenesis, | activity, | | | |
| | signal | heparin | | | |
| | transduction, | binding, | | | |
| | positive | extracellular | | | |
| | regulation of cell | matrix binding | | | |
| | proliferation, | | | | |
| | negative | | | | |
| | regulation of | | | | |
| | apoptosis, cell | | | | |
| | migration, | | | | |
| | neurogenesis | | | | |
| CXCR4 | G-protein | Rhodopsin-like | Chemokine | 11 | 53 |
| NM_001008540 | coupled receptor | receptor | (C—X—C | | |
| NP_001008540 | protein | activity, C-C | motif) | | |
| | Signaling | chemokine | receptor 4 | | |
| | pathway | receptor | | | |
| | | activity, C—X—C | | | |
| | | chemokine | | | |
| | | receptor | | | |
| | | activity | | | |
| SLC16A3 | Transport, | Transporter | Solute carrier | 12 | 54 |
| BC112269 | organic anion | activity, | family 16 | | |
| NP_001035887 | transport, | symporter | (mono | | |
| | monocarboxylic | activity, | carboxylic | | |
| | acid transport | monocarboxylateporter | acid | | |
| | | activity | transporters, | | |
| | | | member 3 | | |
| FST | Development, | Activin | follistatin | 13 | 55 |
| NM_013409 | negative | inhibitor | | | |
| CAG46612 | regulation of | activity | | | |
| | follicle- | | | | |
| | stimulating | | | | |
| | hormone | | | | |
| | secretion | | | | |
| OSM | Regulation of cell | Cytokine | Oncostatin M | 14 | 56 |
| BC011589 | growth, immune | activity | | | |
| CAG30420 | response, | Oncostatin-M | | | |
| | development, | receptor | | | |
| | cell-cell | binding | | | |
| | signaling, | | | | |
| | negative | | | | |
| | regulation of cell | | | | |
| | proliferation | | | | |
| SERPINF1 | Development, | Serine-type | Serine | 15 | 57 |
| BC013984 | cell proliferation, | endopeptidase | peptidase | | |
| AAH13984 | negative | Inhibitor | inhibitor, | | |
| | regulation of | activity | clade F | | |
| | angiogenesis, | | | | |
| | positive | | | | |
| | regulation of | | | | |
| | neurogenesis | | | | |
| | positive | | | | |
| | regulation of | | | | |
| | neurogenesis | | | | |
| CDK5R1 | Regulation of | protein kinase | Cyclin- | 16 | 58 |
| NM_003885 | cyclin-dependent | activity | dependent | | |
| NP_003876 | protein kinase | Cyclin- | kinase 5, | | |
| | activity, | dependent | regulatory | | |
| | Brain | protein kinase | subunit 1 | | |
| | development, cell | 5 activator | (p35) | | |
| | proliferation, | activity | | | |
| | regulation of | | | | |
| | neuron | | | | |
| | differentiation | | | | |
| FCGR3A | Immune response | Receptor | Fc fragment | 17 | 59 |
| NM_000569 | | activity | of IgG, low | | |
| NP_000560 | | IgG binding | affinity IIIa | | |
| | | | receptor | | |
| | | | (CD16a) | | |
| FCGR3B | Immune response | Receptor | Fc fragment | 18 | 60 |
| NM_000570 | | activity | of IgG, low | | |
| NP_000561 | | IgG binding | affinity IIIb | | |
| | | | receptor | | |
| | | | CD16b) | | |
| CLIC3 | Ion transport, | Voltage-gated | Chloride | 19 | 61 |
| NM_004669 | chloride | chloride | intracellular | | |
| NP_004660 | transport, Signal | channel | channel 3 | | |
| | transduction | | | | |
| SMAD7 | Regulation of | Receptor | MAD, | 20 | 62 |
| NM_005904 | transcription, | signaling | mothers | | |
| NP_005895 | DNA dependent | protein | against DPP | | |
| | Response to | Serine/threonine | homolog 7 | | |
| | stress, TGFb | kinase | (drosophila) | | |
| | receptor signaling | signaling, | | | |
| | pathway | protein | | | |
| | | activity/binding, | | | |
| | | TGFb | | | |
| | | receptor, | | | |
| | | inhibitory | | | |
| | | cytoplasmic | | | |
| | | mediator | | | |
| | | activity | | | |
| SLC7A5 | Amino acid | Neutral amino | Solute carrier | 21 | 63 |
| NM_003486 | metabolism | acid transport | family 7, | | |
| NP_003477 | Trasport, amino | activity, amino | member 5 | | |
| | acid transport | acid permease | | | |
| | | activity | | | |
| ITGAV | Cell matrix | Protein binding | Integrin, | 22 | 64 |
| NM_002210 | adhesion | | alpha V | | |
| NP_002201 | Integrin-mediated | | (vitronectin | | |
| | signaling | | receptor, | | |
| | pathway | | alpha | | |
| | | | polypeptide, | | |
| | | | antigen | | |
| | | | (CD51) | | |
| HBEGF | Signal | Receptor | Heparin- | 23 | 65 |
| NM_001945 | transduction, | activity, | binding EGF- | | |
| NP_001936 | muscle | epidermal | like growth | | |
| | development | growth factor | factor | | |
| | Positive | receptor | | | |
| | regulation of cell | binding, | | | |
| | proliferation | growth factor | | | |
| | | activity, | | | |
| | | heparin binding | | | |
| GPR84 | G-protein | Rhodopsin-like | G-protein | 24 | 66 |
| NM_020370 | coupled receptor | receptor | coupled | | |
| NP_065103 | protein | activity, G- | receptor 84 | | |
| | Signaling | protein coupled | | | |
| | pathway | receptor | | | |
| | | activity, | | | |
| | | unknown | | | |
| | | ligand | | | |
| B3GNT5 | Protein amino | Galactosyltransferase | UDP- | 25 | 67 |
| NM_032047 | acid | activity | GlcNAc beta | | |
| NP_114436 | glycosylation, | Transferase | Gal Beta-1,3- | | |
| | central nervous | activity, | N-acetyl- | | |
| | system | transferring | glucosaminyl | | |
| | development, | glycosyl groups | transferase | | |
| | glycolipid | | | | |
| | biosynthesis | | | | |
| TMEPAI | Androgen | | Trans-membrane, | 26 | 68 |
| NM_020182 | receptor, | | prostate | | |
| NP_064567 | signaling | | androgen | | |
| | pathway | | induced RNA | | |
| OLR1 | Proteolysis and | Receptor | Oxidized low | 27 | 69 |
| NM_002543 | peptidolysis | activity | density | | |
| NP_002534 | Circulation | Sugar binding | lipoprotein | | |
| | | | (lectin-like) | | |
| | | | receptor 1 | | |

Table 3. Genes significantly induced by TGFβ: PBMCs were treated with 100 pM TGFβ with or without SD-093 for 0, 2 and 19.5 hours. The effects of TGFβ on PBMC gene expression profiles were determined using Affymetrix Gene Chips® as described in “Materials and Methods”. In order to determine the genes that were sensitive to the inhibitor, we identified those genes that were significantly up-regulated with TGFβ and significantly down-regulated when treated with TGFβ+SD-093. 26 of the known TGFβ induced genes were common at both the 2 hour and 19.5 hour time points.

TABLE 4
Genes Significantly Repressed by TGFβ

| Gene | | | | | |
|---|---|---|---|---|---|
| Symbol/ | | | | SEQ ID | |
| GenBank | | | | NO | SEQ ID |
| accession | | Molecular | Name of | Nucleic | NO |
| numbers | Biological Process | Function | Gene | Acid | Protein |
| COP1 | Proteolysis and | Protein binding, | Caspase-1 | 28 | 70 |
| NM_052889 | peptidolysis | caspase activity | dominant- | | |
| NP_443121 | Regulation of | | negative | | |
| | apoptosis | | inhibitor | | |
| | | | pseudo-ICE | | |
| SEC24D | Intracellular protein | Protein binding | SEC24 | 29 | 71 |
| NM_014822 | transport | | related gene | | |
| NP_055637 | ER to Golgi transport | | family, | | |
| | | | member D | | |
| ZFHX1B | Negative regulation of | Transcription factor | Zinc finger | 30 | 72 |
| NM_014795 | transcription, DNA- | activity, zinc ion | homeobox 1b | | |
| NP_055610 | dependent | binding, | | | |
| | neurogenesis | transcriptional | | | |
| | | repressor activity, | | | |
| | | Smad binding, | | | |
| | | phosphatase | | | |
| | | regulator activity | | | |
| FLI1 | Regulation of | Transcription factor | Friend | 31 | 73 |
| NM_002017 | transcription, DNA | activity | leukemia | | |
| NP_002008 | dependent, | | virus | | |
| | Homeostasis, | | integration 1 | | |
| | organogenesis | | | | |
| PLA2G7 | Inflammatory | Phospholipid | Phospho | 32 | 74 |
| NM_005084 | response | binding, hydrolase | lipase A2, | | |
| NP_005075 | Lipid catabolism | activity, | group VII | | |
| | | 1-alkyl-2- | | | |
| | | acetylglycerophosphocholine | | | |
| | | esterase | | | |
| | | activity | | | |
| CXCL2 | Chemotaxis, | Chemokine activity | Chemokine | 33 | 75 |
| NM_002089 | inflammatory | | (C—X—C | | |
| NP_002080 | response, G-protein | | motif) ligand 2 | | |
| | coupled receptor | | | | |
| | protein, signaling | | | | |
| | pathway | | | | |
| CCR1 | Chemotaxis, | Rhodopsin-like | Chemokine | 34 | 76 |
| NM_001295 | inflammatory | receptor activity, C- | (C-C motif) | | |
| NP_001286 | response, cell | C chemokine | receptor 1 | | |
| | adhesion, G-protein | receptor activity | | | |
| | signaling, coupled to | | | | |
| | cyclic nucleotide | | | | |
| | second | | | | |
| | messenger, positive | | | | |
| | regulation of calcium, | | | | |
| | cell-cell signaling | | | | |
| FUCA1 | Carbohydrate | Alpha-L-fucosidase | Fucosidase, | 35 | 77 |
| NM_000147 | metabolism | activity, hydrolase | alpha-L-1 | | |
| NP_000138 | Glycosaminoglycan | activity, acting on | tissue | | |
| | catabolism | glycosyl | | | |
| | | Bonds | | | |
| CSPG2 | Development, cell | Calcium ion | Chondroitin | 36 | 78 |
| NM_004385 | recognition | binding, sugar | sulfate | | |
| NP_004376 | | binding | proteoglycan 2 | | |
| | | Hyaluronic acid | (versican) | | |
| | | binding | | | |
| MNDA | Regulation of | DNA binding | Myeloid cell | 37 | 79 |
| NM_002432 | transcription, DNA | | nuclear | | |
| NP_002423 | dependent | | differentiation | | |
| | Cellular defense | | antigen | | |
| | response | | | | |
| PAX8 | GTP/UTP/CTP | Transcription factor | Paired box | 38 | 80 |
| NM_003466 | biosynthesis, | activity, ATP | gene 8 | | |
| NP_003457 | regulation of | binding, nucleoside- | | | |
| | transcription, DNA | diphosphate kinase | | | |
| | dependent, | activity, thyroid | | | |
| | morphogenesis, cell | stimulating | | | |
| | differentiation | hormone receptor | | | |
| | | activity | | | |
| THBS1 | Cell motility, cell | Signal transducer | Thrombo | 39 | 81 |
| NM_003246 | adhesion, | activity, calcium ion | spondin 1 | | |
| NP_003237 | development, | binding, protein | | | |
| | neurogenesis, blood | binding, heparin | | | |
| | coagulation | binding, structural | | | |
| | | molecule activity | | | |
| CX3CR1 | Chemotaxis, cellular | Rhodopsin-like | Chemokine | 40 | 82 |
| NM_001337 | defense response, cell | receptor activity, | (C—X3—C | | |
| NP_001328 | adhesion, G-protein | chemokine receptor | motif) | | |
| | coupled receptor | activity, | receptor 1 | | |
| | protein signaling | coreceptor activity | | | |
| | pathway | | | | |
| DHRS9 | Metabolism, epithelial | Alcohol/retinol | Dehydrogenase | 41 | 83 |
| NM_005771 | cell differentiation, | dehydrogenase | reductase | | |
| NP_005762 | progesterone | activity | (SDR | | |
| | metabolism, retinol | Oxidoreductase | family), | | |
| | metabolism, 9-cis- | activity, racemase | member 9 | | |
| | retinoic acid | and epimerase | | | |
| | biosynthesis | activity, NAD+ | | | |
| | | activity | | | |
| SERPINB2 | Anti-apoptosis | Serine-type | Serpin | 42 | 84 |
| NM_002575 | | endopeptidase | peptidase | | |
| NP_002566 | | inhibitor activity | inhibitor | | |
| | | Plasminogen | clade B | | |
| | | activator activity | (ovalbumin), | | |
| | | | member 2 | | |

PBMCs were treated with 100 pM TGFβ with or without SD-093 for 0, 2 and 19.5 hours. The effects of TGFβ on PBMC gene expression profiles were determined using Affymetrix Gene Chips® as described in “Materials and Methods”. In order to determine the genes that were sensitive to the inhibitor, we identified those genes that were significantly down-regulated with TGFβ and significantly up-regulated when treated with TGFβ+SD-093. 15 of the known TGFβ-repressed genes were common at both the 2 hour and 19.5 hour time points.

What is claimed is:

1. A method for determining the optimal biologic dose of a Transforming Growth Factor-beta (TGFβ) receptor kinase inhibitor for administration to a patient in need of such therapy, comprising the steps of:
a) obtaining a tissue or cell sample from said patient prior to initiation of therapy to establish baseline levels of TGFβ receptor kinase activity;
b) processing said sample to enable release of phosphorylated Smad2 and -3 (pSmad2/3) from the cells within the sample;
c) contacting said processed sample with a solid substrate to allow binding of the released pSmad2/3 to said substrate;
d) measuring the amount of pSmad2/3 in said sample by detecting said pSmad2/3 with an antibody specific for pSmad2/3;
e) obtaining a tissue or cell sample from the patient after treatment with a TGFβ receptor kinase inhibitor given at various doses; and repeating steps b) through d);
f) comparing the levels of pSmad2/3 in the tissue sample obtained in step e) to the level of pSmad2/3 in the sample obtained in step a);
wherein a decrease in the levels of pSmad2/3 compared to baseline levels is indicative of achieving the optimal dose of the TGFβ receptor kinase inhibitor.

2. The method of claim 1, wherein said tissue sample is selected from the group consisting of tumor tissue, skin, whole blood, peripheral blood mononuclear cells, gingiva, colon, endometrium and any other accessible tissue or cell of the human body.

3. The method of claim 1, wherein said detecting is accomplished by an immunoassay.

4. The method of claim 3, wherein said immunoassay is an enzyme linked immunoassay, a radioimmunoassay, or a Western blot assay.

5. The method according to any of claims 1-4, wherein said antibody specific for pSmad2/3 is selected from the group consisting of a polyclonal antibody, a monoclonal antibody, a single chain antibody, a human or humanized antibody, and Fab fragments thereof.

6. The method of any of claims 1-5, wherein said patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

7. A method for monitoring the effectiveness of therapy with a TGFβ receptor kinase inhibitor in patients receiving such therapy, comprising the steps of:
a) obtaining a tissue sample from said patient prior to initiation of therapy to establish baseline levels of TGFβ receptor kinase activity;
b) processing said sample to enable release of pSmad2/3 from the cells within the sample;
c) contacting said processed sample with a solid substrate to allow binding of the released pSmad2/3 to said substrate;
d) measuring the amount of pSmad2/3 in said sample by detecting said pSmad2/3 with an antibody specific for pSmad2/3;
e) obtaining a tissue sample from the patient after treatment with a TGFβ receptor kinase inhibitor given at various doses; and repeating steps b) through d);
f) comparing the levels of pSmad2/3 in the tissue sample obtained in step e) to the level of pSmad2/3 in the sample obtained in step a);
wherein a decrease in the levels of pSmad2/3 compared to baseline levels is reflective of the effectiveness of therapy with a TGFβ receptor kinase inhibitor.

8. The method of claim 7, wherein said tissue sample is selected from the group consisting of tumor tissue, skin, whole blood, peripheral blood mononuclear cells, gingiva, colon, endometrium or any other accessible tissue or cell of the human body.

9. The method of claim 7, wherein said detecting is accomplished by an immunoassay.

10. The method of claim 9, wherein said immunoassay is an enzyme linked immunoassay, a radioimmunoassay, or a Western blot assay.

11. The method of any of claims 7-10, wherein said antibody specific for pSmad2/3 is selected from the group consisting of a polyclonal antibody, a monoclonal antibody, a single chain antibody, a human or humanized antibody, and Fab fragments thereof.

12. The method of any of claims 7-11, wherein said patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

13. A method for determining the optimal biologic dose of a TGFβ receptor kinase inhibitor for administration to a patient in need of such therapy, comprising the steps of:
a. obtaining a plasma sample from said patient prior to initiation of therapy to establish baseline levels of TGFβ receptor kinase activity;
b. contacting said sample with TGFβ-responsive test cells in vitro; wherein said cells are pretreated with TGFβ at a dose sufficient to activate TGFβ receptor kinase activity;
c. processing said cells to enable release of pSmad2/3 from the cells;
d. contacting the extract from said processed cells with a solid substrate to allow binding of the released pSmad2/3 to said substrate;
e. measuring the amount of pSmad2/3 in said extract by detecting said pSmad2/3 with an antibody specific for pSmad2/3;
f. obtaining a plasma sample from the patient after treatment with a TGFβ receptor kinase inhibitor given at various doses; and repeating steps b) through e);
g. comparing the levels of pSmad2/3 from test cells incubated with plasma samples from step f) to the level of pSmad2/3 from test cells incubated with plasma samples from step a);
wherein a decrease in the levels of pSmad2/3 compared to baseline levels is indicative of achieving the optimal biologic dose of the TGFβ receptor kinase inhibitor.

14. The method of claim 13, wherein said TGFβ responsive test cells are selected from the group consisting of Sweig cells, BALB/MK cells, HKc/HPV16 cells, Mink lung cells, HaCaT cells, MDA-MB-231 cells, and MDA-MB-435 cells and any other human or rodent, epithelial or lymphoid cell line in which TGFβ reproducibly induces phosphorylation of Smad2/3 in a dose-dependent manner.

15. The method of claim 13, wherein said detecting is accomplished by an immunoassay.

16. The method of claim 15, wherein said immunoassay is an enzyme linked immunoassay, a radioimmunoassay, or a Western blot assay.

17. The method of any of claims 13-16, wherein said antibody specific for pSmad2/3 is selected from the group consisting of a polyclonal antibody, a monoclonal antibody, a single chain antibody, a human or humanized antibody, and Fab fragments thereof.

18. The method of any of claims 13-17, wherein said patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

19. A method for monitoring the effectiveness of therapy with a TGFβ receptor kinase inhibitor in patients receiving such therapy, comprising the steps of:
a) obtaining a plasma sample from said patient prior to initiation of therapy to establish baseline levels of TGFβ receptor kinase activity;
b) contacting said sample with TGFβ responsive test cells in vitro; wherein said cells are pretreated with TGFβ at a dose sufficient to activate TGFβ receptor kinase activity;
c) processing said cells to enable release of pSmad2/3 from the cells;
d) contacting the extract from said processed cells with a solid substrate to allow binding of the released pSmad2/3 to said substrate;
e) measuring the amount of pSmad2/3 in said extract by detecting said pSmad2/3 with an antibody specific for pSmad2/3;
f) obtaining a plasma sample from the patient after treatment with a TβR-1 receptor kinase inhibitor given at various doses; and repeating steps b) through e);
g) comparing the levels of pSmad2/3 from test cells incubated with plasma samples from step f) to the level of pSmad2/3 from test cells incubated with plasma samples from step a);
wherein a decrease in the levels of pSmad2/3 compared to baseline levels is reflective of the effectiveness of therapy with a TGFβ receptor kinase inhibitor.

20. The method of claim 19, wherein said TGFβ responsive test cells are selected from the group consisting of Sweig cells, BALB/MK cells, HKc/HPV16 cells, Mink lung cells, HaCaT cells, MDA-MB-231 cells, and MDA-MB-435 cells and any other human or rodent, epithelial or lymphoid cell line in which TGFβ reproducibly induces phosphorylation of Smad2/3 in a dose-dependent manner.

21. The method of claim 19, wherein said detecting is accomplished by an immunoassay.

22. The method of claim 21, wherein said immunoassay is an enzyme linked immunoassay, a radioimmunoassay, or a Western blot assay.

23. The method of any of claims 19-22, wherein said antibody specific for pSmad2/3 is selected from the group consisting of a polyclonal antibody, a monoclonal antibody, a single chain antibody, a human or humanized antibody, and Fab fragments thereof.

24. The method of any of claims 19-23, wherein said patients are selected from the group consisting of cancer patients, patients having pulmonary fibrosis, patients having liver cirrhosis, patients having chronic glomerulonephritis, patients receiving radiation therapy, patients having arterial restenosis and patients having keloids.

25. A method of identifying by high throughput screening a therapeutic agent that inhibits TGFβ receptor kinase activity, comprising the steps of:
a) incubating a culture of TGFβ responsive cells with increasing concentrations of a test agent, or with control culture medium, for a time sufficient to allow binding of TGFβ to its receptors and to activate the receptor kinases;
b) fixing and permeabilizing the cells in order to allow for antibody binding to the phosphorylated Smad2/3 molecules;
c) incubating the cells with an antibody specific for phosphorylated Smad2/3 (pSmad2/3) for a time sufficient to allow binding of the antibody to pSmad2/3;
d) detecting and quantitating the amount of pSmad2/3 antibody bound by incubating with a labeled second antibody having specificity for the pSmad2/3 antibody;
e) comparing the amount of labeled second antibody bound to TGFβ responsive cells without test compound to the amount of labeled second antibody bound to TGFβ responsive cells with test compound; and
wherein the amount of labeled antibody bound correlates inversely with the potential of the test compound for inhibiting TGFβ receptor kinase activity.

26. A diagnostic test kit for determining the optimal biologic dose of a TGFβ receptor kinase inhibitor to be administered to a patient in need of such therapy, or for monitoring the effectiveness of therapy with a TGFβ receptor kinase inhibitor in patients receiving such therapy, or for predicting whether a subject is a candidate for therapy with a TGFβ receptor kinase inhibitor comprising,
a) a predetermined amount of an antibody specific for pSmad2/3;
b) a predetermined amount of a specific binding partner to said antibody;
c) buffers and other reagents necessary for monitoring detection of antibody bound to pSmad2/3; and
d) directions for use of said kit;
wherein either said antibody or said specific binding partner are detectably labeled.

27. A method of treating patients suffering from a TGFβ-dependent disease or condition comprising treatment with a pharmaceutical composition comprising an anti-pSmad2/3 antibody, or fragments, analogs or mimics thereof that affect downstream signaling events, and a pharmaceutically acceptable carrier.

28. A method for determining the effect of a TGF-β receptor kinase inhibitor on modulation of TGF-β signaling, comprising the steps of:
a. providing a cell that expresses one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPIN2 and SERPINE1.
b. determining the baseline level of expression of one or more of the genes from step a) in the cell;
c. treating the cell with TGF-β alone or with TGF-β plus a TGF-β receptor kinase inhibitor;
d. isolating RNA from the cell of step c); and
e. analyzing the RNA from step d) to determine whether any one or more genes from step a) were up-regulated or down-regulated by treating the cell with TGF-β plus a TGF-β receptor kinase inhibitor, as compared to a cell treated with TGF-β alone;
wherein a change in the level of expression of one or more of the genes from step a) in the TGF-β treated cell compared to the cell treated with TGF-β plus a receptor kinase inhibitor is indicative that the TGF-β receptor kinase inhibitor modulates TGF-β signaling.

29. The method of claim 28, wherein the cell is a tumor cell, a peripheral blood mononuclear cell (PBMC) a skin cell, a bone marrow cell, a cell obtained from a gingival biopsy, a cell obtained from the colon, a cell obtained from the endometrium and any other accessible tissue or cell of the human body.

30. The method of claim 29, wherein the PBMC is a lymphocyte or a monocyte.

31. The method of claim 30, wherein the lymphocyte is a T cell, or a B cell.

32. The method of claim 28, wherein the one or more genes that are down-regulated in the presence of a TGF-β receptor kinase inhibitor are selected from the group consisting of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1.

33. The method of claim 28, wherein the one or more genes that are up-regulated in the presence of a TGF-β receptor kinase inhibitor are selected from the group consisting of COP1, SEC24D, ZFHX1B, FLI1, PLA2G7, CXCL2, CCR1, FUCA1, CSPG2, MNDA, PAX8, THBS1, CX3CR1, DHRS9 and SERPINB2.

34. The method of claim 28, wherein the RNA is analyzed using a method selected from the group consisting of gene expression microarray analysis or by polymerase chain reaction (PCR.)

35. The method of claim 34, wherein the PCR is quantitative real-time PCR.

36. The method of claim 28, wherein the cell is treated with TGF-β alone or TGF-β plus a receptor kinase inhibitor for a time period ranging from about 0 to 24 hours.

37. The method of claim 28 wherein the concentration of TGF-β ranges from about 0 pM to about 400 pM.

38. The method of claim 28, wherein the inhibitor of TGF-β receptor kinase is added at a concentration ranging from about 0 uM to about 2 uM.

39. The method of claim 28, wherein the effect of a TGF-β receptor kinase inhibitor on TGF-β signaling and/or changes in gene expression resulting from exposure of the cell to TGF-β is both time and dose dependent.

40. The method of claim 28, wherein the changes in gene expression in the cell are dependent on the activity of a TGF-β receptor kinase.

41. A method for determining a biologically effective dose of a TGF-β receptor kinase inhibitor, or for determining the effectiveness of therapy with a TGF-β receptor kinase inhibitor in patients receiving such therapy, or for determining whether a TGF-β receptor kinase inhibitor would be effective in treating a patient in need of such therapy, comprising the steps of:
a) obtaining a tissue or cell sample from a patient prior to initiation of therapy with a TGF-β receptor kinase inhibitor to establish a baseline level of one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2, and SERPINE1;
b) obtaining a tissue or cell sample from a patient during the course of therapy with a TGF-β receptor kinase inhibitor and after therapy has ended to establish a change in the level of one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1;
c) treating the cell with TGF-β or with a vehicle control;
d) isolating RNA from the cell of step c); and
e) analyzing the RNA from step d) to determine whether any one or more genes from step a) were up-regulated or down-regulated following treatment with a TGF-β receptor kinase inhibitor;
wherein a change in the level of expression of one or more of the genes from step a) to step b) in a tissue or cell sample indicates that the one or more genes may be used as a biomarker for determining the biologically effective dose of a TGF-β receptor kinase inhibitor, or for determining the effectiveness of therapy with a TGF-β receptor kinase inhibitor in patients receiving such therapy, or for determining whether a TGF-β receptor kinase inhibitor would be effective in treating a patient in need of such therapy.

42. The method of claim 41, wherein the tissue or cell sample is a tumor cell, a peripheral blood mononuclear cell (PBMC) a skin cell, a bone marrow cell, a cell obtained from a gingival biopsy, a cell obtained from the colon, a cell obtained from the endometrium and any other accessible tissue or cell of the human body.

43. The method of claim 42, wherein the PBMC is a lymphocyte or a monocyte.

44. The method of claim 43, wherein the lymphocyte is a T cell, or a B cell.

45. The method of claim 41 wherein the one or more genes that are down-regulated in the presence of a TGF-β receptor kinase inhibitor are selected from the group consisting of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1.

46. The method of claim 41, wherein the one or more genes that are up-regulated in the presence of a TGF-β receptor kinase inhibitor are selected from the group consisting of COP1, SEC24D, ZFHX1B, FLI1, PLA2G7, CXCL2, CCR1, FUCA1, CSPG2, MNDA, PAX8, THBS1, CX3CR1, DHRS9 and SERPINB2.

47. The method of claim 41, wherein the RNA is analyzed using a method selected from the group consisting of gene expression microarray analysis or by polymerase chain reaction (PCR.)

48. The method of claim 47, wherein the PCR is quantitative real-time PCR.

49. The method of claim 41, wherein the changes in gene expression in the cell are dependent on the activity of a TGF-β receptor kinase.

50. A method for determining the ability of a drug candidate to inhibit TGF-β signaling, the method comprising:
a) providing a cell that expresses one or more genes selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2 and SERPINE1;
b) adding to the cell either TGF-β alone, or TGF-β plus a drug candidate;
c) processing the cell to release nucleic acid and cytoplasmic proteins from the cell;
d) determining the expression level of one or more of the genes from step a);
e) comparing the expression level of one or more of the genes in the cell treated with TGF-β alone with the expression level of one or more of the genes in a cell treated with TGF-β plus the drug candidate to determine:
(i) whether expression of KLF10, S100A10, TRIM36, JUN, RAI17, DUSP1, ANKH, UPP1, VEGF, CXCR4, SLC16A3, FST, OSM, SERPINF1, CDK 5R1, FCGR3A, FCGR3B, CLIC3, SMAD7, SLC7A5, ITGAV, HBEGF GPR84, B3GNT5, TMEPAI, OLR1 and SERPINE1 is decreased in the cell treated with TGF-β plus a drug candidate relative to a cell not treated with the drug candidate, or
(ii) whether expression of COP1, SEC24D, ZFHX1B, FLI1, PLA2G7, CXCL2, CCR1, FUCA1, CSPG2, MNDA, PAX8, THBS1, CX3CR1, DHRS9 and SERPINB2 is increased in the cell treated with TGF-β plus a drug candidate relative to a cell not treated with the drug candidate; and
wherein the drug candidate is identified as a potential inhibitor of TGF-β signaling if the expression level of a gene listed in (i) is decreased and/or the expression level of a gene listed in (ii) is increased.

51. The method according to claim 50, further comprising:
f) contacting the processed cell with a solid substrate to allow binding of released Smad2/3 to the substrate;
g) measuring the amount of pSmad2/3 in the cell sample by detecting the pSmad2/3 with an antibody specific for pSmad2/3;
h) comparing the levels of pSmad2/3 in the cell sample obtained from the cell treated with TGF-β alone or from the cell treated with TGF-β plus the drug candidate;
wherein a decrease in the level of pSmad2/3 in the presence of the drug candidate compared to the level of pSmad2/3 in the absence of the drug candidate is indicative that the drug candidate is an inhibitor of TGF-β signaling.

52. The method according to claim 50, wherein the expression level of a plurality of genes is determined and compared.

53. The method according to claim 52, wherein the expression level of at least five genes is determined and compared.

54. The method of claim 50, wherein the expression level is determined from the amount of transcript expressed by the one or more genes.

55. The method of claim 50, wherein the expression level is determined from the amount of protein expressed by the one or more genes.

56. The method of claim 50, wherein the expression level is determined by gene expression microarray analysis, protein expression microarray analysis, polymerase chain reaction, quantitative polymerase chain reaction, or by enzyme-linked immunosorbent assay detection of a protein product of the one or more genes.

57. A diagnostic test kit for determining the effect of a TGF-β receptor kinase inhibitor on modulation of TGF-β signaling, or for determining a biologically effective dose of a TGF-β kinase inhibitor, or for determining the effectiveness of therapy with a TGF-β receptor kinase inhibitor in patients receiving such therapy, or for identifying a TGF-β receptor kinase inhibitor that would be effective in treating a patient in need of such therapy, comprising:
a) one or more nucleic acids encoding one or more of the proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, SERPINB2; and SERPINE1;
b) reagents useful for monitoring the expression level of the one or more nucleic acids or proteins encoded by the nucleic acids of step a);
c) instructions for use of the kit.

58. The test kit of claim 57, wherein the kit comprises at least five nucleic acids encoding at least five proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, and SERPINB2.

59. The test kit of claim 57, wherein the kit comprises at least ten nucleic acids encoding at least ten proteins selected from the group consisting of KLF10, S100A10, TRIM 36, JUN, RAI17, DUSP1, ANKH, UPP1, VEGF, CXCR4, SLC16A3, FST, OSM, SERPINF1, CDK5R1, FCGR3A, FCGR3B, CLIC3, SMAD7, ITGAV, HBEGF, GPR84, B3GNT5, TMEPAI, OLR1, COP1, SEC24D, ZFHX1B, FLI1, PLA2G7, CXCL2, CCR1, FUCA1, CSPG2, MNDA, PAX8, THBS1, CX3CR1, DHRS9, and SERPINB2.

60. A kit for determining the effect of a TGF-β receptor kinase inhibitor on modulation of TGF-β signaling, or for determining a biologically effective dose of a TGF-β kinase inhibitor, or for determining the effectiveness of therapy with a TGF-β receptor kinase inhibitor in patients receiving such therapy, or for identifying a TGF-β receptor kinase inhibitor that would be effective in treating a patient in need of such therapy, comprising:
a first plurality of oligonucleotides, comprising the nucleic acid sequences of five or more SEQ ID NOs; 1-42, or the complements thereof, and a second plurality of oligonucleotides, comprising mismatch oligonucleotides corresponding to the first plurality of oligonucleotides, and wherein each oligonucleotide is attached to a solid support in a determinable location.

61. The kit of claim 60, wherein the solid support is a plurality of beads.

62. The kit of claim 60, wherein the solid support is glass.

63. An array of oligonucleotides comprising the nucleic acid sequences of SEQ ID NOs; 1 through 42 attached to a solid support in a determinable location of the array.

64. A method for diagnosing a disease or condition associated with activated TGF-β signaling in a patient, or for determining whether a patient is prone to developing such disease or condition, comprising the steps of:
(a) obtaining a biological sample from the patient;
(b) releasing nucleic acids from said biological sample;
(c) performing PCR in the presence of a set of primers specific for any one of SEQ ID NOs: 1 through 42 and labeled probes that recognize and bind to any one of SEQ ID NOS: 1 through 42 under conditions wherein the presence or level of a nucleic acid sequence that is modulated as a result of TGF-β signaling results in an amplified and labeled PCR product; and
(d) detecting the presence of a labeled PCR product, wherein the presence of a labeled PCR product indicates the presence of a nucleic acid sequence associated with TGF-β signaling; and
wherein the presence of the labeled PCR product is indicative of the presence of a disease or condition associated with TGF-β signaling.
